(19) United States
(12) Patent Application Publication
(10) Pub. No.: US 2024/0114883 A1
(43) Pub. Date: Apr. 11, 2024

(54) Genetically modified non-human having humanized gamma and delta TCR variable genes

(71) Applicant: inGenious Targeting Laboratories, Ronkonkoma, NY (US)

(72) Inventor: Wei Weng, Sayville, NY (US)

(21) Appl. No.: 18/233,507

(22) Filed: Aug. 14, 2023

Related U.S. Application Data
(60) Provisional application No. 63/397,633, filed on Aug. 12, 2022.

(51) Int. Cl. A01K 67/027; C07K 14/725

(52) U.S. Cl. CPC A01K 67/0278; A01K 67/0276; C07K 14/7051; A01K 2207/12; A01K 2207/15; A01K 2217/052; A01K 2217/072; A01K 2217/075; A01K 2217/15; A01K 2227/105; A01K 2267/01; A01K 2267/0331

(57) ABSTRACT

The invention provides a genetically modified non-human animal that comprises in its genome foreign γ and δ T cell receptor variable gene coding fragments, as well as its tissues, embryos, and cells. Also provided are constructs and methods for making said genetically modified non-human animal. Human T cell receptor (TCR) cloning and repertoire analysis are also included. Applications for using said animal in infectious diseases are also provided.

The present application claims the benefit of priority under 35 U.S.C. § 119(e) of Provisional Applications No. 63/397,633, filed on Aug. 12, 2022, the entire disclosure of which is hereby incorporated by reference.

### SEQUENCE LISTING

The instant application contains a Sequence Listing which has been submitted herewith and is hereby incorporated by reference in its entirety. Said .xml copy, created on Nov. 8, 2023 is named 140457583383, and is 44,476,257 bytes in size.

### BACKGROUND

### Field of the Invention

The present invention relates to a genetically modified non-human animal (e.g., a rodent, e.g., a mouse or a rat) that comprises in its genome human or humanized T Cell Receptor (TCR) variable region of DNA coding and/or gene loci (e.g., TCR δ. and TCR γ. variable gene loci). The invention further relates to a non-human animal engineered to express a human or humanized TCR δ or TCR γ on the surface of γ-δ T cells. The invention further relates to a non-human animal engineered to co-express both human or humanized TCR δ or TCR γ on the surface of γδ T cells. Methods for cloning and analysis of human or humanized TCR repertoire and human or humanized TCR expansion upon infection are provided. Methods for said animal that express human or humanized γδ T cell receptors or co-receptors for further developing therapies for human diseases are also provided. The invention further relates to unrearranged human TCR variable region gene segments (e.g., human V, D, and/or J segments) at endogenous non-human TCR γ 6 gene loci.

### Discussion of the Related Art

Like conventional αβ T cells and B cells, γδ T cells use V(D)J gene rearrangement to generate a set of highly diverse receptors to recognize antigens. This diversity is generated mainly in the complementary-determining region 3 (CDR3) of the T-cell antigen receptor (TCR). Unlike conventional αβ T cells, γδ T cells can be activated and expanded by non-peptide antigens. In contrast to conventional αβ T cells, γδ T cells are not dependent on classical MEC molecules presenting peptides. The efficacy of γδ T cells does not rely on recognizing classical HLA molecules.

These cells possess a distinctive quality in detecting antigen disruptions during various infections or cancers, a trait potentially shared universally among individuals and even across different animal species. As γδ T cells rapidly recognized the antigen disturbance, γδ T cells are considered to partially belong to the first line of immune defense (innate immune system), but they also can create immune memory (adaptive immune system) (Chien, Annu. Rev. Immunol. 32. 121-155 (2014)). Human γδ T cells are shown to be able to kill infected cells and cancers.

However, no non-human animal with the ability to generate diverse human γδ CDR3 has ever been developed. Thus, there is a need in the art for non-human animals (e.g., rodents, e.g., rats or mice) that comprise unrearranged human γδ T cell variable region gene segments capable of rearranging to form genes that encode human γδ T cell receptor variable domains, including domains that are cognate with one another, and including domains that specifically bind an antigen of interest and subsequently initiate the down-stream chemical and/or biological activity.

### SUMMARY

An embodiment of the invention relates to non-human animals, e.g., rodents, comprising unrearranged human or humanized γδ TCR coding DNA fragments and variable gene loci. The variable region further comprises human V, D, and J fragments. Methods of generating said animal are also provided. The genome of immune cells of said animal undergoes V(D)J recombination upon the activity of V(D)J recombinase enzyme (e.g. RAG1 and/or RAG2). The V(D)J recombination comprises processes that generate a large diversity of γ/δ TCR. The diversity increases the variability of the resulting functional genes.

Provided herein is a genetically modified non-human animal (e.g., a rodent, e.g., a mouse or a rat) that comprises in its genome human or humanized T Cell Receptor (TCR) variable region of DNA coding and/or gene loci (e.g., TCR δ and/or TCR γ variable gene loci).

In various embodiments, said animal comprises a nucleotide sequence encoding human TCR γ and/or δ variable region. In one embodiment, the human variable region fuses with non-human γ and/or δ TCR coding region to form a chimeric human/non-human TCR. In one embodiment, the nucleotide sequence of the non-human animal is replaced by the human sequence at an endogenous locus of γ and/or δ T cell receptor.

In one embodiment, the human V(D)J encodes the TCR CDR3 domain as part of a polypeptide which is linked to a non-human portion comprising transmembrane and cytoplasmic domains. In one embodiment, the portion of the polypeptide containing the CDR3 domain on the surface of γδ T cells served as the extra-cellular domain. In another aspect, the chimeric TCR comprises a human variable region and a non-human constant region on the surface of a γδ T cell.

In one aspect, a genetically modified non-human animal (e.g., a rodent, e.g, a mouse or a rat) is provided which comprises in its genome an unrearranged TCR γ and/or unrearranged TCR δ nucleotide sequence. The unrearranged TCR γ comprises at least one human Vγ variable segment and at least one human Jγ segment. The unrearranged TCR comprises at least one human Vδ segment, at least one human Dδ, and at least one human Jδ segment.

In one embodiment, a non-human animal (e.g., a rodent, e.g., a mouse or a rat) that comprises in its genome at least one human Vγ variable segment and at least one human Jγ segment, operably linked to a non-human (e.g., a rodent, e.g., a mouse or a rat) TCR γ constant gene sequence. The genome of said non-human animal further comprises at least one human Vδ segment, at least one human Dδ, and at least one human Jδ segment, operably linked to a non-human (e.g., a rodent, e.g., a mouse or a rat) TCR δ constant gene sequence.

In one embodiment, the human unrearranged TCR γ variable gene locus replaces one or more non-human TCR γ variable genes at an endogenous TCR γ variable gene locus.

In one embodiment, the human unrearranged TCR δ variable gene locus replaces one or more non-human TCR δ variable genes at an endogenous TCR δ variable gene locus.

In one aspect, the human variable region comprising Vγ and Jγ segments is fully or partially capable of rearranging to form a rearranged Vγ and Jγ sequence. In another aspect, the human variable region comprising Vδ, Dδ, and Jδ segments is fully or partially capable of rearranging to form a rearranged V(D)J δ sequence.

In some embodiments, a functional CDR3 domain is generated as a result of rearranging the human Vγ/Jγ sequence upon the activation of the recombinase enzyme. In some embodiments, a functional CDR3 domain is generated as a result of rearranging the human Vδ/Dδ/Jδ sequence upon the activation of the recombinase enzyme.

In some aspects, T cells of the non-human animal undergo T cell development in the thymus to human γδ positive T cells.

In various embodiments, the non-human animal generates a population of γδ T cells in the periphery, spleen, lung, liver, kidney, intestine, and skin.

In one aspect, the invention is related to the expression of human TCRγ and/or stretch of at least 10 amino acids in the human TCRγ of a non-human animal.

In some embodiments, the human TCR comprises the amino acid sequence which is one of the following: (a) human variable Vγ9, 10, 11 comprising SEQ ID NO (amino acid): 62, 63, 64, respectively; (b) a conservative amino acid sequence that is at least 90% identical to SEQ ID NO (amino acid): 62 or 63; and (c) a conservative amino acid sequence that is at least 70%, 71%, 72%, 73%, 74%, 75%, 80%, 85%, 90%, 96%, 97%, 98%, or 99% identical to SEQ ID NO: 62, 63, or 64.

In one aspect, the invention is related to the expression of human TCRδ and/or stretch of at least 10 amino acids in the human TCRδ of a non-human animal. In some embodiments, the human TCR comprises the amino acid sequence which is one of the following: (a) human variable V(δ) 1, 2, 3, 4, 5, 6, 7, or 8 comprising SEQ ID Nos: 65, 66, 67, 68, 69, 70 71, or 72, respectively; (b) a conservative amino acid sequence that is at least 90% identical to SEQ ID (amino acid) NO: 62 or 63; and (c) a conservative amino acid sequence that is at least 70%, 71%, 72%, 73%, 74%, 75%, 80%, 85%, 90%, 96%, 97%, 98%, or 99% identical to SEQ ID (amino acid) NO: 65, 66, 67, 68, 69, 70, 71, or 72.

In one aspect, the disclosure is related to a nucleic acid encoding an amino acid sequence of full or part of the endogenous non-human animal Vγ constant (C) region at the endogenous locus. In some embodiments, the Vγ C region comes from other species, which may be functional when linked to human VJγ. The human Vγ, Jγ, and functional C region can be introduced into the endogenous locus or randomly integrated into the genome of a non-human animal.

In some aspects, the constant region of the TCRδ comprises one or more conserved amino acid sequences: PSVF (SEQ ID No. 50789), MKNG (SEQ ID No. 50790), GTNVACL (SEQ ID No. 50791), SAVKLGQ (SEQ ID No. 50792), SVTCSV (SEQ ID No. 50793), KVNMMSL (SEQ ID No. 50794), VLGLR (SEQ ID No. 50795), LFAK (SEQ ID No. 50796), and/or NFLL (SEQ ID No. 50797).

In some aspects, the constant region of the TCRγ comprises one or more conserved amino acid sequences: PKPT (SEQ ID No. 50798), LCLL (SEQ ID No. 50799), KTKD (SEQ ID No. 50800), MKFSWLT (SEQ ID No. 50801), TSAYY (SEQ ID No. 50802), and/or LLLLLKS (SEQ ID No. 50803).

In one embodiment, the unrearranged TCRγ gene locus in the non-human animal described herein comprises 3 human Vγ segments and 3 human Jγ segments.

In one embodiment, the non-human animal described herein further comprises 8 human Vδ segments, 3 human Dδ segments, and 4 human Jγ segments.

In another embodiment, the unrearranged TCRγ gene locus in the non-human animal can form a functional repertoire of human γ CDR3 domain upon the activity of Rag1 and/or Rag2 or a combination of both or other related enzymes.

In another embodiment, the unrearranged TCRδ gene locus in the non-human animal can form a functional repertoire of human γ CDR3 domain upon the activity of Rag1 and/or Rag2 or a combination of both or other related enzymes.

In an additional embodiment, the non-human animal described herein (e.g., a rodent) further comprises nucleotide sequences of human TCRδ variable segments at a humanized TCRδ locus. In one embodiment, the non-human animal (e.g., rodent) further comprises at least one human Vδ, Dδ, and/or Jδ segment.

In one embodiment, the human TCRγ and mouse TCRγ can form a receptor complex and express on mouse T cells. In one embodiment, the human δ T cell receptor can form a receptor complex with a mouse γ T cell receptor to express on mouse γ/δ T cells.

In some embodiments, the invention related to a method of replacement of the endogenous non-human TCRγ with human TCRγ variable gene locus described herein was made in a single ES (embryonic stem) cell, and the single ES cell is introduced into a non-human (e.g., a rodent, e.g., a mouse or rat) embryo to make a genetically modified non-human animal (i.e., the first non-human animal, e.g., the human Vγ rodent); and the replacement of the endogenous non-human TCRδ with human Vδ variable gene locus described herein was made in a single ES cell, and the single ES cell was introduced into a non-human (e.g., a rodent, e.g., a mouse or rat) embryo to make a genetically modified non-human animal (the Vδ rodent). In one embodiment, the Vγ rodent and the Vδ rodent are bred to form a progeny, wherein the progeny comprises inits germline a humanized TCRγ variable locus and a humanized TCRδ variable locus.

In the method, the non-human animal is a rodent, e.g., a mouse, a genetically modified mouse.

This invention also provided herein cells, e.g., isolated T cells (e.g., γ/δ T cells, helper T cells, memory T cells, etc.), derived from non-human animals (e.g., rodents, e.g., mice or rats) described herein. Tissues and embryos derived from the non-human animals described herein are also provided.

In one aspect, a method for making a human TCR variable domain, includes: genetically modifying a rodent as described herein to comprise a humanized TCR γ locus and/or a humanized TCR δ locus: maintaining the rodent under conditions sufficient to form a T cell, wherein the T cell expresses a human TCR γ and/or a human TCR δ variable domain.

In some aspects, different non-human promoters for human Vδ are also provided, comprising promoters from γ/δ T cell-rich animals (e. g. bovine, rabbit). In other aspect, a non-human promoter for a human Vγ is also provided, comprising a promoter from a rodent (e. g. rat, or mouse). In one aspect, the non-human animal comprises promoters direct from human origin with un-rearranged sequences. In some other aspects, other human Vδ is inserted adjacent to the un-rearranged Vδ sequence.

In some aspects, methods of amplification of DNA fragment coding human TCRγ and TCRδ CDR3 after reverse-transcripted from mRNA are provided. Methods of sequencing PCR products containing the CDR3s by next-generation sequencing are also provided. Methods of Human γ/δ TCR repertoire analysis are further provided.

In some aspects, sequences of TCR(γ/δ) CDR3 from human Vδ1, Vδ2, Vδ3, Vδ4, Vδ6 are provided. In some aspects, sequences of TCRγ CDR3s from human Vγ9 and Vγ10 are provided. In further aspects, the invention also disclosed human γ and δ TCR, and their CDR3s are obtained from various tissues in non-human animals, comprising blood, intestine, thymus, liver, spleen, heart, skin, and lung.

In some aspects, in the non-human animal with human or humanized γ/δ T cell, the peptide sequence of CDR1 from the human Vδ1 comprises a conserved amino acid sequence comprising TSWWSYY (SEQ ID No. 50804), or at least one S, one W, one Y, or one T inside the CDR1 sequence. Mother aspect, the CDR2 sequence of the non-human animal Vδ1 comprises QGS, or at least one Q, one G, or one S. In a further aspect, the peptide sequence of CDR3 from the human Vδ1 comprises a conserved starting and ending sequence. The preferred amino acids for the first three amino acids of the CDR3 of the Vδ1 are ALG. Further preferred amino acids for the first four amino acids are ALGE (SEQ ID No. 5398), where position E can be any of G, A, D, V, E, or R. For the ending sequences, two amino acids out of the last five amino acids of the CDR3 of the Vδ1 are DK, LI, KL, TD, DT, PI, TA, TR, QL, or WD. In some aspects, the preferred amino acids for the last three amino acids of the CDR3 of the Vγ1 are KLI.

In one aspect, in the non-human animal with human or humanized γ/δ T cell, the peptide sequence of CDR1 from the human Vδ2 comprises a conserved amino acid sequence comprising GEAIGNYY (SEQ ID No. 50805), or at least one G, one E, one A, one I, one N, one Y inside the CDR1 sequence. In other aspect, the CDR2 sequence of the non-human animal Vδ2 comprises EKD, or at least one E, one K, or one D. In some aspects, for the starting sequences, amino acids at first three positions of the CDR3 of Vδ2 are ACD, ACG, ACE, ARD, or ASD. In some aspects, the first two amino acids of the CDR3 of the Vδ2 are AW, PV, or DC. Further preferred amino acids for the first four amino acids are ACDS (SEQ ID No. 51034), where the fourth position can be any of T, K, I, P, Y, C, L, M, or R. For the ending sequences, two amino acids out of the last five amino acids of the CDR3 of the Vδ2 are DK, LI, KL, TD, DT, PI, TA, TR, QL, SS, HI, TH, or PD. In some aspects, the preferred amino acids for the last three amino acids of the CDR3 of the Vδ2 are KLI.

CDRs of Human Vδ3

In one aspect, in the non-human animal with human or humanized γ/δ T cell, the peptide sequence of CDR1 from the human Vδ3 comprises a conserved amino acid sequence comprising TVYSNPD (SEQ ID No. 50806), or at least one T, one V, one Y, one S, one N, one P, or one D inside the CDR1 sequence. In other aspect, the CDR2 sequence of the non-human animal Vδ3 comprises GDNSR (SEQ ID No. 51035), or at least one G, one D, one N, one S, or one R. The peptide sequence of CDR3 from the human Vδ3 comprises a conserved starting sequence. The first two starting amino acid sequences of the CDR3 of Vδ3 are AF. In some aspects, the preferred amino acids for the first three amino acids of the CDR3 of the Vδ3 are AL, AS, AC, AY, AI, or AV. Further preferred amino acids for the first two amino acids are PF, TF, LF, GF, or GC.

CDRs of Human Vδ4

In one aspect, in the non-human animal with human or humanized γ/δ T cell, the peptide sequence of CDR1 from the human Vδ4 comprises a conserved amino acid sequence comprising TSDPSYG (SEQ ID No. 50807), or at least one S, one P, one G, one D, one Y, or one T inside the CDR1 sequence. In other aspect, the CDR2 sequence of the non-human animal Vδ4 comprises QGSYDQQN (SEQ ID No. 50808), or at least one Q, one G, one D, one Y, one N, or one S. In one aspect, in the non-human animal with human or humanized γ/δ T cells, the peptide sequence of CDR3 from the human Vδ4 comprises a conserved starting and ending sequence. For the starting sequences, the first three amino acids of the CDR3 of Vδ4 are AMR, ASP, AMS, AMT, AMG, AMI, AIR, AKR, ATR, EMR, VMR, PMR, and ALP. In one aspect, the preferred amino acids for the first three amino acids of the CDR3 of the Vδ4 are AMRE (SEQ ID No. 50809), where the fourth position can be any of G, C, D, V, N, A, L, V, T, or R. For the ending sequences, two amino acids out of the last five amino acids of the CDR3 of the Vδ4 are DK, LI, KL, TD, DT, PI, TA, TR, QL, PD, HI, KI, or SS. In some aspects, the preferred amino acids for the last four amino acids of the CDR3 of the Vδ4 are DKLI (SEQ ID No. 34084) or TRQM (SEQ ID No. 50810).

CDRs of Human Vδ6

In one aspect, in the non-human animal with human or humanized γ/δ T cell, the peptide sequence of CDR1 from the human Vδ6 comprises a conserved amino acid sequence comprising NTAFDY (SEQ ID No. 50811), or at least one N, one T, one A, one F, one D, or one T inside the CDR1 sequence. In other aspect, the CDR2 sequence of the non-human animal Vδ6 comprises IRPDVSE (SEQ ID No. 50812), or at least one I, one R, or one P, one D, one V, one S, or one E inside the CDR2 sequence. The peptide sequence of CDR3 from the human Vδ6 comprises a conserved starting and ending sequence. For the starting sequences, amino acids at the first and second position of the CDR3 of Vδ6 are AA. In some aspects, the preferred amino acids for the first three amino acids of the CDR3 of the Vδ6 are AAS or AAR. Further preferred amino acids for the first four amino acids are AASP, where the 4th position P can be any of T, G, M, V, L, or R. In some other aspects, the preferred amino acids for the first two amino acids of the CDR3 of the Vδ6 are QQ, EA, TA, QH, AV, SK, VA, ES, DA, SA, EG, or EP. For the ending sequences, two contiguous amino acids out of the last five amino acids of the CDR3 of the Vδ6 are DK, LI, KL, TD, DT, PI, TA, TR, PD, HI, HT, KI, or SS. In some aspects, the preferred amino acids for the last three amino acids of the CDR3 of the Vδ6 are TRQ, KLI, or KLN.

CDRs of Human Vγ9

In one aspect, the peptide sequence of CDR1 from the human Vγ9 comprises a conserved amino acid sequence comprising GITISATS (SEQ ID No. 50813), or at least one G, one A, one I, one T, or one S inside the CDR1 sequence. In other aspect, the CDR2 sequence of the non-human animal Vγ9 comprises ISYDGTV (SEQ ID No. 50814), or at least one I, one S, one Y, one G, one T, or one D. In one aspect, in the non-human animal with human or humanized γ/δ T cell, the peptide sequence of CDR3 from the human Vγ9 comprises a conserved starting and ending sequence. For the starting sequences, amino acids at the first four positions of the CDR3 of Vγ9 are ALWE (SEQ ID No. 50815), ALWG (SEQ ID No. 16756), ASWE (SEQ ID No. 50816), ALCE (SEQ ID No. 50817), ALLE (SEQ ID No. 50818), ALRE (SEQ ID No. 50819), PCGR (SEQ ID No. 50820), AWWE (SEQ ID No. 50821), DLWE (SEQ ID No. 50822), ASWE (SEQ ID No. 50816), or AMWE (SEQ ID No. 50823). In some aspects, the first five amino acids of the CDR3 of the Vγ9 are ALWEV (SEQ ID No. 42723), where the fifth position can be any of A, E, or M. In some aspects, in the starting sequence of ALWG (SEQ ID No. 16756), the fourth position can be any of D, V, R, or K. In further aspects, the first four amino acids of the CDR3 of the Vγ9 are TLWE (SEQ ID No. 50829), where the first position can be any of G, V, S, P, or A. In one aspect, the fourth position of CDR3 is an E. For the ending sequences, two contiguous amino acids out of the last five amino acids of the CDR3 of the Vγ9 are KK. In some aspects, the last two amino acids of the CDR3 of the Vγ9 are KTL, KEL, EKL, KNL, IKV, KSR, RNS, KNS, MKL, KRL, RKL, FKI, or KNQG (SEQ ID No. 50830).

CDRs of Human Vγ10

In one aspect, in the non-human animal with human or humanized γ/δ T cell, the peptide sequence of CDR1 from the human Vγ10 comprises a conserved amino acid sequence comprising STRFETDV (SEQ ID No. 50824), or at least one R, one F, one T, one E, one D, one V, or one S inside the CDR1 sequence. In other aspect, the CDR2 sequence of the non-human animal Vγ10 comprises IVSTKSAA (SEQ ID No. 50825), or at least one I, one S, one V, one K, one T, or one A. In some aspects, in the non-human animal with human or humanized γ/δ T cells, the peptide sequence of CDR3 from the human Vγ10 comprises a conserved starting and ending sequence. For the starting sequences, amino acids at the first three positions of the CDR3 of Vγ10 are AAW. In some aspects, the first four amino acids of the CDR3 of the Vγ10 are AAWF (SEQ ID No. 50826), where the fourth position can be any of F, L, A, R, G, C, or V. In some further aspects, the second position is an A and the first position can be any of S, Y, V, E, G, D, T, or P. In some aspects, the second position is an E and the first position can be any of S, D, or A. In some aspects, the first and second positions are PR or SS. In some aspects, the first position is an A, and the second position of any one of V, T, S, G, or E. In some aspects, the first two positions are any of VS, VF, or CE. For the ending sequences, in some aspects, the last three amino acids of the CDR3 of the Vγ10 are FKI. In one aspect, two contiguous amino acids out of the last three amino acids of the CDR3 of the Vγ10 are KK. In one aspect, the second to the last two amino acids of the CDR3 of the Vγ10 is a K. In other aspect, the last amino acid of the CDR3 of the Vγ10 is an I. In one aspect, the last amino acid of the CDR3 of the Vγ10 is an R. In other aspect, the second to the last two amino acids of the CDR3 of the V(γ) 10 is a T.

In one aspect, a method for making a nucleic acid sequence encoding a human γ/δ TCR variable domain that binds an antigen of interest includes: exposing a non-human animal as described herein to an antigen of interest or stimulating expression of internal antigen: and maintaining the non-human animal for developing a humanized γ/δ TCR to a particular antigen.

In some aspects, methods of introducing a foreign and/or endogenous antigen into the non-human γ/δ animal are provided. In one aspect, the antigen is from a biological and/or chemical source. In a further aspect, the antigen comprises a micro-organism or its extract (e. g. E. coli, Staphylococcus extracts), and mammalian organisms comprise mouse cancer (e. g. B16 melanoma) and/or human cancer (e. g. Daudi cells).

In further aspects, after said antigen has been introduced in the human γ/δ T cell mice, a particular population of human γ/δ T cells expanded.

In further aspects, the CDR3 sequences from before and after human γ/δ T cell expansion are sequenced and analyzed.

In one embodiment, the antigen-binding protein comprises a TCR variable domain comprising a human TCRγ and/or human TCRδ variable domain.

In one aspect, the use of a non-human as described herein is provided for making a non-human cell that expresses on its surface a humanized T cell receptor.

A genetically modified non-human animal, whose genome comprises: at least one unarranged human T cell receptor (TCR) Vδ gene segment, at least one unarranged human TCR Dδ gene segment, and at least one unarranged human TCR Jδ gene segment, wherein the at least one unarranged human TCR Vδ gene segment, the at least one unarranged human TCR Dδ gene segment, and the at least one unarranged human TCR Jδ gene segment are operably linked to a functional non-human TCRδ constant gene sequence.

The animal of the preceding embodiment, where the at least one unarranged human TCR Vδ gene segment, the at least one unarranged human TCR Dδ gene segment, and the at least one unarranged human TCR Jδ gene segment are inserted into the endogenous TCRδ variable gene locus.

The animal of any of the preceding embodiments where the at least one unarranged human TCR Vδ gene segment, the at least one unarranged human TCR Dδ gene segment, and the at least one un arranged human TCR Jδ gene segment, replace at least one gene segment of the repertoire of the unarranged endogenous TCR Vδ gene segments, and/or at least one gene segment of the repertoire of the unarranged endogenous TCR Dδ gene segments and/or at least one gene segment of the repertoire of the unarranged endogenous TCR Jδ gene segments.

The animal of any of the preceding embodiments where the at least one unarranged human TCR Vδ gene segment, the at least one unarranged human TCR Dδ gene segment, and the at least one unarranged human TCR Jδ gene segment, replace at least one nucleotide of one gene segment of the repertoire of the unarranged endogenous TCR Vδ gene segments, and/or at least one nucleotide of gene segment of the repertoire of the unarranged endogenous TCR Dδ gene segments and/or at least one nucleotide of one gene segment of the repertoire of the unarranged endogenous TCR Jδ gene segments.

The animal of any of the preceding embodiments where the animal is heterozygous for TCR δ.

The animal of any of the preceding embodiments where the at least one unarranged human TCR Vδ gene segment, the at least one unarranged human TCR Dδ gene segment, and the at least one unarranged human TCR Jδ gene segment replace the complete repertoire of unarranged endogenous TCR Vδ, Dδ and Jδ gene segments.

The animal of any of the preceding embodiments where the at least one unarranged human TCR Vδ gene segment is selected from the unarranged repertoire of the human TCR Vδ gene segments Vδ1, Vδ2, Vδ3, Vδ4, Vδ5, Vδ6, Vδ7, and Vδ8.

The animal of any of the preceding embodiments where the amino acid sequence of the at least one unarranged human TCR Vδ segment has a least 90% sequence identity with SEQ ID NO: 64, 65, 66, 67, 68, 69, 70, 71 and 72.

The animal of any of the preceding embodiments where the at least one unarranged human TCR Vδ gene segment, the at least one unarranged human TCR Dδ gene segment, and the at least one unarranged human TCR Jδ gene segment are capable of rearranging to form a rearranged human VDJ δ sequence.

The animal of any of the preceding embodiments where the animal expresses a humanized TCRδ variable region comprising the rearranged human VDJ δ sequence on the surface of a γδ T cell population.

A genetically modified non-human animal, whose genome the genome comprises: at least one unarranged human TCR Vγ gene segment and at least one human TCR Jγ gene segment, wherein the at least one unarranged human TCR Vγ gene segment and the at least one human TCR Jγ gene segment are operably linked to a functional non-human TCRγ constant gene.

The animal of the preceding embodiment where the at least one unarranged human TCR Vγ gene segment, and the at least one unarranged human TCR Jγ gene segment are inserted into the endogenous TCRγ variable gene locus.

The animal of any of the preceding embodiments where the at least one unarranged human TCR Vγ gene segment, and the at least one unarranged human TCR Jγ gene segment replace at least one gene segment of the repertoire of the unarranged endogenous TCR Vγ gene segments and/or at least one gene segment of the repertoire of the unarranged endogenous TCR Jγ gene segments.

The animal of any of the preceding embodiments where the at least one unarranged human TCR Vγ gene segment, and the at least one unarranged human TCR Jγ gene segment replace at least one nucleotide of one gene segment of the repertoire of the unarranged endogenous TCR Vγ gene segments and/or at least one nucleotide of one gene segment of the repertoire of the unarranged endogenous TCR Jγ gene segments.

The animal of any of the preceding embodiments where the at least one unarranged human TCR Vγ gene segment, and the at least one unarranged human TCR Jγ gene segment replace the complete repertoire of the unarranged endogenous TCR Vγ and Jγ gene segments.

The animal of any of the preceding embodiments where the animal is heterozygous for TCR γ.

The animal of any of the preceding embodiments where the at least one unarranged human TCR Vγ gene segment is selected from the unarranged repertoire of the human TCR Vγ gene segments Vγ2, Vγ3, Vγ4, Vγ5, Vγ8, Vγ9, Vγ10, and Vγ11, preferably Vγ9, Vγ10, and Vγ11.

The animal of any of the preceding embodiments where the amino acid sequence of at least one un arranged human TCR Vγ segment has at least 90% sequence identity with SEQ ID NO: 62, 63 or 64.

The animal of any of the preceding embodiments where the at least one unarranged human TCR Vγ gene segment, and the at least one unarranged human TCR Jγ gene segment are capable of rearranging to form a rearranged human VJ γ sequence.

The animal of any of the preceding embodiments where the animal expresses a humanized TCRγ variable region comprising the rearranged human VJ γ sequence on the surface of a γδ T cell population.

A genetically modified non-human animal, whose genome comprises: at least one unarranged human T cell receptor (TCR) Vδ gene segment, at least one unarranged human TCR Dδ gene segment, and at least one unarranged human TCR Jδ gene segment; and at least one unarranged human TCR Vγ gene segment and at least one human TCR Jγ gene segment, wherein the at least one unarranged human TCR Vδ gene segment, the at least one unarranged human TCR Do gene segment, and the at least one unarranged human TCR Jδ gene segment are operably linked to a functional non-human TCRδ constant gene, and wherein the at least one unarranged human TCR Vγ gene segment and the at least one human TCR Jγ gene segment are operably linked to a functional non-human TCRγ constant gene.

The animal of any of the preceding embodiments where the at least one unarranged human TCR Vδ gene segment, the at least one unarranged human TCR Dδ gene segment, and the at least one unarranged human TCR Jδ gene segment, replace at least one gene segment of the repertoire of the unarranged endogenous TCR Vδ gene segments, and/or at least one gene segment of the repertoire of the unarranged endogenous TCR Dδ gene segments and/or at least one gene segment of the repertoire of the unarranged endogenous TCR Jδ gene segments; and where the at least one unarranged human TCR Vγ gene segment, and the at least one unarranged human TCR Jγ gene segment replace at least one gene segment of the repertoire of the unarranged endogenous TCR Vγ gene segments and/or at least one gene segment of the repertoire of the unarranged endogenous TCR Jγ gene segments.

The animal of any of the preceding embodiments where the at least one unarranged human TCR Vδ gene segment, the at least one unarranged human TCR Dδ gene segment, and the at least one unarranged human TCR Jδ gene segment, replace at least one nucleotide of one gene segment of the repertoire of the unarranged endogenous TCR Vδ gene segments, and/or at least one nucleotide of one gene segment of the repertoire of the unarranged endogenous TCR Do gene segments and/or at least one nucleotide of one gene segment of the repertoire of the unarranged endogenous TCR Jδ gene segments; and where the at least one unarranged human TCR Vγ gene segment, and the at least one unarranged human TCR Jγ gene segment replace at least one nucleotide of one gene segment of the repertoire of the unarranged endogenous TCR Vγ gene segments and/or at least one nucleotide of one gene segment of the repertoire of the unarranged endogenous TCR Jγ gene segments.

The animal of any of the preceding embodiments where the at least one unarranged human TCR Vδ gene segment, the at least one unarranged human TCR Dδ gene segment, and the at least one unarranged human TCR Jδ gene segment are inserted into the endogenous TCRδ variable gene locus, and wherein the at least one unarranged human TCR Vγ gene segment, and the at least one unarranged human TCR Jγ gene segment are inserted into the endogenous TCRγ variable gene locus.

The animal of any of the preceding embodiments where the at least one unarranged human TCR Vδ gene segment, the at least one unarranged human TCR Dδ gene segment, and the at least one unarranged human TCR Jδ gene segment replace the complete repertoire of the unarranged endogenous TCR Vδ, Dδ and Jδ gene segments, and wherein the at least one unarranged human TCR Vγ gene segment, and the at least one unarranged human TCR. Jγ gene segment replace the complete repertoire of the unarranged endogenous TCR Vγ and Jγ gene segments.

The animal of any of the preceding embodiments where the at least one unarranged human TCR Vδ gene segment is selected from the unarranged repertoire of the human TCR Vδ gene segments Vδ1, Vδ2, Vδ3, Vδ4, Vδ5, Vδ6, Vδ7, and Vδ8, and wherein the at least one unarranged human TCR Vγ gene segment is selected from the unarranged repertoire of the human TCR Vγ gene segments Vγ2, Vγ3, Vγ4, Vγ5, Vγ8, Vγ9, Vγ10, and Vγ11.

The animal of any of the preceding embodiments where the at least one unarranged human TCR Vδ gene segment, the at least one unarranged human TCR Dδ gene segment, and the at least one unarranged human TCR Jδ gene segment are capable of rearranging to form a rearranged human VDJ δ sequence, and wherein the at least one unarranged human TCR Vγ gene segment, and the at least one unarranged human TCR Jγ gene segment are capable of rearranging to form a rearranged human VJγ sequence.

The animal of any of the preceding embodiments where the animal expresses a humanized TCRγδ comprising the rearranged human VDJ δ sequence and the rearranged human VJ γ sequence on the surface of a γδ T cell population.

The animal of any of the preceding embodiments where Vγ and Vδ have at least 90% sequence identity with human Vγ and Vδ.

The animal of any of the preceding embodiments where Dδ has at least 90% sequence identity with human Dδ.

The animal of any of the preceding embodiments where the Jγ and Jδ have at least 90% sequence identity with human Jγ and Jδ.

The animal of any of the preceding embodiments where the humanized TCR is expressed with at least one mouse CD3 on the surface of a γδ T cell population.

The animal of any of the preceding embodiments where the animal generates a population of central and effector memory γ/δ T cells to an internal or external antigen.

The animal of any of the preceding embodiments where the animal is heterozygous for TCRδ and/or TCRγ.

A method of producing a humanized TCR includes: administering an antigen of interest to the genetically modified animal of any of preceding embodiments; and obtaining a humanized TCR that recognizes said antigen of interest.

A method of determining and/or analyzing the TCR repertoire of the humanized TCR produced according to the preceding embodiment by next-generation sequencing.

A method of establishing cancer includes: administering a cancer cell to the genetically modified animal of any of the preceding embodiments, and determining cancer growth.

A method of establishing an infection includes: administering an antigen-derived pathogen to the genetically modified animal of any of the preceding embodiments; and determining the repertoire of TCR expansion by sequencing.

A method of making a genetically modified non-human animal, whose genome comprises: at least one unarranged human T cell receptor (TCR) Vδ gene segment, at least one unarranged human TCR Dδ gene segment, and at least one unarranged human TCR Jδ gene segment; and at least one unarranged human TCR Vγ gene segment and at least one human TCR Jγ gene segment, wherein the at least one unarranged human TCR Vδ gene segment, the at least one unarranged human TCR Dδ gene segment, and the at least one unarranged human TCR Jδ gene segment are operably linked to a functional non-human TCRδ constant gene, and wherein the at least one unarranged human TCR Vγ gene segment and the at least one human TCR Jγ gene segment are operably linked to a functional non-human TCRγ constant gene.

Unless otherwise defined, all technical and scientific terms used herein have the common meaning as understood by those skilled in the art. Unless otherwise defined, any of the aspects and embodiments herein can be used in conjunction with one other. Methods and materials are described herein for use in the present invention; other, suitable methods and materials known in the art can also be used. The materials, methods, and examples in the following detailed description are not restrictive of the invention as claimed. The accompanying figures are illustrative only and not intended to be limiting. In case of conflict, the present specification, including definitions, will govern.

### BRIEF DESCRIPTION OF THE DRAWINGS

FIG. 1 shows the strategy for replacing one of the mouse T cell γ locus with one of the human T cell γ locus. The representation in the figure is to the scale as shown by the scale bar on the left-bottom side of the figure. The top panel shows the general organization of endogenous mouse Vγ1, mouse Jγ4, mouse γ constant region C4, and the adjacent 5′ up-stream and 3′ down-stream mouse sequences on chromosome 13. The middle panel depicts the targeting vector used to replace the mousey region. The targeting vector has segments of a) 5′ up-stream mouse homology arm including 5′ UTR of mouse Vγ1, b) human Vγ9, human Vγ10, human Vγ11, human JγP1, human Jγ P, and human Jγ1; c) a selection cassette; d) 3′ down-stream mouse homology arm including mouse constant region C4 (from exon 1 to exon 4). The exons are represented by vertical bars. The bottom panel depicts the targeted event. The targeting vector with human sequence replaced mouse Vγ1 and mouse Jγ4 at the mouse endogenous locus on chromosome 13.

FIG. 2 shows the strategy for replacing part of the mouse T cell δ locus with part of the human T cell δ locus. The representation in the figure is to the scale as shown by the scale bar on the left-bottom side of the figure. The top panel shows the general organization of endogenous mouse Vδ4, mouse Dδ1, Dδ2, Jδ1, Jδ2, and mouse δ constant region C, and the adjacent 5′ up-stream and 3′ down-stream mouse sequences (including mouse Vδ5) on chromosome 14. The middle panel depicts the targeting vector used to replace the mouse δ region. The targeting vector has segments of a) 5′ up-stream mouse homology arm; b) human Vδ8 with a Vδ rabbit promoter 2; c) human Vδ7 with a Vδ rabbit promoter 1; d) human Vδ3 with a Vδ bovine promoter 1; e) human Vδ5 with its own human promoter; f) human Vδ4 with its own human promoter; g) human Vδ6 with its own human promoter; h) human Vδ1 with its own human promoter; i) human Vδ2 with its own human promoter; j) human Dδ1-3; k) human Jδ1-4; I) a selection cassette; m) 3′ down-stream mouse sequences including mouse δ C region (from exon 1 to exon 4). The exons are represented by vertical bars. The bottom panel depicts the targeted event. The targeting vector with human sequence replaced mouse Vδ4, mouse Dδ1, Dδ2, Jδ1, and Jδ2 at the mouse endogenous locus on chromosome 1.

FIG. 3 is a detailed illustration (not to scale) of a progressive strategy for putting together the human segments for the targeting vector. Human variable region gene segments are sequentially Added to a partially deleted human BAC, resulting in an additional 7 human Vδ on the original human Vδ2 locus.

FIG. 4 is the flow chart of humanized γδ T cells from the mouse spleen. In humanized γδ T cells mouse spleen, the majority of the γ/δ T cells are humanized Vδ1/humanized Vγ9 with an abundance of 61%, yet humanized Vδ2/humanized Vγ9 with an abundance of 7.6%.

FIG. 5 is the B16 melanoma cancer development in lungs of WT and humanized γδ T cells mice

FIG. 6 shows further humanization of mouse VγC1 region. Human Vγ1-8 was inserted in the region.

FIG. 7 shows the sequence of PCR product of human gamma TCR sequenced by the Sanger method

FIG. 8 shows the sequence of PCR product of human gamma TCR sequenced by the Sanger method

FIG. 9A-E provide the sequences of CDR3 of Bδ1-DV1

FIG. 10A-D provide the sequences of CDR3 of Vδ3-DV3

FIG. 11A-H provide the sequences of CDR3 of Vδ2-DV2

FIG. 12A-H provide the sequences of CDR3 of Vδ6-DVδ

FIG. 13A-L provide the sequences of CDR3 of Vδ4-DV4

FIG. 14A-B provide the sequences of CDR3 of Vγ9-G32G9

FIG. 15A-C provide the sequences of CDR3 of Vγ9-G33G9

FIG. 16A-G provide the sequences of CDR3 of Vγ10-G35G10

FIG. 17A-E provide the sequences of CDR3 of Vγ10-G37G10

FIG. 18A-J provide the sequences of CDR3 of Vγ10-G36G10

FIG. 19A-B provide the sequences of CDR3 of Vδ3-M4DV3

FIG. 20A-V provide the sequences of CDR3 of Vδ1M4DV1

FIG. 21A-B provide the sequences of CDR3 of Vδ3-M7DV3

FIG. 22A-K provide the sequences of CDR3 of Vδ6-M4DVδ

FIG. 23A-L provide the sequences of CDR3 of Vδ6-M7DVδ

FIG. 24A-U provide the sequences of CDR3 of Vδ1-M7DV1

### DETAILED DESCRIPTION

Some embodiments of the current invention are discussed in detail below and in the accompanying drawings. In describing embodiments, specific terminology is employed for the sake of clarity. However, the invention is not intended to be limited to the specific terminology so selected. The invention is also not intended to be limited to the embodiment depicted by the drawings. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Definitions are included herein for the purpose of understanding the present subject matter and the appended patent claims and drawings. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will occur to those skilled in the art without departing from the invention. It should be understood that various alternatives to the embodiments of the invention described herein may be employed in practicing the invention.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices, and materials similar or equivalent to those described herein can be used in the practice of this invention. The following definitions are provided to facilitate understanding of certain terms used frequently herein and are not meant to limit the scope of the present disclosure.

As used herein. “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein with respect to recombinant proteins, the term “modification” means any insertion, deletion, or substitution of an amino acid residue in the recombinant sequence relative to a reference sequence (e.g., wild-type or a native sequence).

As used herein, the term “homologous recombination” or “HR” refers to the natural, cellular process in which a double-stranded DNA break is repaired using a homologous DNA sequence as the repair template (see, e.g., Cahill et al. (2006), From. Biosci. 11:1958-1976). The homologous DNA sequence may be an endogenous chromosomal sequence or an exogenous nucleic acid that was delivered to the cell.

The terms “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of single or double-stranded polynucleotides, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “human TCR” or “humanized. TCR” refers to a partial or entire TCR that came from a human sequence.

The TCR CDR3 region was defined according to international ImMunoGeneTic (IMGT) nomenclature and a TCR numbering system. Likewise, gene names of V and J regions are designated according to the MGT nomenclature for T cell receptors of human or mouse.

This disclosure relates to genetically modified non-human animals with humanized γ and/or δ T cell receptors.

The term “transgenic and gene-targeted non-human animal,” when used to describe a genetically modified animal (e. g. mice or rat), one skilled in the art would understand that transgenic is achieved by foreign DNA sequence randomly integrated into any chromosome and gene-targeted is achieved by foreign DNA sequence integrated into a designed location of a particular chromosome. Non-human targeted animals may have better control of gene expression and protein translation. But transgenic non-human animals with the right sequence could also produce the correct protein. So adequate modification of the core targeting vector by adding or removing a few genetic segments could lead to a transgenic vector that could be randomly integrated into the non-human animal genome and achieves correct protein expression. One skilled in the art would understand DNA sequence works in gene targeting may also works in transgenic fashion.

The term “conservative” is used to describe an amino acid that can be substituted by another amino acid residue having a side chain R group with similar chemical properties (e.g, charge or hydrophobicity) without significantly changing the biological characters of the protein or peptides. Examples of groups of amino acids that have side chains with similar chemical properties include acidic side chains (e.g., aspartic acids, glutamic acid), basic side chains (e.g., lysine, arginine, histidine), uncharged polar side chains (e.g., asparagine, cysteine, glycine, glutamine, serine, threonine, tyrosine), nonpolar side chains (e.g., alanine, isoleucine, leucine, methionine, phenylalanine, proline, tryptophan, valine), aromatic side chains (e.g., histidine, phenylalanine, tyrosine, tryptophan) and beta-branched side chains (e.g., isoleucine, threonine, valine). Sequence similarity can be measured by if two sequences are identical or not identical but have similar physicochemical properties. The homology percentage, in many cases, is higher than the identity percentage. In some embodiments, alanine scanning mutagenesis can be used to substitute any native residue in a protein.

The term “conserved identical” refers to an amino acid having a similar bio-physical property with another amino acid and substituting an amino acid with a “conserved identical” amino acid will not cause detrimental change to the function of a protein, where the amino acid is in.

One skilled in the art would understand that the degeneracy of the genetic code may encode the polypeptides of the invention. The humanized γ/δ TCR poly peptides described by others may differ from those described herein.

Sequence Identity is the ratio of the number of identical amino acids between the 2 aligned sequences over the aligned length, expressed as a percentage. Two amino acid sequences have “100% amino acid sequence” which means amino acid residues of two amino acid sequences are identical and conserved identical when aligned for maximal correspondence. Sequence comparisons can be performed using standard software programs such as those included in the LASERGENE bioinformatics computing suite (DNASTAR. Madison, Wis.). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Bishop (ed.), Guide to Human Genome Computing (2nd ed., Academic Press, Inc. 1998).) Two amino acid sequences are considered to have “substantial sequence similar or homology” if the two sequences have at least 80%, at least 85%, at least 90%, or at least 95% and above sequence identical relative to each other.

The term “C region” refers to a constant region of a γ/δ T cell receptor, commonly shared among all receptors. The constant region domain has separate gene segments for hinge, transmembrane, and cytoplasmic regions, which provide signal transduction after binding an antigen by the γ/δ T cell receptor.

The term “functional compatible C region” refers to a constant region coming from a specie that is different from the endogenous but retain the function fully or partially of proper signal transduction after binding an antigen by its γ/δ T cell receptor, including sequences that 70%, 75%, 80%, 85%, 90%, 95%, and 100% similar to the original C region of the specie.

The term “synthetical C region” refers to a sequence derived from simulation, substitution, and alternation to form a new C region that retains the function fully or partially of proper signal transduction after binding an antigen by its γ/δ T cell receptor, for example, substituting by a none-γ/δ C region (e.g., alpha/beta C region) or other receptor components.

In this invention the phrase “functional compatible C region” covers the term “synthetic C region” and vice versa.

A “polypeptide” or “polypeptide chain” is a polymer of amino acid residues joined by peptide bonds, that can be produced naturally or synthetically. A peptide commonly means a polypeptide of less than about 10 amino acid residues.

A “humanized γ/δ TCR” is mostly exchangeable with human γ/δ TCR in this invention of the non-human animal models, unless specifically indicated otherwise.

A “humanized γ/δ TCR” is a TCR comprising one or both of a humanized γ domain and a humanized δ domain constant region(s) need not be present, but if they are, they are entirely or substantially from human immunoglobulin constant regions.

This invention is related to an amino acid substituted by a conservative amino acid based on a functional human or humanized γ/δ CRD3. In some embodiments, conservative amino acid substitutions can be in the V region, D region, J region, C region, and CDR3 region. In some aspects, CDRs can come from non-human species, the CDRs herein can be engineered into human-like CDRs. In this regard, the identity of these CDRs in a human-like form is “substantially homology” with corresponding CDRs in human CDRs when at least 70%, at least 75%, at least 80%, at least 85%, at least 90%, at least 95% or 100% of corresponding residues (as defined by Kabat (or IMGT)) are identical between the respective CDRs including amino acids in their conserved group. In particular variations of a humanized TCRγ or TCRδ in which CDRs are substantially homology with human γ/δ TCR, the CDRs of the γ/δ TCR or δ TCR have no more than six (e.g., no more than five, no more than four, no more than three, no more than two, or nor more than one) amino acid substitutions (including conservative substitutions) across all three CDRs relative to the corresponding human γ/δ TCR. The framework sequences of humanized TCRγ or TCRδ variable region are “substantially homology” with a human framework sequence of at least about 80%, at least 85%, at least 90%, at least 95%, or 100% of corresponding residues defined by Kabat numbering convention. A sequence of a humanized γ/δ TCR constant region is homology with rodent (e.g., mouse) constant region when at least about 70%, at least 80%, at least 85%, at least 90%, at least 95%, or 100% of corresponding residues defined by Kabat numbering convention, including conserved amino acids.

All parts of a humanized γ/δ TCR, except the CDRs, are entirely or substantially from corresponding parts of natural human γ/δ TCR sequences.

Percentage sequence identities are determined with γ and/or δ sequences maximally aligned by the Kabat numbering convention. A subject TCR region (e.g., CDR1, CDR2, CDR3, or the entire variable domain of a γ/δ TCR) is being compared with the same region of a reference human TCRs, the percentage sequence identity between the subject and reference TCR regions is calculated by the number of positions occupied by the same amino acid in both the subject and reference TCR region divided by the total number of aligned positions of the two regions, with gaps not counted, multiplied by 100 to convert to percentage. In some aspects, the conserved amino acids (e.g., the same polar group, or the same non-polar group) may be considered identical or substitutable.

The term “homology” in reference to sequences (e.g., nucleotide or amino acid sequences) means two sequences which, upon comparison based on optimal alignment, are identical or substantially identical in at least about 75% of nucleotides or amino acids, at least about 80% of nucleotides or amino acids, at least about 90-95% nucleotides or amino acids (e.g, greater than 97% nucleotides or amino acids).

One skilled in the art would understand that gene targeting is based on homologous recombination occurring between the targeting construct and the targeted endogenous sequence through homology arms, two sequences on the targeting vector that are homologous to endogenous DNA sequences.

The term “operably linked” refers to at least two genetic or protein elements that are joined together in a manner that enables them to carry out their intended function. In this invention, portions of humanized protein may be operably linked to retaining proper folding, processing, transporting, expression, and other functional properties of the protein in the cell. Further, a nucleic acid sequence encoding a protein may be operably linked to DNA sequences (e.g., promoter, enhancer, silencer, insulator, etc.) to retain proper transcription.

The term “replacement” refers to a process comprised of placing exogenous genetic material at an endogenous gene locus, thereby all, a portion, or none of the endogenous genes are removed and an orthologous or homologous nucleic acid sequence is inserted where the endogenous gene is removed. In this invention, the endogenous non-human gene segment is replaced with a corresponding human gene segment. As demonstrated in the Examples below, nucleotide sequences of endogenous non-human TCR γ and δ variable gene loci were replaced by nucleotide sequences corresponding to human TCR γ and δ variable gene loci.

“Functional” as used herein, e.g., in reference to a functional protein, refers to a protein that retains at least one biological activity normally associated with the native protein, e.g., binding with antigen, regulating TCR on T cell surface, activate or de-activate down-stream signaling, inducing cell proliferation, infected cell recognition, and/or cancer cell recognition.

TCR γ/δ locus refers to the genomic DNA comprising the TCR γ/δ coding region including unrearranged V(D)J sequences, enhancer, silencer, insulator, constant domain sequence(s), and upstream or downstream (e.g., 5′ and 3′ UTR, regulatory regions, etc.), or intervening DNA sequence (e.g., introns, etc.), or RNA with regulatory functions. TCR variable locus (e.g., TCR γ variable gene locus or TCR δ variable gene locus), refers to genomic DNA comprising the region that includes TCR variable region segments (V(D)J region) but not includes TCR constant sequences. V region is not narrowly only V element of V(D)J region, instead, V region could mean all of V(D)J region in the following description.

As used herein, the term “chimeric protein” refers to a protein, where two or more portions of the protein are coming from different species. In this invention, a portion of the chimeric protein is of human origin.

V(D)J recombination refers to mechanisms that contribute to the diversity of γ/δ TCR in the vertebrate immune system. The mechanism requires cutting of the DNA boundaries (e.g., at V and J segments for TCRγ and DNA at V, D, and J segments for TCRδ) followed by rejoining of particular pairs of the resulting termini. The imprecision of the joining reaction contributes significantly to increasing the variability of the resulting functional genes. Signal sequences around V, D, and J segments may be diversified, yet still recognizable and processable by one of two or both nuclease RAG1 or/and RAG2, which cleaves the DNA at the border of the signal sequences.

In vivo and in vitro systems of human γ/δ T cells are provided, comprising humanized rodent cells, wherein the rodent cells express one or more humanized immune system molecules.

Unrearranged humanized γ/δ TCR rodent loci that encode humanized γ/δ TCR proteins are also provided. Non-human animals, e.g., rodents, comprising non-human cells that express humanized molecules that have a function in the cellular immune response are also provided.

Immune therapy is centered on identifying targets aberrantly expressed on the surface of infected or cancerous cells. So far, the majority of established and successful targets are proteins or peptides. Protein targets are suited for antibody intervention and peptide target are being recognized by MHC-dependent alpha-beta T cell receptors. As the newly discovered targets become scarce, a new type of non-protein, non-peptides targets are desirable for developing the next generation of immune therapy. As of growing interest in finding new categories of targets, γ/δ T cell receptors can bind to MHC-independent targets e.g., phospholipids, glycolipids, haptens, or small molecules (Willcox., Nat Immunol. (2019) 20:121-128), which αβ T cell receptors will not bind. γ/δ T cells and their receptors have been conserved for 450 million years among jawed vertebrates e.g., fish, mouse, and human. In humans, γ/δ T cell receptor is capable of binding to above mentioned MHC-independent antigens with a natural inclination to react against microbe-infected human cells and human malignant cells, but their molecular and biological interactions are largely unclear, partially because of a lack of human and human-like γ/δ T cell receptor animal model. Primates e.g., monkeys, chimpanzees do have human-like γ/δ T cells, but the availability and ethical issues limited their use in understanding human γ/δ T cell development, infection, and cancer intervention, ultimately γ/δ TCR based immune therapy.

γ/δ T cell receptor comprises a γ polypeptide chain and a δ polypeptide chain, which are bound together by a disulfide bridge. The γ chain comprises lead signal peptides, variable regions (Vγ regions and Jγ regions), a constant domain including a hinge domain, a transmembrane domain, and a cytoplasmic tail domain. The δ chain comprises its lead signal peptides, variable regions (Vδ regions, Dδ region, and Jδ regions), a constant domain including a hinge domain, a transmembrane domain, a cytoplasmic tail domain. 0.76 T cells use V(D)J gene rearrangement with the potential to generate a set of highly diverse receptors capable of recognizing antigens. This diversity is generated in regions of CDR1, CDR2, and CDR3, but mainly in complimentary determining region 3 (CDR3) of the T cell receptor (TCR) via combinatorial and functional diversity.

In humans, γ/δ T cells (including receptors) exist in many tissues e.g., blood, lung, liver, spleen, thymus, intestine, and reproductive tract. Four human γδ T-cell populations can be identified readily by the TCR Vδ expression (Vδ1, Vδ2, Vδ3, and Vδ5) (Zhao., J Transl Med (2018) 16(1)3). Vδ1, Vδ2, Vδ3, and Vγ2, Vγ3, Vγ4, Vγ5, Vγ8, Vγ9, and Vγ1.1 are the most frequently gene segments used in rearrangement of δ and γ chains, respectively (Adams., Cell Immunol (2015) 296(1):31-40). In addition, four subsets of γδ T-cells were detected (Vδ4, Vδ6, Vδ7, and Vδ8) in the peripheral blood of patients with B-cell non-Hodgkin lymphoma indicating their possible involvement of the developing human non-Hodgkin lymphoma. γδ T-cells expressing Vδ1 or Vδ3 TCR chain can be paired with various Vγ chains (Thedrez., Immunol Rev (2007) 215:123-35) and they are predominant in epithelial tissues of skin, lungs, intestine, and reproductive tract (Carding., Nat Rev Immunol (2002) 2(5):336-45), liver, spleen, and thymus (Bonneville., Nat Rev Immunol (2010) 10(7):467-78). In humans, most peripheral bloody T-cells express the Vδ2 TCR chain paired with the Vγ9 chain (Braza., Haematologica (2011) 96(3):400-7).

In wild-type mice, the TCRγ locus spans 205 kb and consists of four clusters of variable elements, one joining element, and one constant element. Cluster one contains Vγ4-7, Jγ1, and Cγ1. Cluster two contains Vγ3, Jγ3, and Cγ3. Cluster three contains Vγ2, Jγ2, and Cγ2. Cluster four contains Vγ1, Jγ4, and Cγ4 according to IMGT annotation (www.igmt.org). The Cγ4 gene is distinguished from the other Cγ genes in that it has an additional hinge-encoding exon. Enhancer elements are located downstream from the Cγ genes responsible for the regulation of the mouse γ TCR production.

In contrast, the human 7 locus spans 175 kb and consists of only one cluster of genes and gene segments on chromosome 7. Within the cluster, it has a stretch of Vγ1-11 and two sets of Jγs and Cγs elements downstream. Set one contains JPγ1, JPγ, Jγ1, and Cγ1 constant region and set two contains JPγ2, Jγ2, and Cγ2 constant region according to IMGT annotation. Enhancer elements are located downstream from the Cγ1 and Cγ2 genes responsible for the regulation of the human γ TCR production.

Very different from the γ locus, both mouse and human δ locus are located inside the α TCR locus, but have their own Vδ, Dδ, Jδ regions, their own Cδ region, and downstream enhancers, which are solely responsible for the regulation of δ TCR production. In the maturation process of αβ T cells, γ/δ locus will be completely deleted, so there is no γ/δ TCR in mature αβ T cells.

In wild-type mice, δ locus spans 2100 kb and consists of Vδ1-9 variable elements, two D elements (Dδ1 and Dδ2), two joining elements (Jδ1 and Jδ2), one constant element, and enhancers located on mouse chromosome 14 according to IMGT annotation.

The human δ locus has a similar structure to the mouse but has more Ds and Js. Human δ locus spans 1000 kb and consists of Vδ1-8 variable elements, three D elements (Dδ1, Dδ2, and Dδ3), four joining elements (Jδ1, Jδ2, Jδ3, and Jδ4), and one constant element (C6), and enhancers located on human chromosome 14 according to IMGT annotation.

γδ TCR complex is comprised of γδ TCR and various CD3 chains following the stoichiometry: TCRγδCD3δ2γδζ2 in humans and TCRγδCD3δ2γ2ζ2 in mice (Siegers., J Exp Med (2007) 204:2537-44). Through its constant region, γ/δ T cell receptor interacts with a variety of signaling proteins to initiate the phosphorylation of immunoreceptor tyrosine-based activation motifs (ITAMS) in the CD3 cytoplasmic domains by the Src-family kinases (SFKs) Lck and Fyn (Kuhns., Immunol Rev (2012) 250:120-43), which leads to recruitment, phosphorylation, and activation of Zap70 that facilitates phosphorylation of the scaffolding proteins SLY-76 and LAT. These cascade events lead to the formation of a supramolecular signalosome that recruits the phospholipase PLCγ1, resulting in the propagation of downstream signaling events (Smith-Garvin., Annu Rev Immunol (2009) 27:591-619). Unlike αβ T-cells, γ/δ T cell TCR complex is CD4 and CD8 co-receptor independent (Kuhns., Immunol Rev (2012) 250:120-43) and the development of γ/δ T cells are not blocked by the mutations on the binding site of PLCγ1 on LAT. In contrary, αβ T-cells, mutations on the binding site of PLCγ1 on LAT resulted in a severe block in murine αβ thymocyte development (Hayes., Immunol Rev (2003) 191:28-37 and Sullivan., J Immunol (2014) 192:2865-74), indicating the C region of γ/δ and αβ are quite different, indicating γ/δ t cell receptor C region plays a unique and dedicated role in the γ/δ t cell associated cascade signaling.

The present invention provides a genetically modified non-human animal (e.g., rodents, e.g., mice, rats) including in its genome an unrearranged human or humanized γ TCR locus which further comprises human Vγ and Jγ gene segments.

The present invention also provides genetically modified non-human animals (e.g., rodents, e.g., mice, rats) comprising in its genome an unrearranged human or humanized δTCR locus which further comprises human Vδ, Dδ, and Jδ gene segments.

The present invention further provides genetically modified non-human animals (e.g, rodents, e.g., mice, rats) comprising in its genome unrearranged human or humanized γ/δTCR locus which further comprises human Vγ, Jγ, Vδ, Dδ, and Jδ gene segments.

Upon enzymatic reaction (e.g., Rag1, Rag2, or other suitable recombinases), the unrearranged human or humanized γ/δ TCR is capable of rearranging to create a nucleotide sequence that encodes a γ/δ TCR protein sequence comprising a V region, a D region (only for δ), and a J region and a non-human Cγ and δ region. The generated γ/δ TCR comprises variable CDR1, CDR2, and CDR3. The present invention also provides that the non-human animal is capable of generating a diverse repertoire of human or humanized γ/δ TCRs before and after antigen stimulation, including but not limited to (e.g., E. coli, Staphylococcus extract).

In one embodiment, the DNA fragment of human Vγ and Jγ replaced endogenous non-human Vγ and Jγ at its endogenous Vγ and Jγ locus to generate a humanized γ TCR locus capable of producing humanized γ TCR with a non-human Cγ region. In other embodiment, the non-human Cγ region can be derived from a different non-human Cγ region other than the endogenous Cγ region, provided that these Cγ regions are functionally compatible, for example, including but not limited to replacing an endogenous Cγ region (a rat) with a rabbit Cγ region, replacing an endogenous Cγ region (a mouse) with a primate Cγ region without significantly hindering the function of γ TCR signaling.

In another embodiment, DNA fragment comprising human Vγ, Jγ, and non-human Cγ region is at a site in the genome other than the endogenous non-human γ TCR locus. The resulting non-human animal is a form of transgene. The non-human Cγ region can come from a functionally compatible Cγ region, not limited to, e.g., primates, rabbits, cows, mice, rats, a mix of different non-human, or a synthetic Cγ region.

In one embodiment, the invention provides a genetically modified non-human animal (e.g., rodent, e.g., rat or mouse) comprises in the germline of the non-human animal the human or humanized unrearranged Vγ and Jγ operably linked to a compatible Cγ region at an endogenous non-human TCR γ locus. The non-human animal comprises a single copy of the human or humanized unrearranged Vγ and Jγ. In some embodiments, the non-human animal comprises two copies of the human or humanized unrearranged Vγ and Jγ. In some embodiments, the non-human animal is a heterozygous or homozygous of the human or humanized unrearranged Vγ and Jγ.

In one embodiment, the human or humanized unrearranged Vγ and Jγ replaces the corresponding endogenous V and J region of non-human animals. In some embodiments, the human or humanized unrearranged Vγ and Jγ inserts, but does not replace the corresponding endogenous V and J region of non-human animals.

In one embodiment, the genetically modified non-human animal (e.g., rodent, e.g., rat or mouse) comprises a further genetically modified genome comprising removed Vγ of non-human and/or Jγ of non-human regions partially or fully. In other embodiment, the non-human animal is incapable of rearranging endogenous Vγ and/or Jγ but capable of rearranging human or humanized unrearranged Vγ and Jγ.

In some embodiments, the Vγ of the genetically modified non-human animal (e.g., rodent, e.g., rat or mouse) comprises a promoter that is from the endogenous animal Vγ. In other embodiment, the Vγ of the genetically modified non-human animal (e.g., rodent, e.g., rat or mouse) comprises a promoter that is from a Vγ promoter derived from a non-human animal other than the endogenous animal.

Similar to γ TCR, at the TCR δ TCR locus, in one embodiment, the DNA fragment of human V δ, Dδ, and Jδ replaced endogenous non-human V δ, Dδ, and Jδ at the endogenous V δ, Dδ, and Jδ locus to generate a humanized δ TCR locus with the capability of producing humanized δ TCR with a non-human Cδ region. In other embodiment, the non-human Cδ region can be derived from a different non-human Cδ region other than the endogenous C region, provided that these Cδ regions are functionally compatible, for example, including but not limited to replacing an endogenous Cδ region (a rat) with a rabbit Cδ region, replacing an endogenous Cδ region (a mouse) with a primate Cδ region without significantly compromising the function of δ TCR signaling.

In another embodiment, DNA fragment comprising human V δ, Dδ, Jδ, and non-human Cδ region is at a site in the genome other than the endogenous non-human TCR δ locus. The resulting non-human animal is transgenic. The non-human Cδ region can come from a functionally compatible Cδ region, not limited to, e.g., primates, rabbits, cows, mice, rats, a mix of different non-humans, or a synthetic Cδ region.

In some embodiments, the invention provides a genetically modified non-human animal (e.g., rodent, e.g., rat or mouse) comprises in the germline of the non-human animal, in which the human or humanized unrearranged Vδ, Dδ and Jδ, operably linked to a compatible Cδ region at an endogenous non-human TCR δ locus. The non-human animal comprises a single copy of the human or humanized unrearranged Vδ, Dδ, and Jδ. In some embodiments, the non-human animal comprises two copies of the human or humanized unrearranged Vδ, Dδ, and Jδ. In some embodiments, the non-human animal is a heterozygous or homozygous of the human or humanized unrearranged Vδ, Dδ, and Jδ.

In one embodiment, the human or humanized unrearranged Vδ, Dδ, and Jδ replace the corresponding endogenous Vδ, Dδ, and Jδ region of non-human animals. In some embodiments, the human or humanized unrearranged Vδ, Dδ, and Jδ inserts, but does not, or partially, replace the corresponding endogenous Vδ, Dδ, and Jδ region of non-human animals.

In one embodiment, the genetically modified non-human animal (e.g., rodent, e.g., rat or mouse) comprises a further genetically modified genome comprising removed Vδ, Dδ, and/or δ of non-human regions partially or fully. In some embodiments, the non-human animal is incapable of rearranging endogenous Vδ, Dδ, and/or Jδ but capable of rearranging human or humanized unrearranged Vδ, Dδ, and Jδ.

In one embodiment, the genetically modified non-human animal (e.g., rodent, e.g., rat or mouse) comprises a contiguous human sequence comprising human Vδ, Dδ, and Jδ. In some embodiments, the genetically modified non-human animal (e.g., rodent, e.g., rat or mouse) comprises a gene segment of the human sequence comprising human Vδ, Dδ, and Jδ. In some embodiments, the genetically modified non-human animal (e.g., rodent, e.g., rat or mouse) comprises a mix of small fragments comprising human Vδ in an order that is not naturally present in human V δ locus. In some embodiments, the Vδ of the genetically modified non-human animal (e.g., rodent, e.g., rat or mouse) comprises a promoter that is from the endogenous animal Vδ. In other embodiment, the Vδ of the genetically modified non-human animal (e.g., rodent, e.g., rat or mouse) comprises a promoter that is derived from a non-human animal other than the endogenous animal Vδ promoter.

In one embodiment, the genetically modified non-human animal (e.g., rodent, e.g., rat or mouse) comprises an inverted human Vδ, which is in the opposite direction as the endogenous C. In further embodiment, the inverted human Vδ is genetically modified to locate in the same direction as the endogenous Cδ.

In one embodiment, the genetically modified non-human animal (e.g., rodent, e.g., rat or mouse) comprises unrearranged human TCR γ (e.g., human Vγ and Jγ) with endogenous Cγ region or functionally compatible Cγ region and TCR δ (e.g., human Vδ, Dδ, and Jδ) with endogenous C region or functionally compatible C δ region. The human TCR γ and TCR δ are located at their corresponding TCR γ and TCR δ loci of the non-human animal. In some embodiments, the non-human animal is capable to rearrange the human Vγ and Vδ with its Ds, or Js to generate γ/δ TCR repertoires before and after antigen stimulation, including but not limited to e.g., E. coli and staphylococcus extract).

In one embodiment, the genetically modified non-human animal (e.g., rodent, e.g., rat or mouse) comprises unrearranged human TCR γ (e.g., human Vγ and Jγ) with endogenous C region or functionally compatible C region and TCR δ (e.g., human Vδ, Dδ, and Jδ) with endogenous C region or functionally compatible Cδ region. The human TCR γ and TCR δ are not located at their corresponding TCR γ and TCR δ loci of the non-human animal as transgenes. In some embodiments, the non-human animal is capable to rearrange the human V γ and Vδ with its Ds, or Js to generate γ/δ TCR repertoires before and after antigen stimulation, including but not limited to e.g., E. coli and staphylococcus extract). The non-human animal with human TCR γ and TCR δ can be double homozygous, one homozygous/one heterozygous, and both heterozygous.

In one embodiment, the genetically modified non-human animal (e.g., rodent, e.g., rat or mouse) comprises a genetic modification in the γ, and/or δ TCR locus.

In some embodiments, in the genetically modified non-human animal (e.g., rodent, e.g., rat or mouse), human Vγ and/or Vδ can have opposite orientations of Cγ and/or C6.

In some embodiments, in the genetically modified non-human animal (e.g., rodent, e.g., rat or mouse), the sequence between two human Vγ and/or Vδ can be shortened and in some embodiments, the sequence between two Vγ and/or Vδ can be lengthened.

In some embodiments, the natural order of human V γ and/or V δ in their locus (e.g., going towards the human Cδ region, following the order of Vδ4, Vδ1, Vδ5, Vδ7, Vδ8, Vδ3) can be followed. In some embodiments, the natural order of human Vγ and/or Vδ in their locus (e.g., the order of Vδ4, Vδ1, Vδ5, Vδ7, Vδ8, Vδ3) are not followed. Thus, a new order of human V γ and/or V δ is created, e.g., including but not limited to Vδ7, Vδ8, Vδ5, Vδ4, Vδ3, Vδ1, and Vδ2.

In some embodiments, the genetically modified non-human animal (e.g., rodent, e.g., rat or mouse) comprises as many as human Vγ and/or δ as in the human γ/δ TCR locus. In some embodiments, the genetically modified non-human animal (e.g., rodent, e.g., rat or mouse) can have more than in the human, e.g., including but not limited to, by duplication of Vγ and/or δ.

In some embodiments, the introduced sequences of Vγ and Jγ, and/or, Vδ, Dδ, and Jδ are highly homology to human Vγ and Jγ, and/or, Vδ, Dδ, and Jδ (e.g., at least 70%, 71%, 72%, 73%, 74%, 75%, 80%, 85%, 90%, 96%, 97%, 98%, or 99% identical to the amino acid sequence of human Vγ and Jγ and/or Vδ, Dδ, and Jδ including conserved amino acids described in the definition.

In some embodiments, the genetically modified non-human animal (e.g., rodent, e.g., rat or mouse) comprises less human Vγ and/or δ as in the human V γ/δ locus.

In some embodiments, the genetically modified non-human animal (e.g., rodent, e.g., rat or mouse) comprises at least one human Vγ and/or Jγ.

In some embodiments, the genetically modified non-human animal (e.g., rodent, e.g., rat or mouse) comprises at least one human Vγ and/or Jγ operably linked to a non-human Cγ region.

In some other embodiments, the genetically modified non-human animal (e.g., rodent, e.g., rat or mouse) comprises at least one human Vδ, Dδ, and/or Jδ.

In some other embodiments, the genetically modified non-human animal (e.g., rodent, e.g., rat or mouse) comprises at least one human Vδ, Dδ, and/or Jδ operably linked to a non-human Cδ region.

In some embodiments, in the genetically modified non-human animal (e.g., rodent, e.g., rat or mouse), as known to the skill of the art, the Vγ and/or δ pseudogene e.g., including but not limited to human Vγ5P, human Vγ6, human Vγ7, or human Vγ11 can turn into a mature protein by substituting the stop codon with a non-stop codon.

In some embodiments, in the genome of the genetically modified non-human animal (e.g., rodent, e.g., rat or mouse), endogenous Vγ and/or δ may be removed, as known to the skill of the art.

In some embodiments, in the genetically modified non-human animal (e.g., rodent, e.g., rat or mouse), human or humanized γ/δ TCR locus may comprise about 10%, about 20%, about 30%, about 40%, about 50%, about 60%, about 70%, about 80%, about 90%, about 95%, or 100% of human Vγ and/or V.

In one embodiment, the genetically modified non-human animal (e.g., rodent, e.g., rat or mouse) comprises a contiguous human sequence of human Vγ9, human Vγ10, human Vγ11, human JPγ1, human JPγ, and human Jγ1. In other embodiment, the genetically modified non-human animal (e.g., rodent, e.g., rat or mouse) comprises a non-contiguous human sequence of Vγ9, human Vγ10, human Vγ11, human JPγ1, human JPγ, and human Jγ1 and operably linked to an endogenous Cγ 4 region.

In one embodiment, the genetically modified non-human animal (e.g., rodent, e.g., rat or mouse) comprises a contiguous human sequence of human Vδ8 with a Vδ rabbit promoter 2, human Vδ1 with a Vδ rabbit promoter, human Vδ3 with a Vδ bovine promoter 1, human Vδ5 with its human promoter, human Vδ4 with its human promoter, a contiguous human sequence of human Vδ6 and Vδ1, a contiguous human sequence of human Vδ2, human Dδ, and human Jδ1-4.

In one embodiment, the genetically modified non-human animal (e.g., rodent, e.g., rat or mouse) comprises a contiguous human sequence of human Vδ8 with a Vδ rabbit promoter 2, human Vδ1 with a Vδ rabbit promoter, human Vδ3 with a Vδ bovine promoter 1, human Vδ5 with its native human promoter, human Vδ4 with its native human promoter, a contiguous human sequence of human Vδ6 and Vδ1, a contiguous human sequence of human Vδ2, human Dδ, human Jδ1-4 and operably linked to an endogenous Cδ region. In one aspect, the human Vδ3 is engineered to be located in the same direction as the endogenous Cδ region.

In some embodiments, the genetically modified non-human animal (e.g., rodent, e.g., rat or mouse) comprises a repertoire of human TCR γ variable segments. The genetically modified non-human animal (e.g., rodent, e.g., rat or mouse) is capable of using enzymatic activity, e.g., including but not limited to Rag1, and/or Rag2, and/or other suitable enzymatic activity, to remove the DNA sequence between Vγ and Jγ and also remove (similar to splicing) the DNA sequence between Jγ and endogenous Cγ region to generate a TCR coding repertoire. In one aspect, the order of removing DNA sequence between segments can remove sequence between Jγ and endogenous Cγ region prior to removing sequence between V γ and Jγ. In other aspect, the endogenous Cγ region can be replaced by a functionally compatible Cγ derived from a non-endogenous source other than from the non-human animal.

In similar embodiments, the genetically modified non-human animal (e.g., rodent e.g., rat or mouse) comprises a repertoire of human TCR δ variable segments. The genetically modified non-human animal (e.g., rodent, e.g., rat or mouse) is capable of using enzymatic activity, e.g., including but not limited to Rag1, and/or Rag2, and/or other suitable enzymatic activity, to remove the DNA sequence between Vδ, Dδ, and Jδ and also remove the DNA sequence between Jδ and endogenous Cδ region to generate a TCR coding repertoire. In one aspect, the order of removing DNA sequence between segments can vary, e.g., remove sequence between Jδ and endogenous C region prior to removing sequence between Vδ and D. In other aspect, the endogenous Cδ region can be replaced by a functionally compatible Cδ derived from a non-endogenous source other than from the non-human animal.

In some embodiments, in a cell of a genetically modified non-human animal (e.g, rodent, e.g., rat or mouse), the endogenous γ/δ T cell expressed human or humanized γ/δ T cell receptor on its surface.

In some embodiments, the antigen can be introduced into the genetically modified non-human animal (e.g., rodent, e.g., rat or mouse) through various routes, including but not limited to e.g., injection, spraying, skin absorption and grafting, eye dropping, or grafting. In some embodiments, antigens can be from various sources, including but not limited to e.g., a single or mixture of lipid, pyrophosphate, protein, sugar, or man-made molecules or materials. In some embodiments, the antigen can be a living or non-living form. In some embodiments, the antigen can be a virus. In some embodiments, the antigen can be on the surface or inside of an object, including but not limited to e.g., an oil or lipid drop, a liposome, a virus, a cell, including but not limited to e.g., a prokaryotic cell, a eukaryotic cell, a bacteria, a yeast, or fungi. In some embodiments, the antigen can be an infected or non-infected cell. In some embodiments, the antigen can be a cancerous or non-cancerous cell. In some embodiments, the antigen can be a DNA or RNA.

CDRs of Human Vδ1

According to IgBlast TCR CDR3 calculation (https://www.ncbi.nlm.nih.gov/igblast/) (Ye et al., “IgBLAST an immunoglobulin variable domain sequence analysis tool,” Nucleic Acids Research 41 (Web Server issue):W34-W40, 2013), in some aspects, in the non-human animal with human or humanized γ/δ T cell, the peptide sequence of CDR1 from the human Vδ1 comprises a conserved amino acid sequence comprising TSWWSYY (SEQ ID No. 50804), or at least one S, one W, one Y, or one T inside the CDR1 sequence. Mother aspect, the CDR2 sequence of the non-human animal Vδ1 comprises QGS, or at least one Q, one G, or one S.

According to the IgBlast TCR CDR3 calculation (https://www.ncbi.nlm.nih.gov/igblast/), in one aspect, in the non-human animal with human or humanized γ/δ T cell, the peptide sequence of CDR3 from the human Vδ1 comprises a conserved starting and ending sequence. For the starting sequences, amino acids at the second and third position of the CDR3 of Vδ1 are L and G, respectively. In other aspect, the preferred amino acids for the first three amino acids of the CDR3 of the Vδ1 are ALG. A further preferred aminoacids for the first four amino acids are ALGE (SEQ ID No. 5398), where position E can be any of G, A, D, V, E, or R. For the ending sequences, two amino acids out of the last five amino acids of the CDR3 of the Vδ1 are DK, LI, KL, TD, DT, PI, TA, TR, QL, or WD. In some aspects, the preferred amino acids for the last three amino acids of the CDR3 of the Vδ1 are KLI.

CDRs of Human Vδ2

According to the IgBlast TCR CDR3 calculation (https://www.ncbi.nlm.nih.gov/igblast/), in one aspect, in the non-human animal with human or humanized γ/δ T cell, the peptide sequence of CDR1 from the human Vδ2 comprises a conserved amino acid sequence comprising GEAIGNYY (SEQ ID No. 50805), or at least one G, one E, one A, one I, one N, one Y inside the CDR1 sequence. In other aspect, the CDR2 sequence of the non-human animal Vδ2 comprises EKD, or at least one E, one K, or one D.

According to the IgBlast TCR CDR3 calculation (https://www.ncbi.nlm.nih.gov/igblast/), in one aspect, in the non-human animal with human or humanized γ/δ T cell, the peptide sequence of CDR3 from the human Vδ2 comprises a conserved starting and ending sequence. For the starting sequences, amino acids at the first three positions of the CDR3 of Vδ2 are ACD, ACG, ACE, ARD, or ASD. In some aspects, the first two amino acids of the CDR3 of the Vδ2 are AW, PV, or DC. A further preferred amino acids for the first four amino acids are ACDS (SEQ ID No. 51034), where the fourth position can be any of T, K, I, P, Y, C, L, M, or R. For the ending sequences, two amino acids out of the last five amino acids of the CDR3 of the Vδ2 are DK, LI, KL, TD, DT, PI, TA, TR, QL, SS, HI, TH, or PD. In some aspects, the preferred amino acids for the last three amino acids of the CDR3 of the Vδ2 are KLI.

CDRs of Human Vδ3

According to the IgBlast TCR. CDR3 calculation (https://www.ncbi.nlm.nih.gov/igblast/), in one aspect, in the non-human animal with human or humanized γ/δ T cell, the peptide sequence of CDR1 from the human Vδ3 comprises a conserved amino acid sequence comprising TVYSNPD (SEQ ID No. 50806), or at least one T, one V, one Y, one S, one N, one P, or one D inside the CDR1 sequence. In other aspect, the CDR2 sequence of the non-human animal V (δ)3 comprises GDNSR, or at least one G, one D, one N, one S, or one R.

According to the IgBlast TCR CDR3 calculation (https://www.ncbi.nlm.nih.gov/igblast/), in one aspect, in the non-human animal with human or humanized γ/δ T cell, the peptide sequence of CDR3 from the human Vδ3 comprises a conserved starting sequence. The first two starting amino acid sequences of the CDR3 of Vδ3 are AF. In some aspects, the preferred amino acids for the first three amino acids of the CDR3 of the Vδ3 are AT, AS, AC, AY, AI, or AV. A further preferred amino acids for the first two amino acids are PF, TF, LF, GF, or GC.

CDRs of Human Vδ4

According to the IgBlast TCR CDR3 calculation (https://www.ncbi.nlm.nih.gov/igblast/), in one aspect, in the non-human animal with human or humanized γ/δ T cell, the peptide sequence of CDR1 from the human Vδ4 comprises a conserved amino acid sequence comprising TSDPSYG (SEQ ID No. 50807), or at least one S, one P, one G, one D, one Y, or one T inside the CDR1 sequence. In other aspect, the CDR2 sequence of the non-human animal Vδ4 comprises QGSYDQQN (SEQ ID No. 50808), or at least one Q, one G, one D, one Y, one N, or one S.

According to the IgBlast TCR CDR3 calculation (https://www.ncbi.nlm.nih.gov/igblast/), in one aspect, in the non-human animal with human or humanized γ/δ T cell, the peptide sequence of CDR3 from the human Vδ4 comprises a conserved starting and ending sequence. For the starting sequences, the first three amino acids of the CDR3 of Vδ4 are AMR, ASP, AMS, AMT, AMG, AMI, AIR, AKR, ATR, EMR, VMR, PMR, and ALP. In one aspect, the preferred amino acids for the first three amino acids of the CDR3 of the Vδ1 are AMRE (SEQ ID No. 50809), where the fourth position can be any of G, C, D, V, N, A, L, V, T, or R. For the ending sequences, two amino acids out of the last five amino acids of the CDR3 of the Vδ4 are DK, LI, KL, TD, DT, PI, TA, TR, QL, PD, HI, KI, or SS. In some aspects, the preferred amino acids for the last four amino acids of the CDR3 of the Vδ4 are DKLI (SEQ ID No. 34084) or TRQM (SEQ ID No. 50810).

CDRs of Human Vδ6

According to the IgBlast TCR CDR3 calculation (https://www.ncbi.nlm.nih.gov/igblast/), in one aspect, in the non-human animal with human or humanized γ/δ T cell, the peptide sequence of CDR1 from the human Vδ6 comprises a conserved amino acid sequence comprising NTAFDY (SEQ ID No. 50811), or at least one N, one T, one A, one F, one D, or one T inside the CDR1 sequence. In other aspect, the CDR2 sequence of the non-human animal Vδ6 comprises IRPDVSE (SEQ ID No. 50812), or at least one I, one R, or one P, one D, one V, one S, or one E inside the CDR2 sequence.

According to the IgBlast TCR CDR3 calculation (https://www.ncbi.nlm.nih.gov/igblast/), in one aspect, in the non-human animal with human or humanized γ/δ T cell, the peptide sequence of CDR3 from the human Vδ6 comprises a conserved starting and ending sequence. For the starting sequences, amino acids at the first and second position of the CDR3 of Vδ6 are AA. In some aspects, the preferred amino acids for the first three amino acids of the CDR3 of the Vδ6 are AAS or AAR. A further preferred amino acids for the first four amino acids are AASP (SEQ ID No. 50827), where the 4th position P can be any of T, G, M, V, L, or R. In some other aspects, the preferred amino acids for the first two amino acids of the CDR3 of the Vδ6 are QQ, EA, TA, QH, AV, SK, VA, ES, DA, SA, EG, or EP. For the ending sequences, two contiguous amino acids out of the last five amino acids of the CDR3 of the Vδ6 are DK, LI, KL, TD, DT, PI, TA, TR, PD, HI, HT, KI, or SS. In some aspects, the preferred amino acids for the last three amino acids of the CDR3 of the Vδ6 are TRQ, KLI, or KLN.

CDRs of Human Vγ9

According to the IgBlast TCR CDR3 calculation (https://www.ncbi.nlm.nih.gov/igblast/), in one aspect, in the non-human animal with human or humanized γ/δ T cell, the peptide sequence of CDR1 from the human Vγ9 comprises a conserved amino acid sequence comprising GMSATS (SEQ ID No. 50813), or at least one G, one A, one I, one T, or one S inside the CDR1 sequence. In other aspect, the CDR2 sequence of the non-human animal Vγ9 comprises ISYDGTV (SEQ ID No. 50828), or at least one I, one S, one Y, one G, one T, or one D.

According to the IgBlast TCR CDR3 calculation (https://www.ncbi.nlm.nih.gov/igblast/), in one aspect, in the non-human animal with human or humanized γ/δ T cell, the peptide sequence of CDR3 from the human Vγ9 comprises a conserved starting and ending sequence. For the starting sequences, amino acids at the first four positions of the CDR3 of Vγ9 are ALWE (SEQ ID No. 50815), ALWG (SEQ ID No. 16756), ASWE (SEQ ID No. 50816), ALCE (SEQ ID No. 50817), ALLE (SEQ ID No. 50818), ALRE (SEQ ID No. 50819), PCGR (SEQ ID No. 50820), AWWE (SEQ ID No. 50821), DLWE (SEQ ID No. 50822),

ASWE (SEQ ID No. 50816), or AMWE (SEQ ID No. 50823). In some aspects, the first five amino acids of the CDR3 of the Vγ9 are ALWEV (SEQ ID No. 42723), where the fifth position can be any of A, E, or M. In some aspects, in the starting sequence of ALWG (SEQ ID No. 16756), the fourth position can be any of D, V, R, or K. In further aspects, the first four amino acids of the CDR3 of the Vγ9 are TLWE (SEQ ID No. 50829), where the first position can be any of G, V, S, P, or A. In one aspect, the fourth position of CDR3 is an E. For the ending sequences, two contiguous amino acids out of the last five amino acids of the CDR3 of the Vγ9 are KK. In some aspects, the last amino acids of the CDR3 of the Vγ9 are KTL, KEL, EKL, KNL, IKV, KSR, RNS, KNS, MKL, KRL, RKL, FKI, or KNQG (SEQ ID No. 50830).

CDRs of Human Vγ10

According to the IgBlast TCR CDR3 calculation (https://www.ncbi.nlm.nih.gov/igblast/), in one aspect, in the non-human animal with human or humanized γ/δ T cell, the peptide sequence of CDR1 from the human Vγ10 comprises a conserved amino acid sequence comprising STRFETDV (SEQ ID No. 50824), or at least one R, one F, one T, one E, one D, one V, or one S inside the CDR1 sequence. In other aspect, the CDR2 sequence of the non-human animal Vγ10 comprises IVSTKSAA (SEQ ID No. 50825), or at least one I, one S, one V, one K, one T, ozone A.

According to the IgBlast TCR CDR3 calculation (https://www.ncbi.nlm.nih.gov/igblast/), in some aspects, in the non-human animal with human or humanized γ/δ T cells, the peptide sequence of CDR3 from the human Vγ10 comprises a conserved starting and ending sequence. For the starting sequences, amino acids at the first three positions of the CDR3 of Vγ10 are AAW. In some aspects, the first four amino acids of the CDR3 of the Vγ10 are AAWF (SEQ ID No. 50826), where the fourth position can be any of F, L, A, R, G, C, or V. In some further aspects, the second position is an A and the first position can be any of S, Y, V, E, G, D, T, or P. In some aspects, the second position is an E and the first position can be any of S, D, or A. In some aspects, the first and second positions are PR or SS. In some aspects, the first position is an A, and the second position of any one of V, T, S, G, or E. In some aspects, the first two positions are any of VS, VF, or CE. For the ending sequences, in some aspects, the last three amino acids of the CDR3 of the Vγ10 are FKI. In one aspect, two contiguous amino acids out of the last three amino acids of the CDR3 of the Vγ10 are KK. In one aspect, the second to the last two amino acids of the CDR3 of the Vγ10 is a K. In other aspect, the last amino acid of the CDR3 of the Vγ10 is an I. In one aspect, the last amino acid of the CDR3 of the Vγ10 is an R. In other aspect, the second to the last two amino acids of the CDR3 of the V(γ) 10 is a T.

An embodiment of the invention provides a TCR comprising two polypeptides (i.e., polypeptide chains), such as a γ chain of a TCR, a δ chain of a TCR, or a combination thereof. The polypeptides of the inventive TCR can comprise any amino acid sequence, provided that the TCR has antigen-binding specificity.

In an embodiment of the invention, the TCR comprises two polypeptide chains, each of which comprises a variable region comprising a complementary determining region CDR1, a CDR2, and a CDR3 of a γ/δ TCR.

The first group of TCR comprises 8 human or humanized δ chains.

The inventive TCRs further comprise a constant region from a non-human animal (e.g., rodent, murine-mouse). The inventive TCRs may further comprise a constant region derived from any suitable species such as, e.g., rabbit or goat.

In an embodiment of the invention, the TCRs further comprise a rabbit, cow, and maybe human constant region.

In an embodiment of the invention, the TCR comprises a murine constant region. For example, the TCR may be a chimeric TCR comprising a human variable region and a murine constant region. The murine constant region for TCR δ chain comprises SEQ ID (amino acid) NO: 73 (constant region of a murine δ TCR chain); The murine constant region for TCR γ chain comprises SEQ ID (amino acid) NO: 74 (constant region of a murine γ TCR chain); There are more than one γ constant regions.

In some aspects, the constant region of the TCR δ comprises one or more conserved amino acid sequences: PSVF (SEQ ID No. 50789), MKNG (SEQ ID No. 50790), GTNVACL (SEQ ID No. 50791), SAVKLGQ (SEQ ID No. 50792), SVTCSV (SEQ ID No. 50793), KVNMMSL (SEQ ID No. 50794), VLGLR (SEQ ID No. 50795), LFAK (SEQ ID No. 50796), and/or NFLL (SEQ ID No. 50797).

In some aspects, the constant region of the TCR γ comprises one or more conserved amino acid sequences: PKPT (SEQ ID No. 50798), LCLL (SEQ ID No. 50799), KTKD (SEQ ID No. 50800), IVIKFSWLT (SEQ ID No. 50801), TSAYY (SEQ ID No. 50802), and/or LLLLLKS (SEQ ID No. 50803).

It is as understood by those skilled in the art one or more amino acid substitution(s) in the murine or non-human animal constant region of the γ and/or δ chain can still yield a protein with functional 7 and/or δ chain.

Genetically Modified Non-Human Animal

As used herein, the term “genetically modified non-human animal” refers to a non-human animal having exogenous DNA in at least one chromosome of the animal's genome, in some aspects, existing as extra-chromosome DNA fragment (e.g., plasmid, mitochondria DNA). In some embodiments, at least one or more cells, e.g., at least 1%, 2%, 3%, 4%, 5%, 10%, 30%, 40%, or 50% of cells of the genetically modified non-human animal have the exogenous DNA in its genome. The cells having exogenous DNA comprise various kinds of cells, e.g., a somatic cell, a germ cell (e.g., a sperm, an egg, a blastocyst), an immune cell (e.g., a T cell, a B cell, a natural killer T cell, a mast cell), an antigen-presenting cell (e.g., a macrophage, a dendritic cell), or an endogenous tumor cell, or a brain cell. In some embodiments, the genetically modified non-human cell can also be introduced into a species that does or does not have the same genetically modified gene.

In some embodiments, genetically modified non-human animals are provided that comprise a modified endogenous γ/δ TCR locus that comprises an exogenous sequence (e.g., a human sequence), e.g., an insertion of exogenous sequence or a replacement of one or more non-human sequences with one or more human sequences through homologous recombination (HR) in mouse ES cells. The animals are generally able to pass the modification to progeny, i.e., through germline transmission.

The genetic modification of humanization of γ/d TCR may be created in other animals, e.g., a rat, rabbit, pig, bovine, deer, sheep, goat, chicken, cat, dog, ferret, primate (e.g., marmoset, rhesus monkey) by several techniques that are known in the art, including, e.g., nonhomologous end joining (NHEJ), homologous recombination (HR), zinc finger nucleases (ZFNs) (reviewed in Durai et al. (2005), Nucleic Acids Res 33, 5978), transcription activator-like effector-based nucleases (TALEN) (reviewed in Mak et al. (2013), Curr Opin Strict Biol. 23:93-9), and the clustered regularly interspaced short palindromic repeats (CRISPR)-Cas system (Ran et al. (2013), Nat Protoc. 8:2281-2308; Mali et al. (2013). Nat Methods 10:957-63). Many other methods are also provided and can be used in genome editing, e.g., micro-injecting a genetically modified nucleus into an enucleated oocyte or fusing an enucleated oocyte with another genetically modified cell, in further aspects, transferring the modified genome to a suitable cell, e.g., an oocyte, and gestating the modified cell (e.g., the modified oocyte) in a non-human animal under suitable conditions to form an embryo.

In one aspect, the animal is a mammal, e.g., of the superfamily Dipodoidea or Muroidea. In some embodiments, the genetically modified animal is a rodent. The rodent comprises a mouse, a rat, and/or a hamster. In some embodiments, the genetically modified animal is from a family selected from Platacanthomyidae, Spalacidae, and Eumuroida which further comprises Calomyscidae, (e.g., mouse-like hamsters), Cricetidae (e.g., hamster, New World rats and mice, voles), Muridae (true mice and rats, gerbils, spiny mice, crested rats), Nesomyidae (climbing mice, rock mice, with—tailed rats, Malagasy rats and mice). In some embodiments, the genetically modified rodent is selected from a true mouse or rat (family Muridae), a gerbil, a spiny mouse, and a crested rat. In some embodiments, the non-human animal is a mouse. In some embodiments, the mouse, a rat, and/or a hamster has at least 80%, 90%, 95% homology with C57BL strain mouse on the nucleotide sequence level.

In some embodiments, the animal is a mouse of a C57BL strain selected from C57BL/6, C57BL/6ByJ, C57BL/6J, C57BL/10, C57BL/6NJ, C57BL/6NIH, B6NTac, C57BL/A, C57BL/KaLwN, C57BL/GrFa, C57BL/10Cr, C579L/10ScSn, C57BL/An, and C57BL/Ola.

In some embodiments, the mouse is selected from the group of 129 strains comprising 129P1, 129P2, 129P3, 129X1, 129S1, 129S1/SV, 129S1/SvIm, 12952, 12954, 12955, 129S9/SvEvH, 129S6, 129/SvEvTac, 129S7, 129S8, 129T1, 129T2. These mice are documented in the full or part description, e.g., Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, as aforementioned, the genetically modified mouse is a mix of the 129 strain and the C57BL/6 strain. In some embodiments, the mouse is a mix of the 129 strains or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, a Long-Evans Agouti strain, a Sprague Dawley strain, a Fischer strain, F344, F6, and Dark Agouti strain. In some embodiments, the rat strain is a mix of two or more strains selected from the aforementioned group of rats.

Use of Genetically Modified Human or Humanized γ/δ T Cell Animals

There is growing interest in γ/δ T cell-based therapy in the art, as γ/δ TCRs recognize stress-induced self-antigens (Groh V, Science (1998) 279:1737-40 and Uldrich A R, Nat Immunol (2013) 14:1137-45), lipids or pyrophosphates secreted by microbes or overproduced in tumor cells (Hayday A. J Immunol. (2019), 203(2):311-320)

There is a special interest in Vγ9/Vδ2 T cell, as it is the main subset of γ/δ T cell in human blood, which accounts for 2-10% of all T cells. The γ/δ TCR recognizes self or foreign non-peptidic phosphorylated molecules called phosphoantigens (Espinosa, JBC (2001) 276:18337-44). Evidence suggests that V γ 9/V δ 2 T cell may exhibit pathogen specificity (Worku, (2001) J. Infect. Dis 184:525-532) as well as immune memory (Shen, Science (2002) 295:2255-58), two characteristics of adaptive immunity. After microbial exposure, γ/δ T cell of an infant experiences polyclonal expansion as well as Vδ1 expansion (Ravens, PNAS (2020) 117:18649-60), indicating TCR's recognition of specific pathogen antigens. Bioinformatic analyses of large meta-genomic datasets determined the relative abundance of γ/δ T cells within tumors significantly correlated with patient outcomes. Tumor-infiltrating γ/δ T cells were found in all tumor entities. A correlation between the relative abundance of γ/δ TILs and favorable response to immune checkpoint therapy in various cancers was demonstrated (Gentles, Nat. Med (2015)1-12). V γ 9/V δ 2 T cells have been shown to be able to recognize various stress markers (Dai, JBC (2012) 287:16812-19 and Gober, J. Exp. Med (2003) 197:163-8. γ/δ cells are also stimulated by certain tumor cells, such as the Daudi B lymphoma (Fisch, Eur. J. Immunol (1997) 27:3368-79).

But Vγ9/Vδ2 T cell and other human or humanized γ/δ subclasses are not existed in most experimental animals including but not limited to, rodents e.g., mouse, rat hamster, cat, dog, sheep, cow, chicken. The unavailability of human or humanized V γ Vγ9/Vδ2 T cell has hindered the research development of many areas including but not limited to, vaccine development, cancer therapy development, infection disease prevention, and slowing aging.

More specifically, the human or humanized γ/δ TCR mice can be a good model for human vaccine development. Small animals (such as mice) played an important role in vaccine development, as of low cost, easy to generate in large quantities, and capable of generating adaptive immune responses, yet provided limited value as a vast amount of clinical trials for infection disease failed with great variable results among individuals, even though most vaccine candidates had already tested and evaluated in such small animals (Dantzler, Clinical & Translational Immunology 2019; e1072). The adaptive immune responses generated in this animal is a result of a lengthy process, particularly, the bridging between innate and adaptive responses, which is not well understood in both human and animals. Among various immune cells, γδ T cell is unique in its category. It has a repertoire of tens of thousands of TCRs, public and private, representing group and individualism. γδ T cell has been well proven to play a role in innate (clone expanding, clone focusing, cytokine releasing, and immune cell calling) and adaptive (memory cell formation). In fact, γδ T cell-deficient mice had significantly delayed recovery from many bacteria and yeast infections.

The rationale of replacing mouse γδ TCR with human γδ TCR to make a better vaccine test model is based on the following reasons: 1) Mouse and human γδ TCR are not quite different. A particular vaccine may be able to generate a mouse γδ TCR, but the same vaccine may not generate a human γδ TCR. So the subsequent clinical trial is solely based on the results from mouse γδ TCR studies and mouse γδ TCR-initiated innate, bridging, and adaptive reactivates. In fact, γ9δ2, one of the most studied γδ TCR in human, do not even exist in the mouse. Using WT animals for vaccine study completely ignored the Vγ9Vδ2 or other human Vδ initiated immune responses. 2) Different from α/β, γδ TCR recognized non-protein antigens, such as phosphoantigen, which may share among many animals including humans. A cloned γδ TCR from a humanized mouse may directly interact with infected human cells, and vice versa, infected human cells can stimulate the expansion and focusing of human γδ T cells, which makes in vivo testing highly possible. 3) The humanized mouse offers further translational value as the sequences obtained from expanded and memory γδ T cells can be directly compared with the vast amount of existing human γδ repertoires. In fact, in the preliminary data provided with this application, numerous γ or δ TCRs are identical to human γδ TCRs discovered in previous human studies. 4) the formation of memory γδ T cells is one of the hallmarks of vaccine development. This human γδ T cell mouse can be challenged repetitively and monitored closely the process of γδ memory T cell formation, which could not be done in any other animals. In sum, the proposed humanized γδ T cell model offered us a unique platform for studying human or human-like innate, bridging, and adaptive immune response of the vaccine development.

In various embodiments, the present invention solves this problem by providing a genetically modified non-human animal comprising human or humanized γδ T cell receptors and expression of these TCRs, particularly human or humanized Vγ9Vδ2 T cells. Inside the non-human animal, a diversified γδ TCR repertoire was established which encodes for human or humanized γδ T cell receptors. With antigen stimulation including but not limited to e.g., E coli, Staphylococcus aureus aqueous suspension. The non-human animal undergoes clonal expansion and clonal focusing, indicating the non-human animal is capable of utilizing its internal mechanisms to process the introduced human genomic DNA to generate functional γδ T cell receptors. As γδ T cells have proven to have a broad application for adoptive cell immunotherapy, the antigen-specific sequences of human or humanized receptors can be cloned and expressed on cells of choice.

In some embodiments, the cells of choice can be including but are not limited to a non-immune cell, an immune cell, or a cell line.

In some embodiments, cancer cells from human and non-human origin are introduced into the non-human animal, tumor growth is monitored and provided in the present invention. The cancer cell lines include but are not limited to a mouse cancer cell line e.g., B16 mouse melanoma cancer cell line, and a human cell line e.g., a lymphoma Daudi B cell line.

A big advantage γδ T cell vs αβ T-cell is that αβ T-cell immune systems cannot be transferred because of the large diversity of HLA among individuals unless all of the HLA molecules are precisely matched. On the contrary, transferring γδ T-cell immune systems between individuals is less restricted because of common antigens, which allows the use of γδ T-cells from normal volunteers who would serve as “universal donors.” Therefore, well-characterized γδ T-cells and their receptors could, in principle, be an “off the shelf” product with one donor providing a T-cell product for a larger population reducing the developmental cost and time.

In some embodiments, the human or humanized γδ T cell comprises one human or humanized γ TCR and one human or humanized δ TCR. In other aspect, the human or humanized γδ T cell comprises two human or humanized γ TCR or two human or humanized δ TCR. In other aspect, the human or humanized γδ T cell comprises more than two human or humanized γ TCRs or more than two human or humanized δ TCRs.

In other aspect, the human or humanized γδ T cell comprises one or human or humanized γ TCR combined with other receptors to form a receptor complex. In other aspect, the human or humanized γδ T cell comprises one or two human or humanized δ TCR combined with other cell surface receptors to form a receptor complex.

In some embodiments, the human or humanized δ TCR can be fused through a molecular linker to a second entity that provides biochemical or cytotoxicity to kill infected cells or cancer cells. The second entity includes but is not limited to e.g., small molecules, radioisotopes, toxins, light-activated drugs, antibodies, prodrug, target activating molecules, enzyme-based drug release molecules, chemokine, cytokine, immune activation and inhibition molecules, an alpha. TCR, a beta TCR, an IgG heavy chain, or an IgG light chain.

In humans, γδ NKT cells develop in the neonatal thymus and migrate to peripheral sites such as the spleen, lymph nodes, and, most prominently, the liver (Azuara, V, 1997, Eur. J. Immunol. 27: 544-553). γδ NKT cells are considered an innate-like lineage based on the expression of the innate signature transcription factor PLZF (Kreslaysky, T, 2009, Proc. Natl. Acad. Sci. USA 106: 12453-124584 Mature γδ NKT cells are phenotypically CD24lowCD44highNK1.1+ and are capable of secreting various cytokines of IFN-γ, IL-4, and IL-13 upon TCR stimulation (Alonzo, E, Curr. Opin. Immunol. 23: 220-227.). These innate features provide γδ NKT cells with important functions in immune protection (Belles, C. 1996, J. Immunol. 156: 4280-4289.) as well as in immune disorders such as Sjögren's syndrome (Belle, I. 2014 Cytokine 69: 226-233.), dermatitis (Kreslaysky, T, 2009, Proc. Natl. Acad. Sci. USA 106: 12453-12458.), and asthma (Felices, M., Proc. Natl. Acad. Sci. USA 106: 8308-8313.). The γδ NKT cells were also identified in mice and the population size of γδ NKT cells is tightly regulated during thymic development and typically represents 10% of total γδ T cells in C57BL/6 mice (Verykokakis, M., 2010. PLoS One 5: e9303), so human or humanized γδ T mouse in this invention can also include this type of NKT cells with human γδ TCR. If this human γδ TCR-based mouse NKT can recognize human infected or cancerous cells, then the human TCR can be cloned and expressed on various cell types to fight against these degenerated cells.

### EXAMPLES

The invention is further described in the following examples, which do not limit the scope of the invention. The conventional methods that are known in the art are not described in detail in the Examples such as PCR, molecular cloning techniques, bacteria BAC recombineering, embryonic stem cell culture, micro-injection, animal breeding, etc. The CDR3 clone numbers were coming from actual experiments. It is understandable by the art that experimental data may vary according to different mice, different PCR and NGS procedures, different repertoire analysis software, and different ways of data presentation. The data shown herein are to support the claims, but not limit the claims.

### Example 1

Generation of Human or Humanized TCRγ Mice (Introducing Human Vγ9-11)

In this example, a non-human animal (such as a mouse) was modified so that the non-human animal contained a nucleic acid sequence comprising human TCRγ further comprising human TCR Vγ and Jγ. Specifically, an endogenous mouse sequence containing mouse TCR Vγ and Jγ was replaced with a sequence containing human TCR Vγ and Jγ and operably linked to a mouse TCR Cγ region.

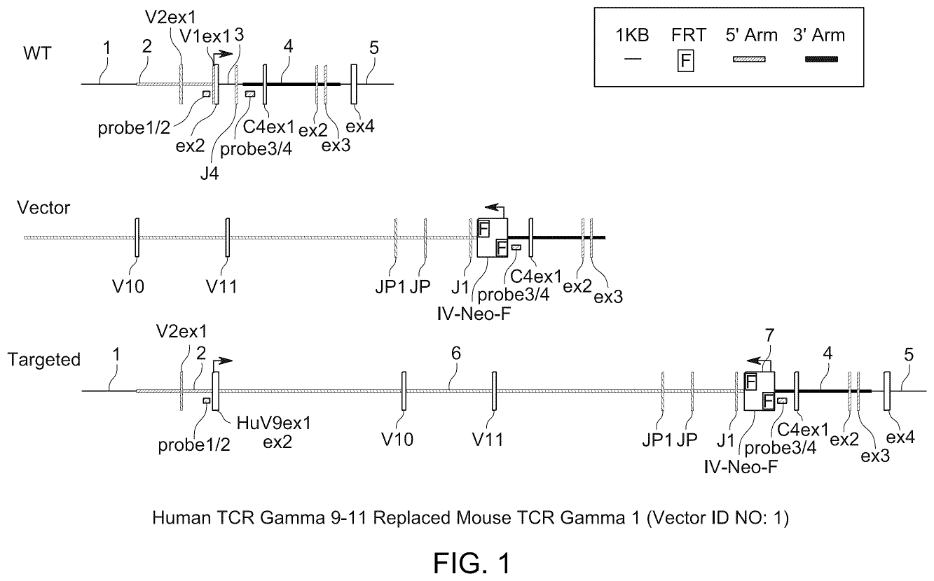

More specifically, 2680 bp of mouse sequence including mouse TCR. Vγ1 and mouse Jγ4 was replaced with 48732 bp of human sequence corresponding to 3 human Vγ (Vγ9, Vγ10, and Vγ11) and 3 human Jγ (JPγ1, JPγ, and Jγ1). The human sequence ended 5′ upstream of the mouse endogenous Cγ4 region, so human γ Vs and Js were genetically linked to the mouse Cγ4 region. The humanization strategy was summarized in FIG. 1. Junctional nucleic acid sequences inside the targeting vector among mouse, human, and prokaryotic/eukaryotic selection cassettes are summarized in Table 1 and also provided in the Sequence Listing (Seq ID NO: 1-5).

TABLE 1
Junctional Nucleic acid sequences for
Vgamma Targeting Vector (vector ID NO: 1)

| SEQ ID NO | Description |
|---|---|
| 1 | 5' starting nucleic acid sequence of DNA segment |
| | NO: 2 of the 5' mouse homology arm |
| | inside mouse TCRgamma locus |
| 2 | Junctional nucleic acid sequence between 3' end |
| | of mouse S'arm (DNA segment NO: 2) |
| | and the starting of the 5' end of human |
| | sequence (DNA segment NO: 6) |
| 3 | Junctional nucleic acid sequence between 3' end of |
| | human sequence (DNA segment NO: 6) and 5' |
| | end of the selection cassette (DNA segment NO: 7) |
| 4 | Junctional nucleic acid sequence between 3' end of the |
| | selectioncassette (DNA segment NO: 7) and S' end of 3' |
| | mousehomology arm (DNA segment NO: 4) |
| 5 | 3' ending nucleic acid sequence of DNA segment |
| | NO: 4 of the 3' mouse homology arm inside |
| | mouse TCRgamma locus |

The first step of generation of a genetically modified mouse with human TCRγ was to construct a TCRγ targeting vector (Vector ID NO: 1), The targeting vector (Vector ID NO: 1) has four DNA segments: a 5′ mouse homology arm including mouse Vγ1 promoter (DNA segment NO: 2), a human sequence having human Vγ9, Vγ10, and Vγ11 and Vγ1, JPγ, and Jγ1 (DNA segment NO: 6), an antibiotic cassette used for eukaryotic selection flanked by Frt sites (DNA segment NO: 7), and a 3′ mouse homology, arm including mouse Cγ4 exon 1-3 (DNA se anent NO: 4).

Two BAC clones were used for the construction of the TCR γ targeting vector (Vector ID NO: 1) (a mouse RP23-475Cδ and a human CTD-256309, Invitrogen). The 5′ mouse homology arm including the mouse Vγ1 promoter (DNA segment NO: 2) has a DNA sequence length of 7052 bp. The 3′ mouse homology arm including mouse Cγ4 exon)-3 (DNA segment NO: 4) has a DNA sequence length of 8868 bp. Both 5′ and 3′ homology arms were from mouse BAC clone RP23-475Cδ and were modified by bacteria BAC recombineering. The human replacement sequence (DNA segment NO: 6) with a DNA sequence length of 48732 bp was cloned from human BAC clone CTD-256309 and was also modified by bacteria BAC recombineering. The antibiotic cassette DNA segment NO: 7) used for eukaryotic selection has a ubiquities promoter driving a Neo selection cassette flanked by Frt sites. The five DNA segments (DNA segment NO: 2, 6, 7, 4) and a BAC-based vector was put together (Vector ID NO: 1) through DNA ligation and bacteria homology recombination using a homology arm with an average length of 130 bp (various from 50-200 bp). The bacteria homology recombination was helped by using bacterial Spec and Kan selection cassettes. During the targeting vector construction process, a unique (not cutting into DNA segment NO: 2, 6, 7, 4) enzyme cutting site AscI was introduced to allow linearization of the targeting vector (Vector ID NO: 1). The linearized targeting vector (Vector ID NO: 1) was electroporated into a hybrid mouse embryonic stem (ES) cell line derived from a hybrid strain by crossing mice of two different inbred strains (B6 and 129). ES cells with correct targeted clones were identified and confirmed by a combination of various methods (e.g., PCR-based assays, long arm PCR, long-range PCR, southern, and/or human replacement PCR) known in the art. Targeted ES clones were expanded and micro-injected into mouse blastocysts to generate chimera mouse with human targeted gene segment (DNA segment NO: 6). Germline mouse was obtained by further mating the chimera with B6 inbreed and additional PCR confirmation was performed. The Neo cassette was removed by the activity of Flp recombinase ES cells or mouse tissues.

### Example 2

Generation of Human or Humanized TCR Mice

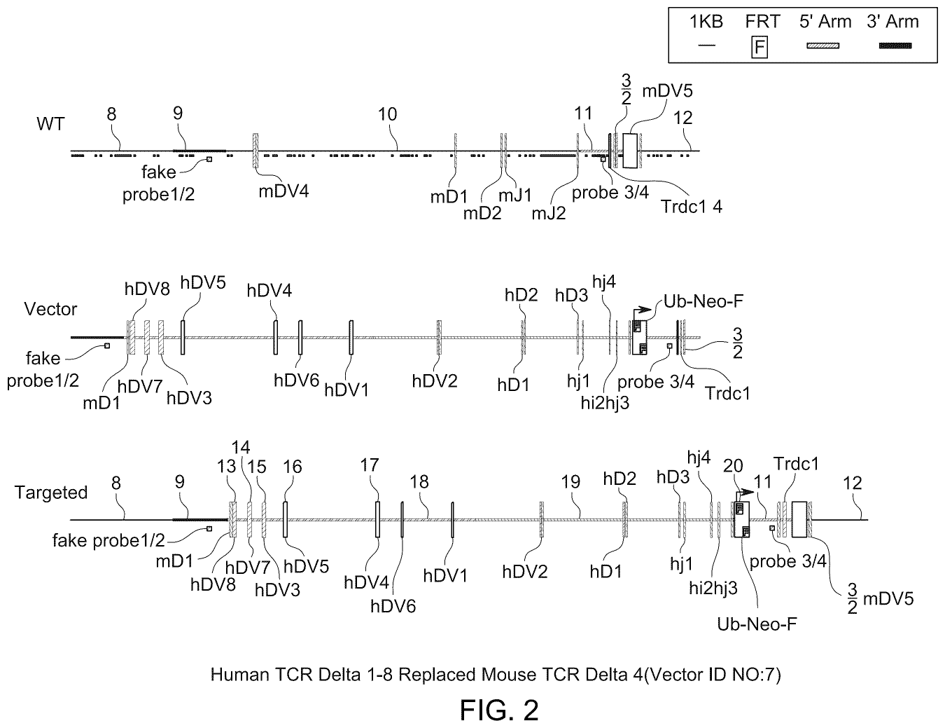

In this example, a non-human animal (such as a mouse) was modified so that the non-human animal contained a nucleic acid sequence comprising human TCRδ further comprising human TCR δ V, D, and J. Specifically, an endogenous mouse sequence containing mouse TCR δ V, D, and J. was replaced with a sequence containing human TCR δ V, D, and J. and operably linked to a mouse TCR Cδ region (FIG. 2).

More specifically, 67562 bp of mouse DNA sequence (DNA segment ID NO: 10) including mouse TCR Trdv4 (mouse Vδ1) corresponding to mDV4, mouse TCR TrDδ 1, 2 and Trdj 1, 2 corresponding to mD1, 2 and mJ1, 2 (respectively) was replaced with a total of 97,129 by of hybrid DNA sequences (82.4% is a human sequence) including 8 human Vs (hVδ1-8), 3 human Ds (hDδ1-3), and 4 human Js (hδJ1-4). The hybrid sequence ended 5′ upstream of the mouse endogenous Cδ region, so human δ Vs, Ds, and Js were genetically linked to the mouse Cδ region. The humanization strategy was summarized in FIG. 2. Junctional nucleic acid sequences inside the targeting vector among rabbit, bovine, mouse, human, and prokaryotic/eukaryotic selection cassettes are summarized in Table 2 and also provided in the Sequence Listing (Seq ID NO: 6-16).

TABLE 2
Junctional Nucleic acid sequences for Vdelta
Targeting Vector (vector ID NO: 7)

| SEQ ID NO | Description |
|---|---|
| 6 | 5' starting nucleic acid sequence of DNA segment NO: |
| | 9 of the 5 mouse homology arm inside mouse |
| | TCRdelta locus |
| 7 | Junctional nucleic acid sequence between 3' end of |
| | mouse S'arm (DNA segment NO: 9) and the starting |
| | of the S' end of rabbit VD2 Promoter sequence (DNA |
| | segment NO: 13) |
| 8 | Junctional nucleic acid sequence between 3' end of |
| | human DV8 sequence (DNA segment NO: 13) and S' |
| | end of the rabbit VD1 promoter sequence (DNA |
| | segment NO: 14) |
| 9 | Junctional nucleic acid sequence between 3' end of |
| | human DV7 sequence (DNA segment NO: 14) and 5' |
| | end of the bovine VD.1. promoter sequence |
| | (DNA segment NO: 15) |
| 10 | Junctional nucleic acid sequence between 3' end of |
| | human DV3 sequence (DNA segment NO: 15) and S' |
| | end of human VDS including promoter sequence |
| | (DNA segment NO: 16) |
| 11 | Junctional nucleic acid sequence between 3' end of |
| | human DV5 sequence (DNA segment NO: 16) and 5' |
| | end of human VD4 including promoter sequence |
| | (DNA segment NO: 17) |
| 12 | Junctional nucleic acid sequence between 3' end of |
| | human DV4 sequence (DNA segment NO: 17) and S' |
| | end of human VD6 including promoter |
| | sequence (DNA segment NO: 18) |
| 13 | Junctional nucleic acid sequence between 3' end of |
| | human DVI sequence (DNA segment NO: 18) and 5' |
| | end of human VD2 including promoter sequence |
| | (DNA segment NO: 19) |
| 14 | Junctional nucleic acid sequence between 3' end of |
| | human sequence (DNA segment NO: 19) and S' end of |
| | the selection cassette (DNA segment NO: 20) |
| 15 | Junctional nucleic acid sequence between 3' end of the |
| | selection cassette (DNA segment NO: 20) and 5' end of |
| | 3' mouse homology arm (DNA segment NO: 11) |
| 16 | 3' ending nucleic acid sequence of DNA segment NO: |
| | 11 of the 3' mouse homology arm inside mouse |
| | TCRdelta locus |

The initial step of generation of a genetically modified mouse with human TCR δ was to construct a TCR δ targeting vector (Vector ID NO: 7), which has ten DNA segments (DNA segment ID NO: 9, 13-20, 11) detailed description in Table 3.

TABLE 3
DNA Segments for Vdelta Targeting Vector (vector ID NO: 7)

| Segment ID | NO Description |
|---|---|
| 9 | A 5' mouse homology arm located 5281 bp up-stream |
| | of mouse Trdv4 (mouse V delta 1) |
| | has a DNA length of 10308 bp |
| 13 | A rabbit V delta promoter (VD2) with DNA length of |
| | 1161 bp linked with human V delta 8 with a |
| | DNA length of 950 bp |
| 14 | A rabbit V delta promoter (VD1) with DNA length of |
| | 1914 bp linked with human V delta 7 with a DNA |
| | length of 742 bp |
| 15 | A bovine V delta promoter (VD1) with ONA |
| | length of 2042 bp linked with human |
| | V delta 3 with a DNA length of |
| 16 | A human DNA sequence including human V |
| | 753 bp delta 5 with a DNA length of 9729 bp. |
| 17 | A human DNA sequence including human V delta 4 |
| | with a DNA length of 13417 bp |
| 18 | A human DNA sequence including human V delta |
| | 6 and 1 with a DNA length of 16820 bp |
| 19 | A human DNA sequence including human V delta 2, |
| | human D Delta 1-3, and human J delta 1-4 |
| | with a DNA length of 49601 bp |
| 20 | coding sequence and flanked by frt sites with a DNA |
| | length of A selection cassette including an Ub |
| | promoter and Kan/Neo 2727 bp |
| 11 | A 3' mouse homology arm located up-stream of mouse |
| | C delta Region with a DNA length of 10001 bp |

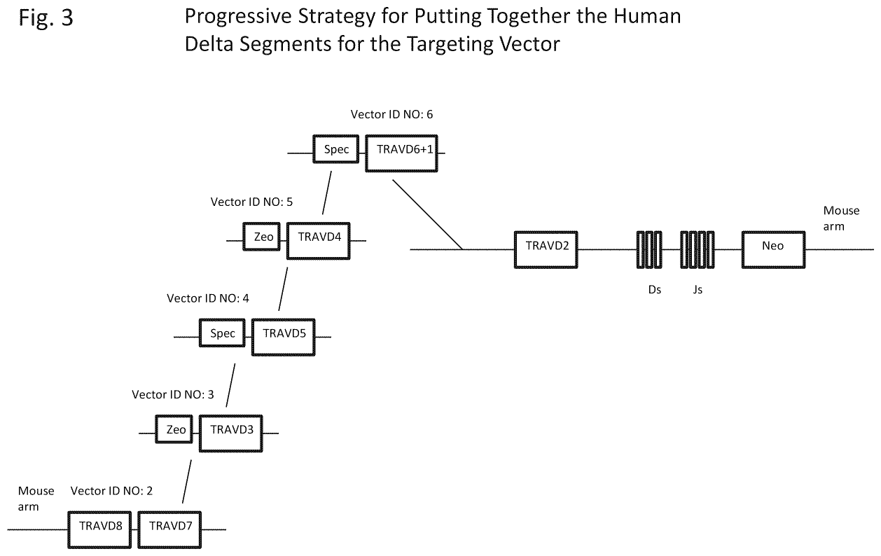

BAC clones were used for the construction of the TCR δ targeting vector (Vector ID NO: 7) including a mouse RP23-6A14 and human CTD-3112B11, CH17-153K15, CTG-3064K1, CTD-2012J23, CTD-2521H9, CTD-2124P16, CTD-3012C18, CTD-3112B11, CTD-2382D16, CTD2382D16 (Invitrogen). Prior to completing the TCR δ targeting vector (Vector ID NO: 7), five mini DNA vectors flanked with bacterial homology arms were constructed through DNA ligation and bacteria homology recombination so that they all share the same 5′ arm and the 3′ arm has sequences shared with DNA sequence of 5′ end of next insertion site (FIG. 3). The detailed description of the five mini DNA vectors was shown in Table 4. To complete the TCR δ targeting vector (Vector ID NO: 7), a strategy of alternative antibiotic selection (e.g., first Spec selection, second Zeo selection, third Spec selection, fourth Zeo selection, etc.) to introduce and connect different DNA segments was based on bacterial homology recombination and PCR confirmation to achieve a lineal sequence composed of DNA segment 9, 13-19. Based on bacterial homology recombination, DNA segments 20 and 11 were also connected to DNA segment 19 to complete the TCR δ targeting vector (Vector ID NO: 7). During the targeting vector construction process, a unique (not cutting into DNA segment NO: 9, 13-20, 11) enzyme cutting site AscI was introduced to allow linearization of the targeting vector (Vector ID NO: 7). The linearized targeting vector (Vector ID NO: 7) was electroporated into a hybrid mouse embryonic stem (ES) cell line derived from a hybrid strain by crossing mice of two different inbred strains (B6 and 129). ES cells with correct targeted clones were identified and confirmed by a combination of various methods (e.g., PCR-based essays, long arm PCR, long-range PCR, southern, and; or human replacement PCR) known in the art. Targeted ES clones were expanded and micro-injected into mouse blastocysts to generate chimera mice with human targeted gene segment (DNA segment NO: 13-19). Germline mouse was obtained by further mating the chimera with B6 inbreed and additional PCR confirmation was performed. The Neo cassette was removed by the activity of Flp recombinase in ES cells or mouse tissues.

TABLE 4
Vector IDs contributing to Vdelta
Targeting Vector (vector ID NO: 7)

| Vector ID NO | Description |
|---|---|
| 2 | Containing a S' bacterial homology arm, |
| | a Spec selection cassette, DNA sequence |
| | with DNA segments ID NO: 9, |
| | 13, 14, and a 3' bacterial homology arm |
| 3 | Containing a S' bacterial homology arm, |
| | a Zeo selection cassette, DNA sequence |
| | with DNA segments ID NO: 15, |
| | and a 3' bacterial homology arm |
| 4 | Containing a 5' bacterial homology arm, a |
| | Spec selectioncassette, DNA sequence |
| | with DNA segments ID NO: 16, |
| | and a 3' bacterial homology arm |
| 5 | Containing a S' bacterial homology arm, a |
| | Zeo selection cassette, DNA sequence |
| | with DNA segments ID NO: 17, |
| | and a 3' bacterial homology arm |
| 6 | Containing a S' bacterial homology arm, a |
| | Spec selection cassette, DNA sequence with |
| | DNA segments ID NO: 18,and a 3' bacterial |
| | homology arm which shared sequence |
| | with 5' end of DNA segment ID NO: 19 |

### Example 3

Generation of Double Homozygous for Humanized γ TCR

Humanized γ and δ heterozygous mice achieved in Examples 1 and 2 were inter-bred. The resulting progeny included WT, homozygous human γ TCR/heterozygous δ TCR mice, homozygous human δ TCR/heterozygous γ TCR mice, heterozygous human γ TCR, heterozygous δ TCR mice, and double homozygous human γδ TCR mice by PCR amplification of tail DNA from these mice. The disappearance of the WT PCR band at the replaced region of TCR γ and δ loci was the indication of homozygosity of human transgene.

### Example 4

Expression Analysis of TCRδ from Humanized TCRδ Mice

The TCRδ CDR3 repertoire diversity reflects TCRδ T cell development, maturation, clonal composition, potential antigenic recognition spectrum, and quantity of available T-cell responses. The humanized TCRδ mice were able to use the endogenous mouse VDJ recombination enzymatic system to recombine human Vδ, human Dδ, and human Jδ to form human TCRδ CDR3, which was fully contributed from human genomic sequence introduced by targeted replacement of mouse endogenous sequence at the TCRδ locus. Specifically, blood and various tissues were collected from a homozygous human Vδ mouse. Blood was first treated with Red Blood Cell Lysis buffer (0.8 g NH4Cl and 0.11 g NaHCO₃ in 100 ml water-based solution) to remove red blood cells and white blood cells including T cells were collected by centrifugation, which were processed the same way as rest of the tissues. Tissues including 1-Peritoneal fluid, 2-brain (olfactory), 3-ear (middle part), 4-nose, 5-tough, 6-lymph nodes, 7-throat, 8-lung, 9-spleen, 10-bone marrow, 11-Eye (eyeball), 12, 13, 14 (three beginning, middle, end) parts of the small intestine, 15-uterus track, 16-kidney, 17-thymus, 18-muscle, 19-heart, 20-liver, 21-skin, 22-blood were collected and total RNA was isolated by using RNeasy Mini Kit (Qiagen) according to manufacture suggestions. Total RNA was translated into cDNA by using random priming oligos provide with the Transcriptor First Strand cDNA Synthesis Kit (Roche) in the total volume of 15 ul. One microliter of resulting cDNA was used as a template to conduct a first round (round 1) of the PCR reaction (total 40 ul) with primer pairs covering human Vδ to mouse C region including CRD3 region. For each Vδ CDR3 detection, one primer was located at the 5° end of Vδ and a second primer was designed and located inside the exon 1 of mouse C region also served as a common anti-sense primer for all the Vδ1-8 PCR reaction (Seq ID NO: 18). For example, as shown in table 5, first round PCR of CDR3 of human Vδ1 was amplified between a primer (Seq ID NO: 19) and the common Cδ primer (Seq ID NO: 18). First round PCR of CDR3 of human Vδ2 was amplified between a primer (Seq ID NO: 20) and the common Cδ region primer (Seq ID NO: 18) and continued in the same fashion. Because of the un-purify of the total RNA, a second round of PCR is generally needed.

TABLE 5
Primers for detecting RNA Expression of human DV1-8 in humanized mouse
(PCR round 1)

| Human Vdelta | Primer sequence (5′-3′) | Seq ID NO | New SEQ ID No. |
|---|---|---|---|
| hDV1-8 | GTCAGTCGAGTGCACAGTTTCACTG | 18 | 51002 |
| | (common anti-sense) | | |
| hDV1 | CCCAGTCATCAGTATCCATGCCAG | 19 | 51003 |
| hDV2 | TGAACACCAAACAGTGCCTGTGTC | 20 | 51004 |
| hDV3 | GTTCCCCGGACCAGACGGTGGCGAG | 21 | 51005 |
| hDV4 | ACCCAACCAGGAATGTTCGTGCAG | 22 | 51006 |
| hDV5 | AACAGTCAACAGAAGAATGATGACCAG | 23 | 51007 |
| hDV6 | GAGAAAAGTGACCAGCAGCAGGTG | 24 | 51008 |
| hDV7 | TGAAGACAAGGTGGTACAAAGCCCTC | 25 | 51009 |
| hDV8 | AGACAGTCACTCAGTCTCAACCAGAG | 26 | 51010 |

For the second round of PCR, two primers were designed within the previous primers described in the first round of PCR. Typically, a primer 50-100 bp down-stream of the A 1 first round primer and a second common primer (Seq ID NO: 27), which is 50-100 bp up-stream of the common primer of the first round (Seq ID NO: 18). One microliter PCR product from round 1 was introduced as a template for second round PCR in a total volume of 40 ul. For example, as shown in Table 6, second round. PCR of CDR3 of human A 1 was amplified between a primer (Seq ID NO: 28) and the common C region primer (Seq ID NO: 27). Second round PCR of CDR3 of human Vδ2 was amplified between a primer (Seq ID NO: 29) and the common C region primer (Seq ID NO: 27) and continued in the same fashion (Table 7). The final PCR products of Vδ1-8 from various tissues were Sanger sequenced and the diversity of human TCR was estimated by examining the reduction of heights vs normal peak on the DNA sequencing chromatogram. H, M, and L represent high, medium, and low diversity of human δ TCR, respectively. As shown in Table 8, RNA of human TCR Vδ1-8 was expressed in the majority of tissues examined and many tissues represented high diversity TCRδ. Particularly, human Vδ1-4 and 6 expressed with high diversification in tissues including peritoneal fluid, spleen, bone marrow, small intestine, and thymus, where γδ T cells were highly presented also in native humans (Silva-Santos, B., 2021. nature reviews immunology 21:221-232 and Hayday, A., 2019. 203: 311-320), In sum, the mouse model with human δ locus was able to recapitulate VDJ recombination as in native humans.

TABLE 6
Primers for detecting RNA Expression of human DV1-8 in humanized mouse
(PCR round 2)

| Human Vdelta | Primer sequence (5′-3′) | Seq ID NO | New SEQ ID No. |
|---|---|---|---|
| hDV1-8 | CATACTGACCAAGCTTGACAGCACTG | 27 | 51011 |
| | (common anti-sense) | | |
| hDV1 | TGCCTGTATGAAACAAGTTGGTGGTC | 28 | 51012 |
| hDV2 | GGTACAGGAAGACCCAAGGTAACAC | 29 | 51013 |
| hDV3 | GGTGGTACTGCTCTGCACTTACGAC | 30 | 51014 |
| hDV4 | TTCTGGTACAAGCAGCCCAGCAGTG | 31 | 51015 |
| hDV5 | ACCATCCCTGAGCGTCCAGGAAG | 32 | 51016 |
| hDV6 | TGCTTATGAGAACACTGCGTTTGAC | 33 | 51017 |
| hDV7 | TCCACGAGGGAGACACCGTAACTC | 34 | 51018 |
| hDV8 | TGCACATATGACACCAGTGAGAGTG | 35 | 51019 |

TABLE 7
Human Vdelta Expression in Different Mouse Tissues

| Human | Tissues of Human TCRdelta Mice | | | | | | | | | | | | | | | | | | | | | |
|---|---|---|---|---|---|---|---|---|---|---|---|---|---|---|---|---|---|---|---|---|---|---|
| Vdelta | 1 | 2 | 3 | 4 | 5 | 6 | 7 | 8 | 9 | 10 | 11 | 12 | 13 | 14 | 15 | 16 | 17 | 18 | 19 | 20 | 21 | 22 |
| hDV1 | + | | + | + | + | | + | + | + | + | | + | + | + | + | + | | + | + | + | + | |
| hDV2 | + | | + | | | | | | + | + | | | + | + | | | + | | | | | + |
| hDV3 | + | | | | | | | | + | + | | + | + | + | | | + | | + | + | | + |
| hDV4 | + | | + | | | | + | + | + | + | | + | + | + | + | + | | | + | | | |
| hDV5 | | | | | | | | | + | | | | + | | | | + | | | | | |
| hDV6 | + | | + | | | | + | + | + | + | | + | + | + | | + | + | | | | + | + |
| hDV7 | | | | | | | | | | | | | + | + | | | | + | | | + | + |
| hDV8 | | | | | | | + | + | | | | | | + | + | | | | | | | |

1-Peritoneal fluid, 2-brain (olfactory), 3-ear (middle part), 4-nose, S-tough, 6-lymph
nodes, 7-throat, 8-lung, 9-spleen, 10-bone marrow, 11-Eye (eve ball), 12, 13, 14
(three beginning, middle, end) parts of small intestine, 15-uterus track, 16-kidney,
17-thymus, 18-muscle, 19-heart, 20-liver, 21-skin, 22-blood. Positive sign
represents expression of human delta CDR3.

TABLE 8
Human Vdelta Diversity in Different Mouse Tissues

| Human | Tissues of Human TCRdelta Mice | | | | | | | | | | | | | | | | | | | | | |
|---|---|---|---|---|---|---|---|---|---|---|---|---|---|---|---|---|---|---|---|---|---|---|
| Vdelta | 1 | 2 | 3 | 4 | 5 | 6 | 7 | 8 | 9 | 10 | 11 | 12 | 13 | 14 | 15 | 16 | 17 | 18 | 19 | 20 | 21 | 22 |
| hDV1 | H | | L | L | M | | H | H | H | H | | H | H | H. | M | M | H | | M | H | L | H |
| hDV2 | L | | L | | | | | | H | L | | M | M | | | | H | | | | | H |
| hDV3 | H | | | | | | | | H | H | | L | H | H | | | H | | L | L | | M |
| hDV4 | M | | M | | | | M | M | H | H | | H | H | H | L | M | | | H | | | |
| hDV5 | | | | | | | | | M | | | | L | | | | H | | | | | |
| hDV6 | M | | L | | | | H | M | H | H | | H | H | H | | L | H | | | | | H |
| hDV7 | | | | | | | | | | | | | L | L | | | L | | | L | | |
| hDV8 | | | | | | | L | L | | | | | L | | | | | | | | | |

1-Peritoneal fluid, 2-brain (olfactory), 3-ear (middle part), 4-nose, 5-tongue, 6-
lymph nodes, 7-throat, 8-lung, 9-spleen, 10-bone marrow, 11-Eye (eve ball), 12, 13,
14 (three beginning, middle, end) parts of small intestine, 15-uterus track, 16-
kidney, 17-thymus, 18-muscle, 19-heart, 20-liver, 21-skin, 22-blood. H-high
diversity, M-medium diversity, L-low diversity of human delta TCR.

### Example 5

Repertoire Analysis of TCRδ from Humanized TCRδ Mice

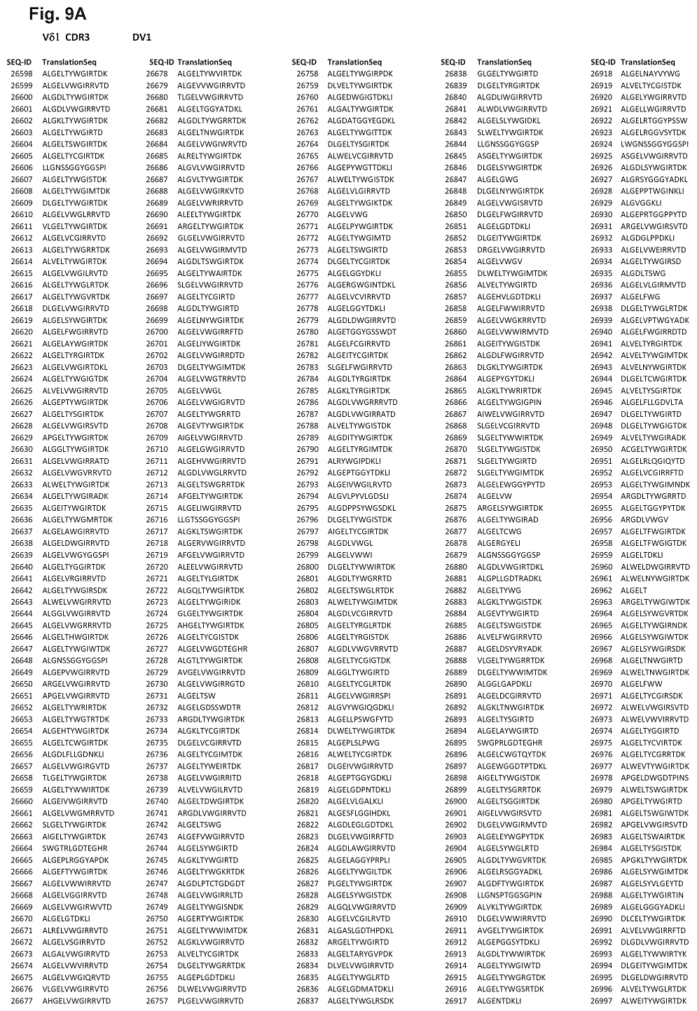

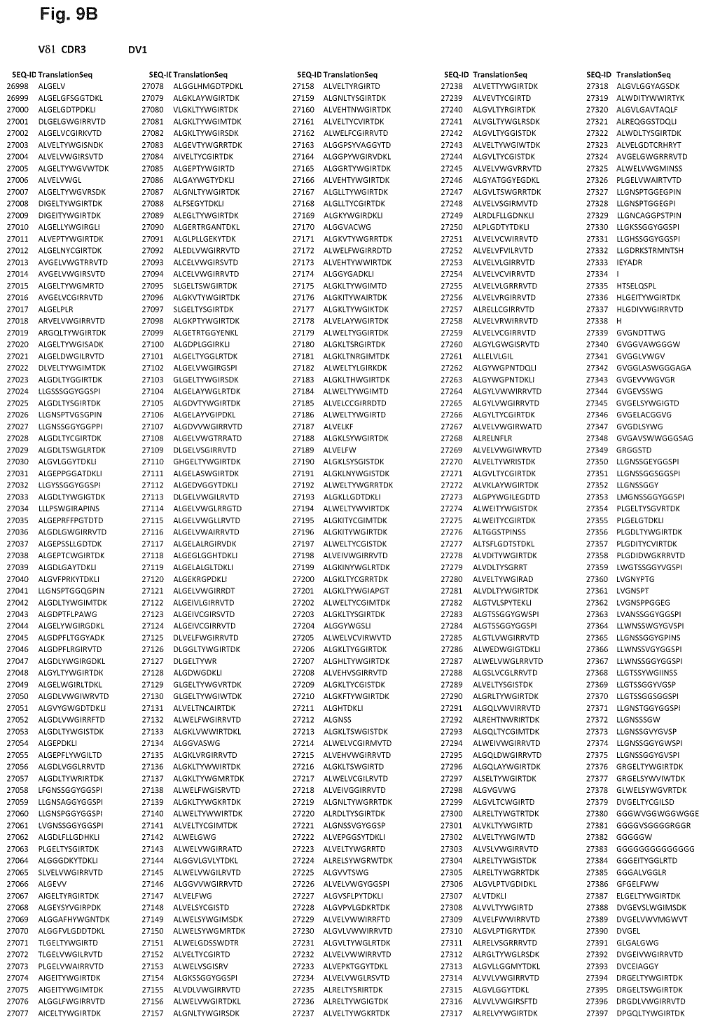

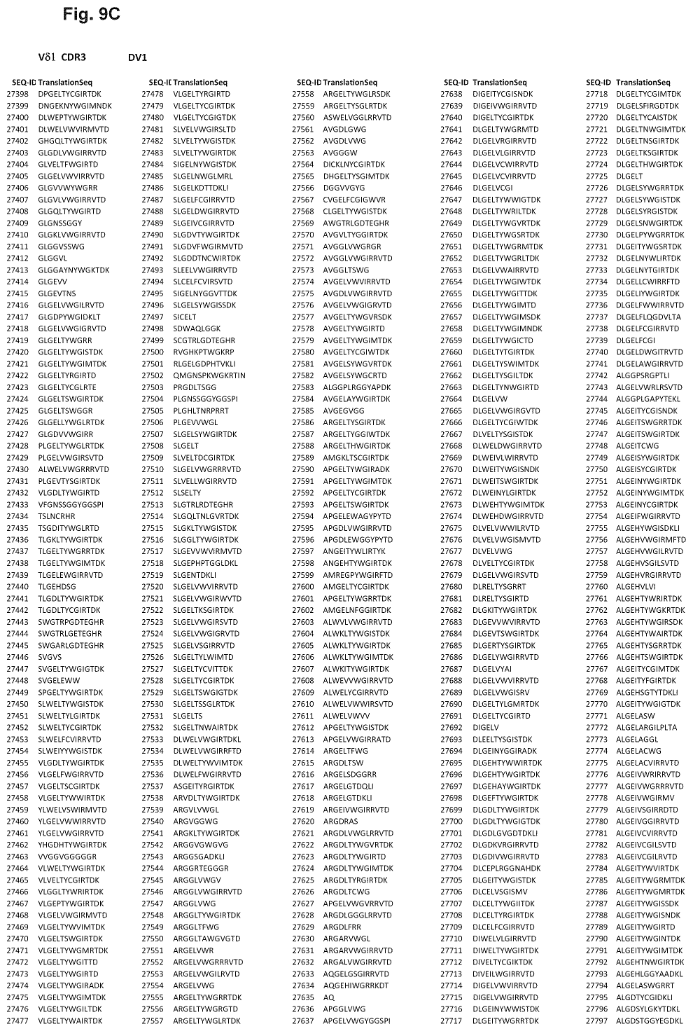

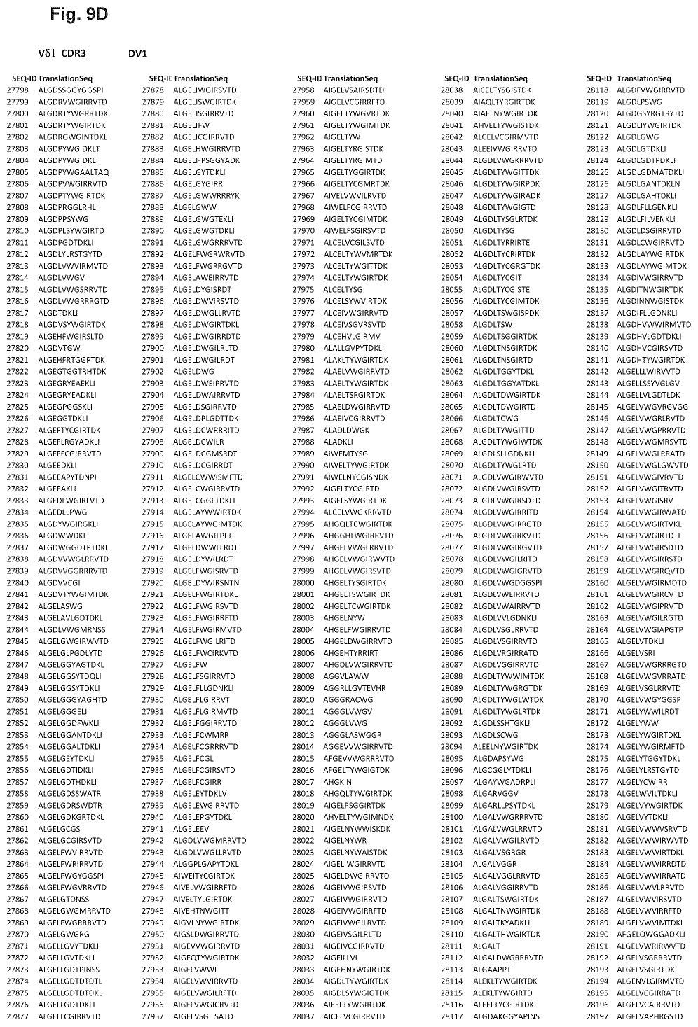

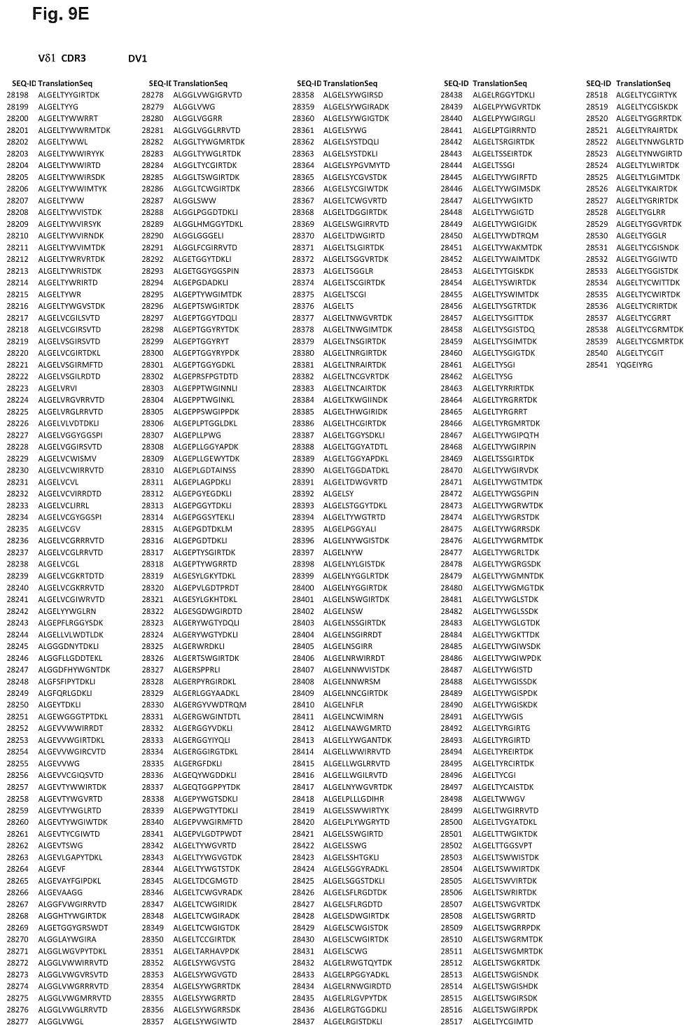

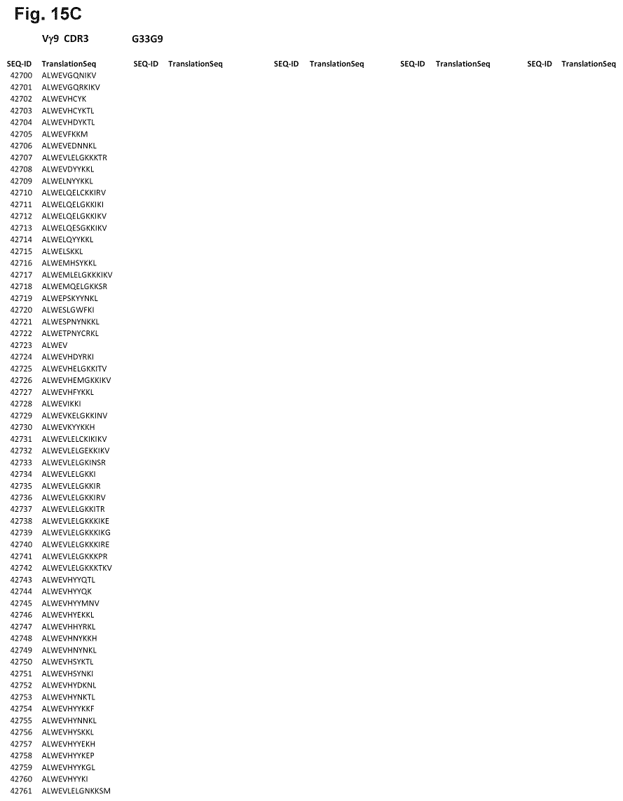

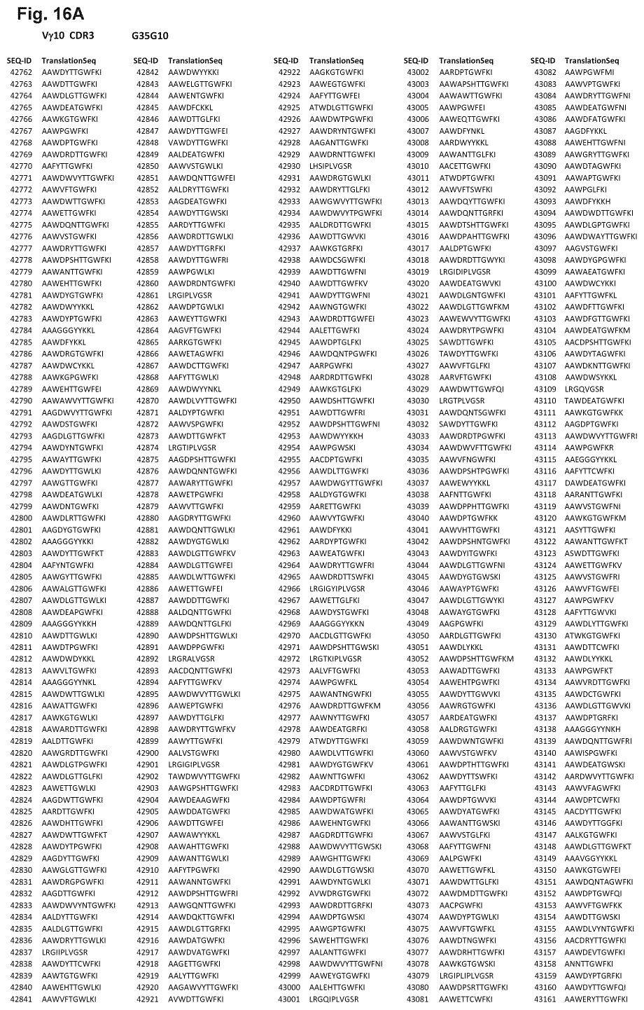

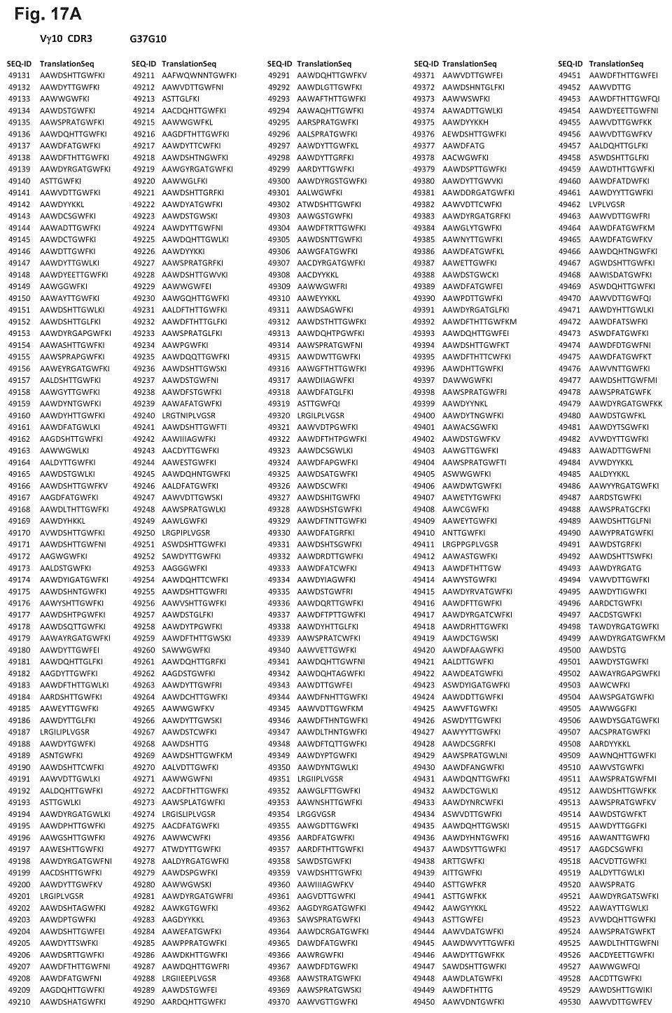

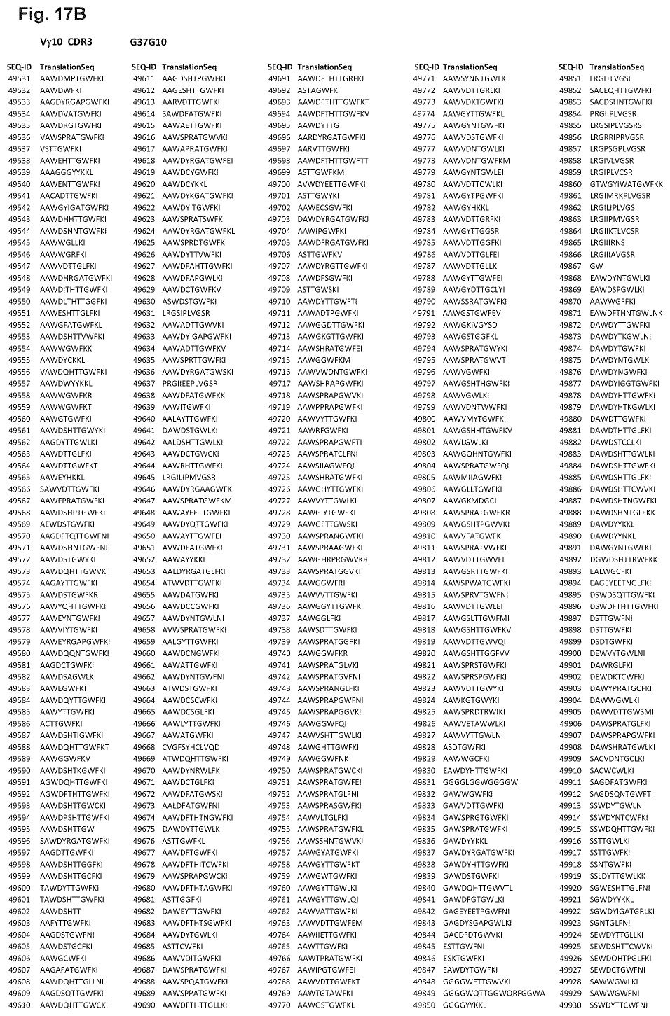

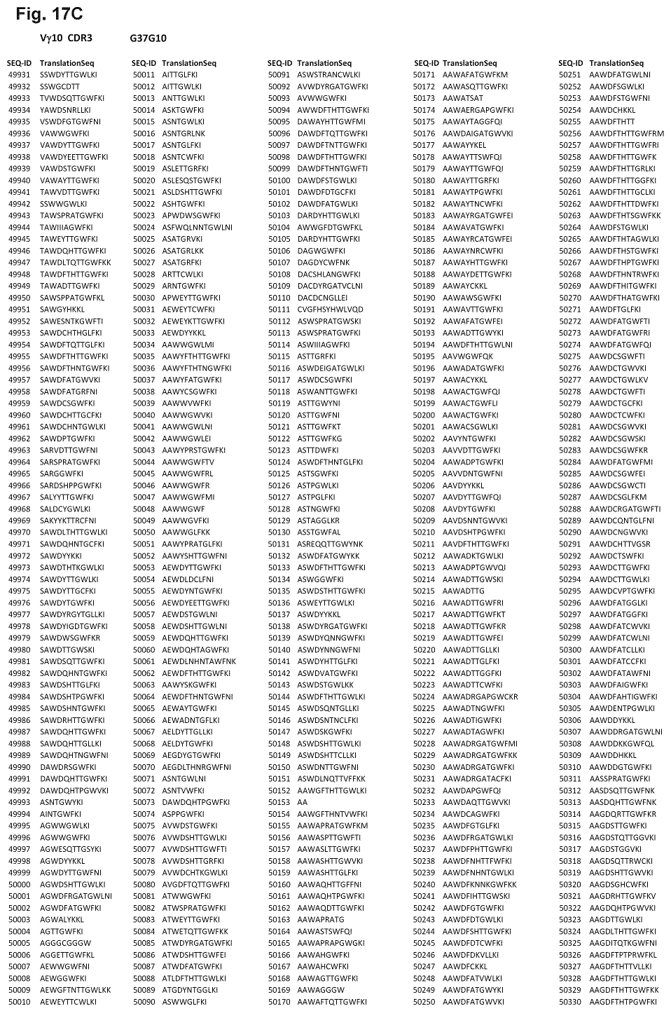

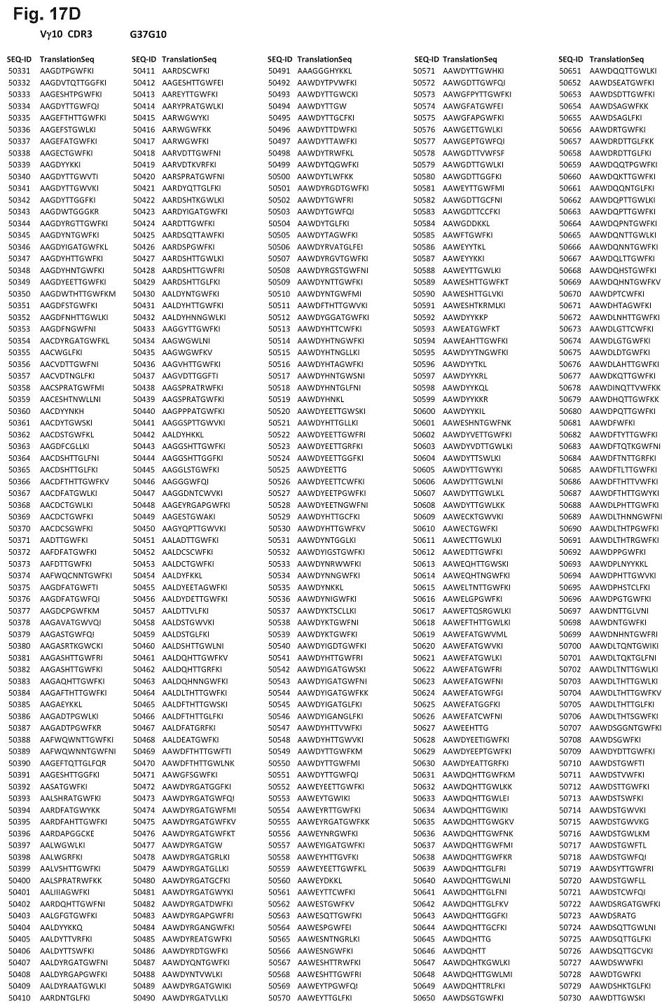

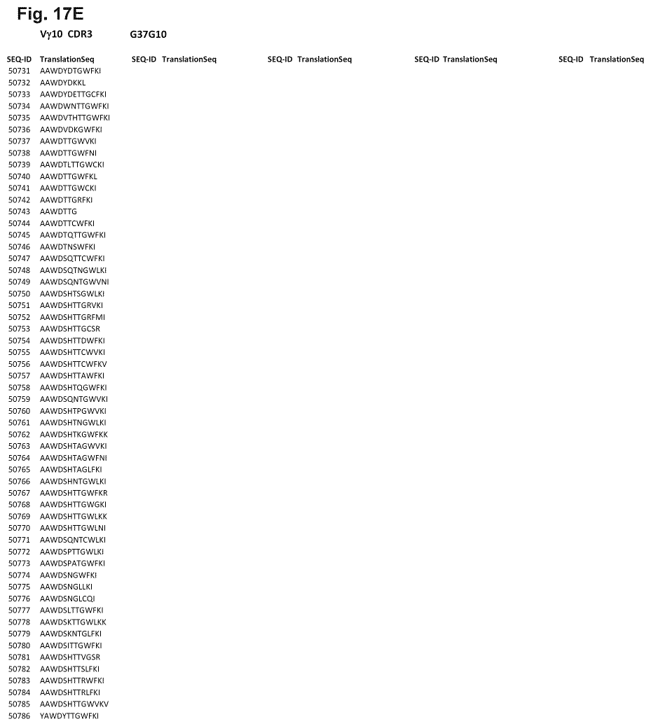

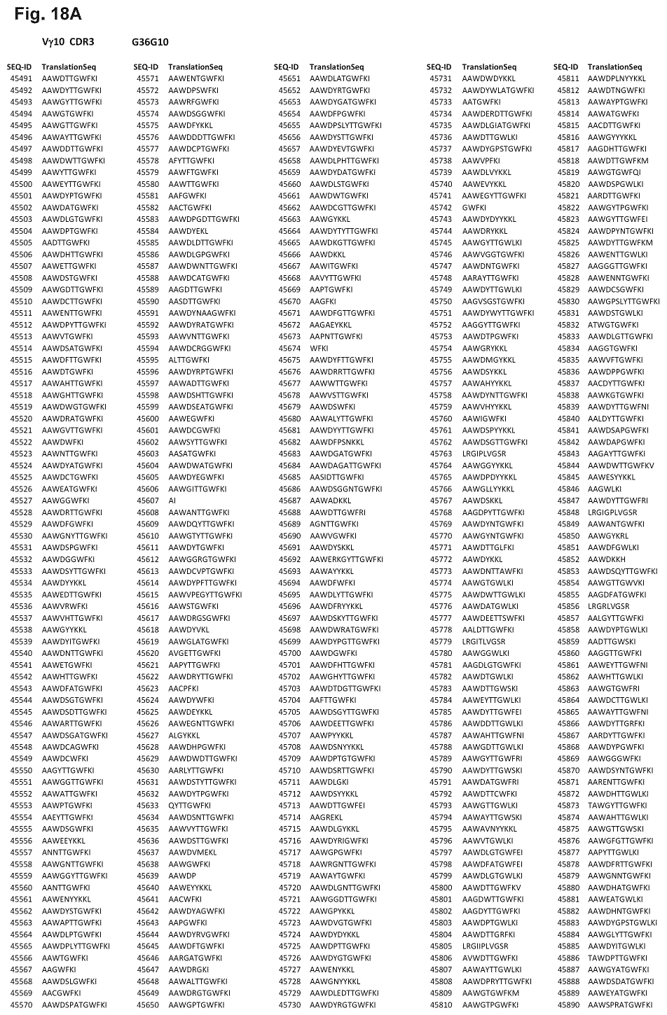

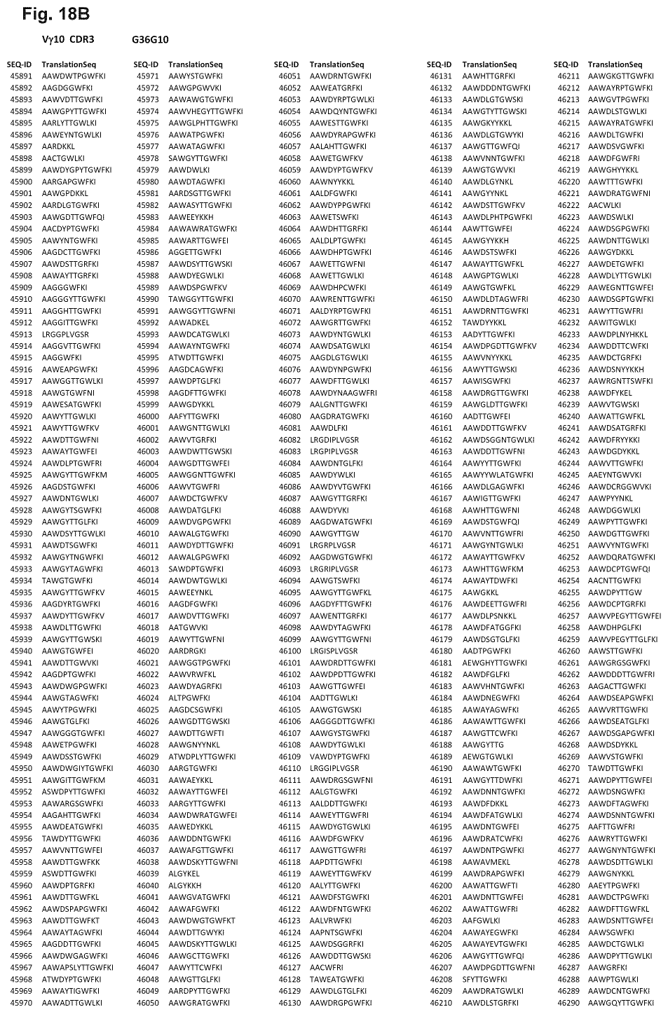

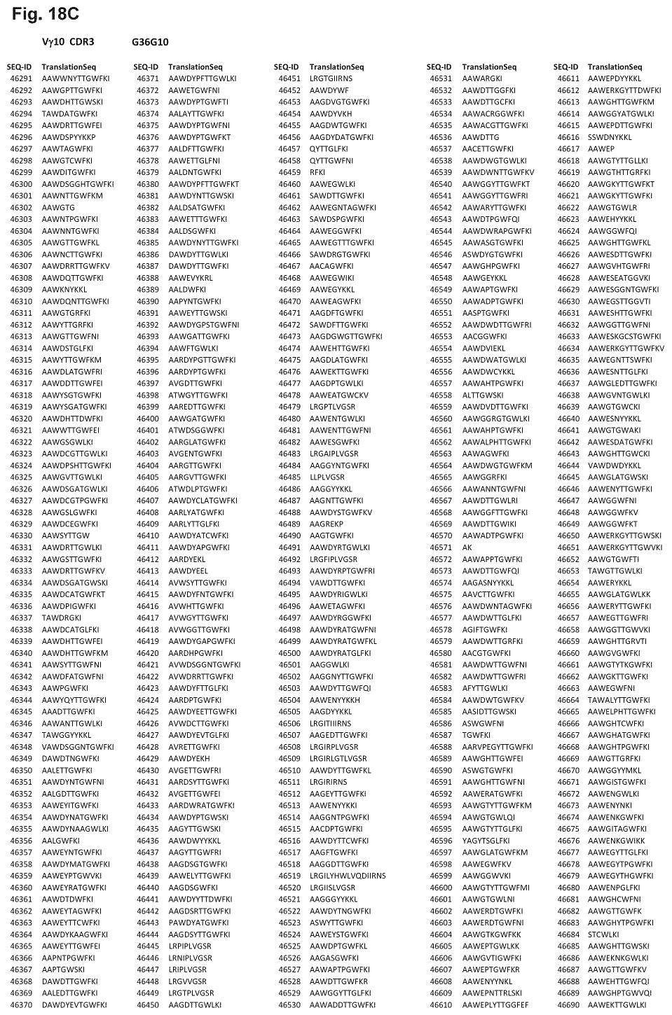

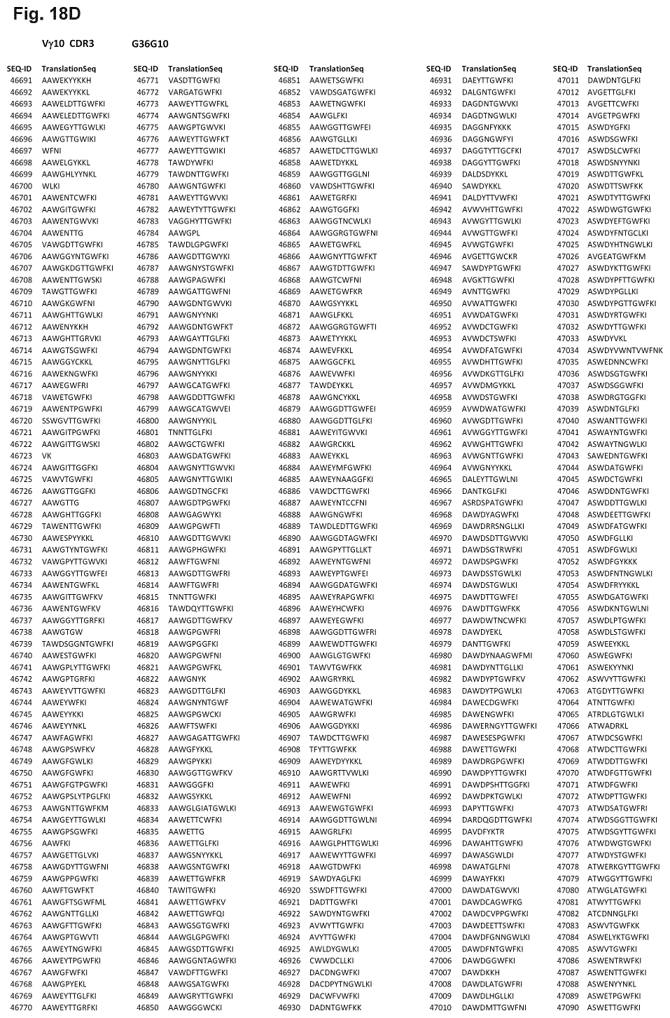

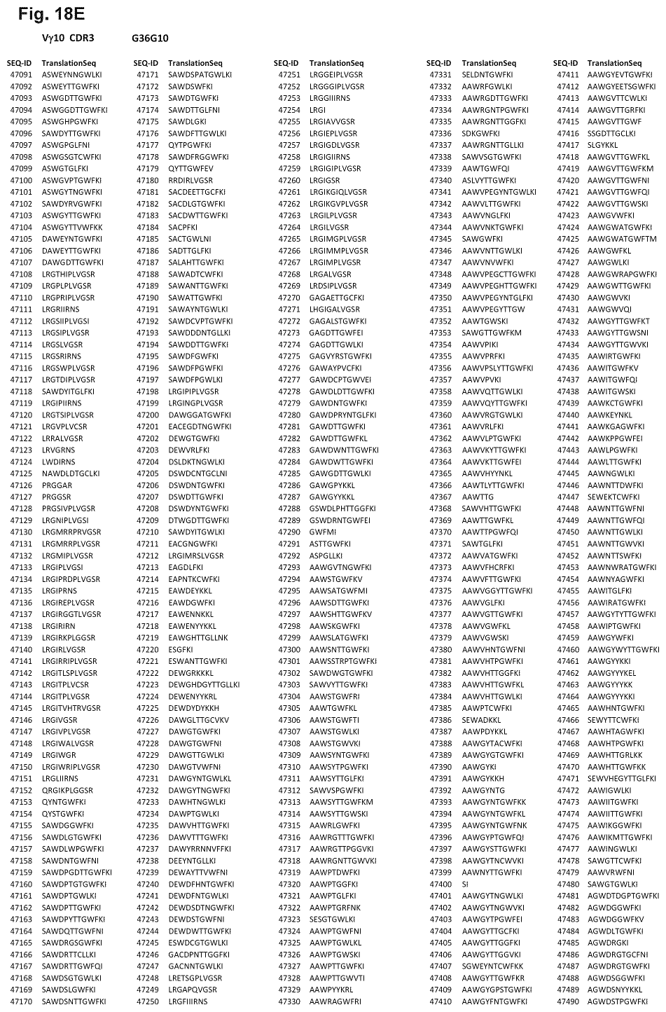

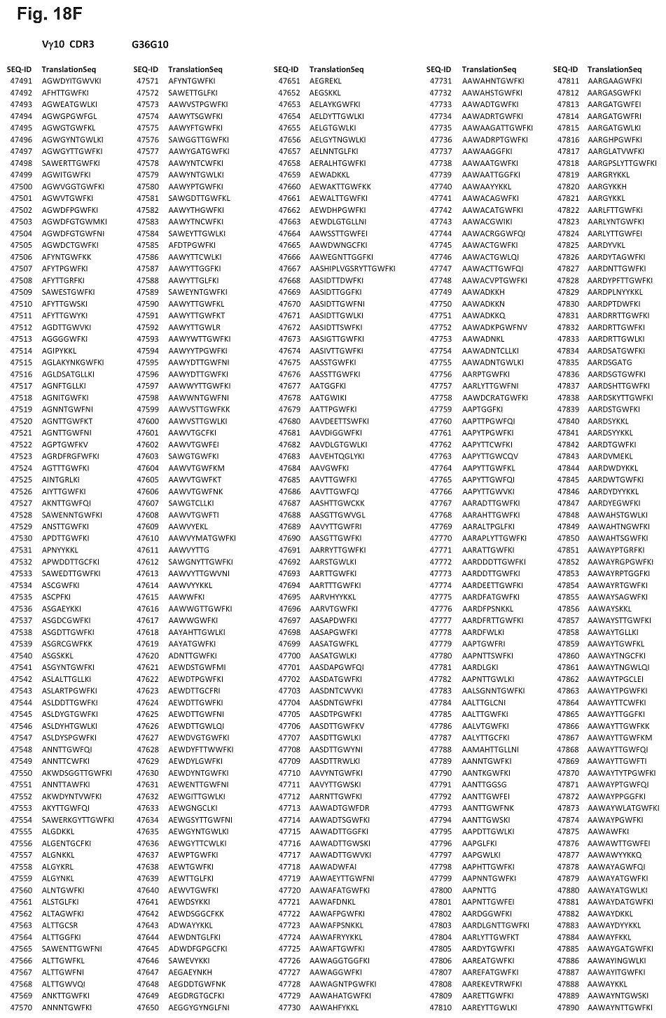

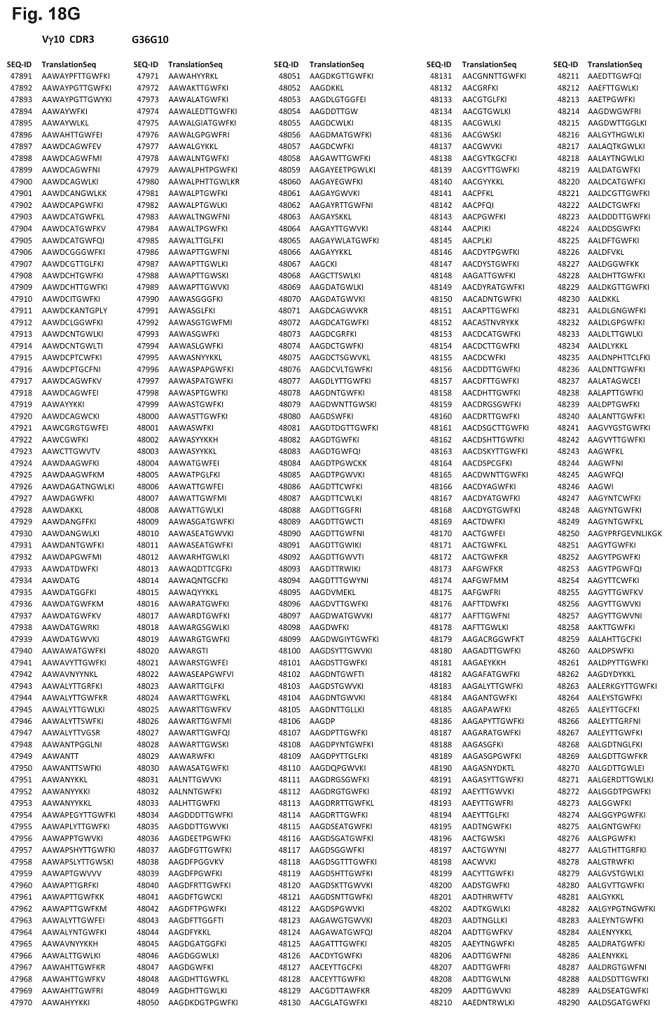

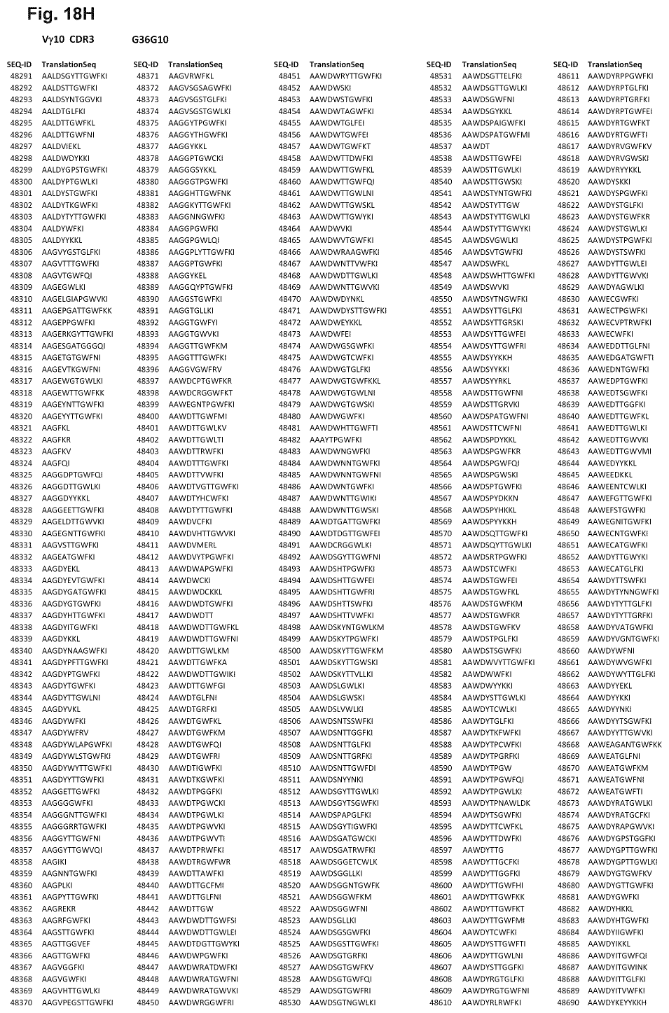

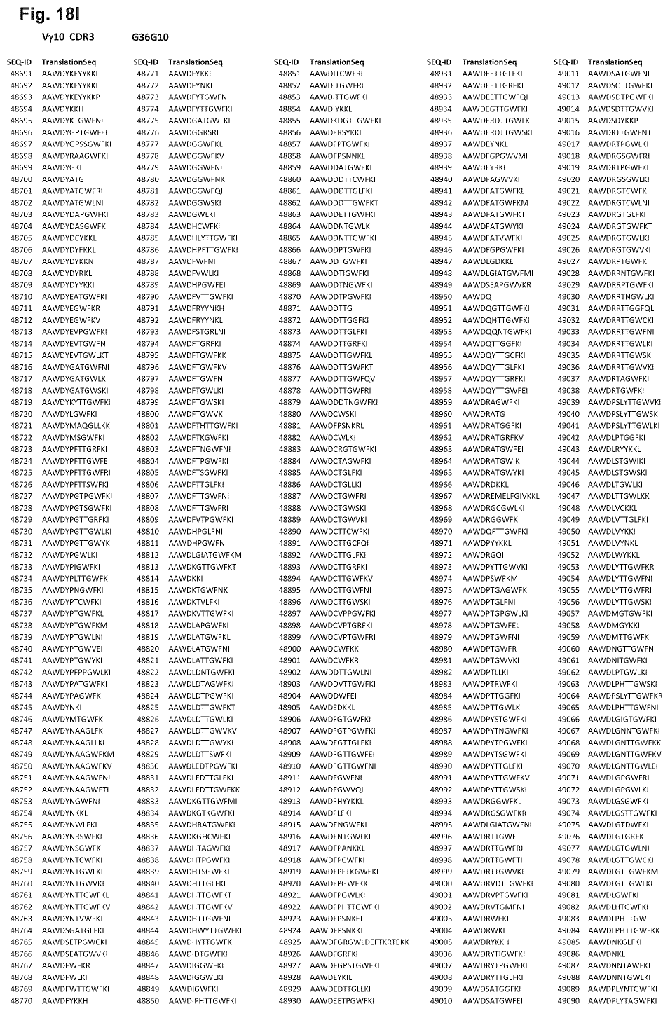

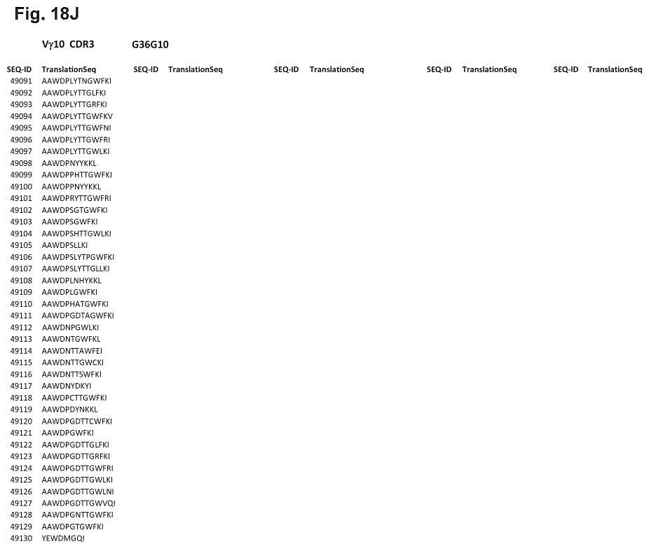

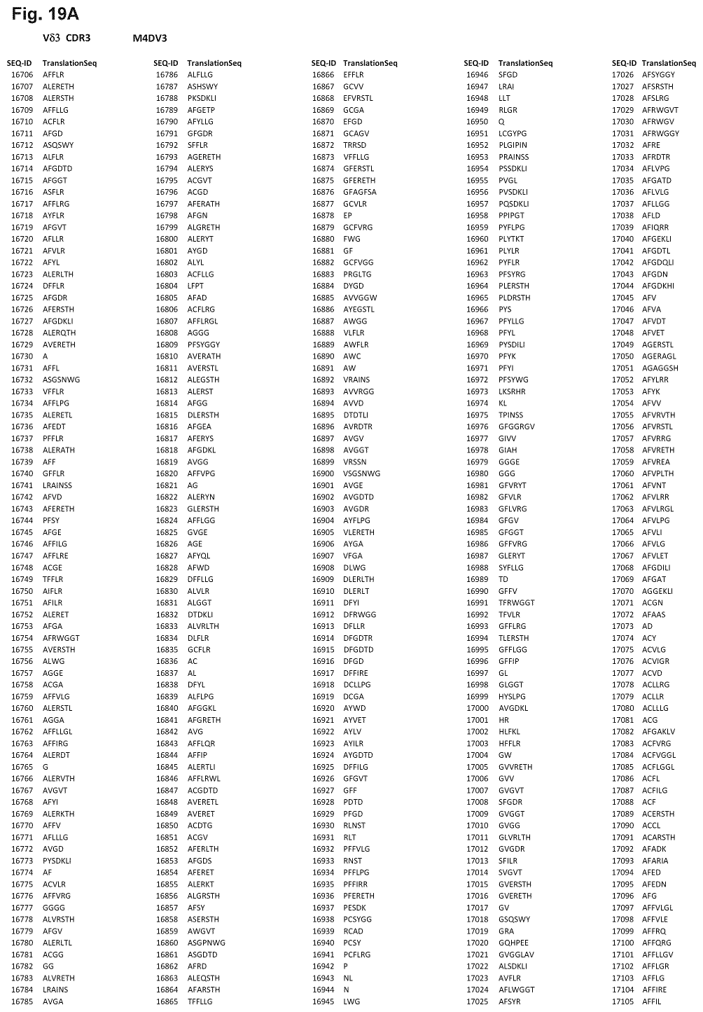

The TCRδ CDR3 repertoire diversity was confirmed by PCR (Example 3) and the CDR3-containing sequence was obtained by subsequently next-generation sequencing (NGS). (FIGS. 9-24)

Specifically, PCR products from the second round PCR using human TCRδ specific primer and mouse Cδ region primer were purified and submitted for next-generation sequencing (Genewiz). Because of over-sized sequence data, only the first 100,000 nucleotide sequences were subjected to repertoire analysis using a function of Analyze T cell receptor (TR) Sequences from IgBlast (https://www.ncbi.nlm.nih.gov/igblast/) to calculate the border and actual amino acid sequence of δ CDR3s. Amino acid sequences of various TCRδ CDR3s along with their respective percent abundance in the tissues of the human TCRδ mouse are listed in the table with their unique sequence IDs (There are other TCR CDR3 analysis platforms and will generate CDR3 sequences with slightly different on the beginning and ending of the border sequence. They can be re-analyzed by IgBlast to have comparable sequences with that of the invention described here).

CDR3s were generated from two experiments with two sets of different human TCR mice.

In the first experiment, blood was collected from two homozygous human TCR mice. PCR products of human TCRδ were obtained for human Vδ1, 3, and 6. The NGS data showed, inside the blood, the CDR3 amino acid sequences were highly diversified.

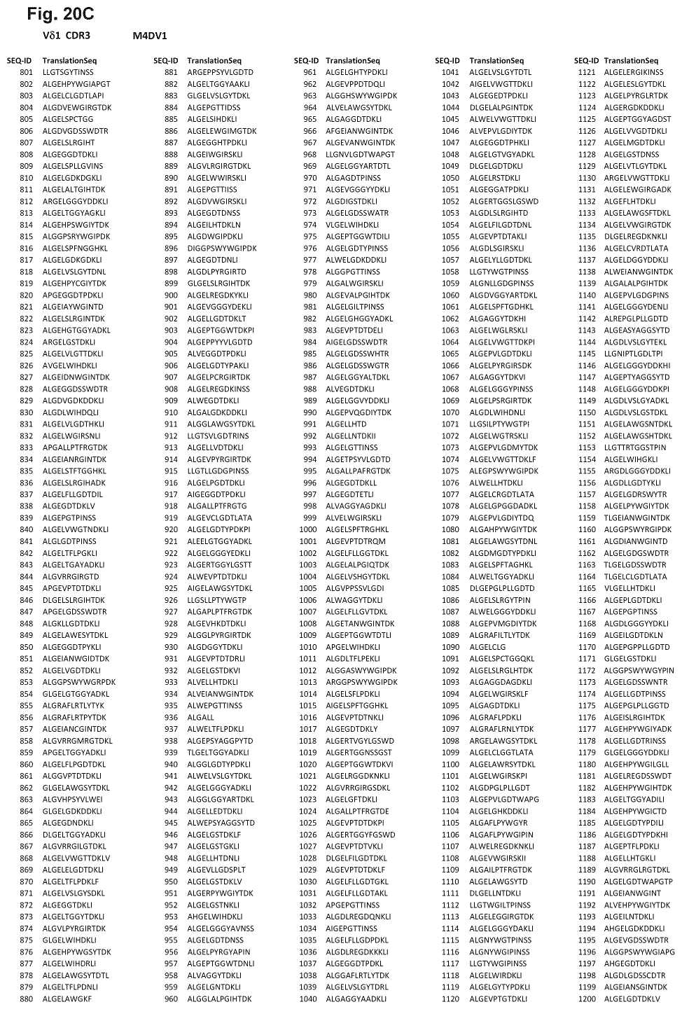

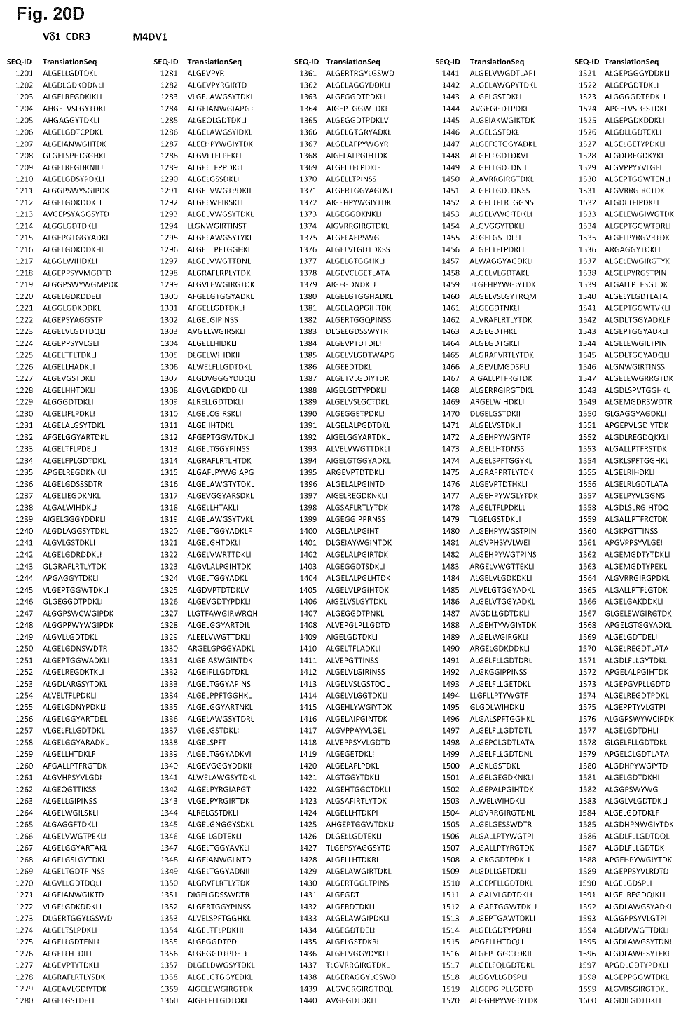

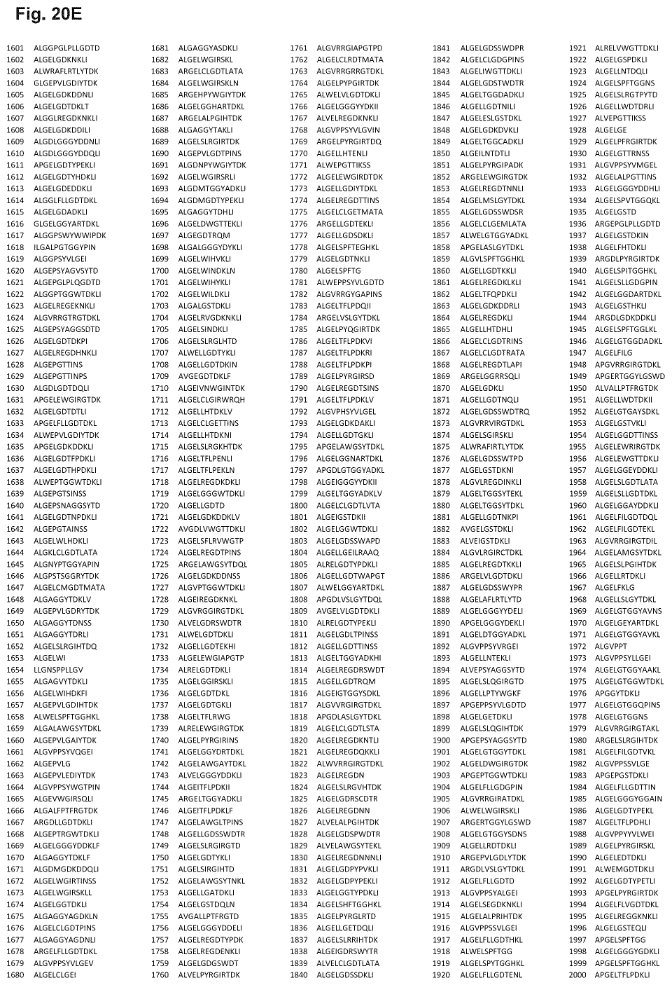

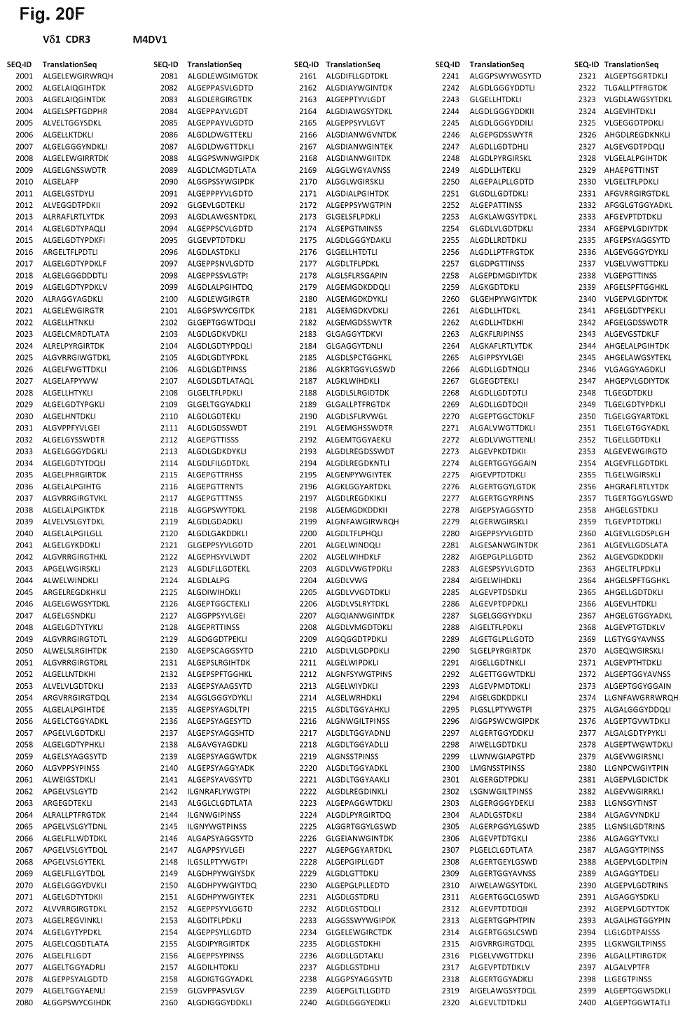

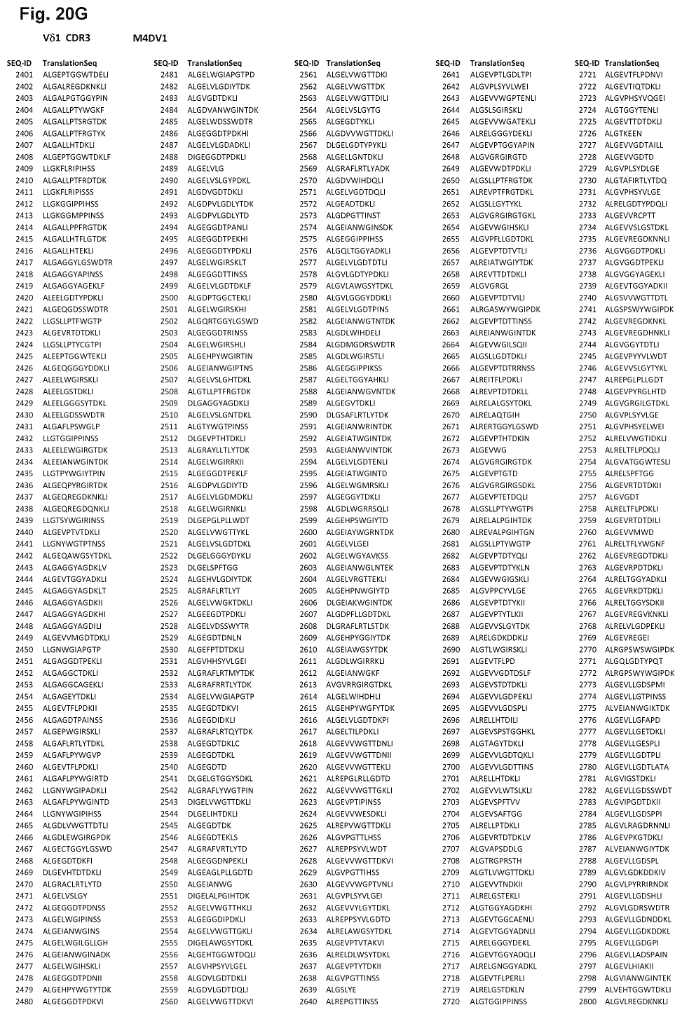

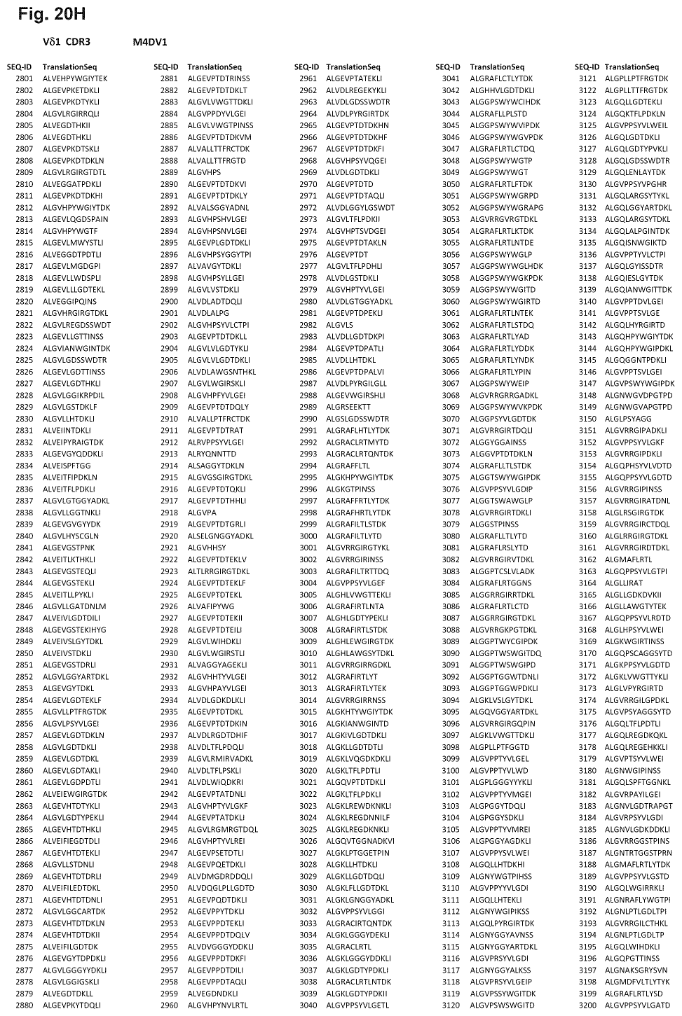

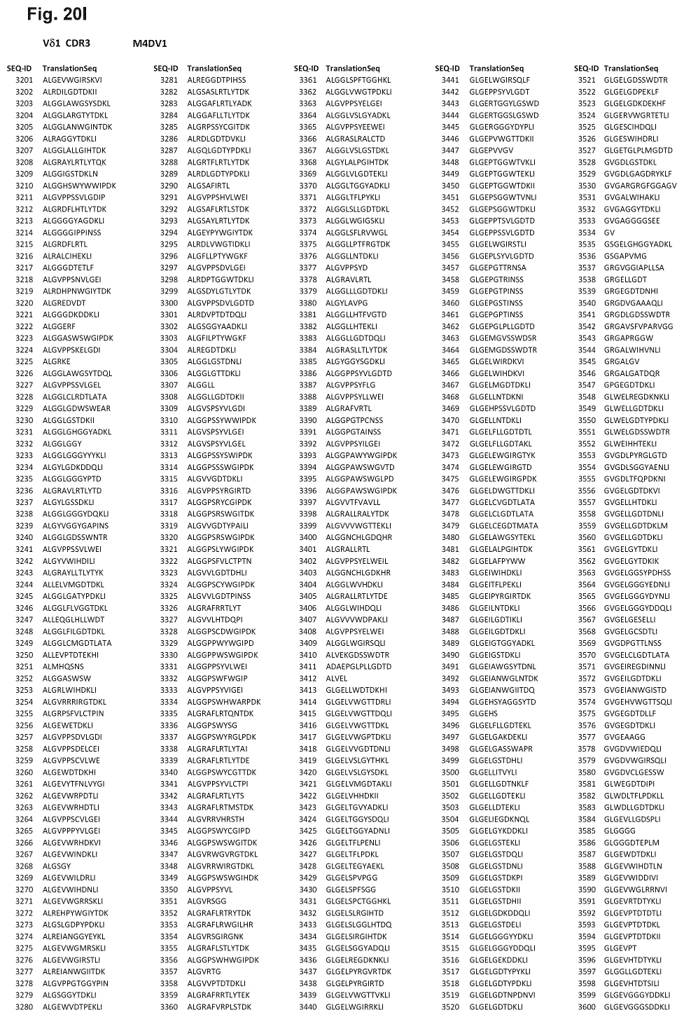

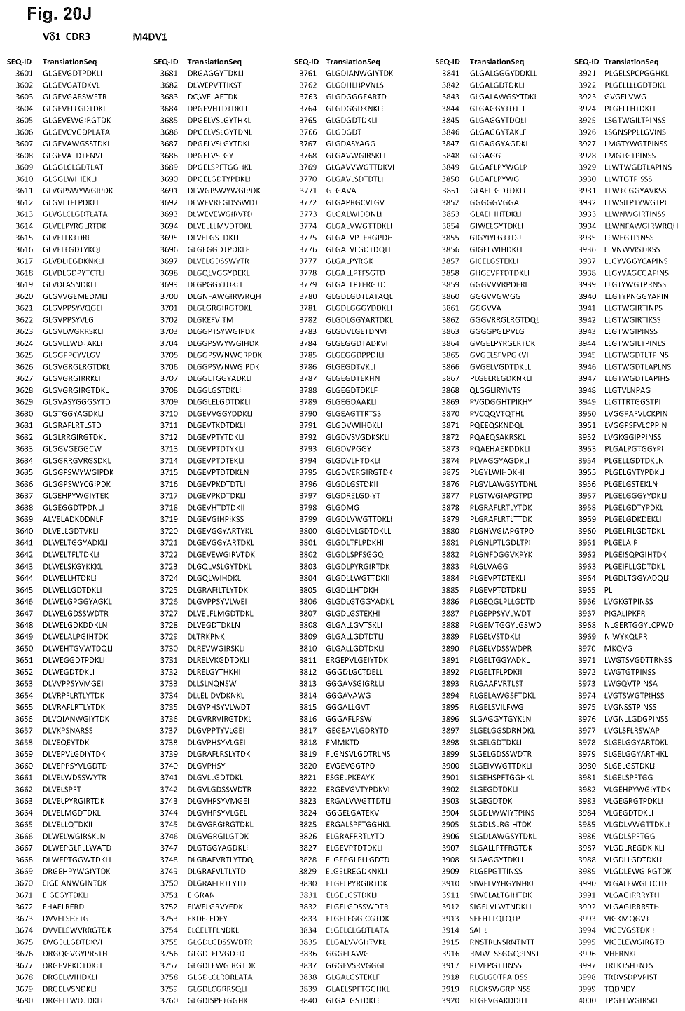

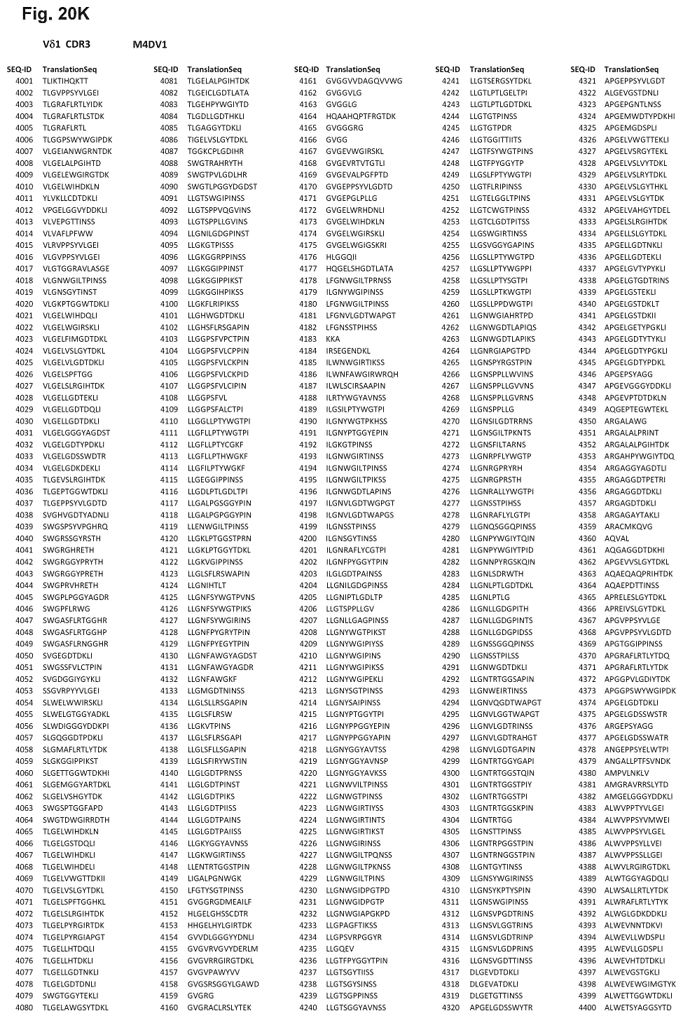

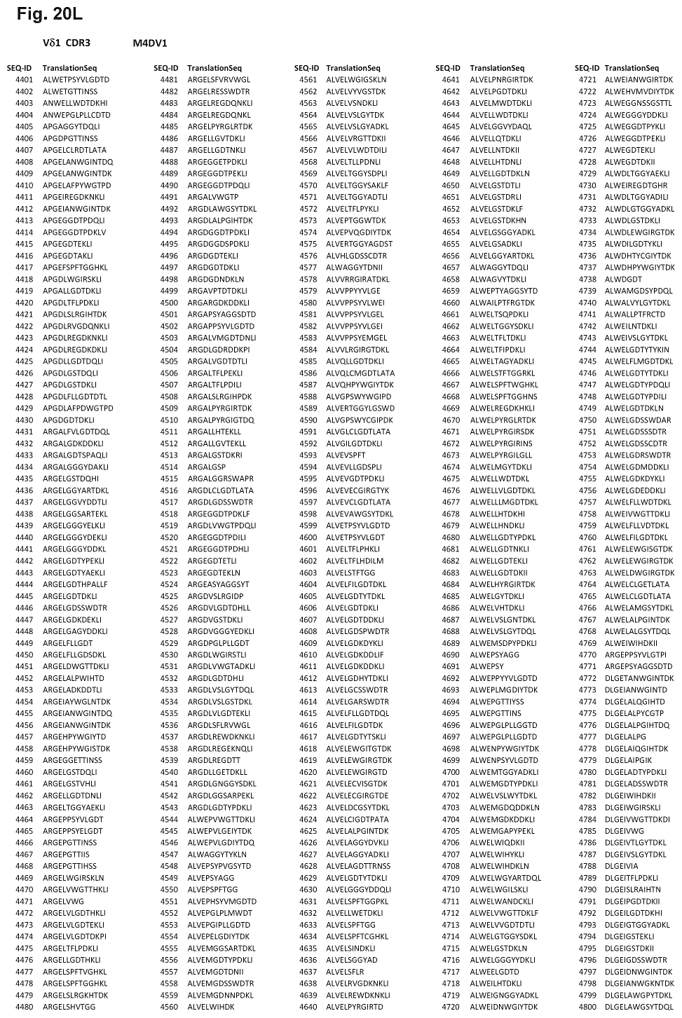

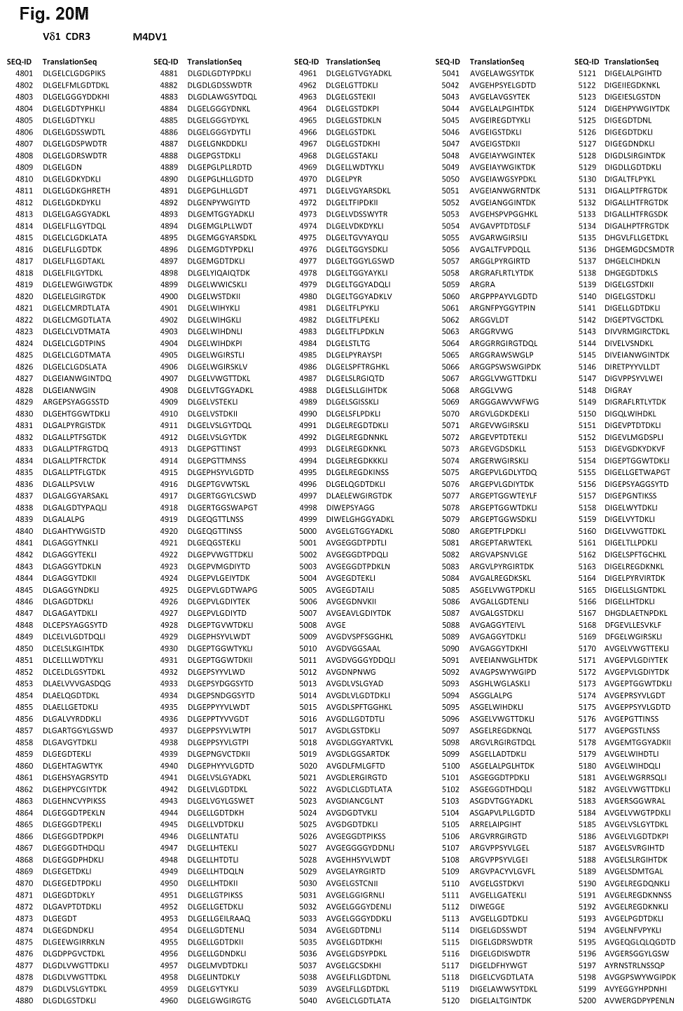

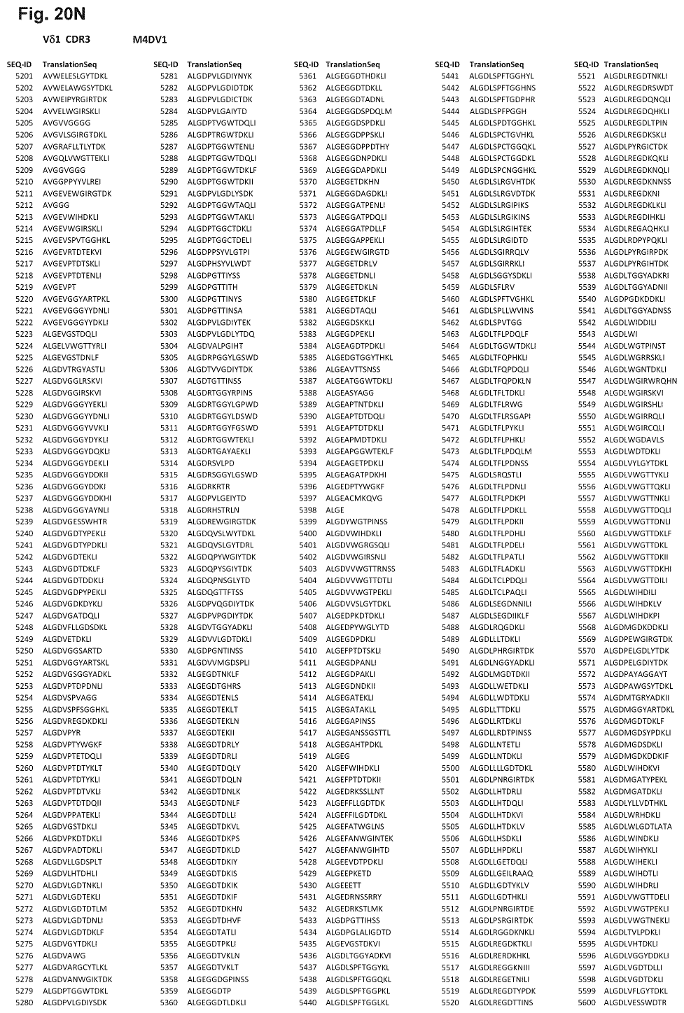

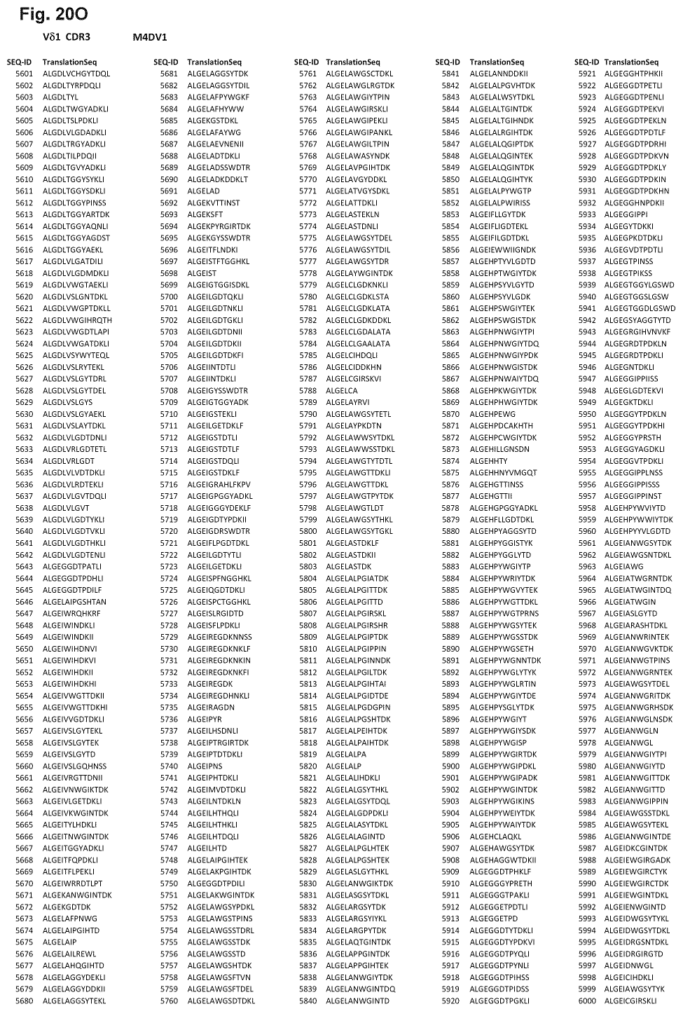

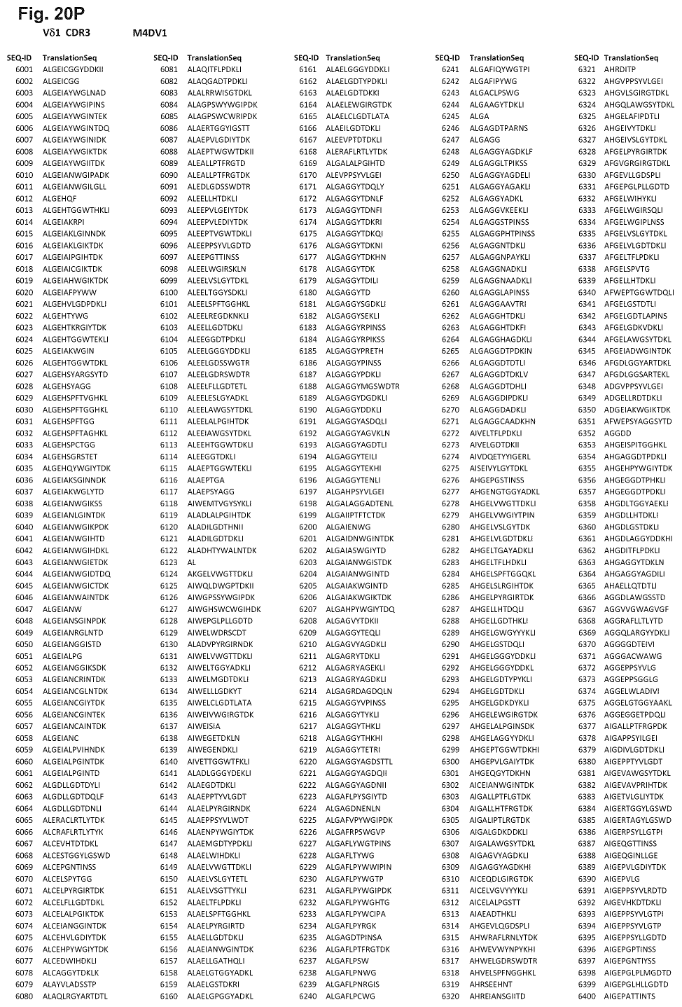

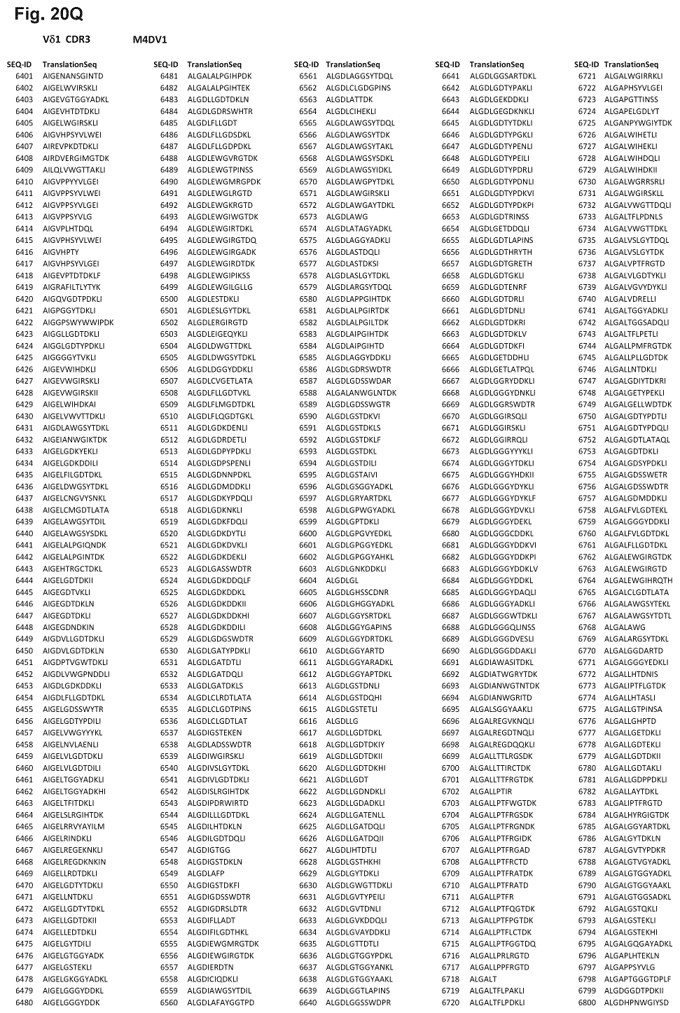

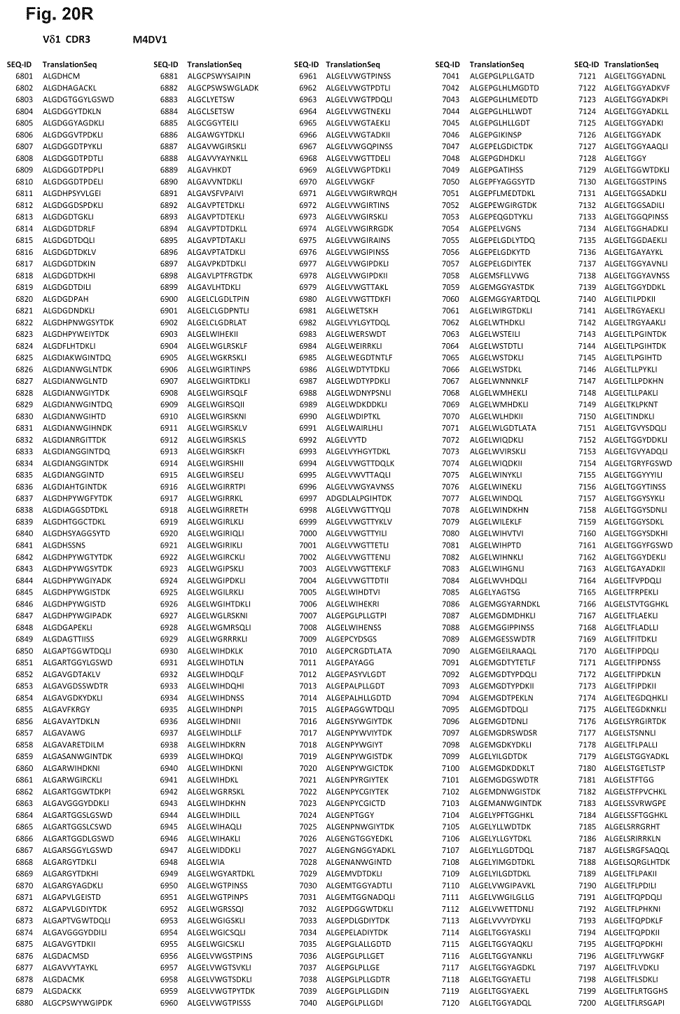

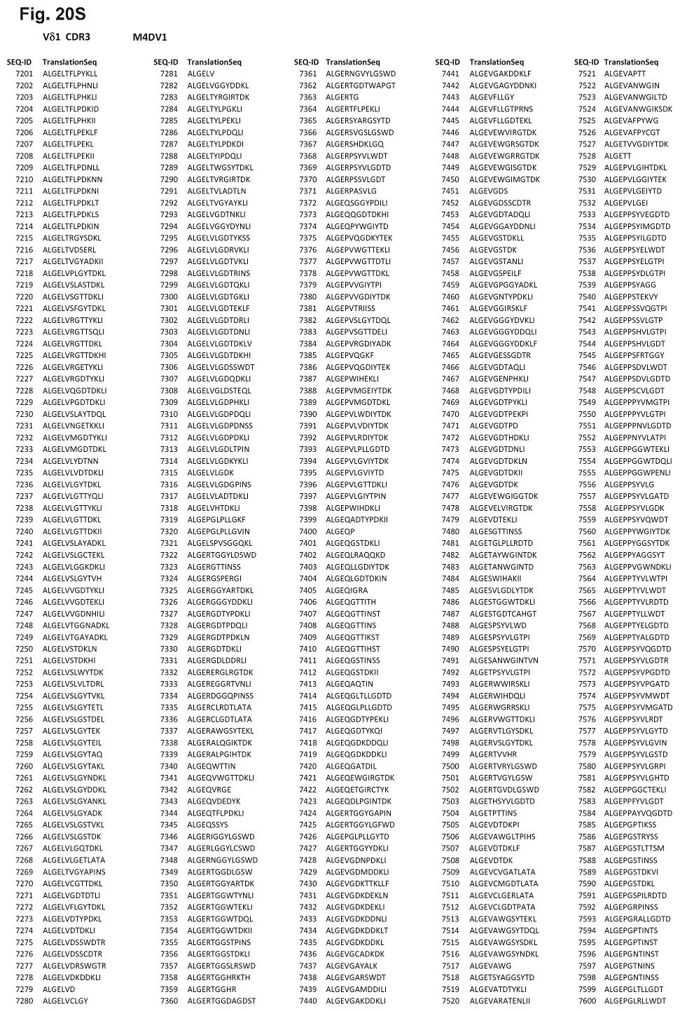

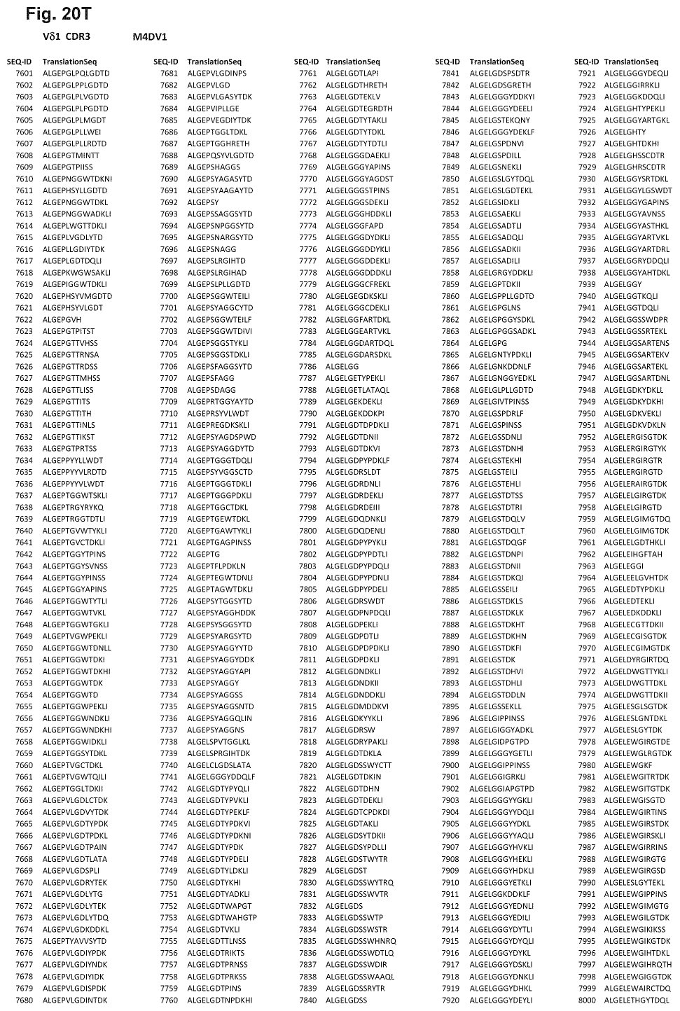

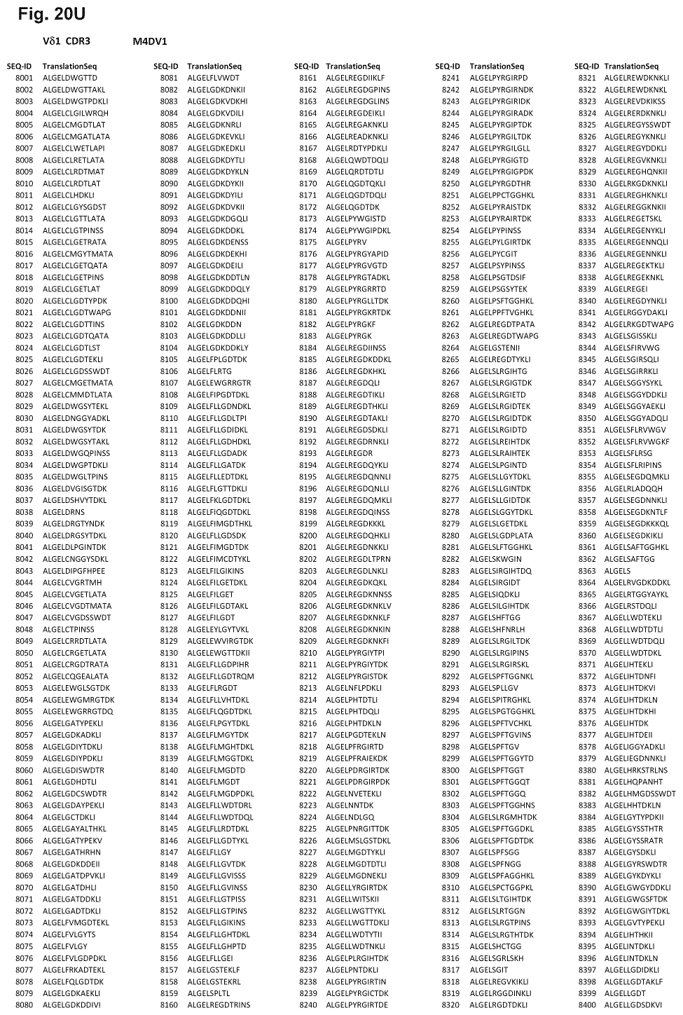

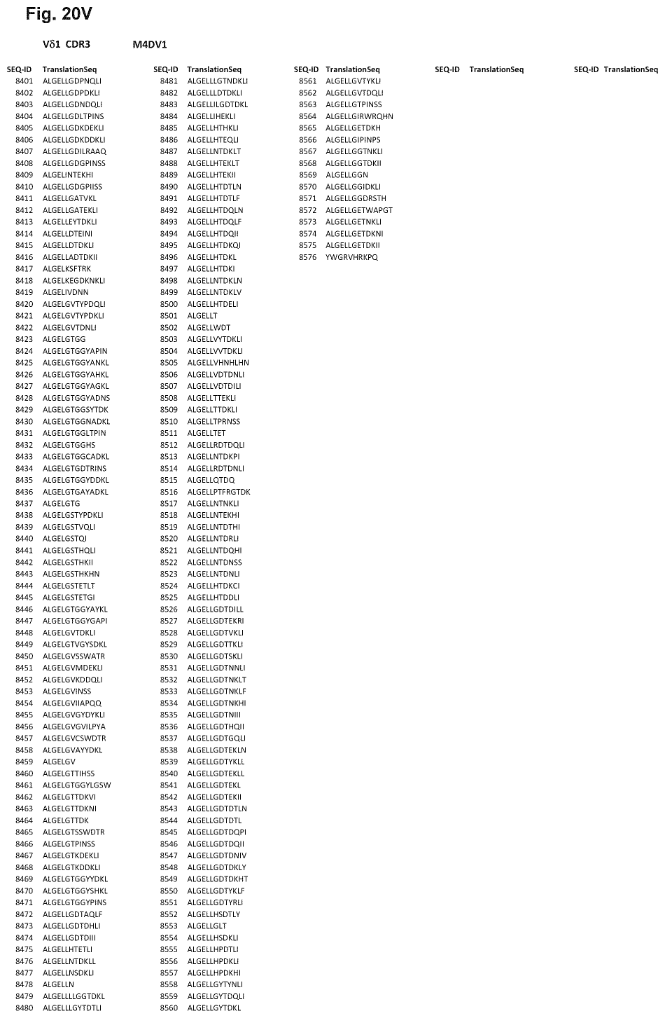

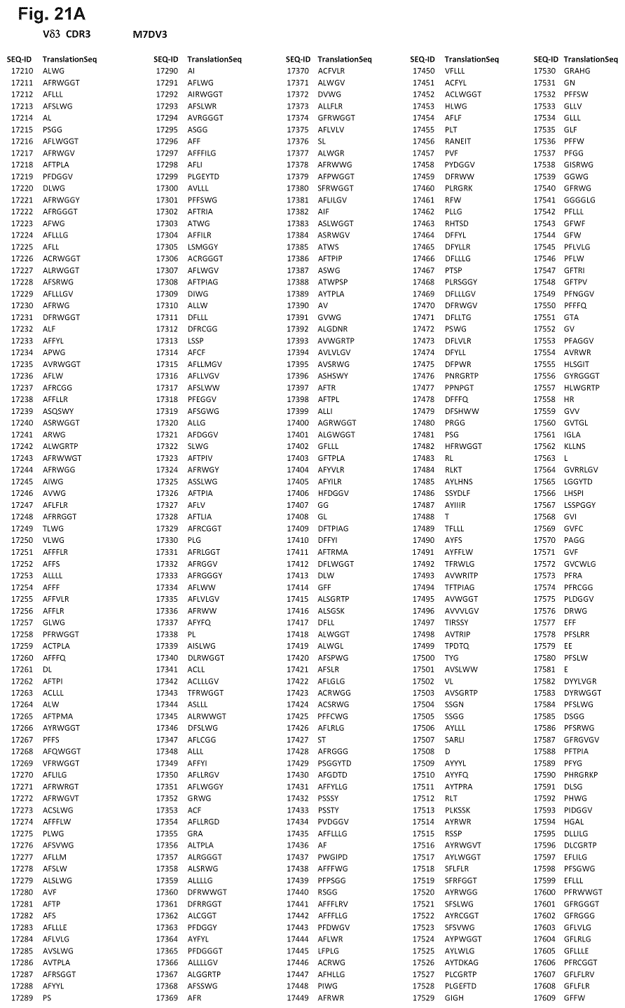

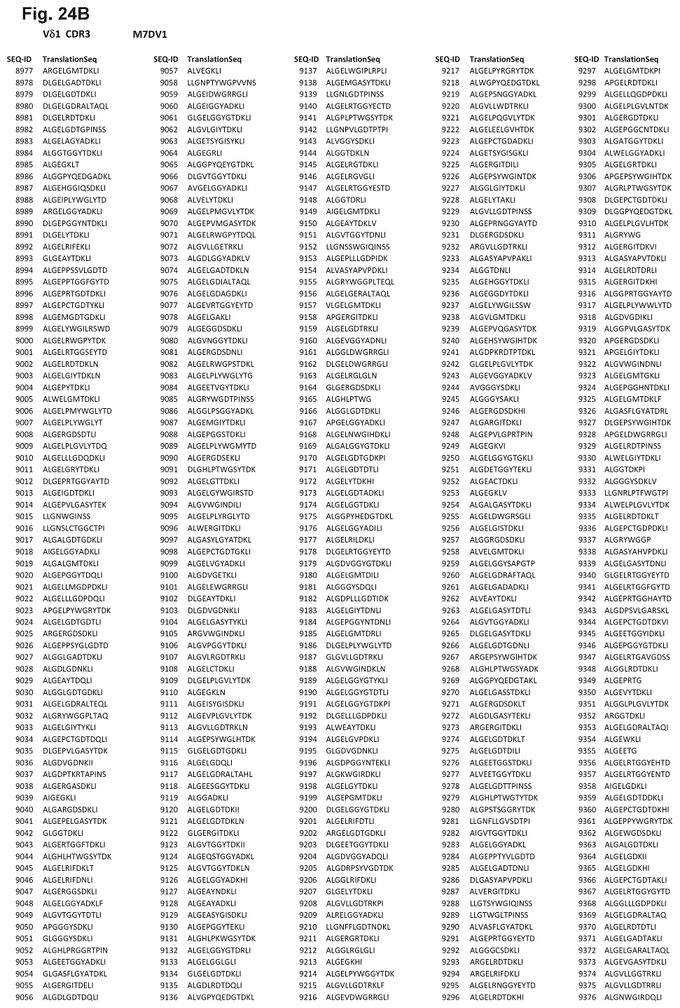

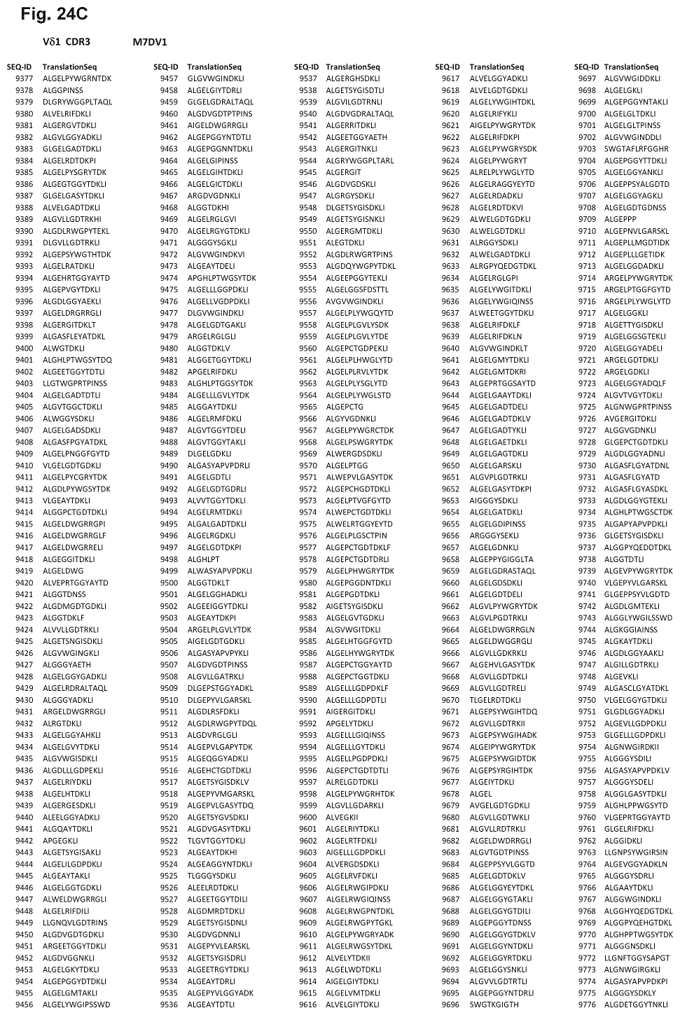

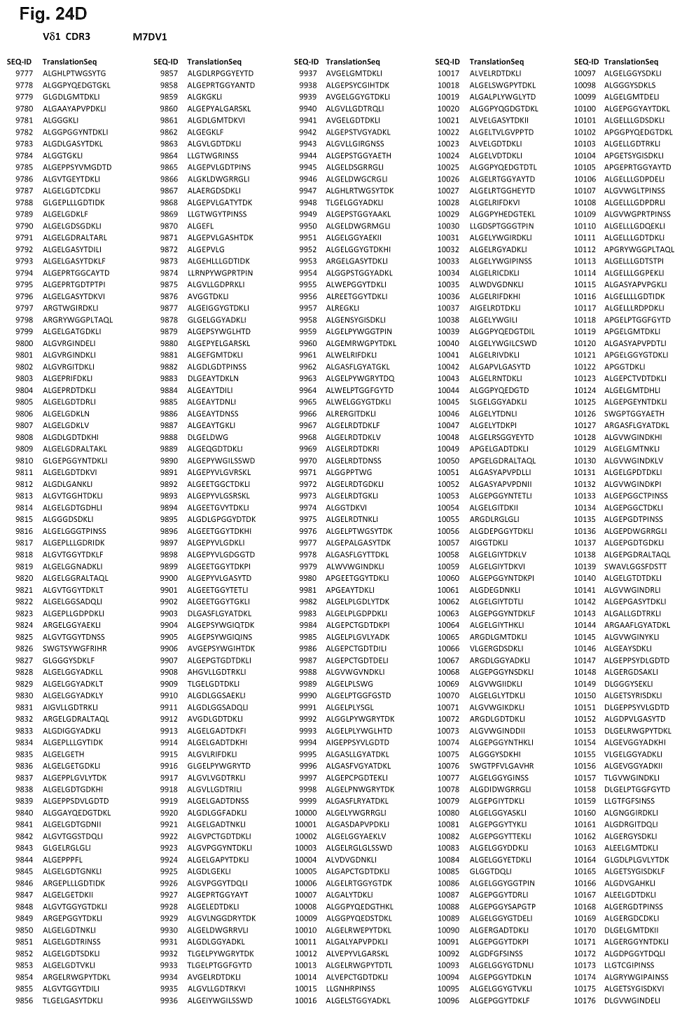

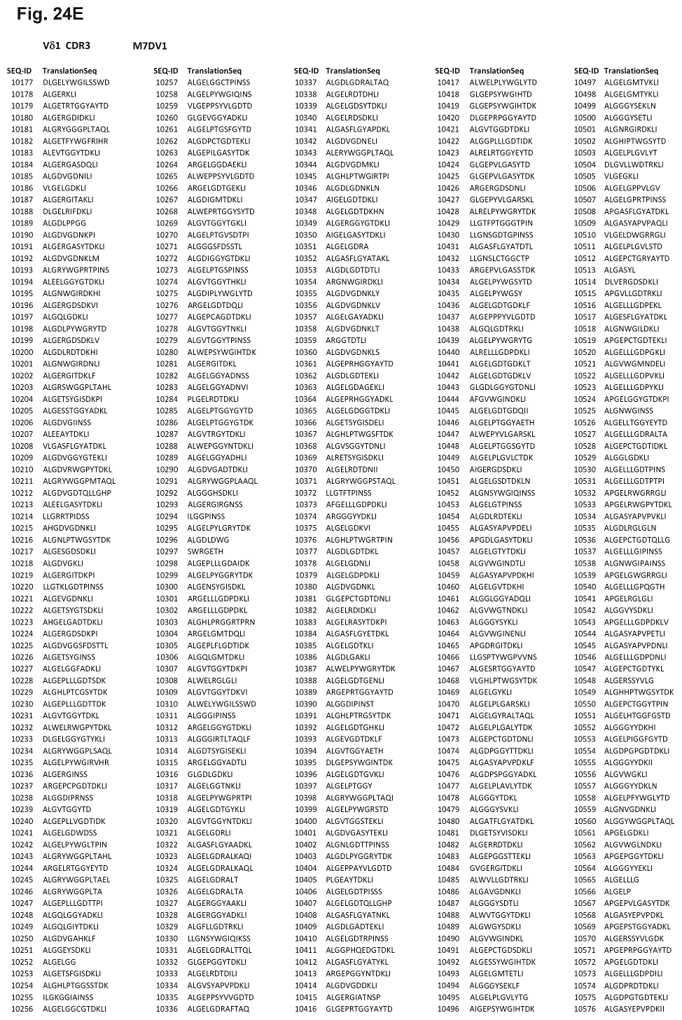

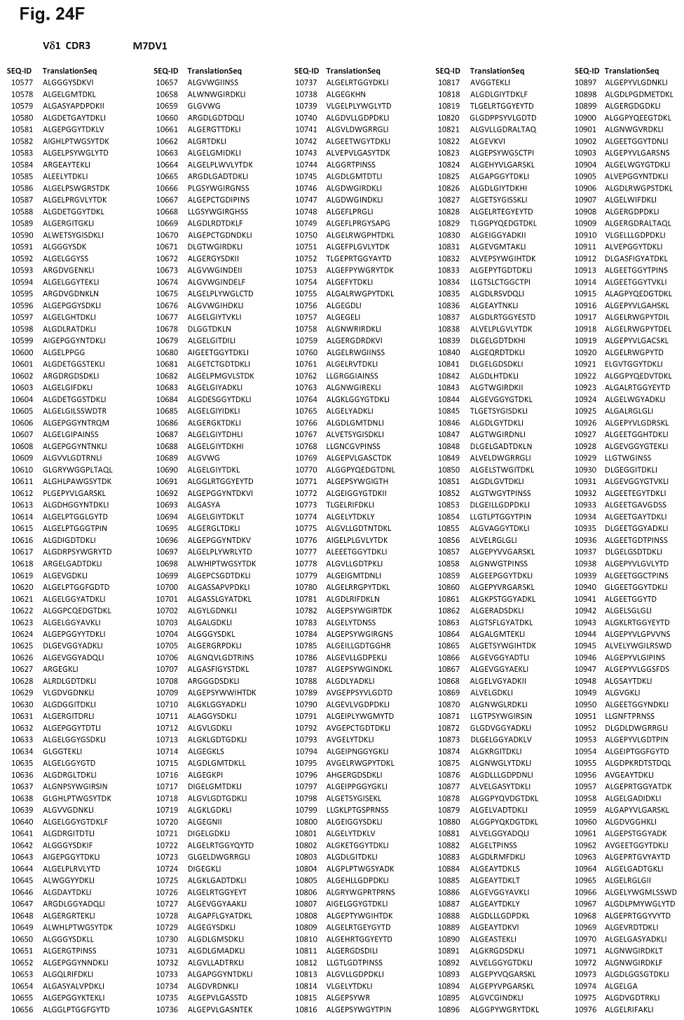

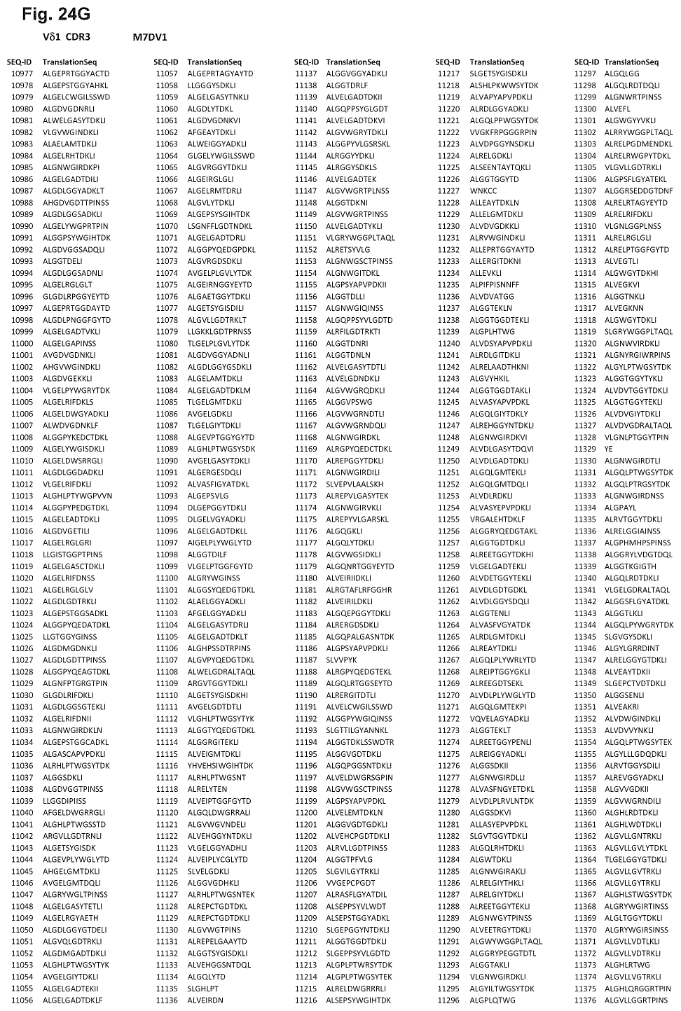

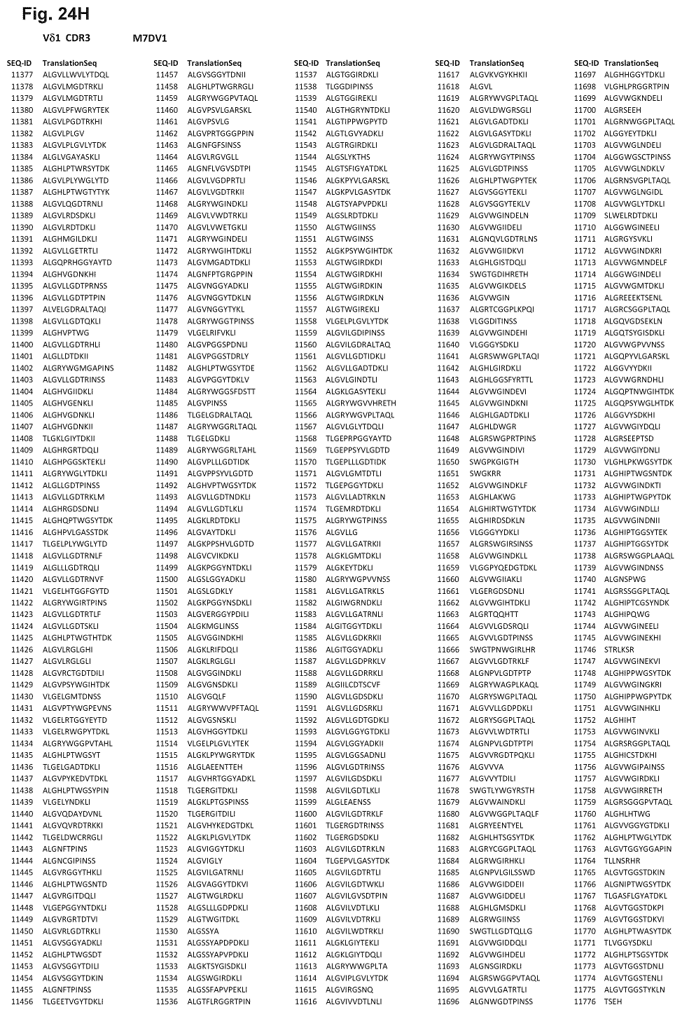

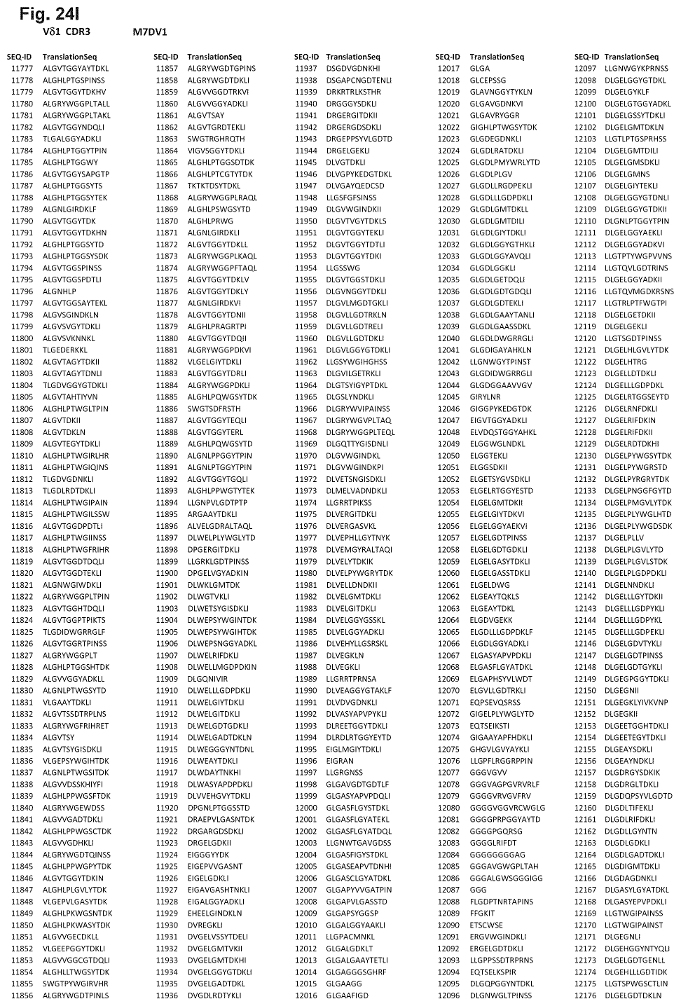

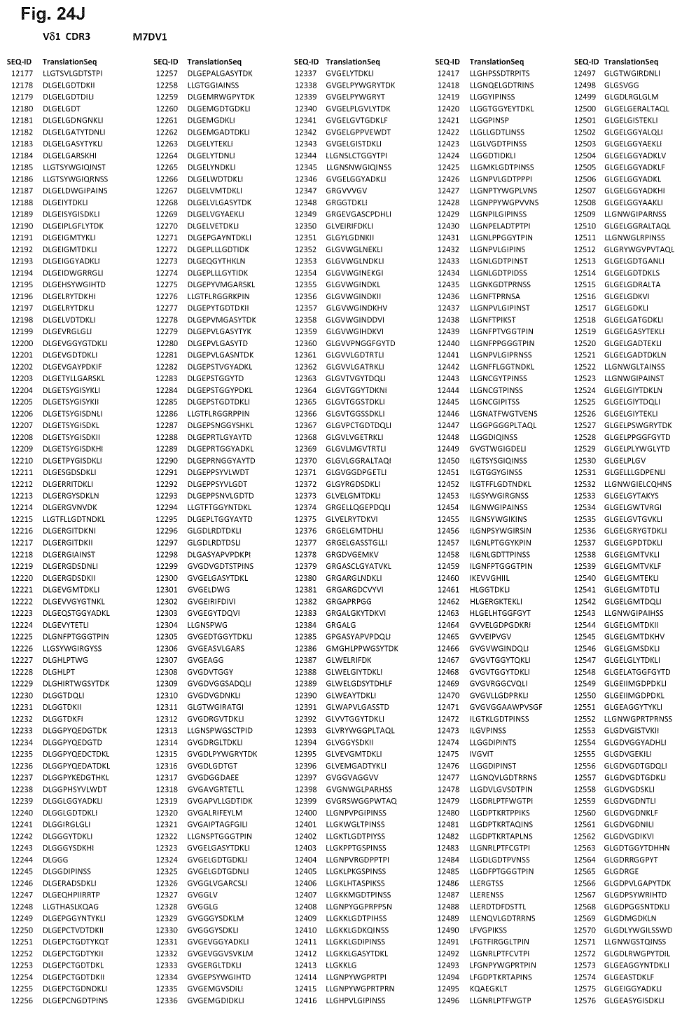

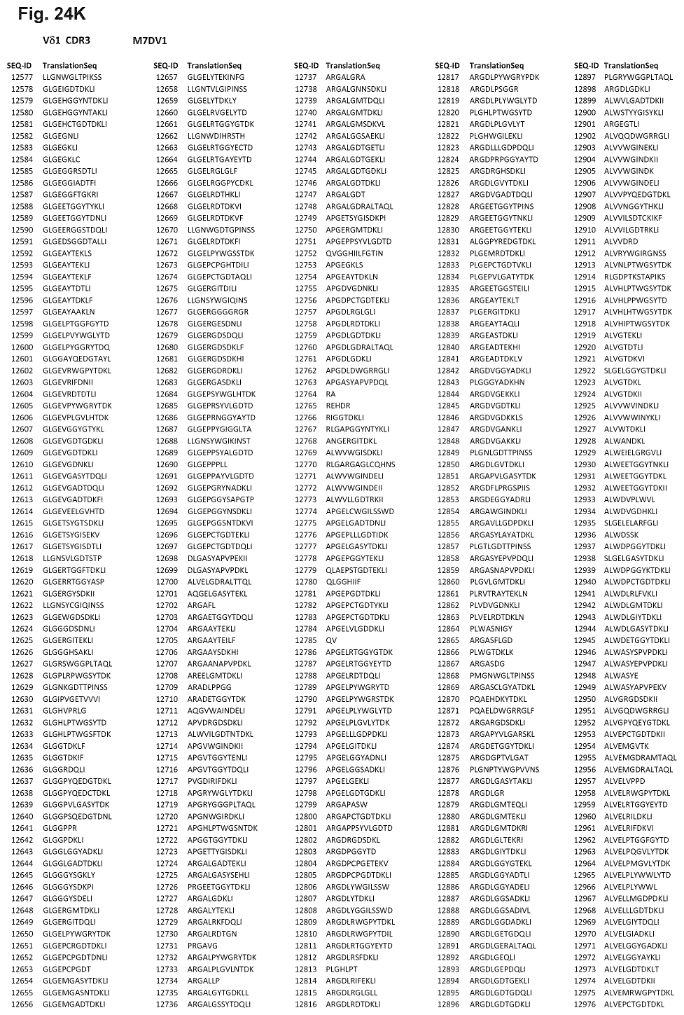

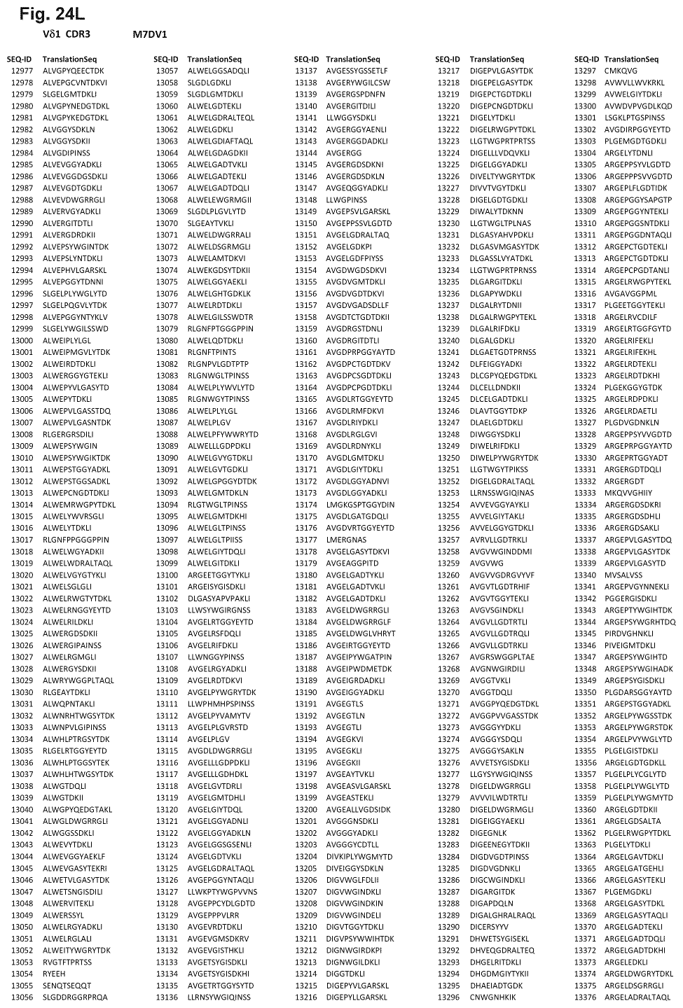

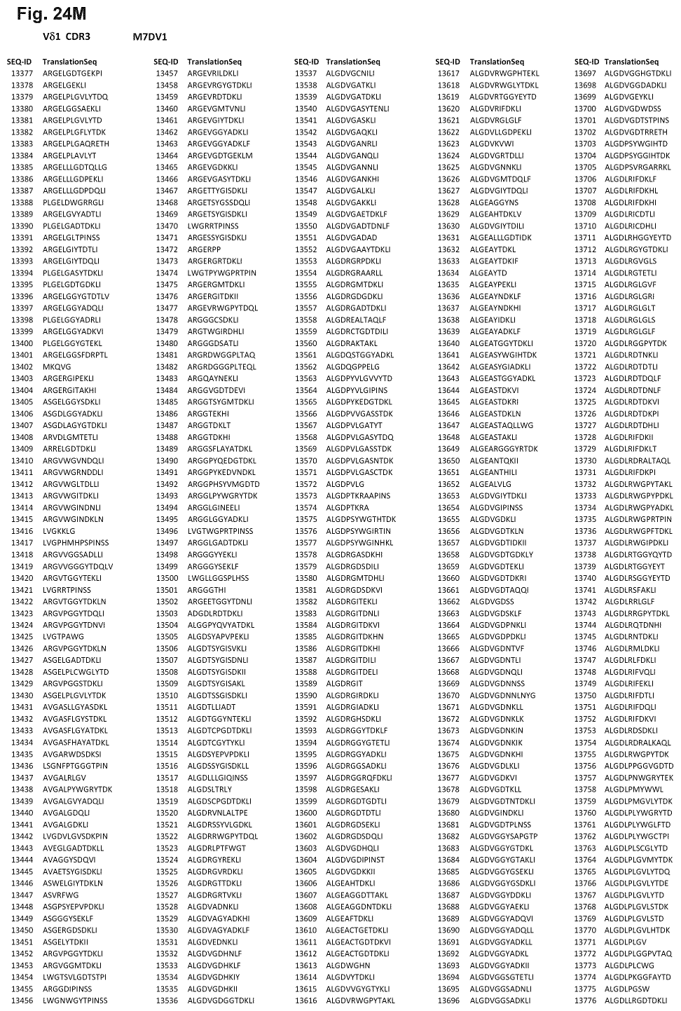

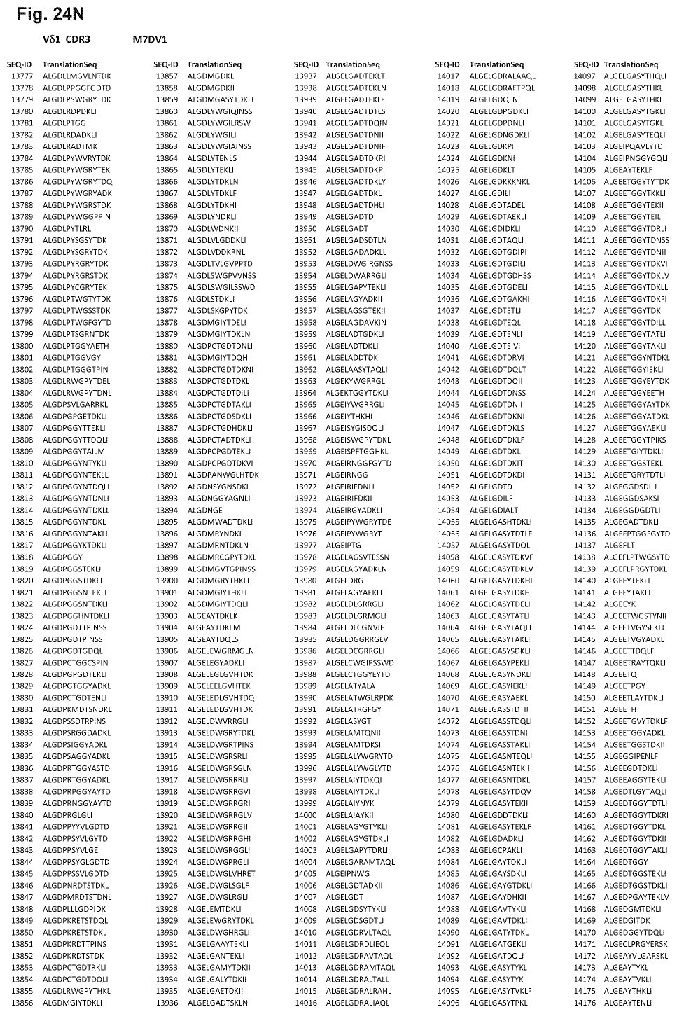

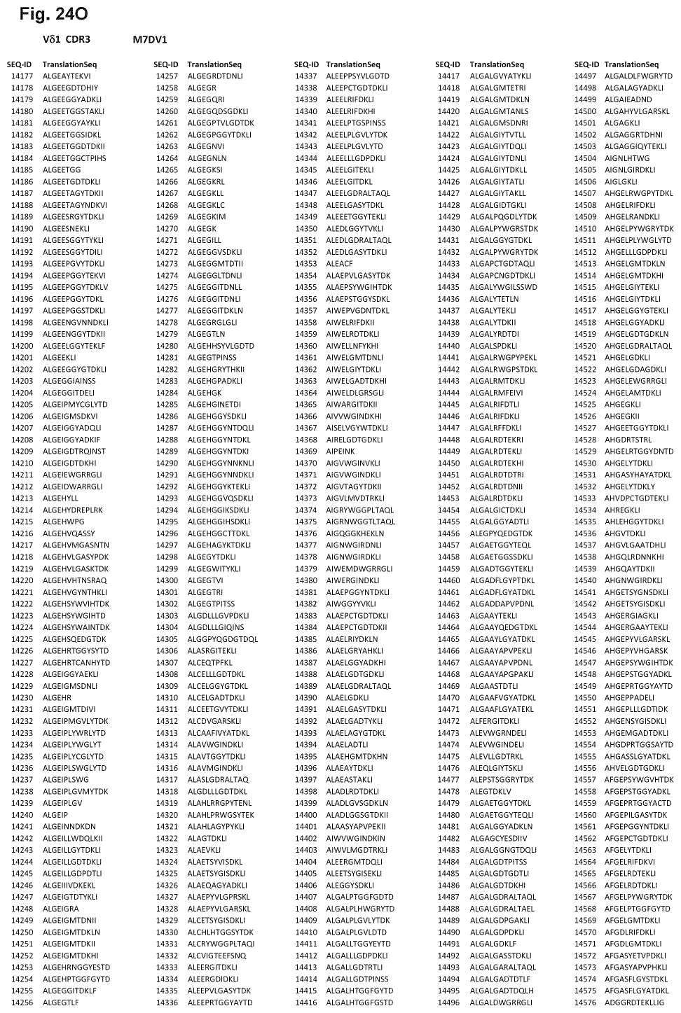

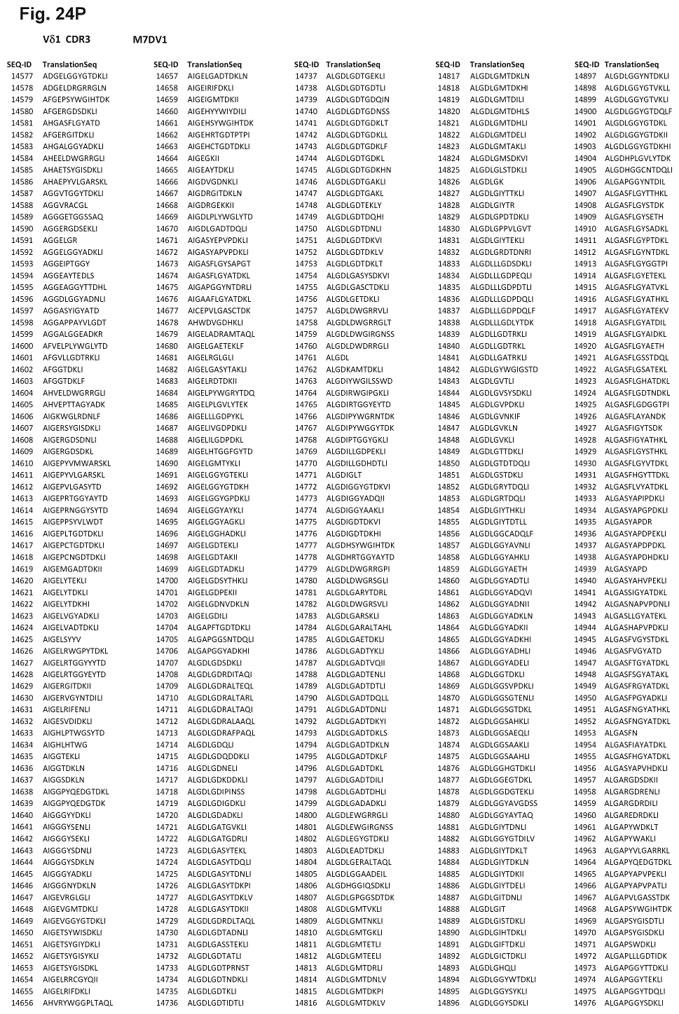

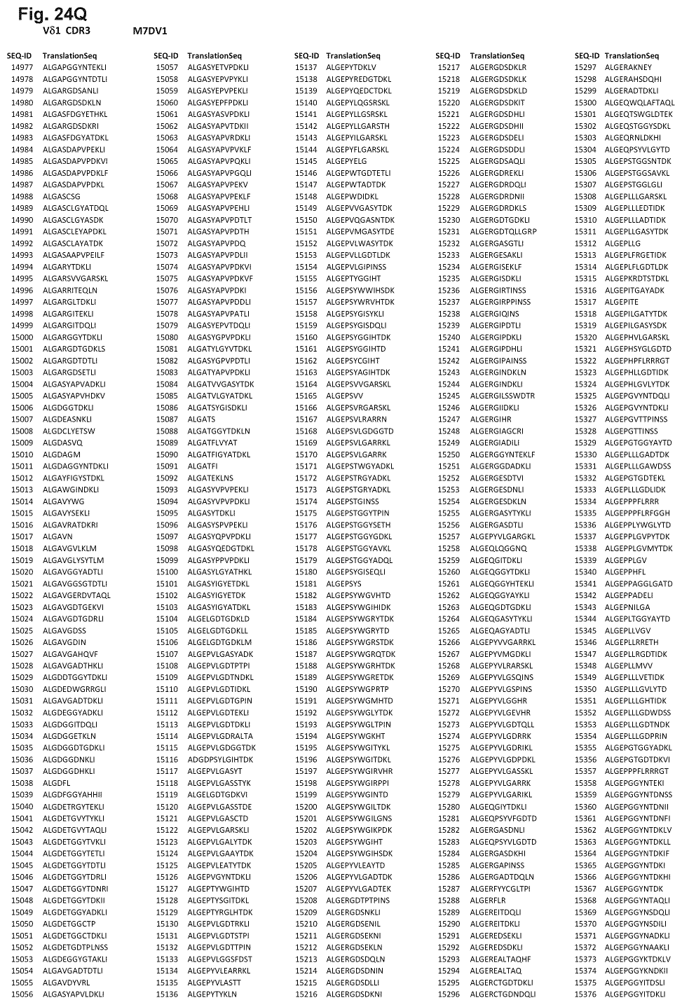

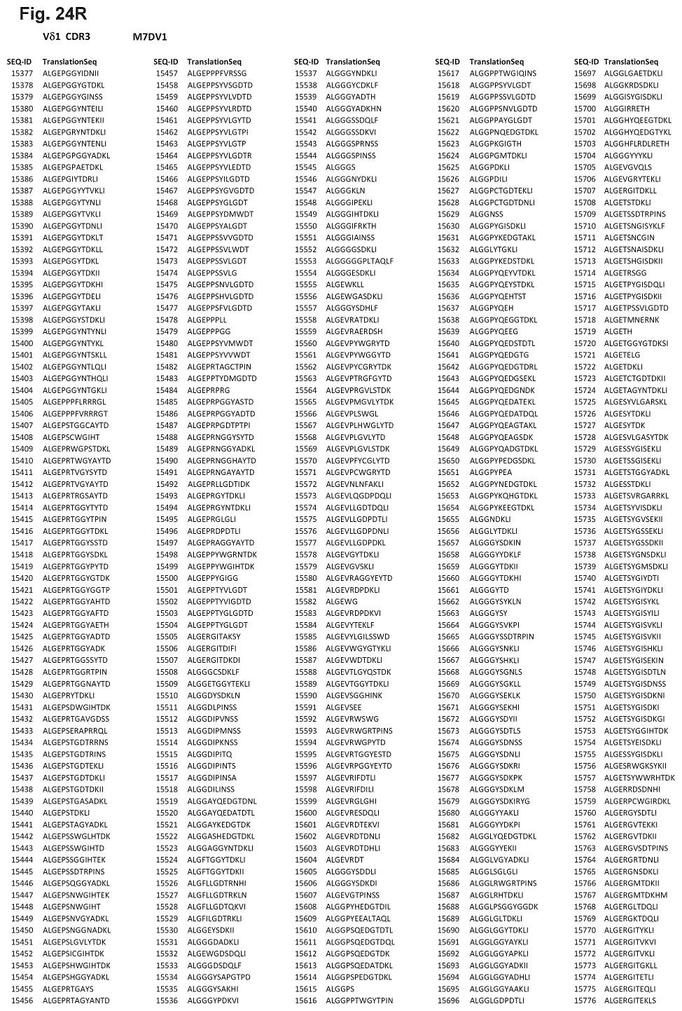

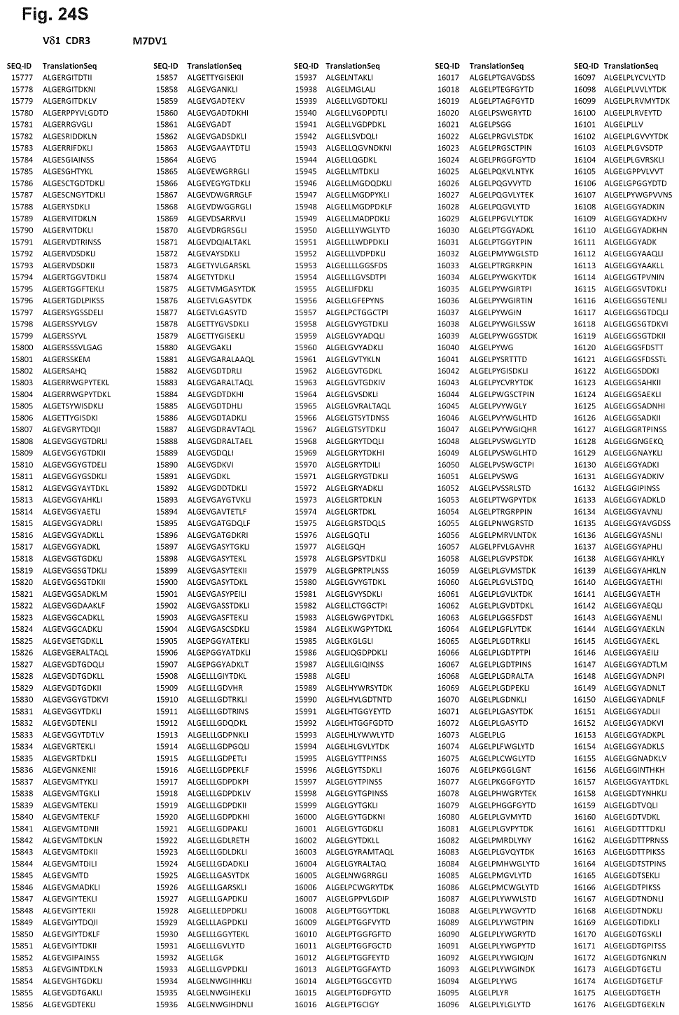

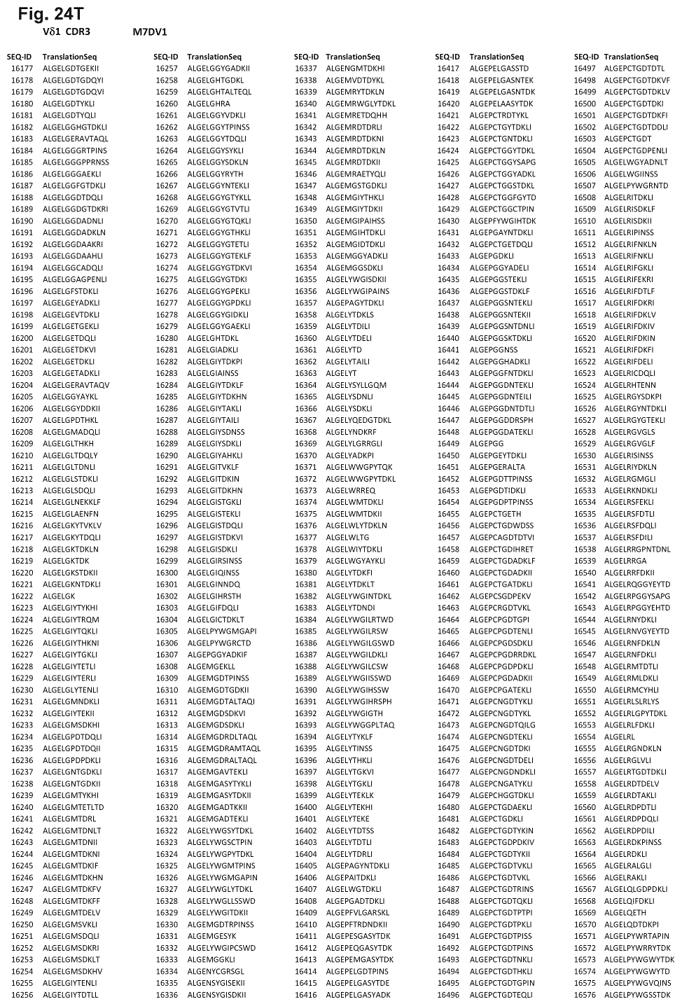

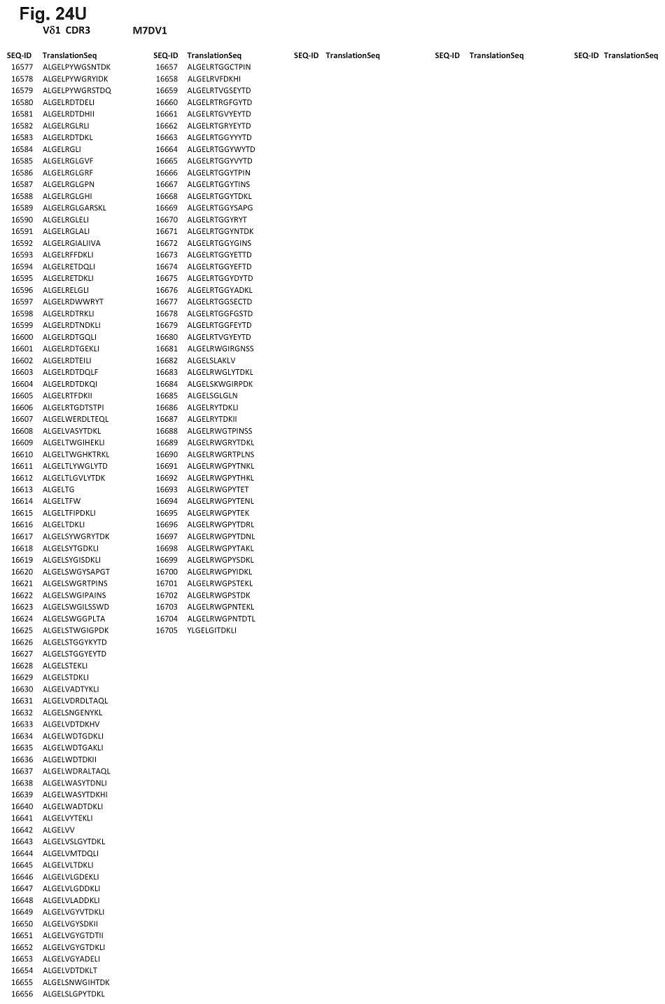

In mouse number one (M4), there was 8576 unique human Vδ1 CDR3s from only 100,000 DNA sequences from NGS-IgBlast analysis (NGS Seq ID NO: 1-8576) (FIG. 20A-V), and the top ten CDR3s are shown in Table 9. In mouse number two (M7) (Table 10), similarly, there was 8129 unique human Vδ1 CDR3s (NGS Seq ID NO: 8577-16705) (FIG. 24A-U). The Vδ1 CDR3s were highly individualized as there was very little common CDR3s shared by these two mice. In fact, among the top twenty CDR3s of mouse number one (M4), only one CRD3 matches with one from more than eight thousand unique sequences, indicating the VDJ recombination is not germline, but rather a somatic recombination with hypermutations including additions and deletions around the border and inside the D region. Furthermore, in mouse number one (M4) and mouse number two (M7), the CDR3 was not focused before encountering highly stimulating antigens. It is evidenced by the fact that the top two Vδ1 CDR3 from mouse one (M4) only counted for 3.87% and 3.71% respectively and there was a total of 38 CDR3s has more than 1% of total CRD3 s and 80.1% of CDR appeared only one or two times in the total CDR3s. Similarly, in mouse number two (M7), the top two Vδ1 CDR3 from mouse one (M4) only counted for 4.44% and 3.94% respectively and there was a total of 40 CDR3s has more than 1% of CDR3s (Table 10) and 79.7% of CDR3s appeared only one or two times in the total CDR3 s. The CDR3 diversification also reflected on the individual mouse level, as among the top twenty δ CDR3s from mouse number one (M4) only one matches with one CDR3 sequence from mouse number two (M7), and only two from M7 shared with M4. Individualized diversification is also observed in humans (Chen, H., J Immunol Methods. 2017. 443:9-17 and Ravens, S., Frontiers in Immunology 2018. https://doi.org/1.3389/fimmu.2018.00510, and Davey, M., Trends in immunology 2018. 36:446-459), indicating the human TCR mice in large part able to recapitulate what happened in humans.

TABLE 9
Top Ten TCR delta CDR3s in Blood of human V delta Mouse (M4)

| | | | Abundance | | | Abundance | |
|---|---|---|---|---|---|---|---|
| SEQ ID No. | CDR3 | | (%) | CDR3 | | (%) | SEQ ID No. |
| 1 | hDV1 | ALGELGSTDKLI | 3.9 | hDV3 | AFFLR | 83 | 16706 |
| 2 | | ALGELLGDTDKLI | 3.7 | | ALERETH | 2.7 | 16707 |
| 3 | | ALGEGDTDKLI | 2.6 | | ALERSTH | 2.2 | 16708 |
| 4 | | ALGEGGDTPDKLI | 2.3 | | AFFLLG | 0.8 | 16709 |
| 5 | | ALGEPGTTINSS | 2.2 | | ACFLR | 0.8 | 16710 |
| 6 | | ALGELWIHDKLI | 2 | | AFGD | 0.6 | 16711 |
| 7 | | ALGELPYRGIRTDK | 2 | | ASQSWY | 0.6 | 16712 |
| 8 | | ALGELLHTDKLI | 1.9 | | ALFLR | 0.6 | 16713 |
| 9 | | ALGELGDSSWDTR | 1.9 | | AFGDTD | 0.3 | 16714 |
| 10 | | ALGELGDTYPDKLI | 1.8 | | AFGGT | 0.3 | 16715 |

| | | | |
|---|---|---|---|
| 17859 | hDV6 | AASRGDTDKLI | 21 |
| 17860 | | AASPGGSDKLI | 21 |
| 17861 | | AASTYTDKLI | 15 |
| 17862 | | AARGGGYGWGLGTD | 11 |
| 17863 | | AASMRWGIRSWDTRQM | 6.8 |
| 17864 | | AASPGGSDKPI | 1.5 |
| 17865 | | AASRGDTEKLI | 1.1 |
| 17866 | | AASPGGSEKLI | 1 |
| 17867 | | AARGGGYGYWGLHT | 0.6 |
| 17868 | | AARGGGYGSWGLHTD | 0.4 |

TABLE 10
Top Ten TCR delta CDR3s in Blood of human V delta Mouse (M7)

| SEQ ID No. | CDR3 | | (%) | CDR3 | | (%) | SEQ ID No. |
|---|---|---|---|---|---|---|---|
| 8577 | hDV1 | ALGELGGYADKLI | 4.4 | hDV3 | ALWG | 51 | 16756 |
| 8578 | | ALGELGMTDKLI | 3.9 | | AFRWGGT | 34 | 16754 |
| 8579 | | ALGGGYSDKLI | 2.3 | | AFLLL | 1.7 | 17212 |
| 8580 | | ALGGTDKLI | 2.3 | | AFSLWG | 1.1 | 17213 |
| 8581 | | ALGERGITDKLI | 2.1 | | AL | 0.8 | |
| 8582 | | ALGEGKLI | 2 | | PSGG | 0.7 | 17215 |
| 8072 | | ALGELGADTDKLI | 1.9 | | AFLWGGT | 0.7 | 17024 |
| 8084 | | ALGELRDTDKLI | 1.8 | | AFRWGV | 0.7 | 17030 |
| 8585 | | ALGVLLGDTRKLI | 1.8 | | AFTPLA | 0.6 | 17218 |
| 8586 | | ALGEAYTDKLI | 1.8 | | PFDGGV | 0.4 | 17219 |

| | | | |
|---|---|---|---|
| 18040 | hDV6 | AAERGPDKLI | 15.0 |
| 18043 | | AAVLGDTVKLI | 15.0 |
| 18077 | | AAPTGGYGYTDKLI | 13.0 |
| 18158 | | ASRGLYTDKLI | 11.0 |
| 18099 | | AASVLRWWGIKLI | 10.0 |
| 18249 | | AAAKAFLRRGAPYTD | 9.0 |
| 18670 | | AASGLPSLGVHTDKLI | 2.6 |
| 18567 | | AASVRLWWGSKLI | 1.3 |
| 22129 | | AAAKAFLRRGAPYT | 0.6 |
| 22130 | | AAAKAFLRRGAPSTD | 0.6 |

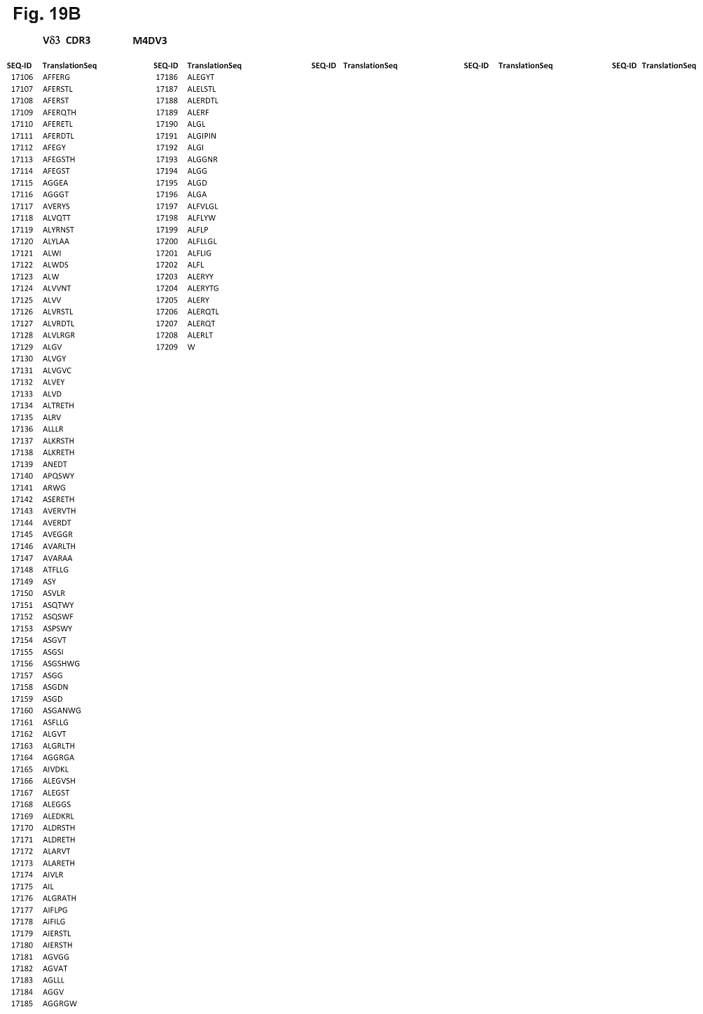

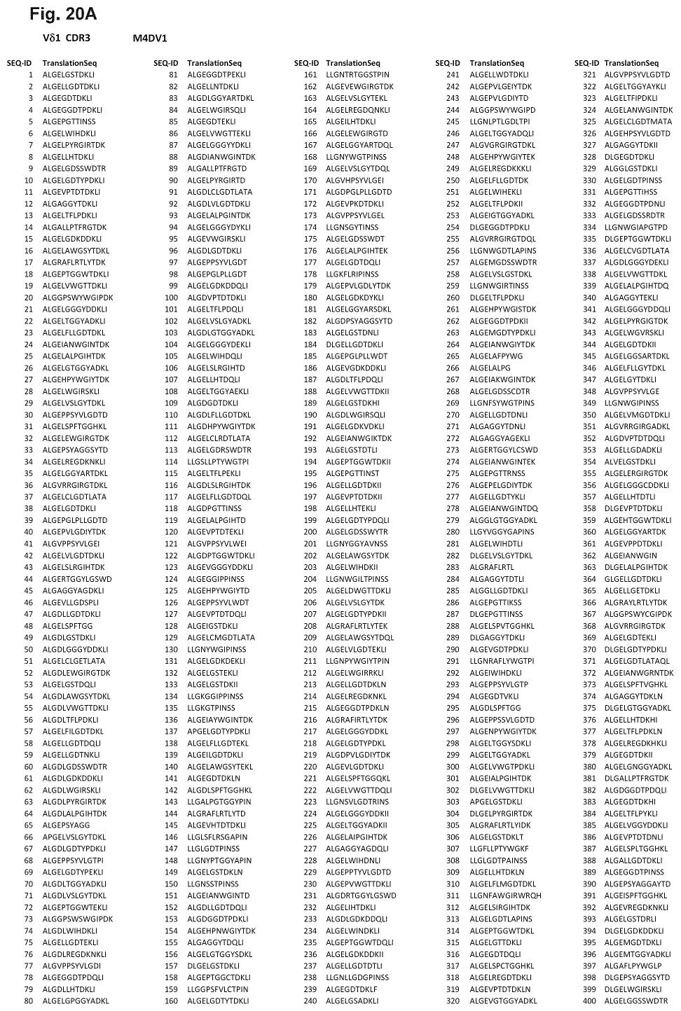

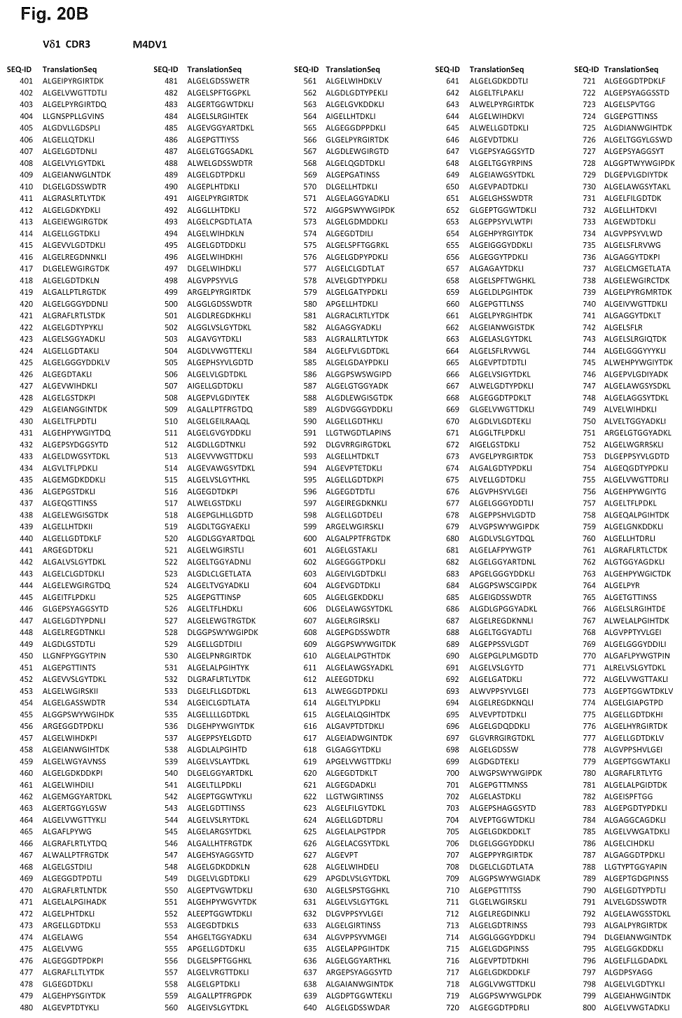

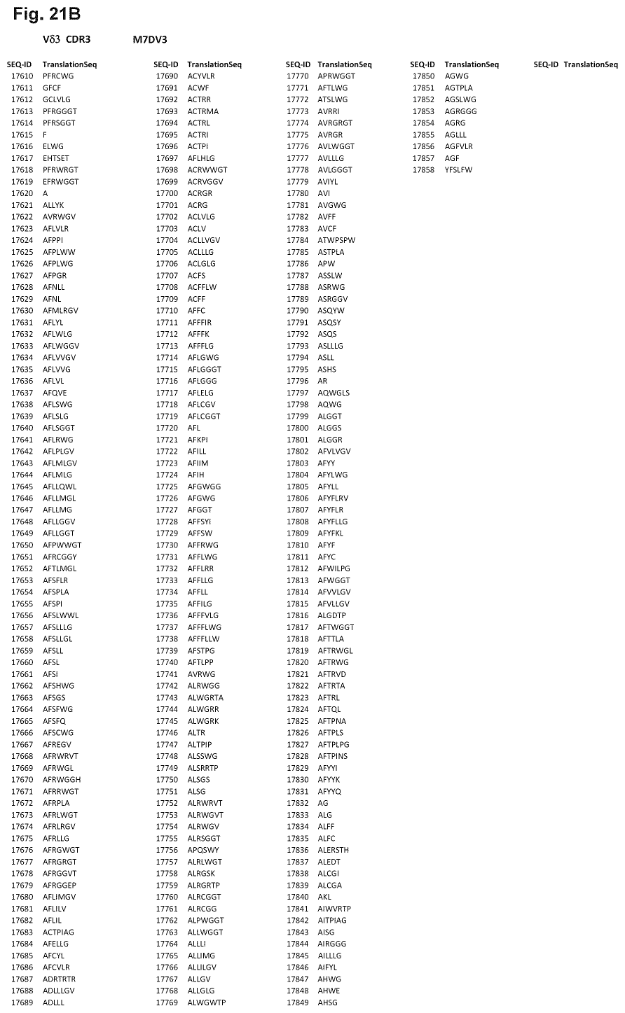

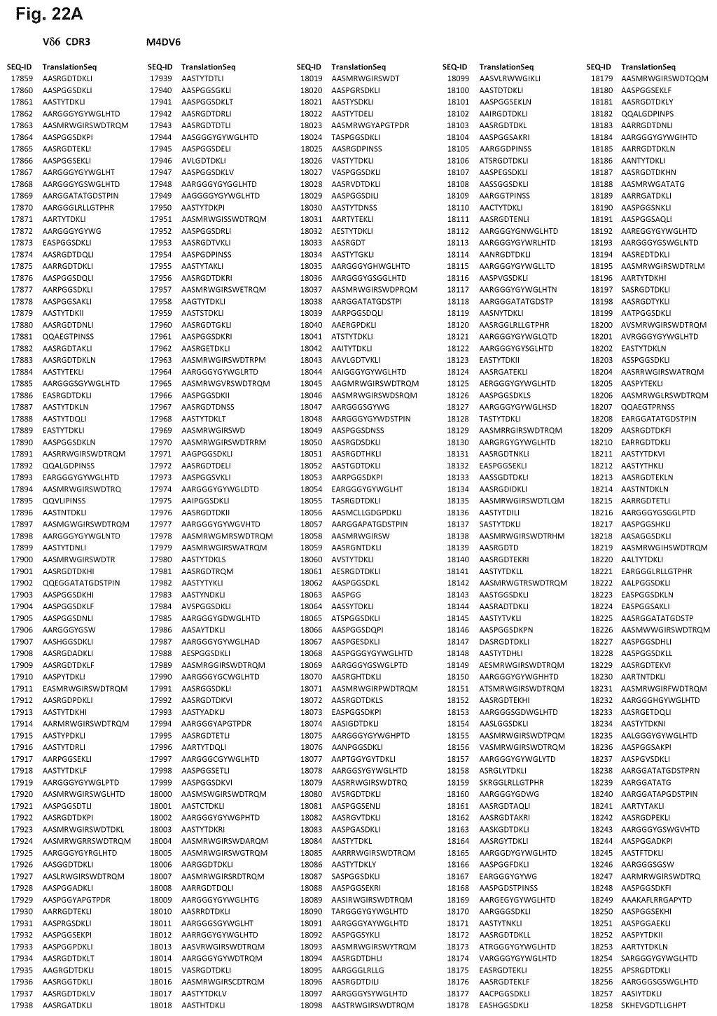

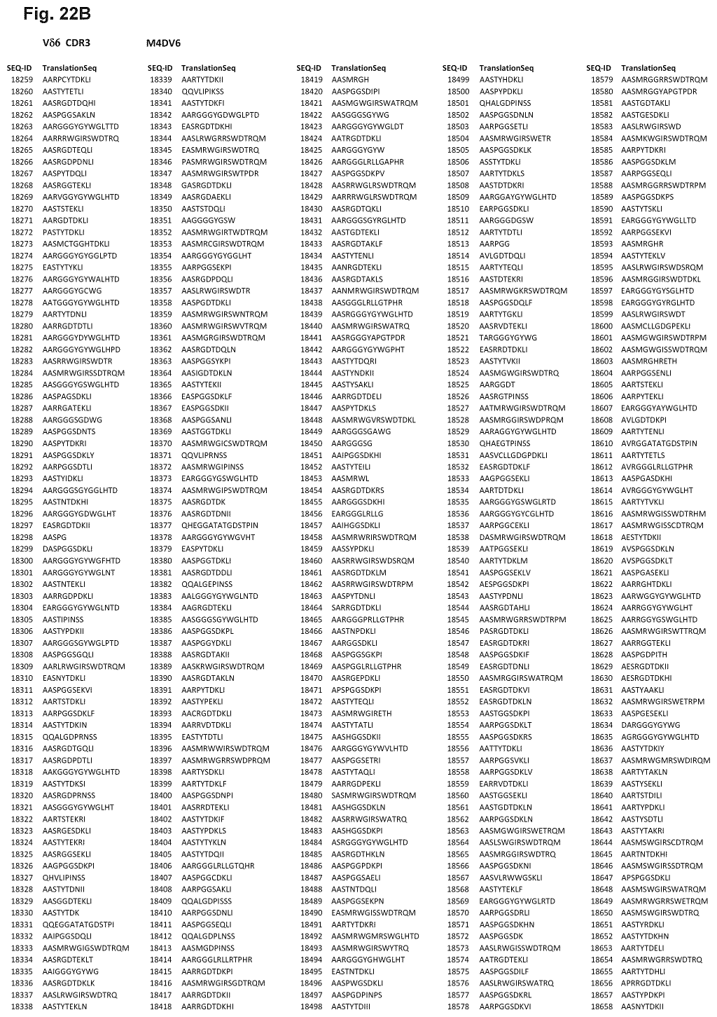

In terms of human TCR Vδ3, CRD3s were less diversified as shown in Tables 9 and 10. In mouse number one (M4), there were only 504 unique Vδ3 CDR3s from only 100,000 DNA sequences from NGS-IgBlast analysis (NGS Seq ID NO: 16706-17209) (FIG. 19A-B). In mouse number two (M7), similarly, there were 649 unique Vδ3 CDR3s (NGS Seq ID NO: 17210-17858) (FIG. 21A-B). The Vδ3 CDR3s were highly shared among these two mice. In fact, among the top twenty CDR3s of mouse number one (M4), nine CRD3s matched with that of mouse number two (M7). The Vδ3 CDR3s were also focused as the top one CDR3 from mouse number one (M4) counted for 83% of total CDR3. Similarly, the top two CDR3 from mouse number two (M7) counted for 50% and 34% respectively, reflecting possibly early exposure to a particular antigen(s). As not much information is available from humans, a comparative study can be very limited.

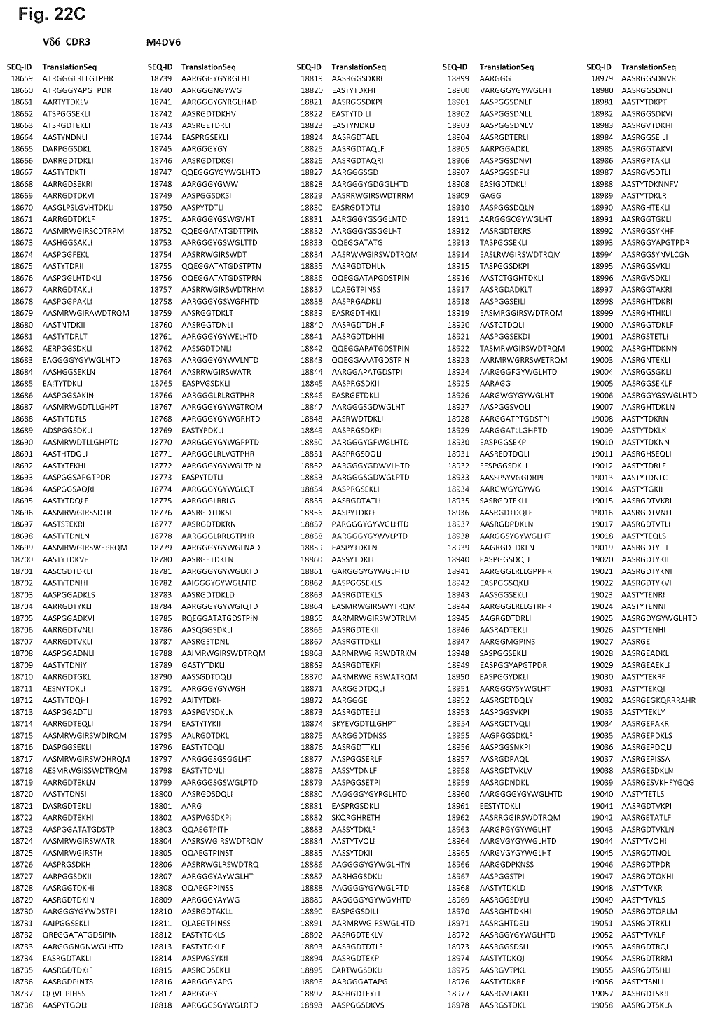

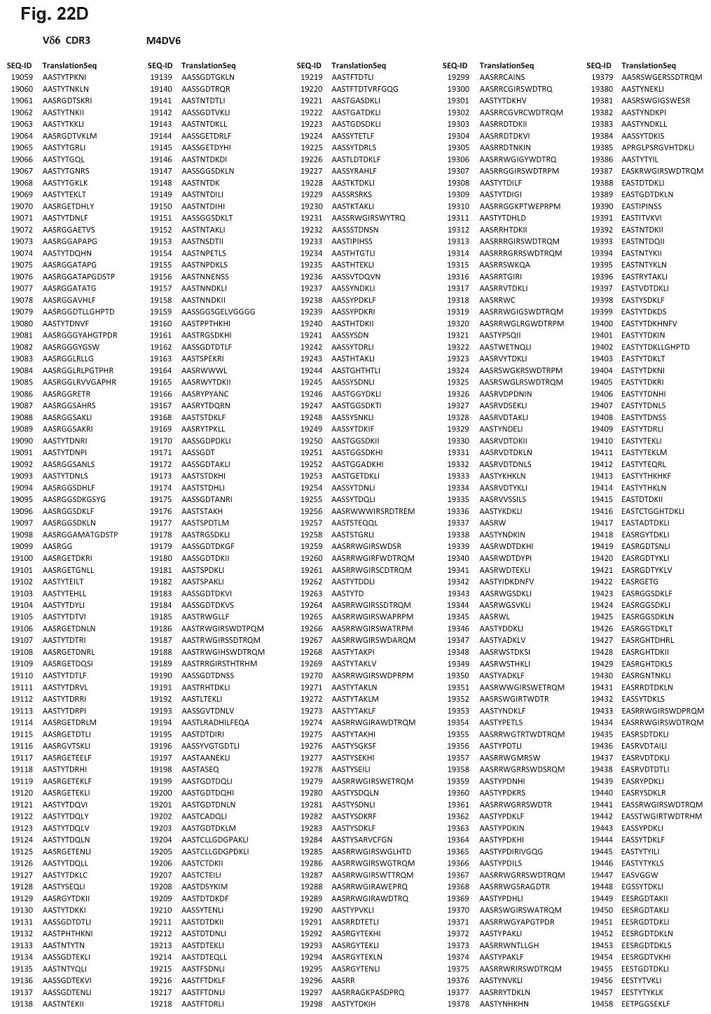

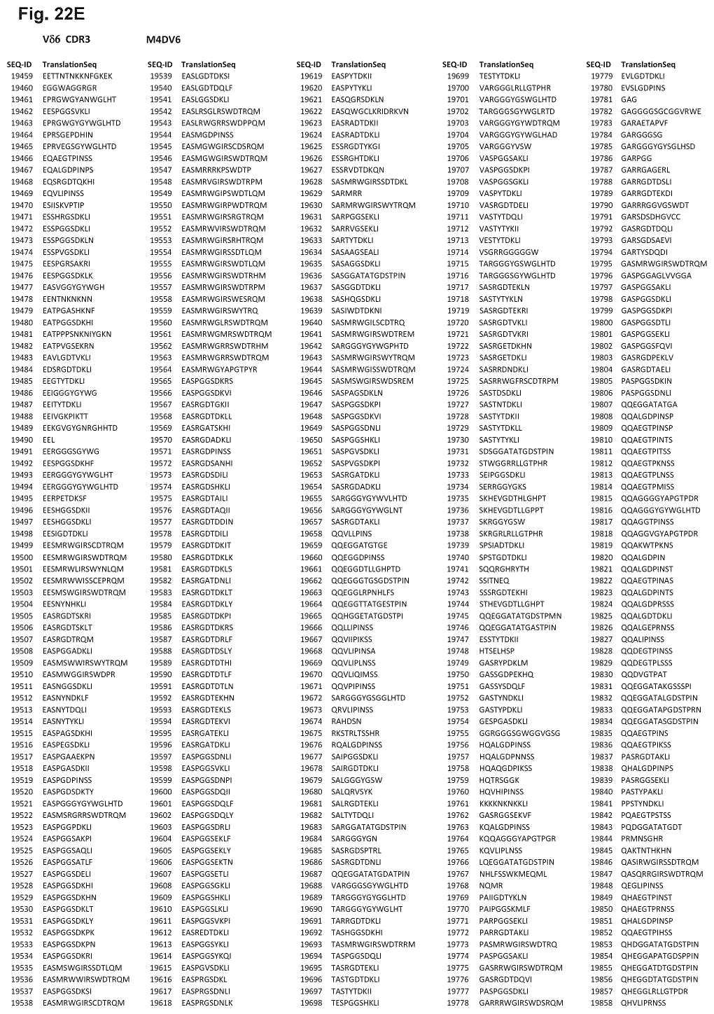

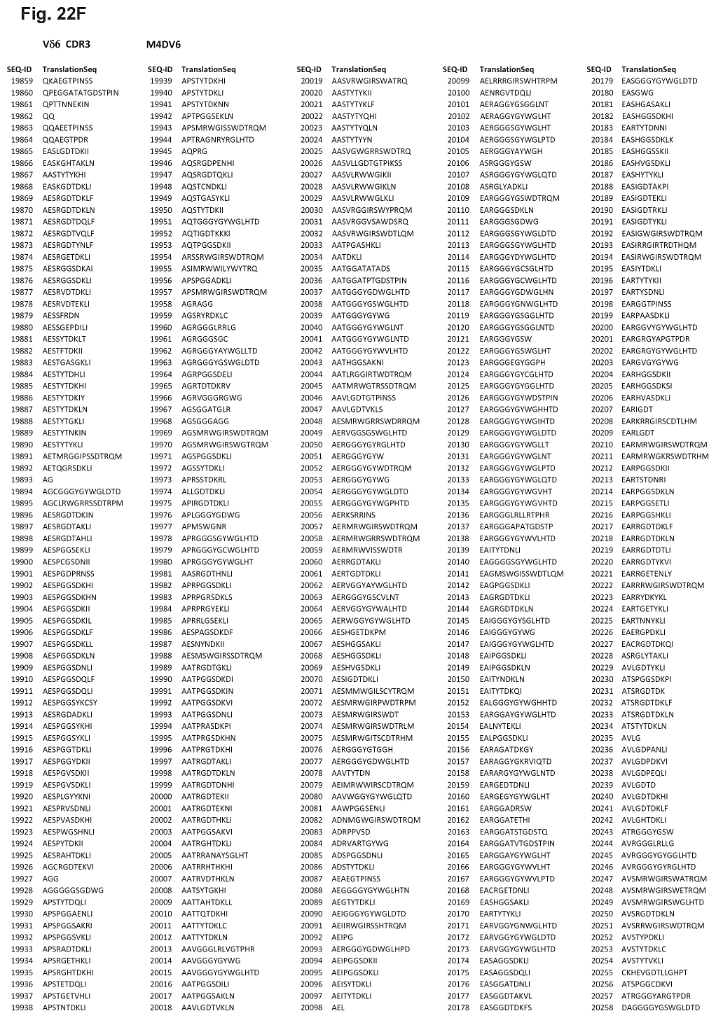

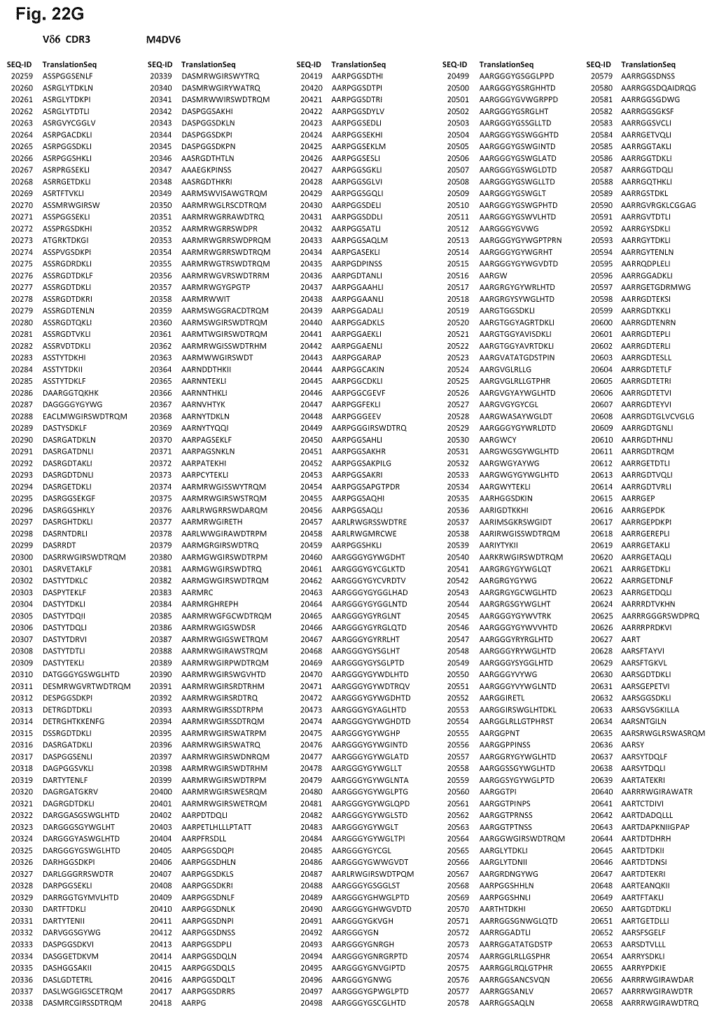

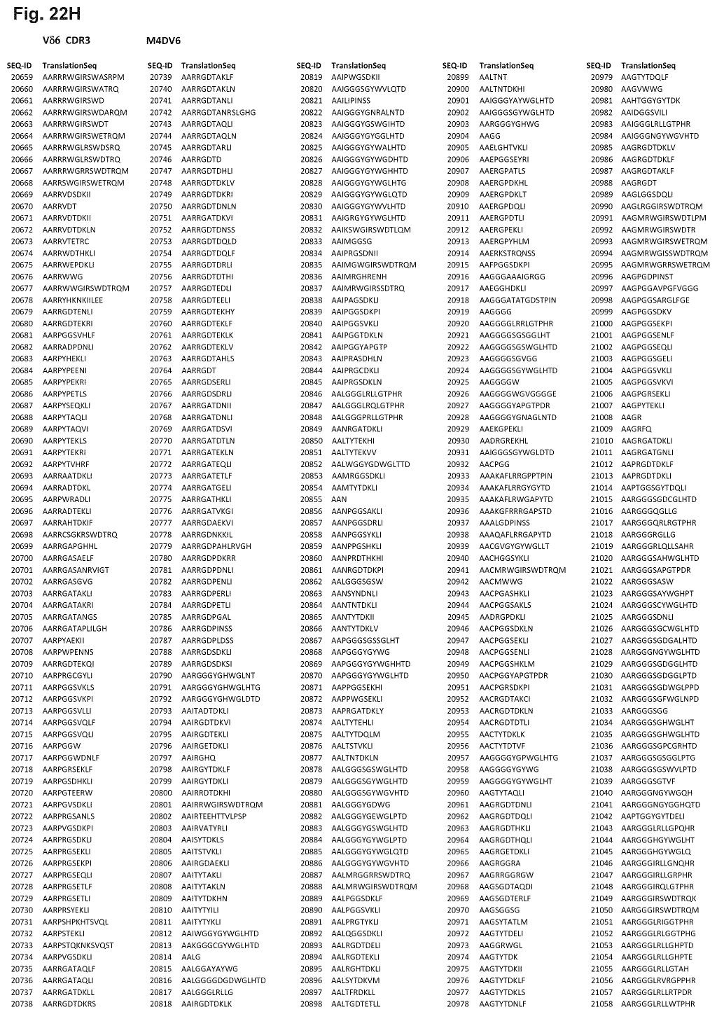

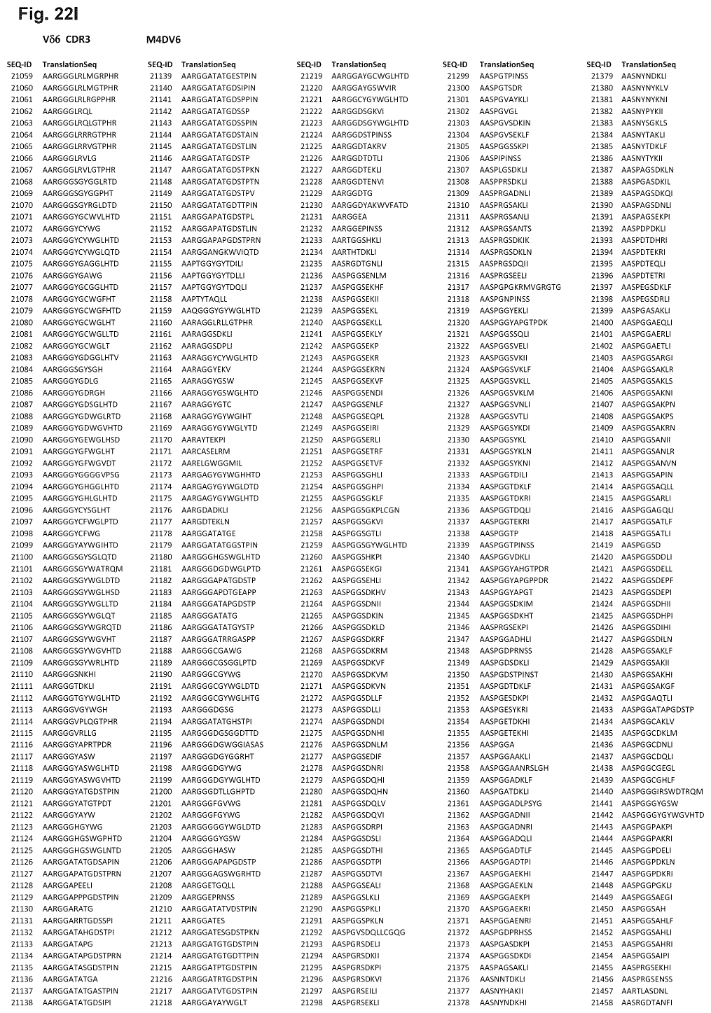

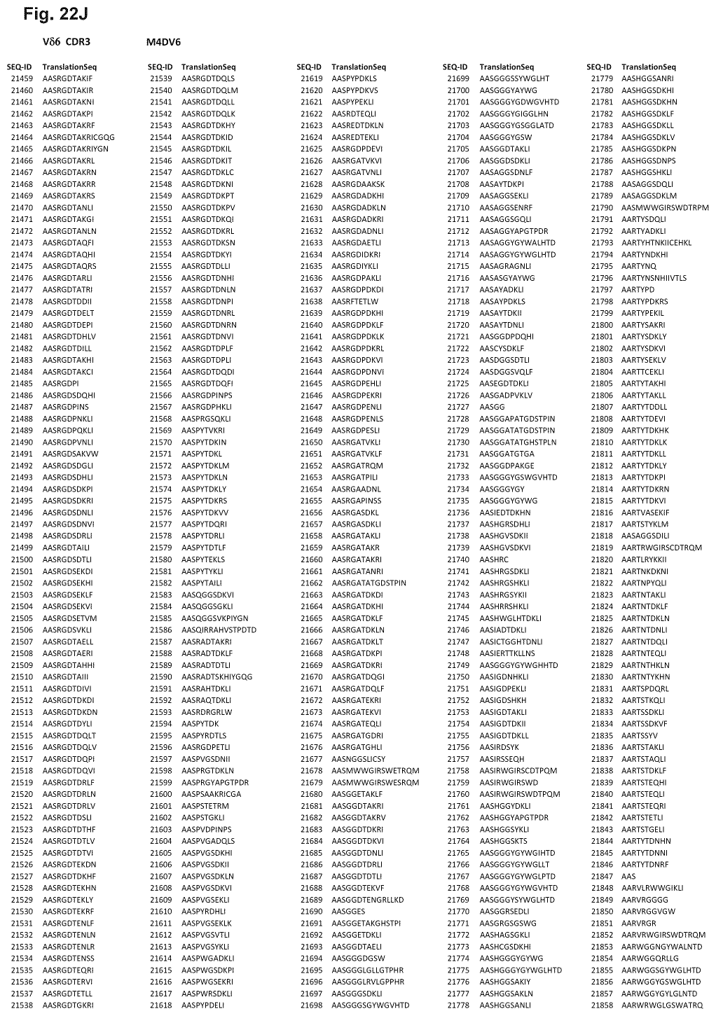

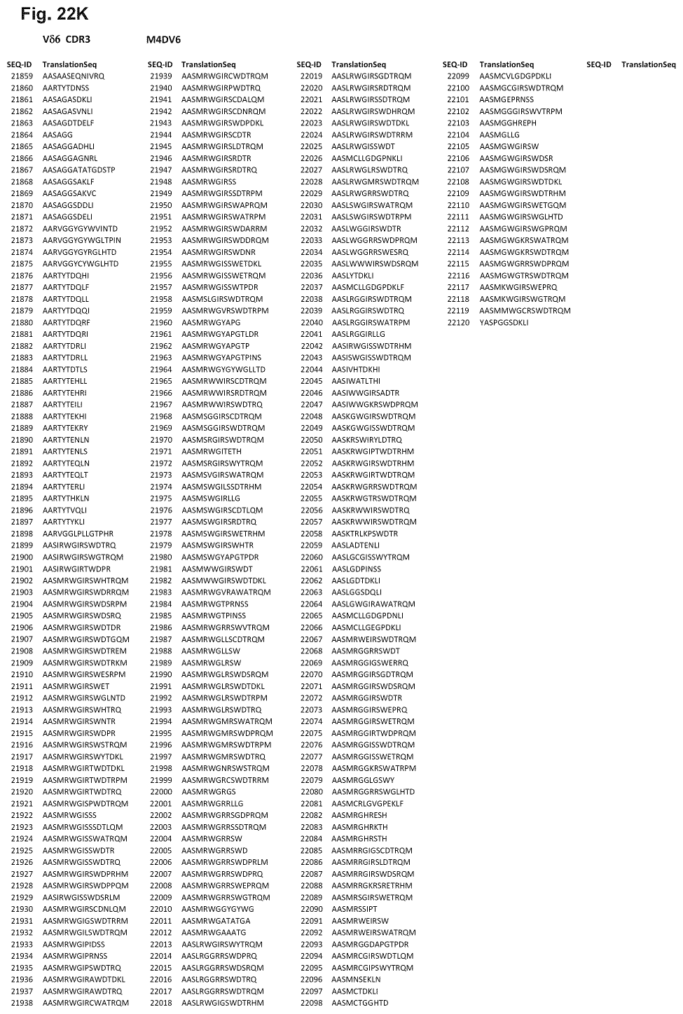

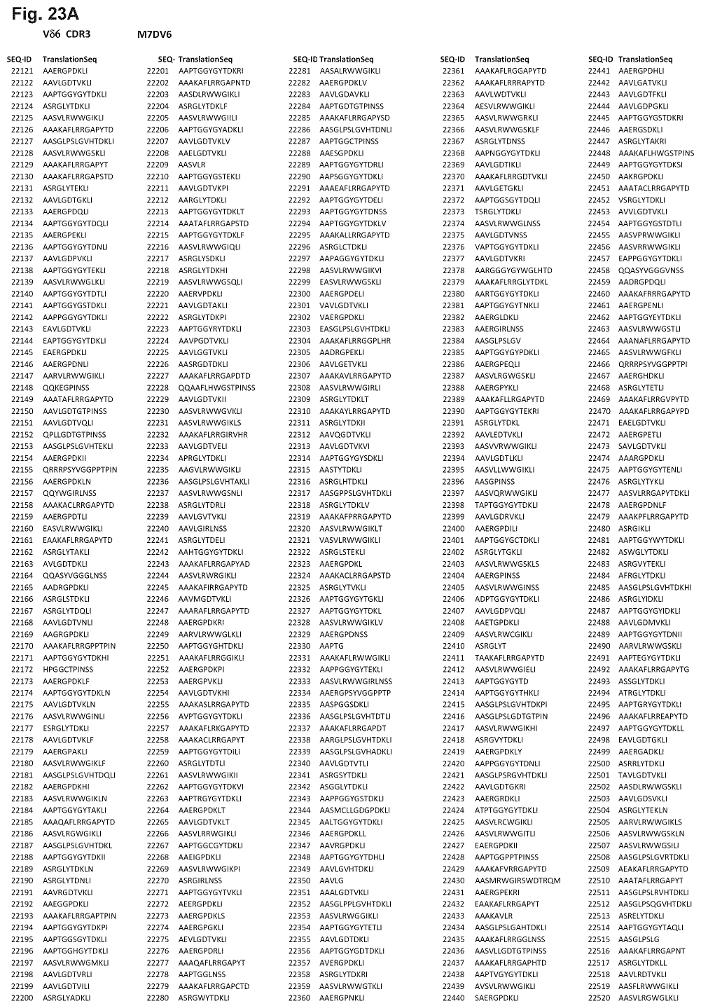

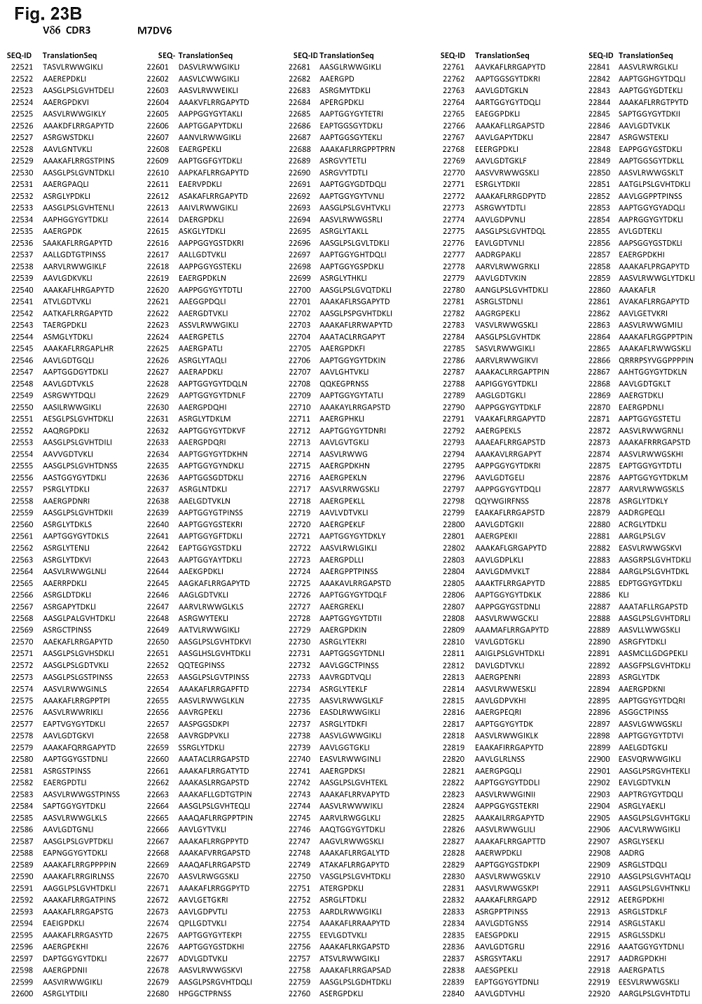

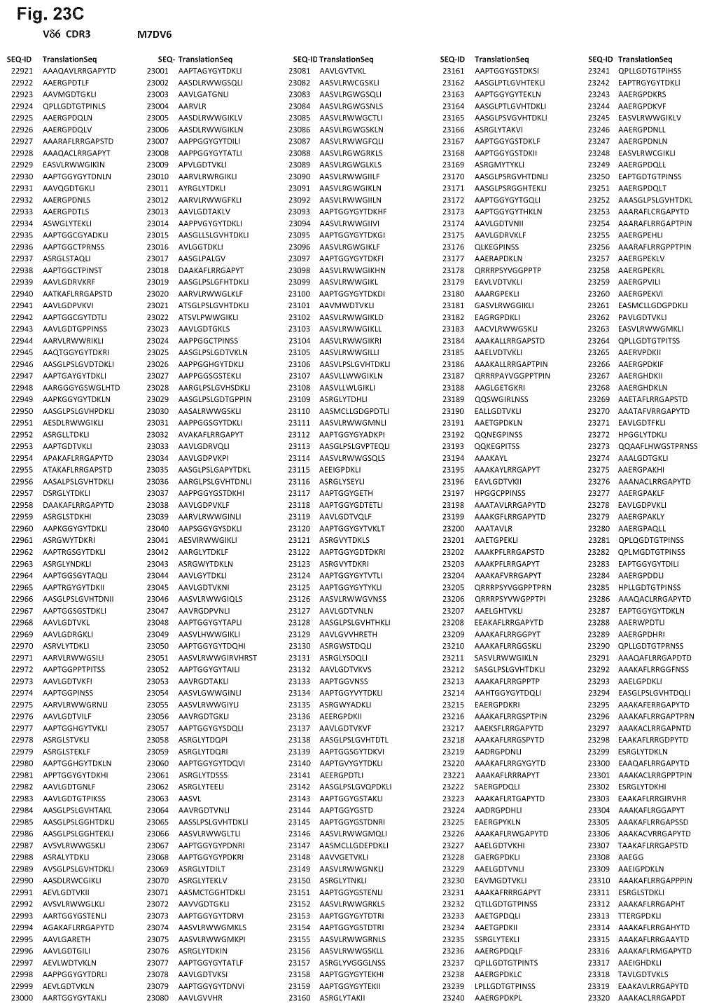

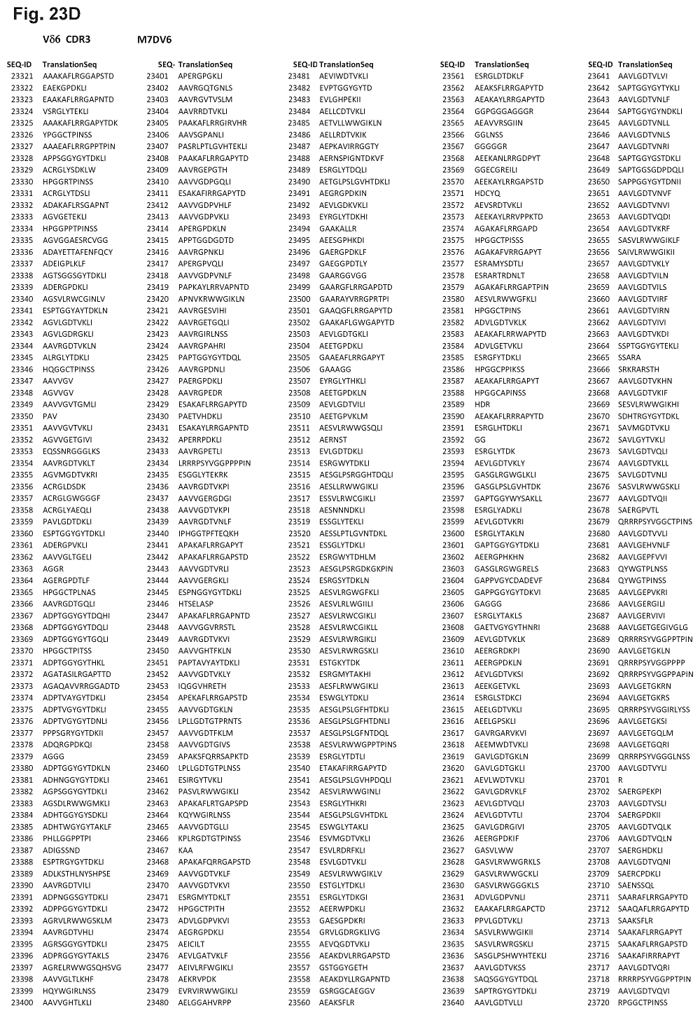

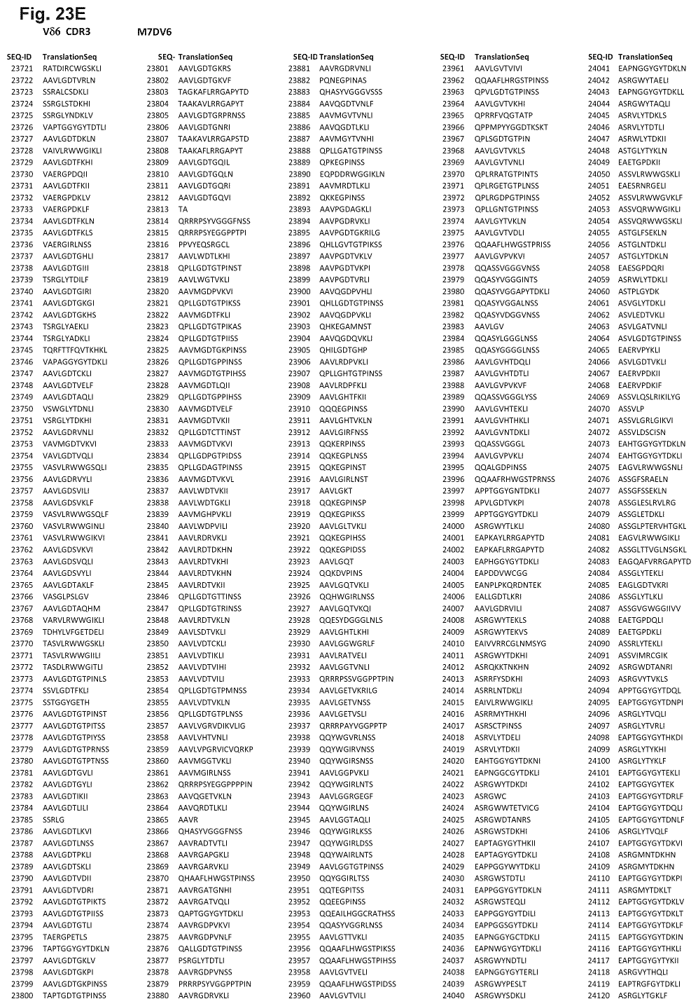

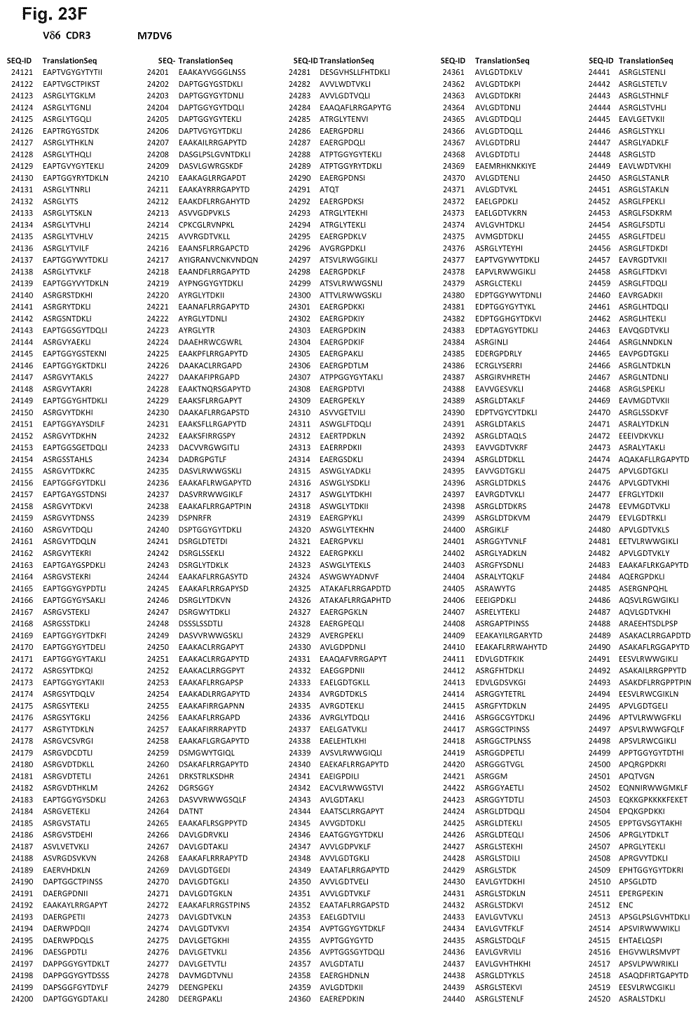

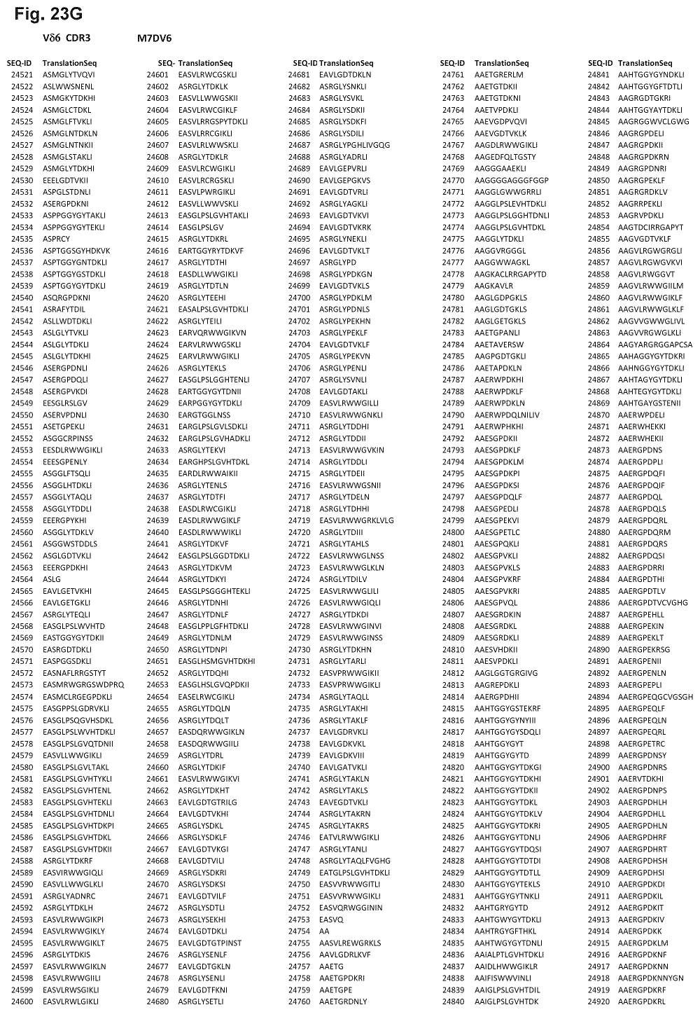

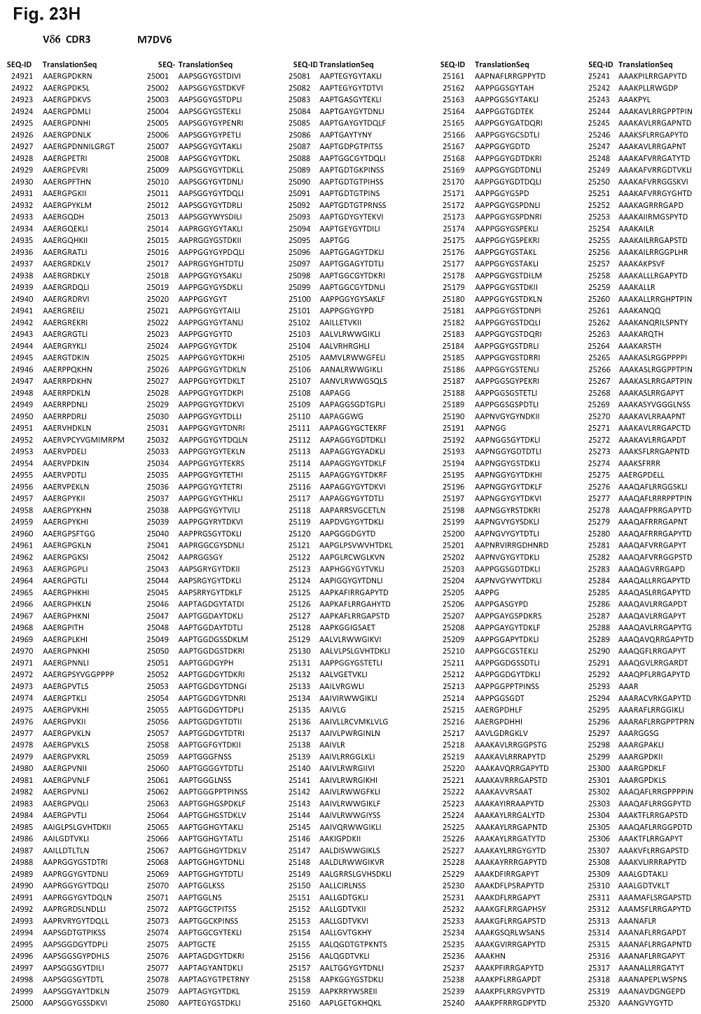

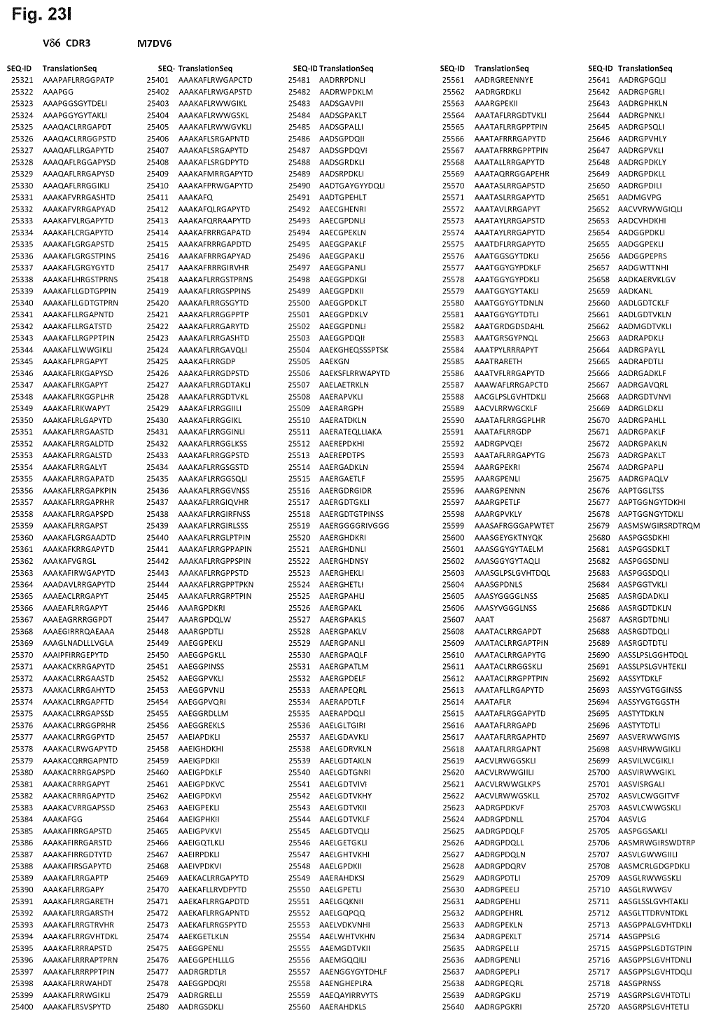

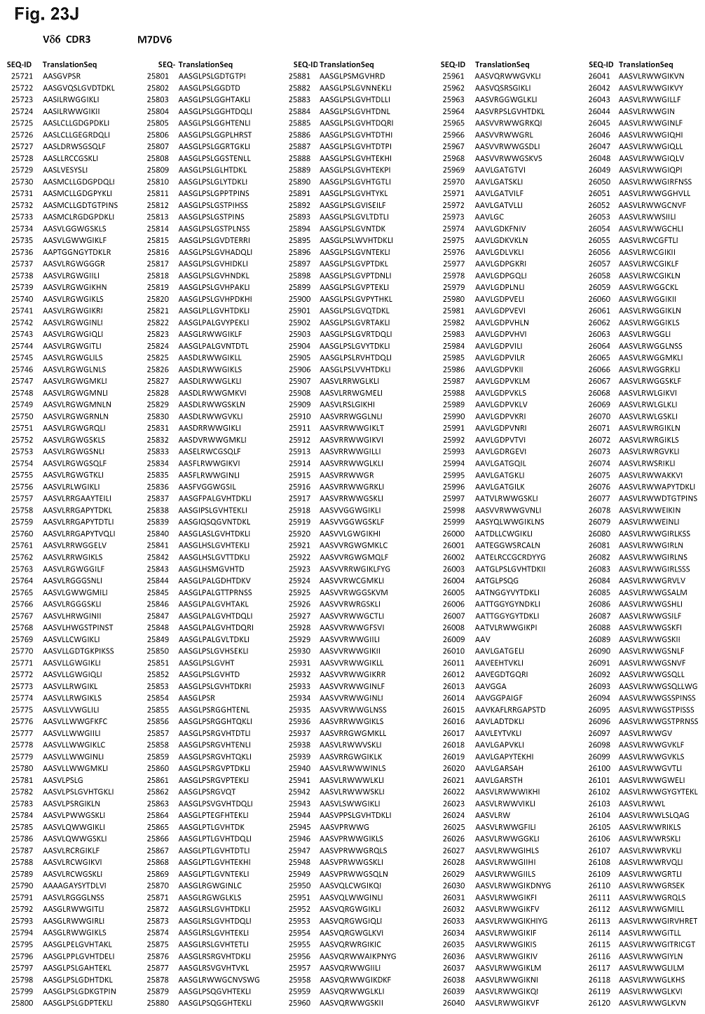

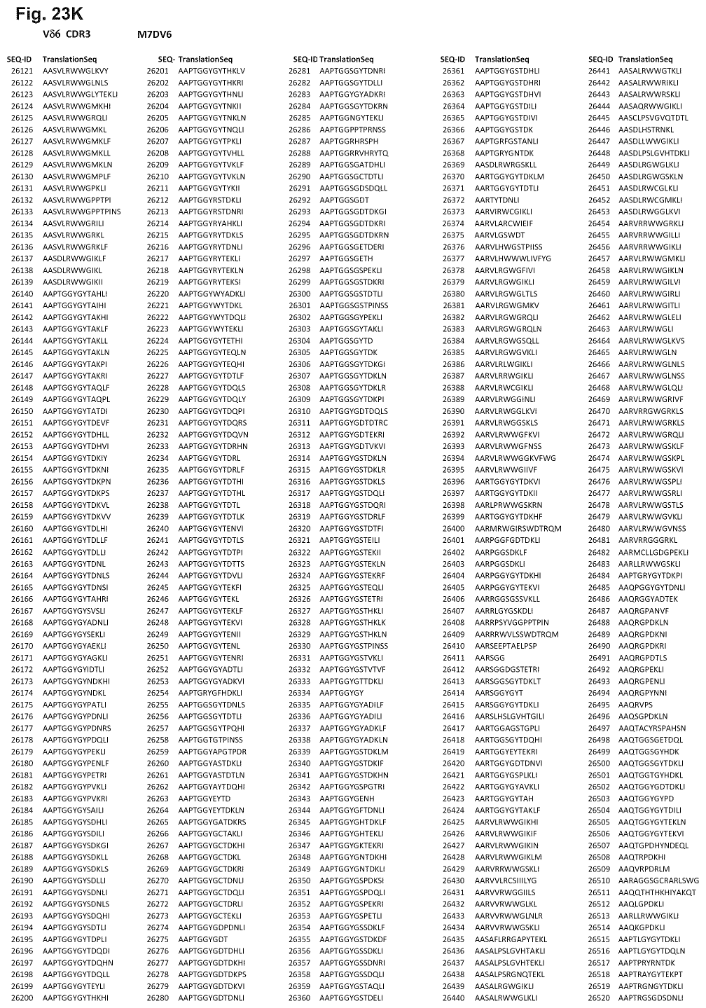

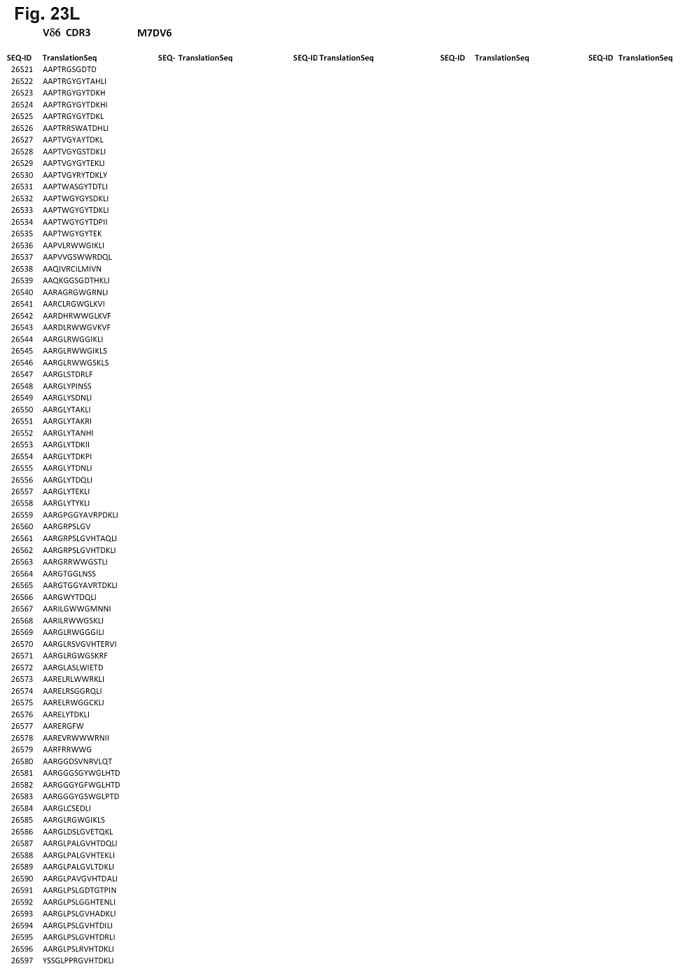

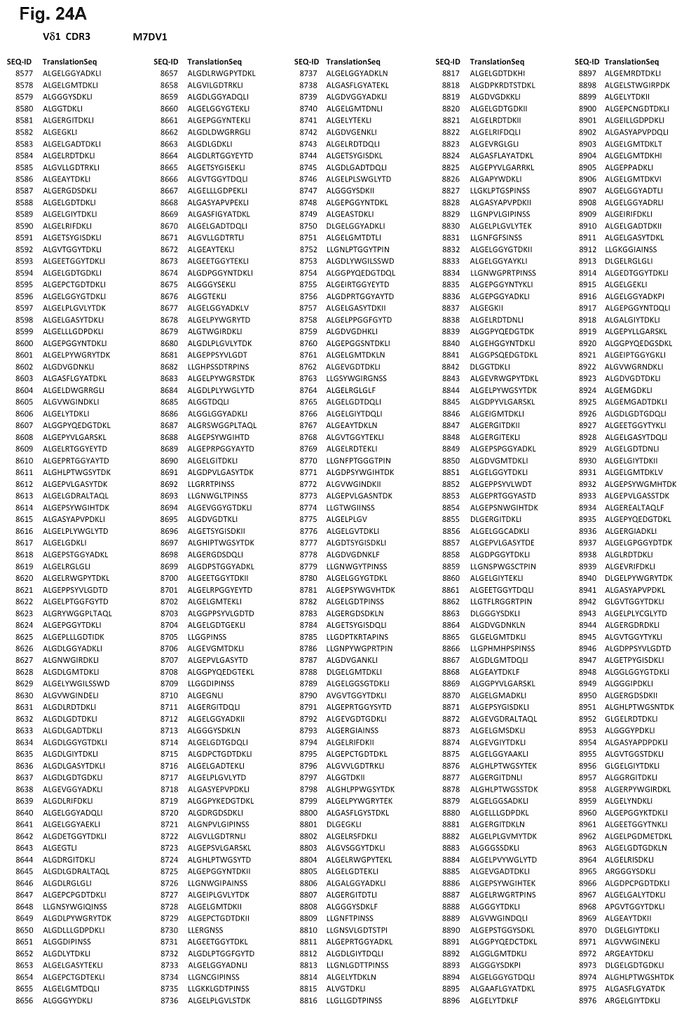

Human TCR Vδ6 is also highly diversified but to a lesser degree than that of TCR Vδ1. In the number one mouse (M4), there were 4263 unique Vδδ CDR3s from only 100,000 DNA sequences from NGS-IgBlast analysis (NGS Seq ID NO: 17859-22120) (FIG. 22A-K). In mouse number two (M7), similarly, there were 4478 unique Vδδ CDR3s (NGS Seq ID NO:22121-26597) (FIG. 23A-L). The Vδδ CDR3s were longer, some up to 16 amino acids. Many CDR3s of Vδ6 are shared between M4 and M7 as among the top twenty CDR of mouse number one (M4), 14 CRD3 s matched with that from 8yyyy, although no CDR3 shared between the top 20 of both mice. The top five CDR3 of human TCR Vδ6 in mouse number one (M4) has a percentage of 21%, 20%, 15%, 11%, and 7%. The top five CDR3 in mouse number two (M7) have a percentage of 15%, 15%, 13%, 11%, 10%, and 9%, indicating a semi-focused repertoire, yet still keeping a large pool of low abundance CDR3 s. In fact, in M4, 84% of CDR3s appeared only one or two times in the total CDR3s, while in M7, 82%. The observation herein indicates that human Vδ6 has a role in certain home-keeping functions, albeit maintaining its readiness to deal with different internal and external challenges faced by each mouse. The observation herein also indicates that human TCRs can work with mouse-based antigens among them many are shared (e.g, lipid-based and sugar-based), so these human TCR mice can serve as a model close to humans and can be used to identify new therapeutic targets and ultimately meaningful human based TCRs for human or animal treatment.

### Example 6

Repertoire Analysis of TCRδ1-4, 6 in Mouse Spleen

In wildtype mouse, γδ T cells reside in spleen as one of major sites (van der Heyde., H. 2006. Infect Immun 75:2717-2725). In the present invention, Human δ TCR CDR3s including Vδ1, Vδ2, Vδ3, Vδ4, and Vδ6 were further studied using next generation sequencing (NGS) approach. The NGS results showed that all the CDR3s were highly diversified. Similar to Example 4, only the first 100,000 sequence reads were subjected to IgBlast analysis. The top ten CDR3s from each δ subclass were listed in Table 11.

TABLE 11
Top Ten CDR3s of TCR delta in Spleen of Human Delta Mice

| SEQ ID No. | Top ten CDR3 | | (%) | Top ten CDR3 | | (%) | SEQ ID No. |
|---|---|---|---|---|---|---|---|
| 26598 | hDV1 | ALGELTYWGIRTDK | 46.0 | hDV2 | ACDSVLGETDKLI | 3.8 | 28542 |
| 26599 | | ALGELVWGIRRVTD | 33.0 | | ACDTLGDTDKLI | 3.1 | 28543 |
| 26600 | | ALGDLTYWGIRTDK | 1.2 | | ACDTSGGLLI | 2 | 28544 |
| 26601 | | ALGDLVWGIRRVTD | 1.1 | | ACGDTKTDKLI | 2 | 28545 |
| 26602 | | ALGKLTYWGIRTDK | 1.1 | | APHKLI | 2 | 28546 |
| 26603 | | ALGELTYWGIRTD | 0.7 | | ACDTGRGPDKLI | 2 | 28547 |
| 26604 | | ALGELTSWGIRTDK | 0.6 | | ACDTSGDKLI | 1.9 | 28548 |
| 26605 | | ALGELTYCGIRTDK | 0.5 | | ACDTEGQTH | 1.9 | 28549 |
| 26606 | | LLGNSSGGYGGSPI | 0.5 | | ACDTGDTTDKLI | 1.9 | 28550 |
| 26607 | | ALGELTYWGISTDK | 0.4 | | ACDTGWGITDKLI | 1.9 | 28551 |
| 32584 | hDV3 | AFTGG | 1.1 | hDV4 | AMERGLGDTDKLI | 0.4 | 33869 |
| 17282 | | AFS | 0.8 | | AMREGDSTDKLI | 0.4 | 33870 |
| 16706 | | AFFLR | 0.7 | | AMREGWGIPDKLI | 0.3 | 33871 |
| 32587 | | AFPGG | 0.7 | | AMREGDGGYTDKLI | 0.3 | 33872 |
| 32588 | | AFLGV | 0.6 | | AMREPGGPDKLI | 0.3 | 33873 |
| 17641 | | AFLRWG | 0.6 | | ASPGYTDKLI | 0.3 | 33874 |
| 32590 | | AFILTP | 0.6 | | AMREVLGDTDKLI | 0.3 | 33875 |
| 32591 | | ASSTG | 0.6 | | AMREGEGYTDKLI | 0.3 | 33876 |
| 32592 | | AFRLG | 0.5 | | AMRENTDKLI | 0.2 | 33877 |
| 16857 | | AFSY | 0.5 | | AMREGLGATINSS | 0.2 | 33878 |
| | | | | hDV6 | AASMSGGYNTDKLI | 0.6 | 38497 |
| | | | | | AARLGDTDKLI | 0.6 | 38498 |
| | | | | | AASLGDTDKLI | 0.6 | 22062 |
| | | | | | AARVSWVDKLI | 0.5 | 38500 |
| | | | | | AATDKLI | 0.5 | 38501 |
| | | | | | AASTGGYTDKLI | 0.5 | 38502 |
| | | | | | AASSGIRNDKLI | 0.4 | 38503 |
| | | | | | AASPVRTGGL | 0.4 | 38504 |
| | | | | | AVGDTWLI | 0.4 | 38505 |
| | | | | | AANWVPDKLI | 0.4 | 38506 |

For human Vδ1 (NGS Seq ID NO:26598-28541), there is a total of 1944 unique CRD3 sequences (FIG. 9A-E). The top 2 CRD3s count for 46% and 32%, respectively. The CDR3s which appeared only once and two count for 83% of total 1944 CDR3s, indicating relatively focused repertoire which is quite different than the results from Vδ1 from blood.

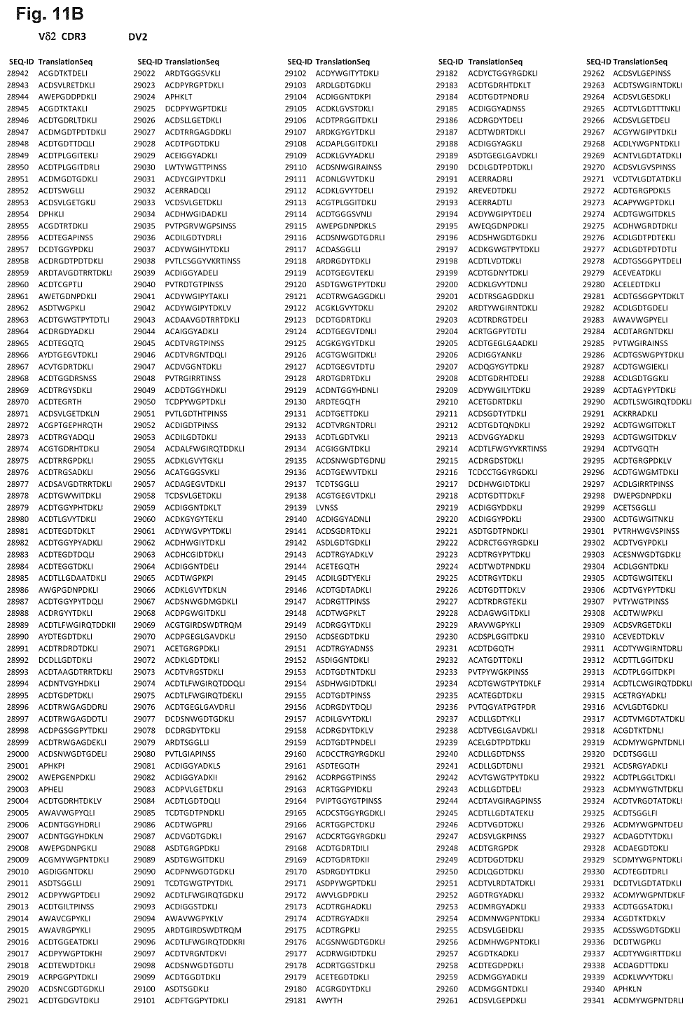

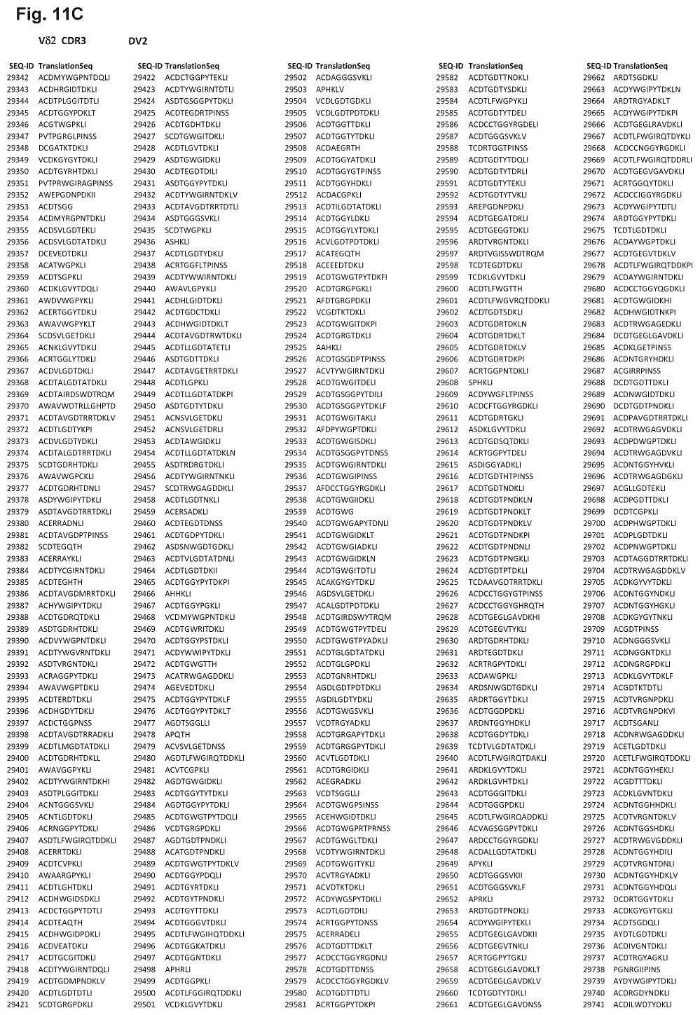

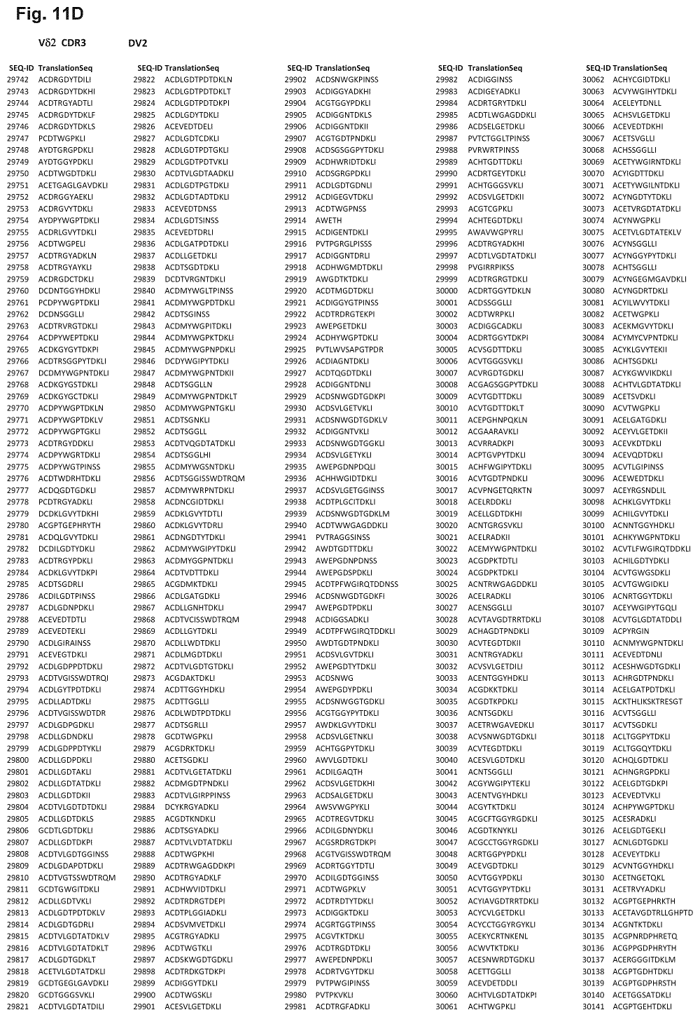

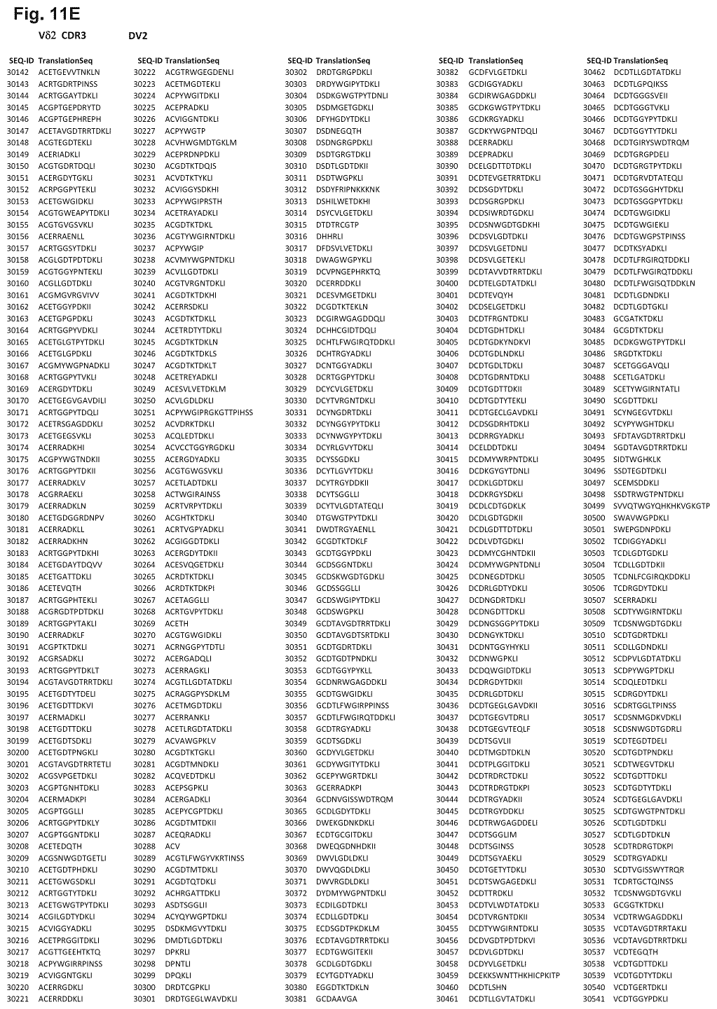

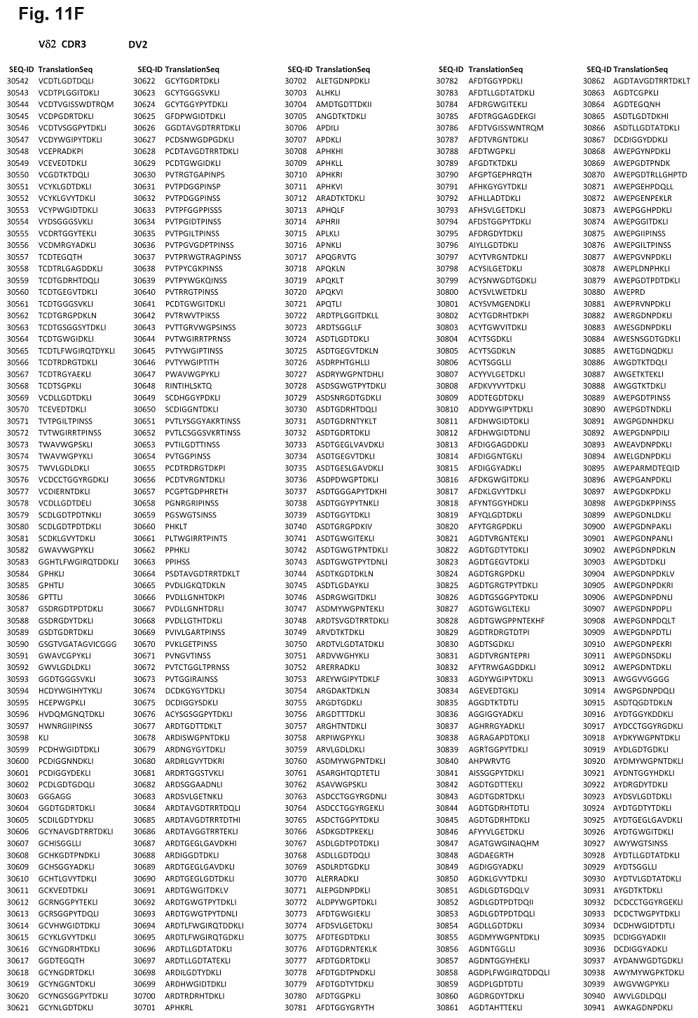

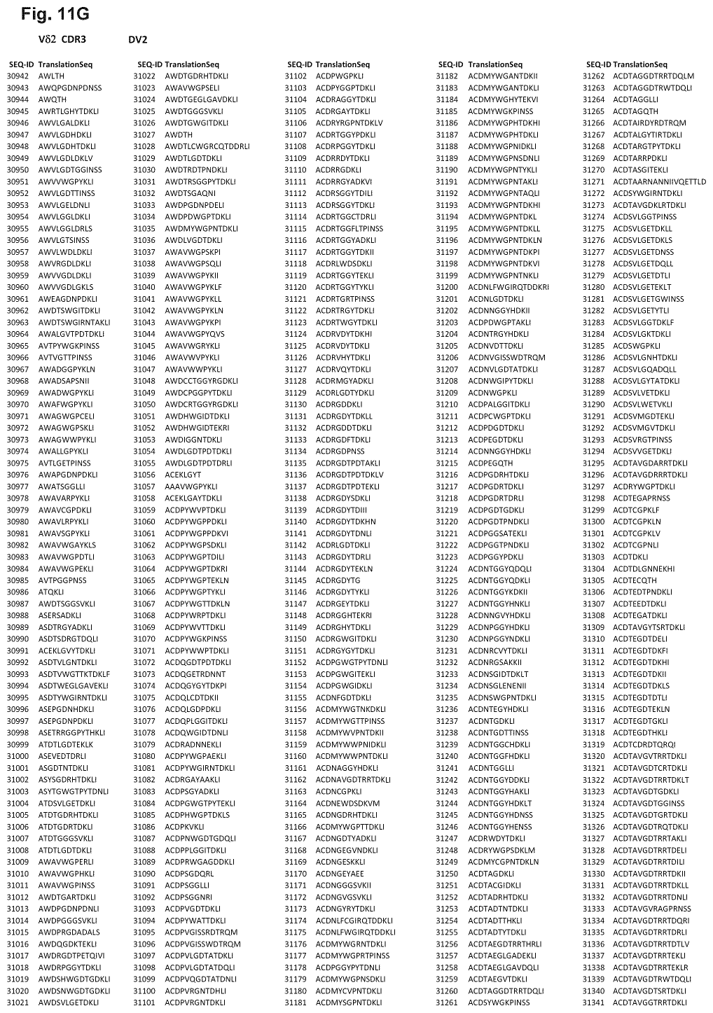

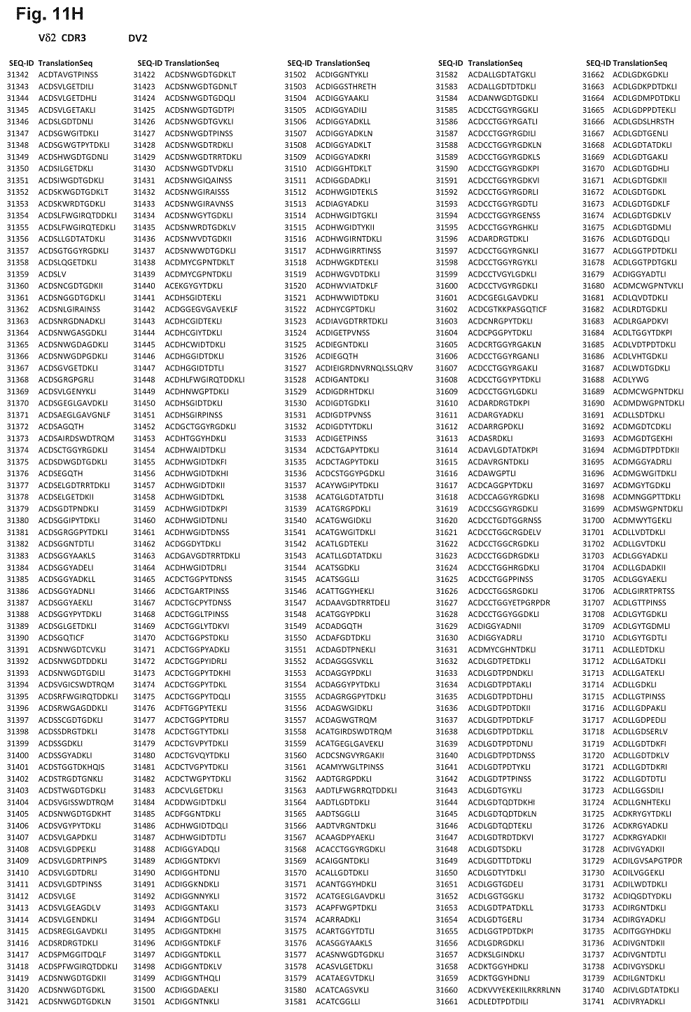

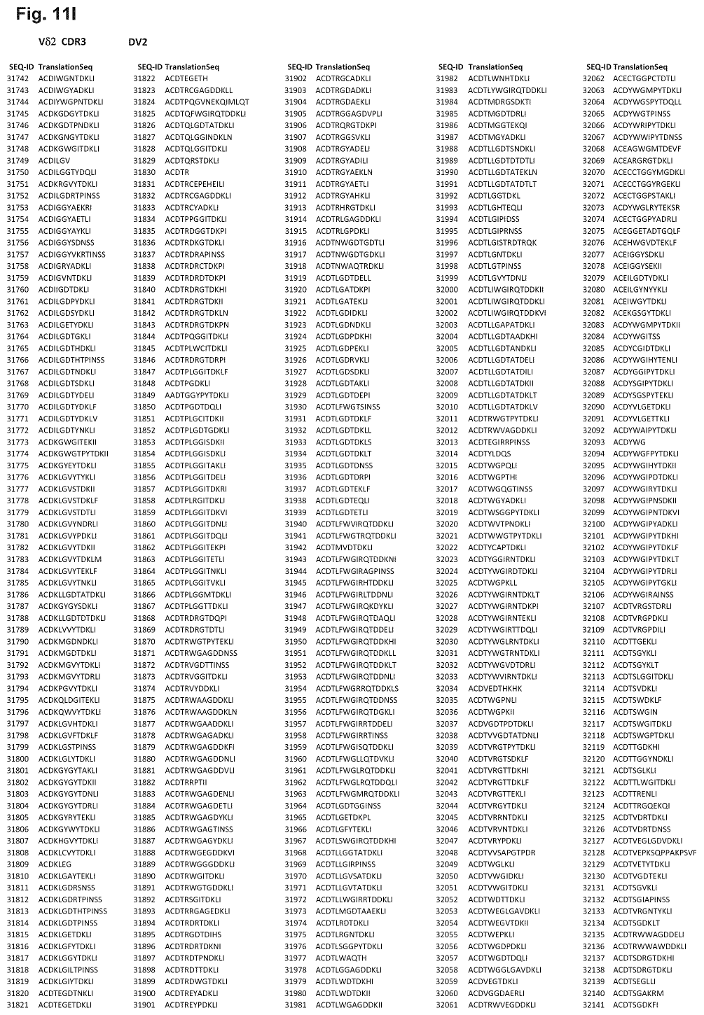

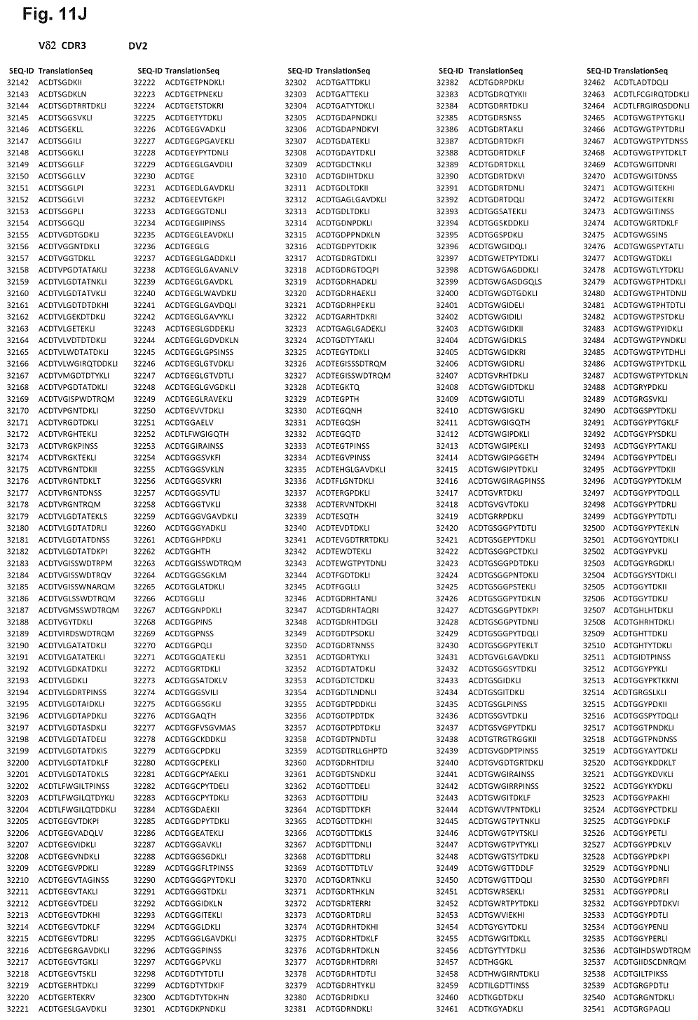

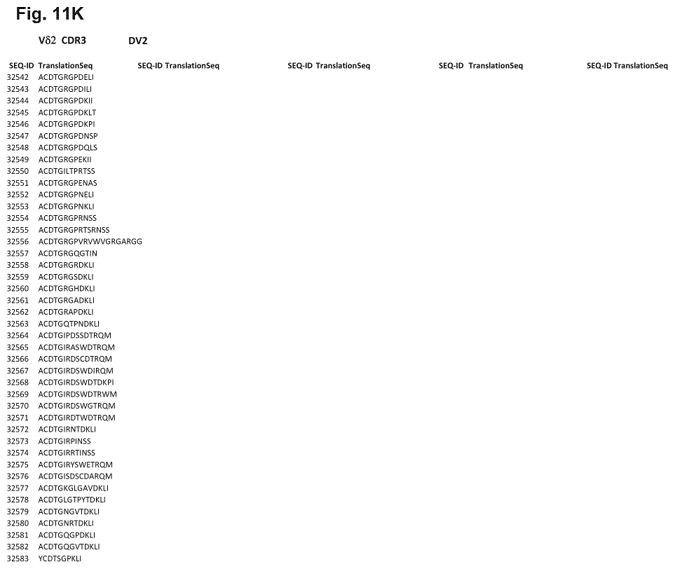

For human Vδ2 (NGS Seq ID NO:28542-32583), there is a total of 4043 unique CDR3 sequences (FIG. 11A-K). The top 2 CRD3s count for 3.8% and 3.1%, respectively. The CDR3s which appeared only once and two count for 80% of the total 4043 CDR3s, indicating a relatively un-focused repertoire.

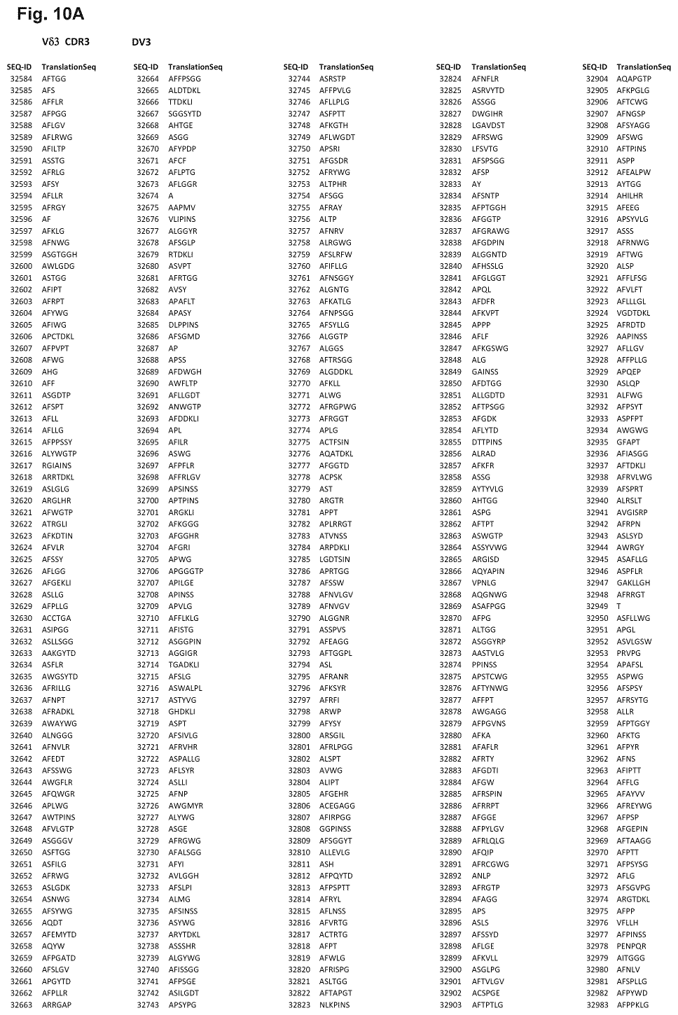

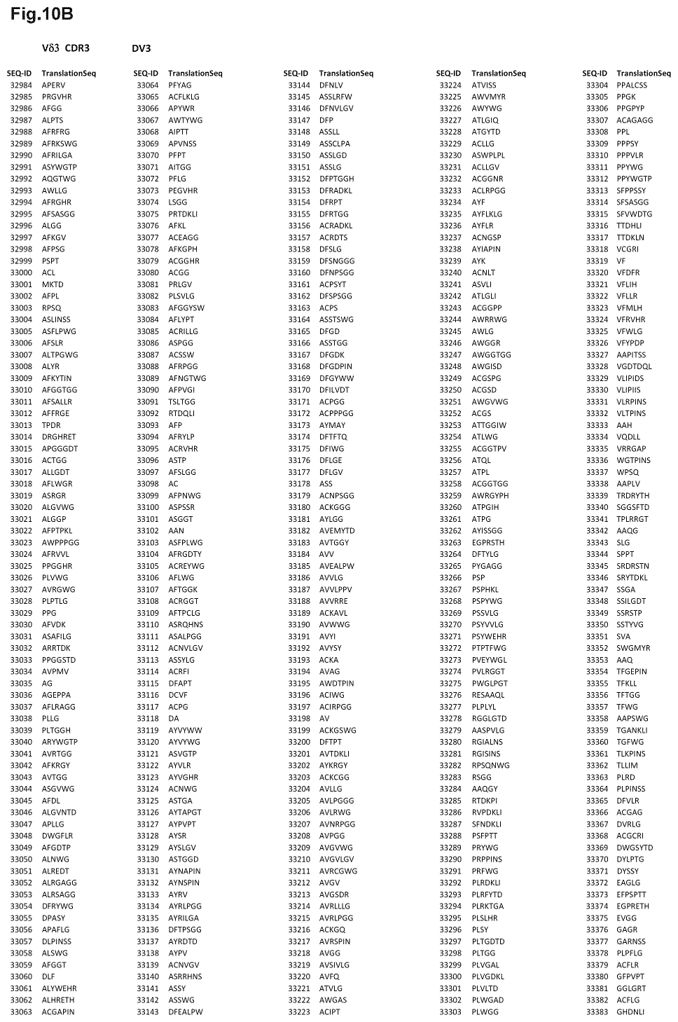

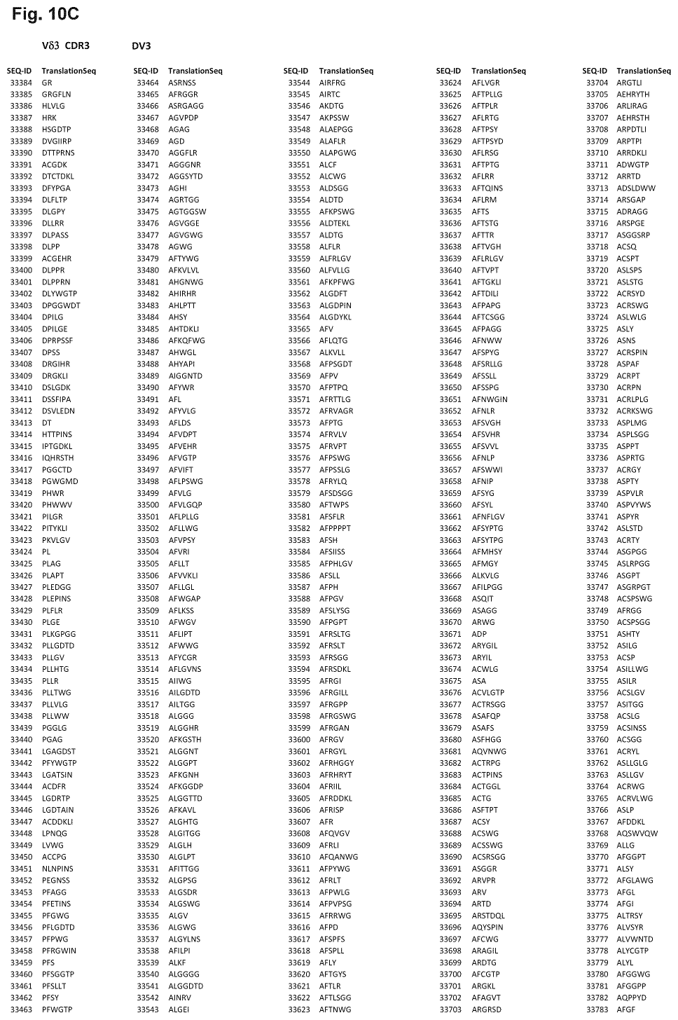

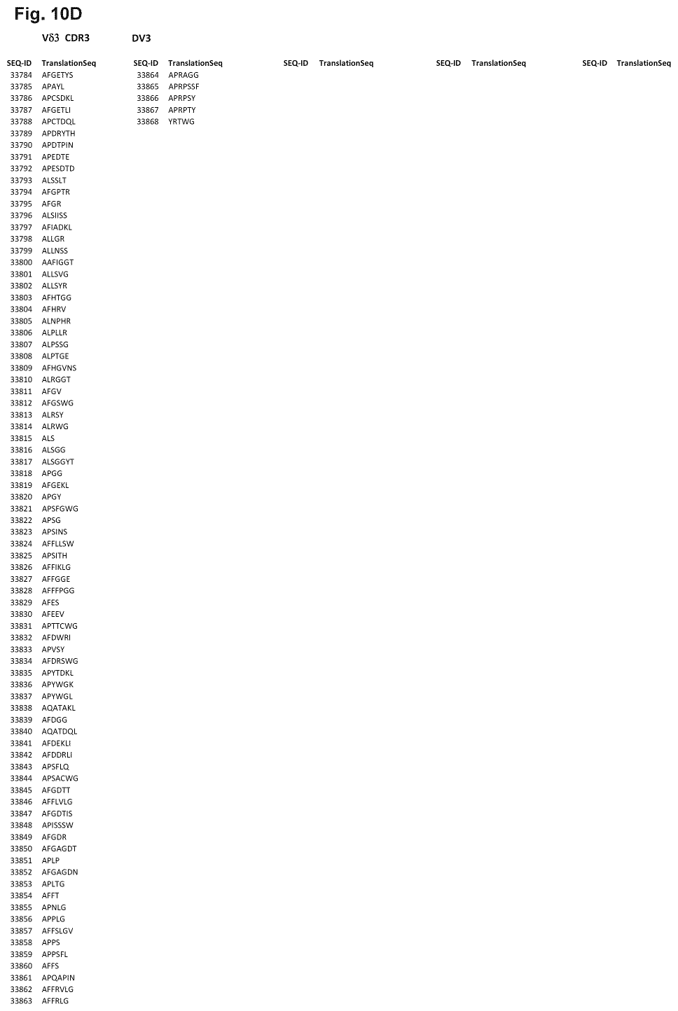

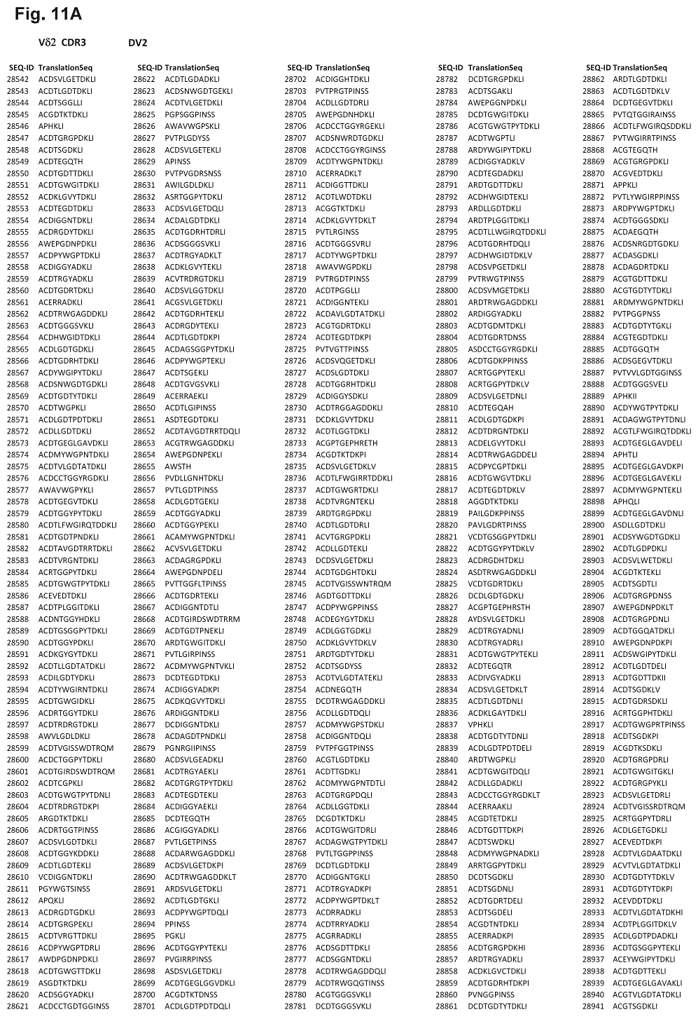

For human Vδ3 (NGS Seq ID NO:32584-33868), there is a total of 1285 unique CRD3 sequences (FIG. 10A-D). The top 2 CRD3s count for 1.1% and 0.8%, respectively. The CDR3s which appeared only once and two counted for 65% of the total 1285 CDR3s, indicating an un-focused repertoire, in sharp contrast to Vδ3 CDR3s in blood where the top two counted for 83% of all the CDR3s.

The results from Vδ1 and Vδ3 also demonstrated that different organs may have their own characteristic repertoire which is highly adaptive and flexible.

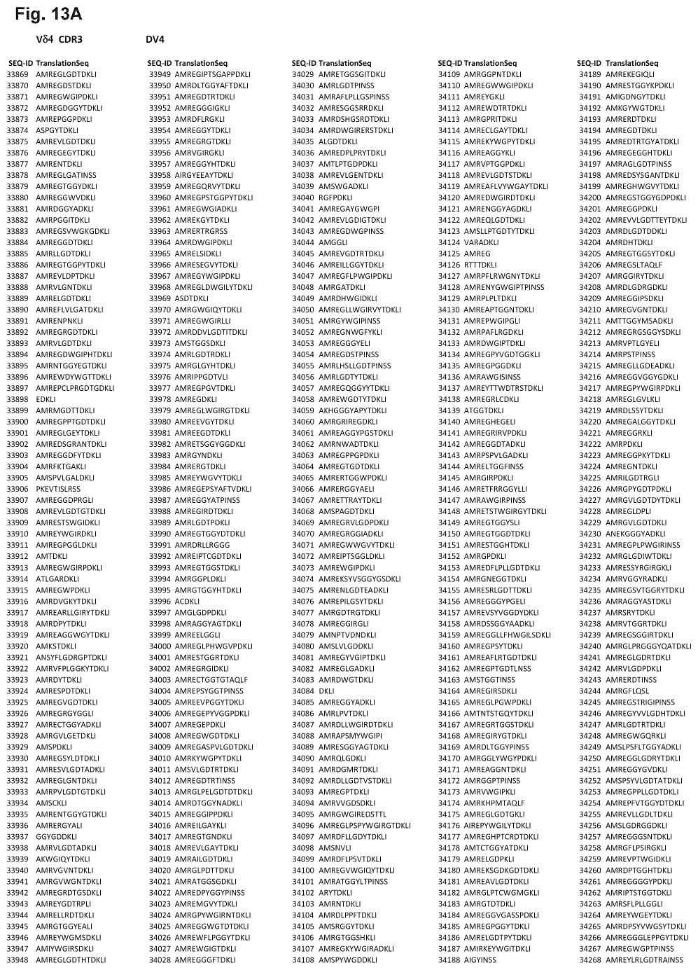

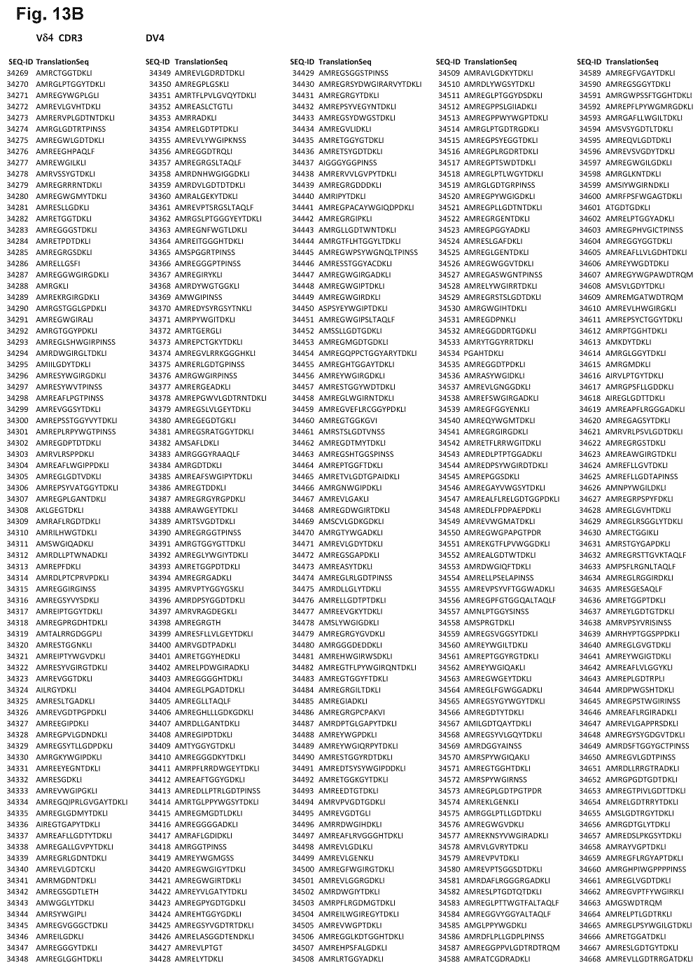

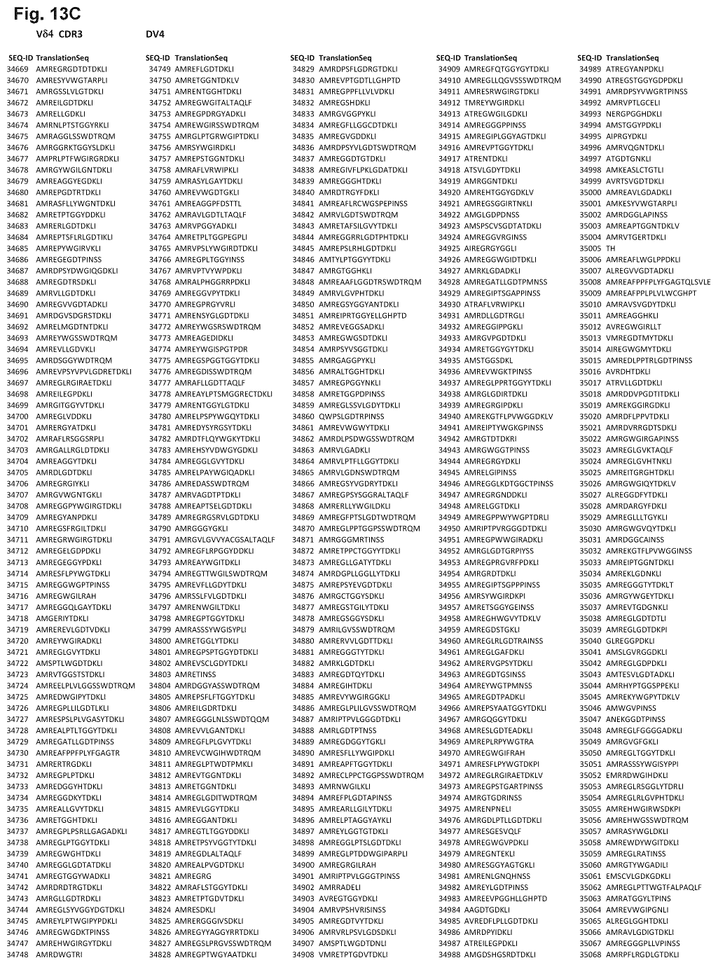

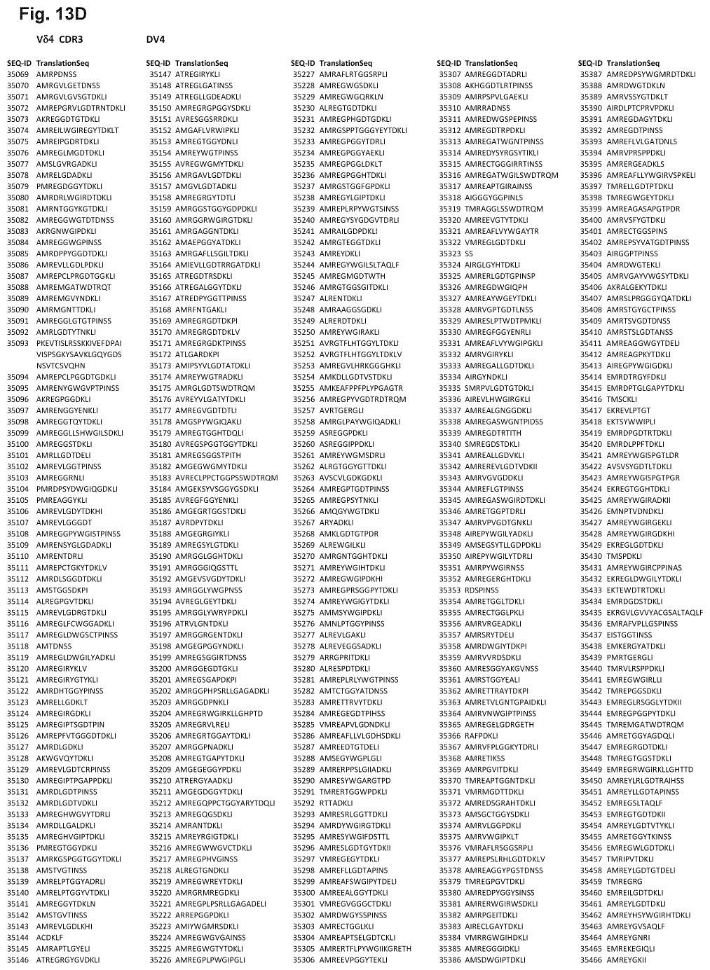

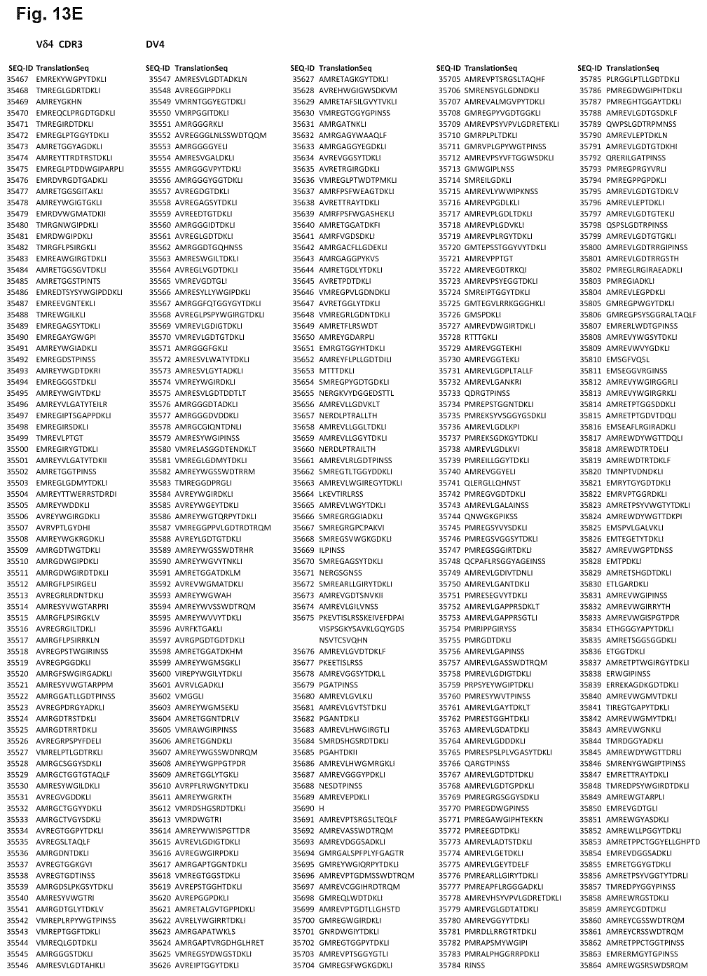

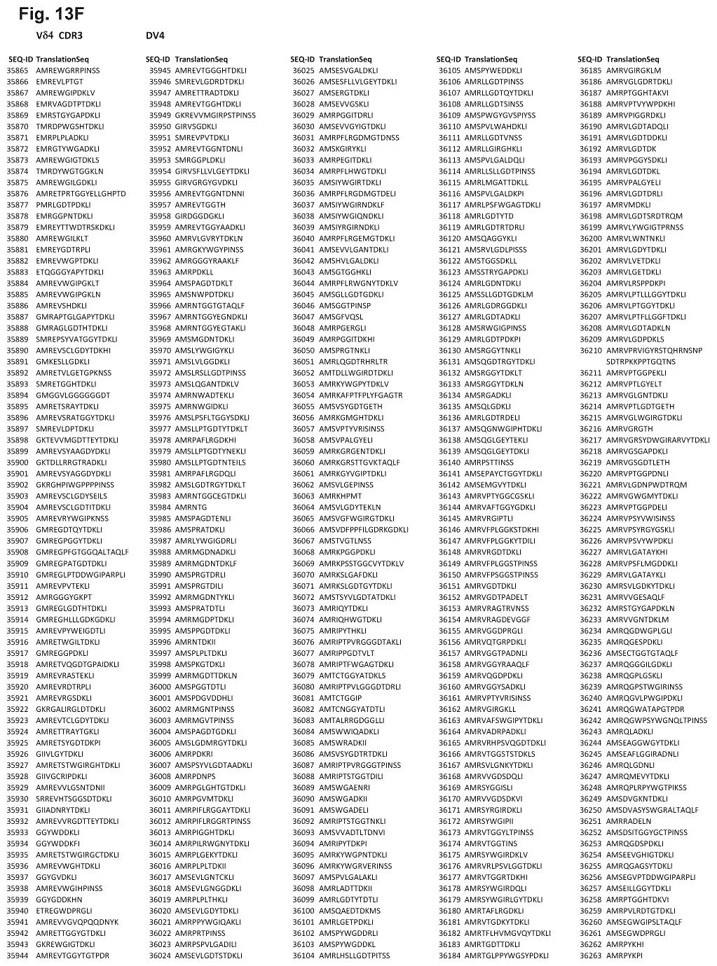

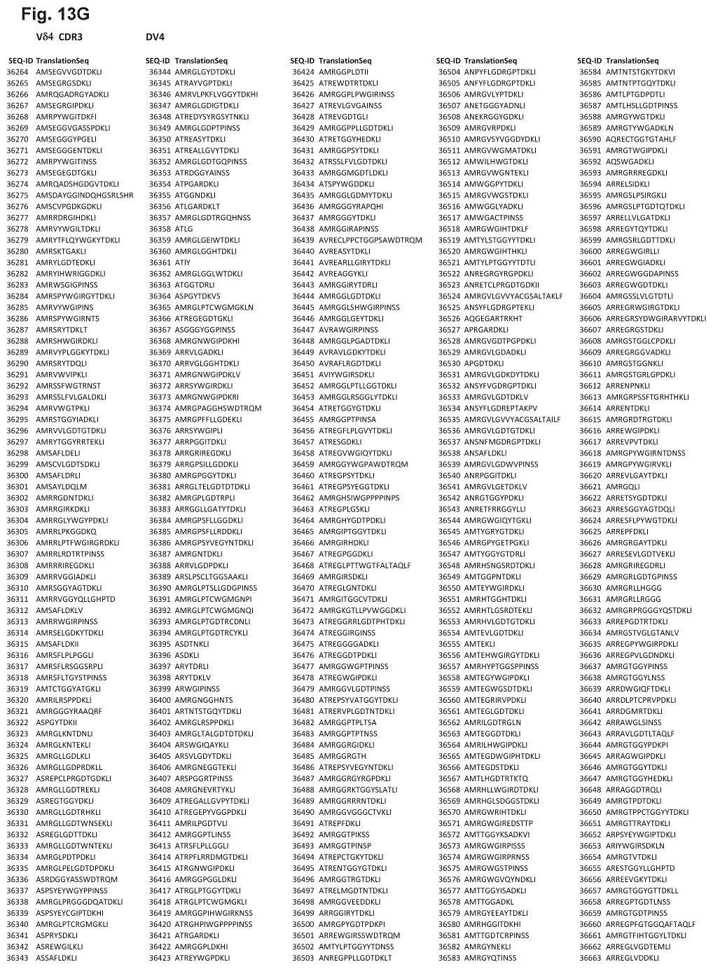

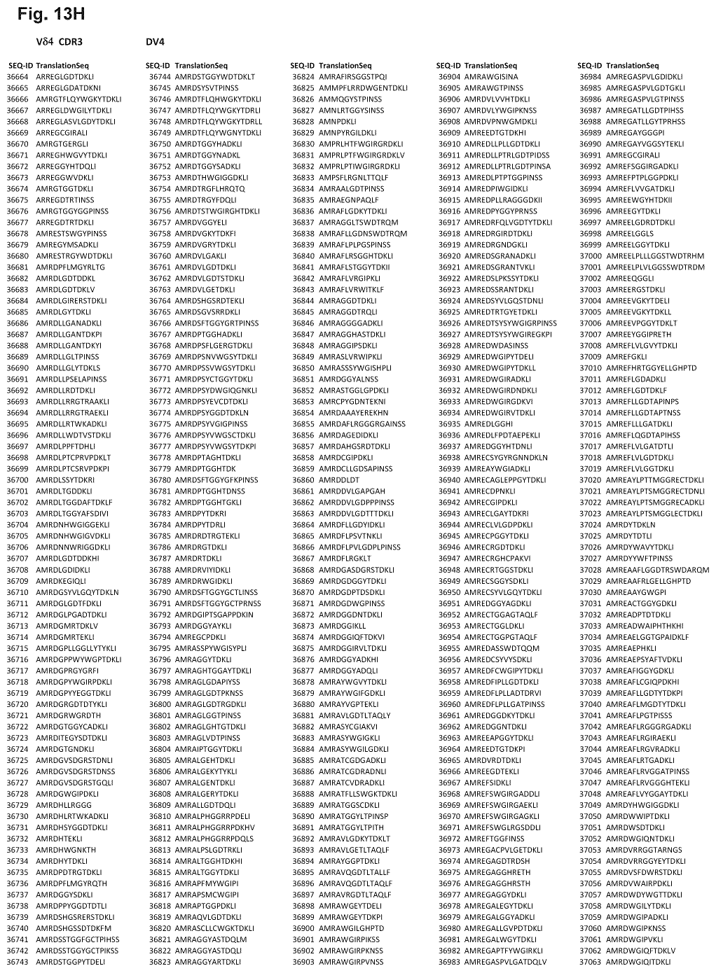

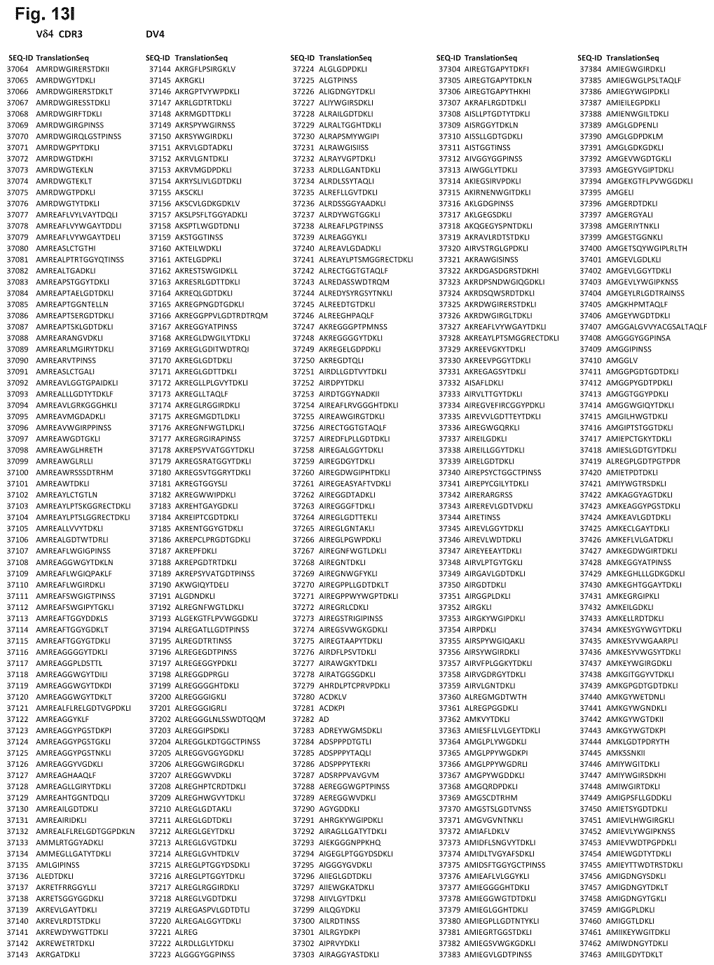

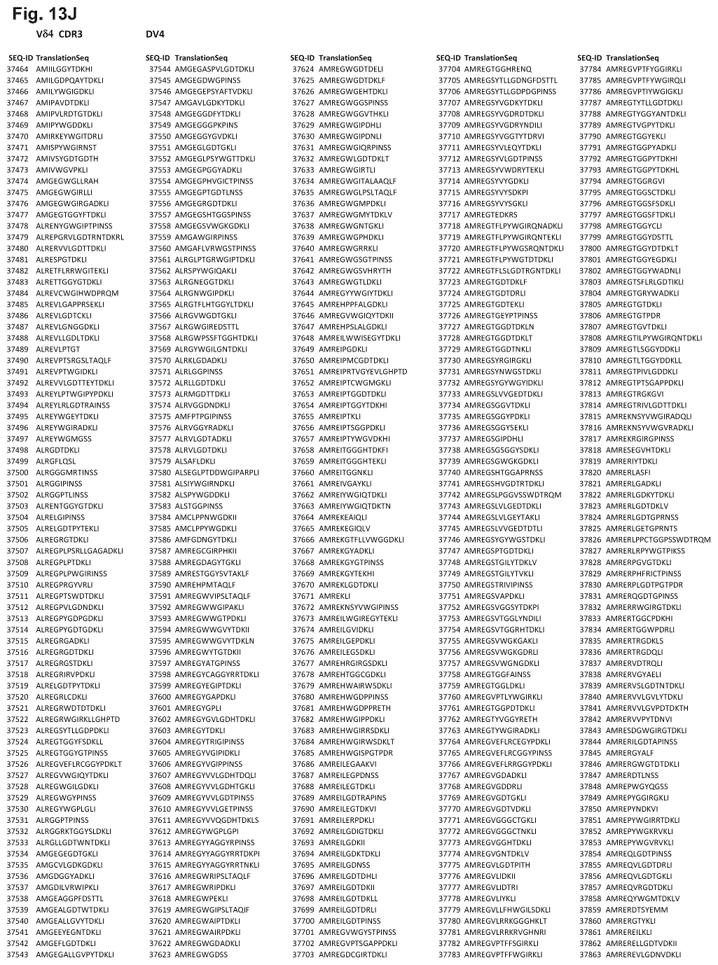

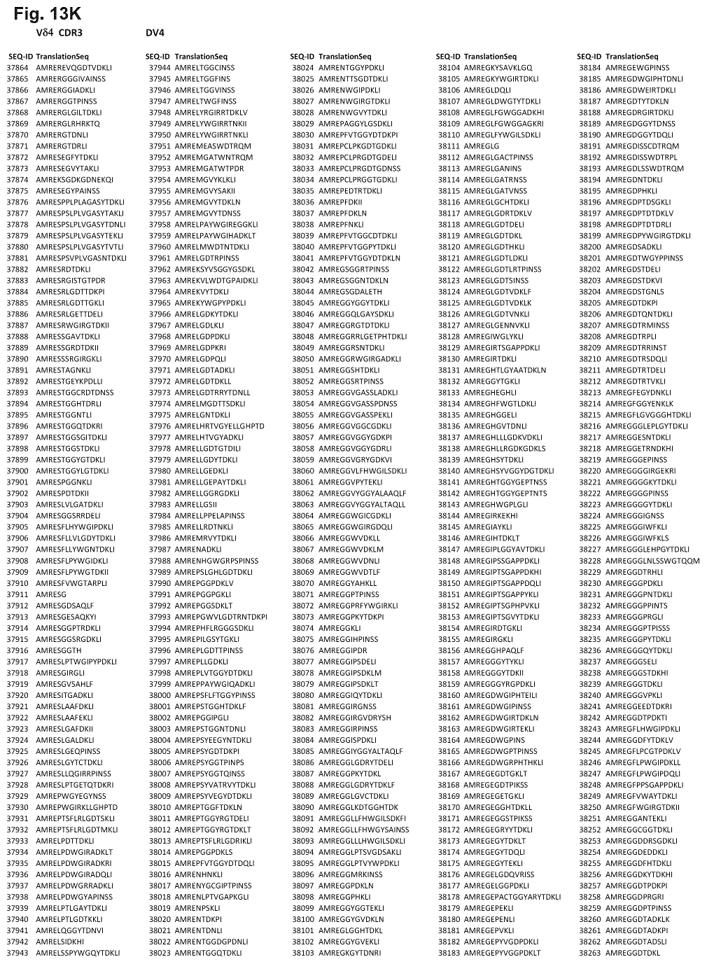

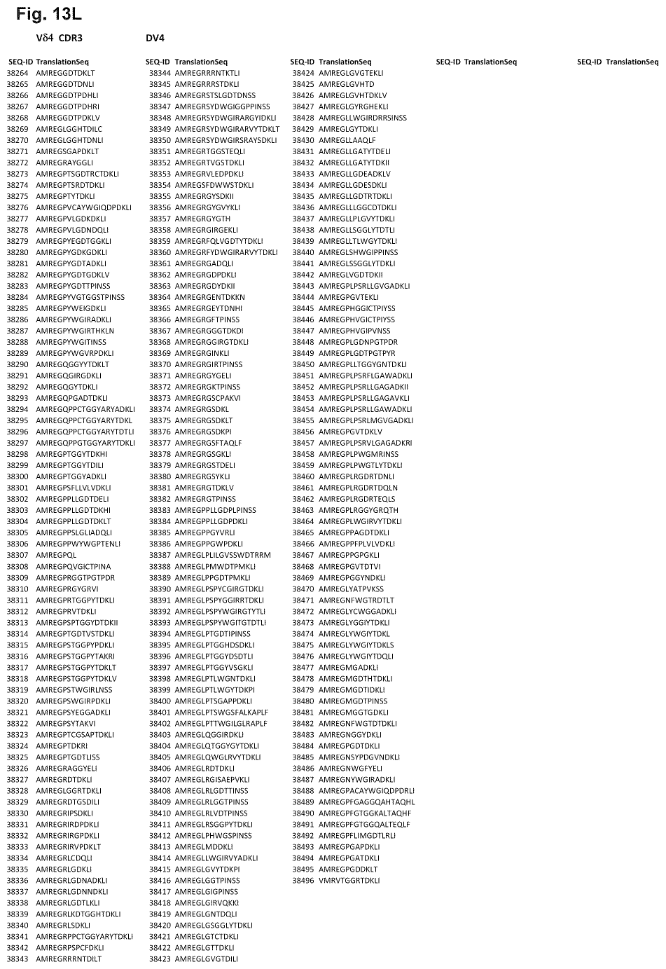

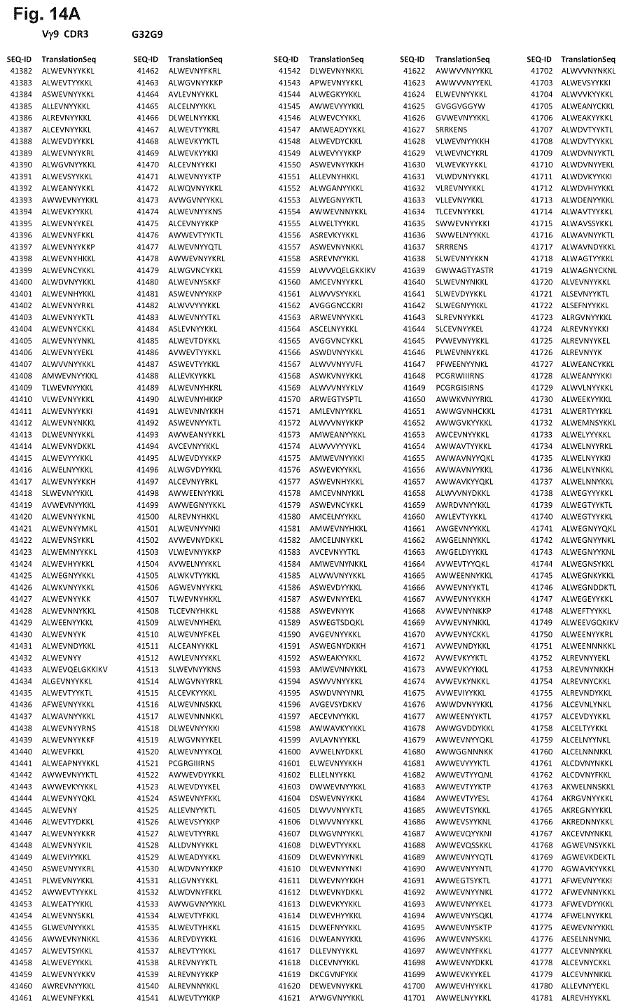

For human Vδ4 (NGS Seq ID NO:33869-38496), there is a total of 4628 unique CRD3 sequences (FIG. 13A-L). Both of the top 2 CRD3s count for 0.4% each. The CDR3s which appeared only once and two count for 75% of the total 4628 CDR3s, indicating an un-focused repertoire. The results herein also revealed that the diversification and quantity of Vδ4 are equivalent to Vδ2, which offers new research opportunities, especially to study tissues where access in humans was very limited.

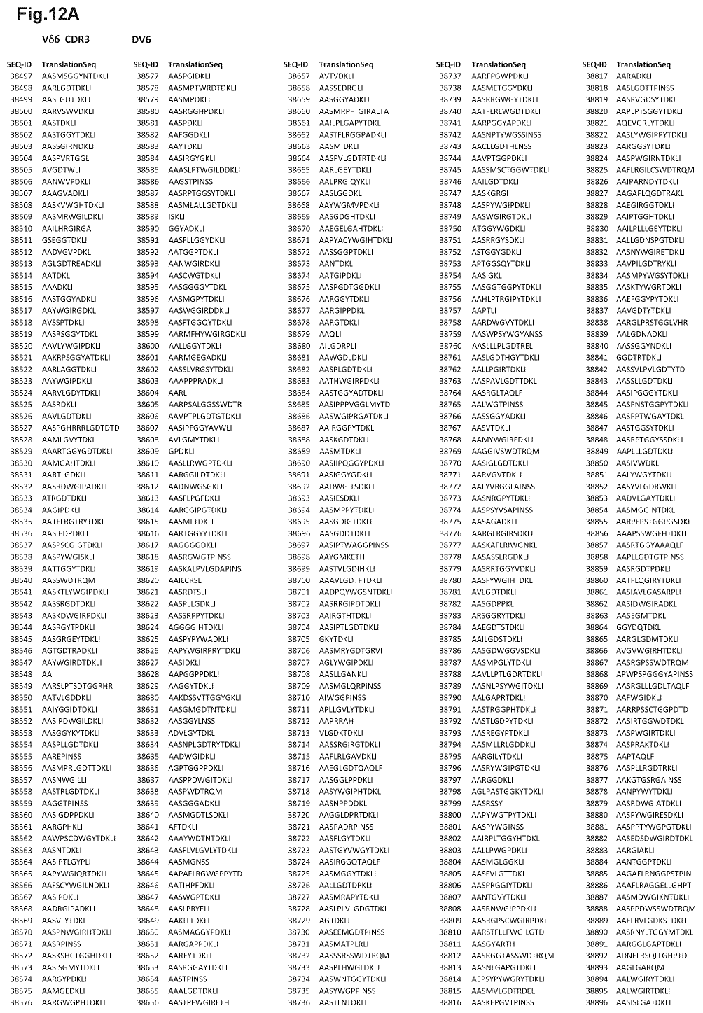

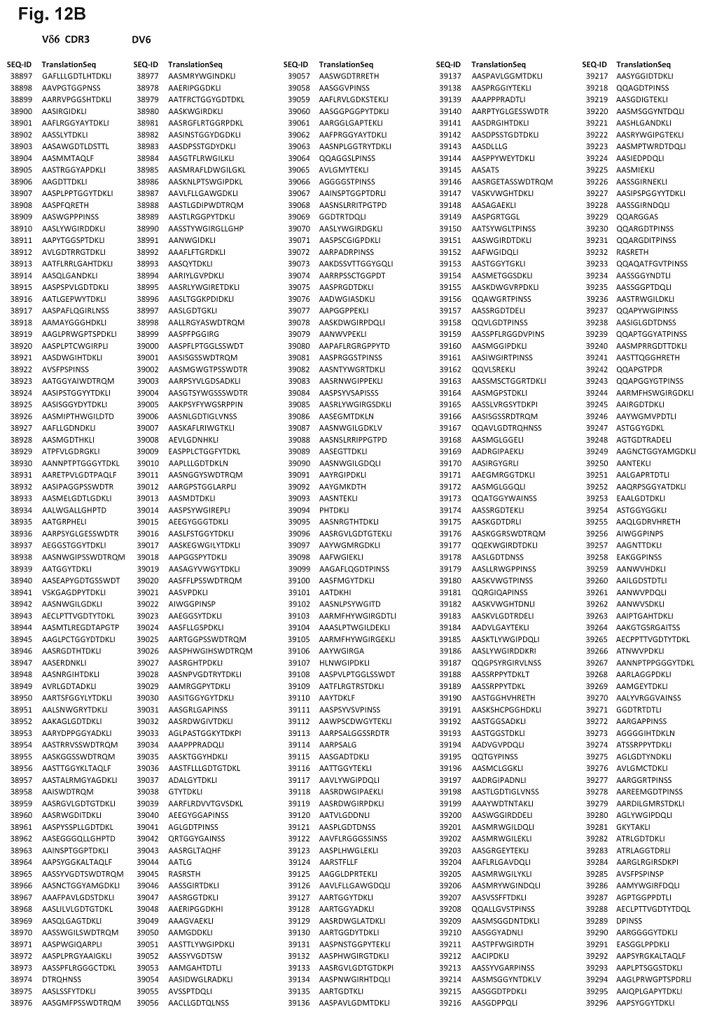

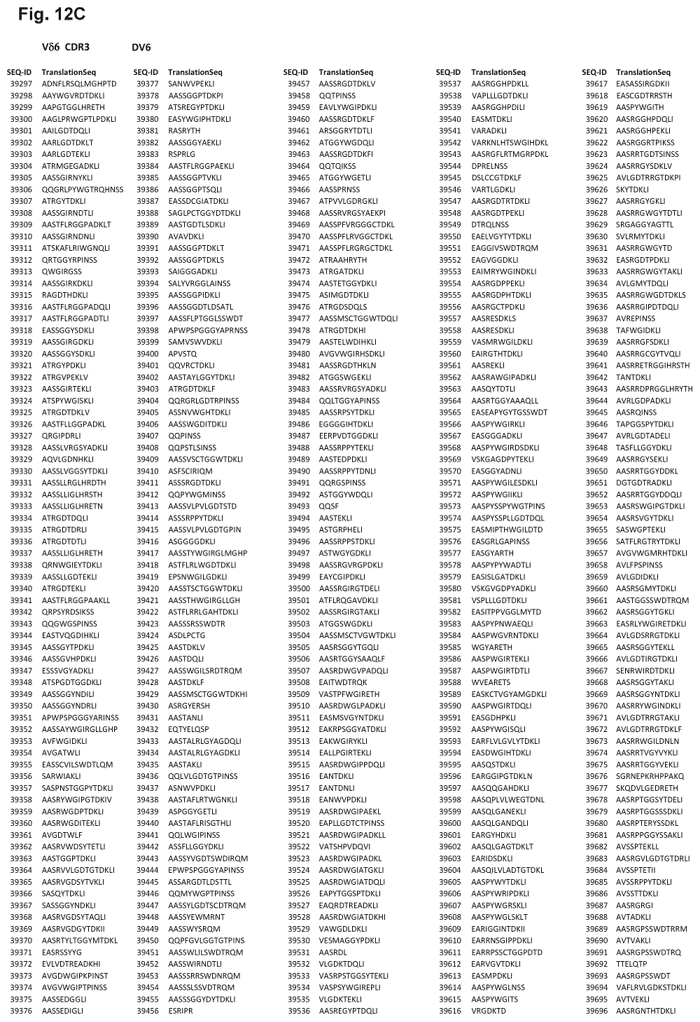

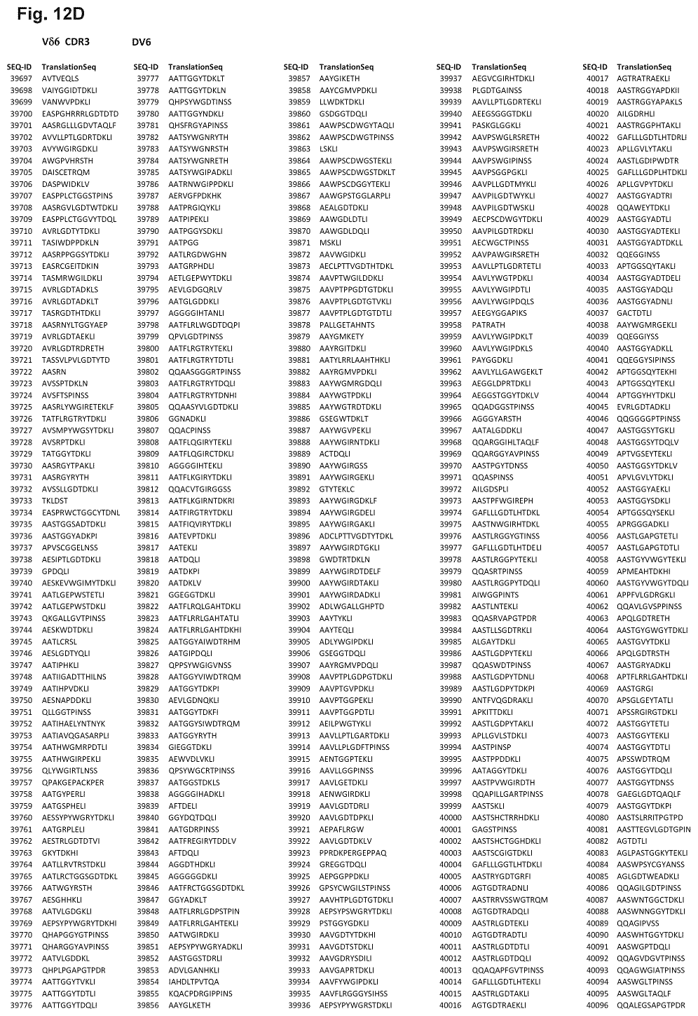

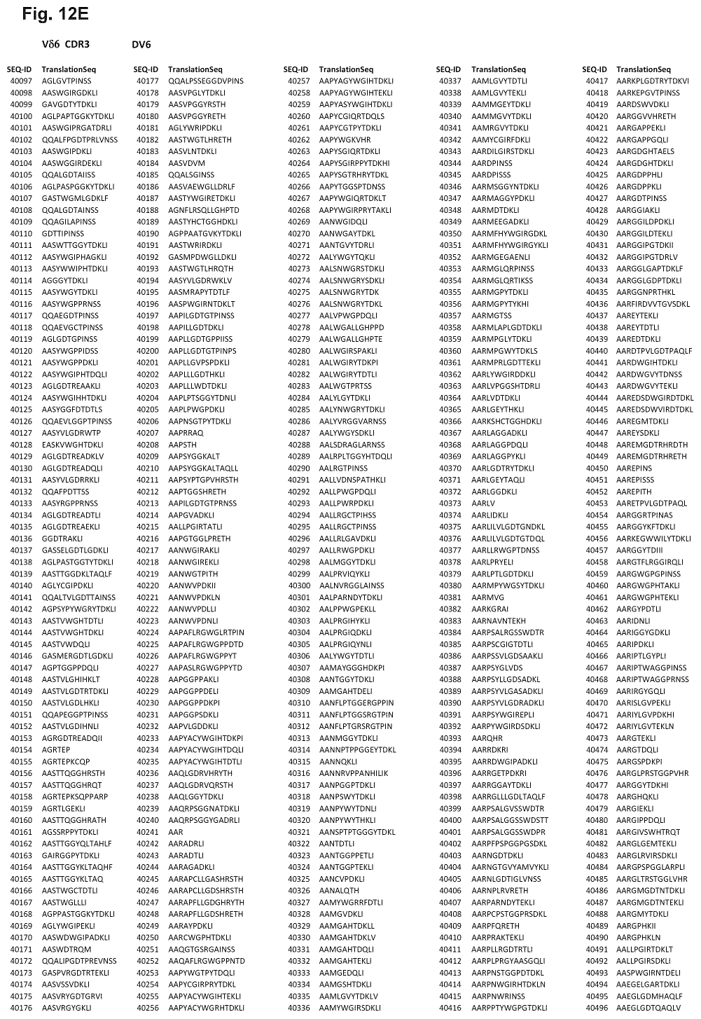

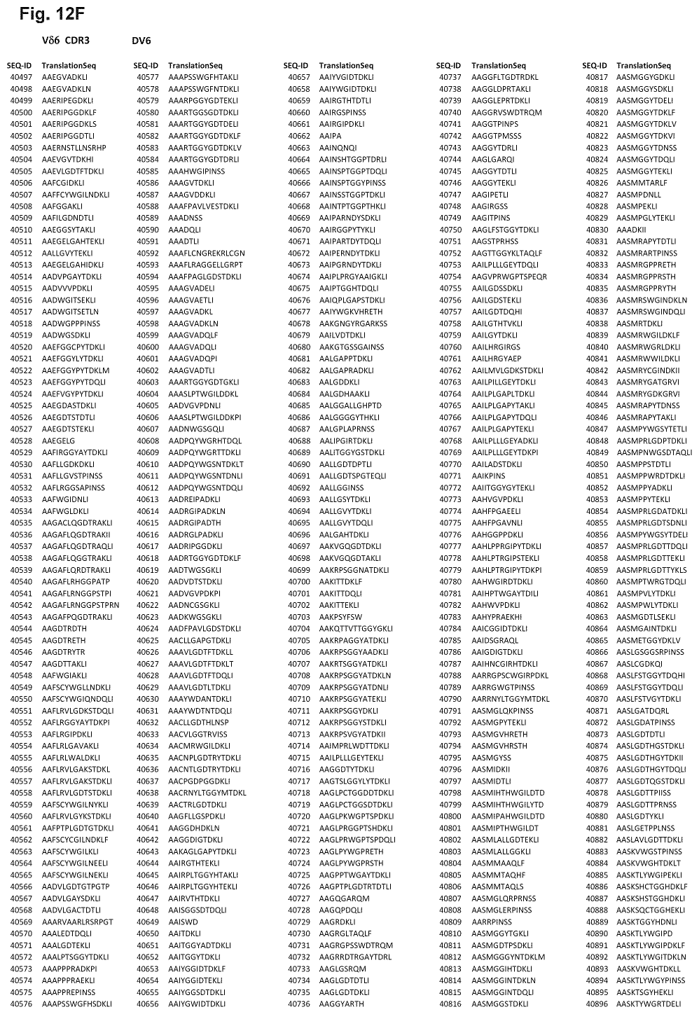

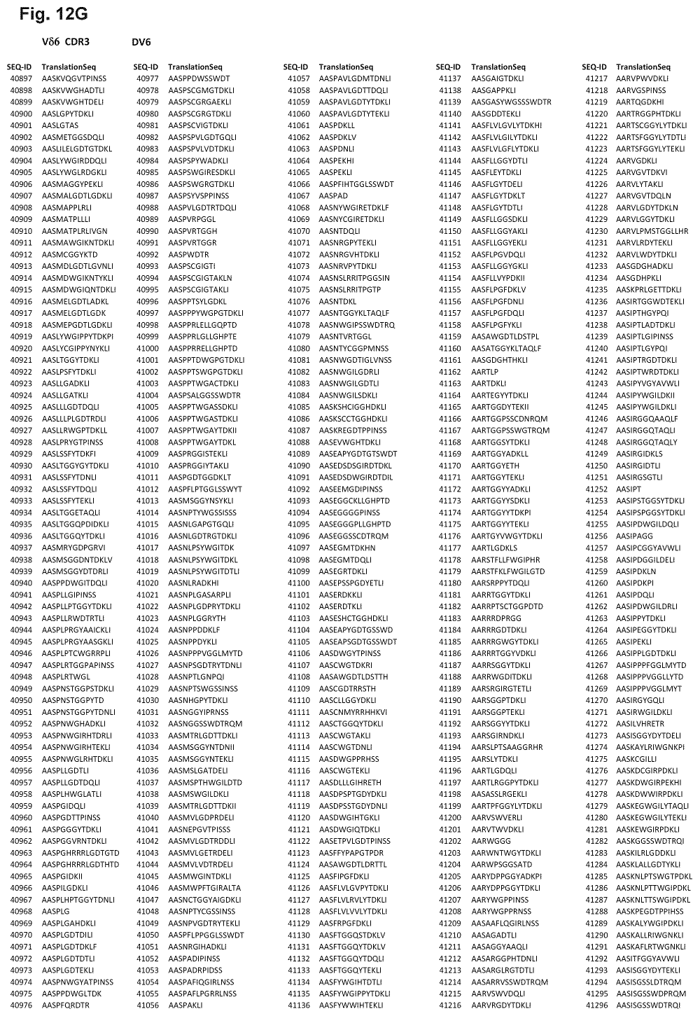

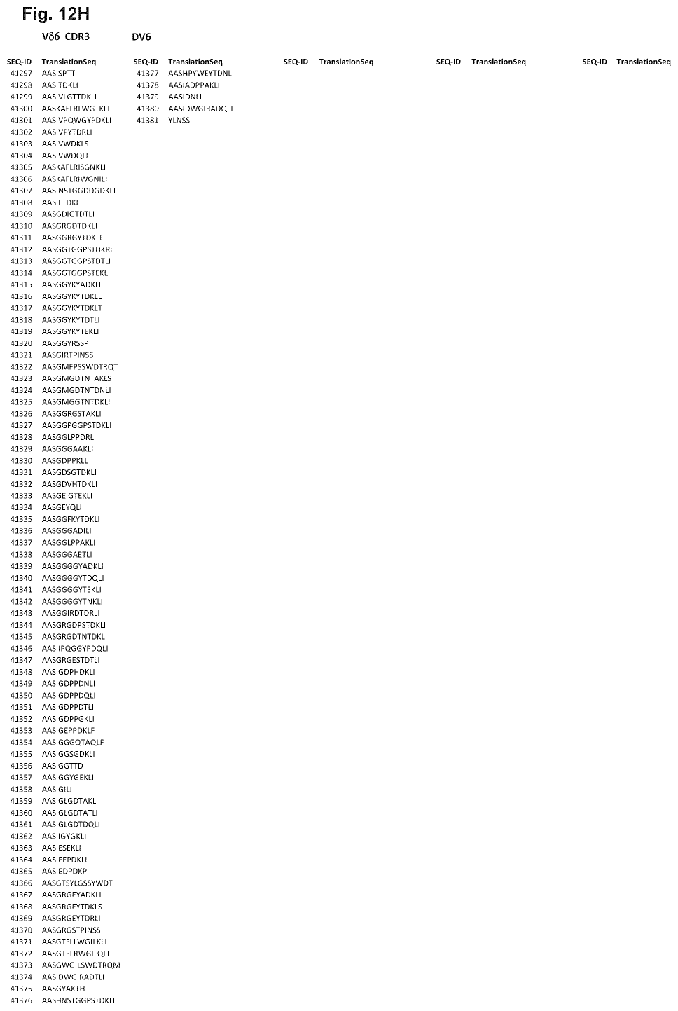

For human Vδ6 (NGS Seq ID NO:38497-41381), there is a total of 2885 unique CRD3 sequences (FIG. 12A-H). Both of the top 2 CRD3s count for 0.6% each. The CDR3s which appeared only once and two count for 80% of the total 2885 CDR3s, indicating an un-focused repertoire.

A strong indication of these human TCR γ/δ mice able to produce human or human-like TCR was demonstrated by comparison of the top 12 most abundant TCRδ CDR3s found in 10-wk-old postpartum human fetal cells (Papadopoulou et al. PNAS 2020. 117:18638-18648) to the TCR Vδ2 CDR3 of 4043 unique CDR3 sequences described. The comparison revealed that, among the top 12 human CDR3 s, 4 CDR3s were identical to the sequences listed in (NGS Seq ID 28542-32583). As shown in Table 12, clone ACDTLGDTDKLI (SEQ ID No. 28543) was ranked as number 2 in mouse spleen with an abundance percentage of 3.1%. Another clone, ACDTVGDTDKLI (SEQ ID No. 51036), had N addition used during the formation of CDR3 by VDJ recombination, indicating that the human sequence comprising human Vs, Ds, and Js are fully suitable for mouse VDJ recombination system, which is surprising as mouse and human have a large genetic divergence.

TABLE 12
Identical CDR3 delta Sequences Between Human and Humanized Mouse

| | Most abundant CDR3 | | | Found in | | |
|---|---|---|---|---|---|---|
| SEQ ID No. | delta in human | | N addition | Mouse spleen | Rank | (%) |
| 50831 | hDV2 | ACDILGDTDKLIF* | 0 | + | 513 | 0.007 |
| 50832 | | ACDTVLGDTWDTRQMF | 0 | | | |
| 50833 | | ACDTVLGDSSWDTRQMF | 0 | | | |
| 29367 | | ACDVLGDTDKLI* | 0 | + | 827 | 0.004 |
| 50834 | | ACDILGDTAQLF | 0 | | | |
| 50835 | | ACDTWGYTDKLI | 0 | | | |
| 50836 | | ACDTWGTDKLI | 0 | | | |
| 28543 | | ACDTLGDTDKLI* | 0 | + | 2 | 3.065 |
| 50837 | | ACDILGDTLTAQLF | 0 | | | |
| 50838 | | ACDSTGGYSWDTRQMF | 0 | | | |
| 50839 | | ACDTAGGYSWDTRQMF | 0 | | | |
| 29246 | | ACDTVGDTDKLI* | 1 | + | 706 | 0.006 |

### Example 7

Human Vδ Functional Test Through E coli. Infection

Tagawa et al. showed that mouse Vδ1 plays an important role in bacterial clearance. In mouse Vδ1 knockout mice, the clearance of intraperitoneal injected (IP) bacteria E coli. was significantly delayed as compared to wild-type mice (Tagawa., T. 2004 J immuno 173:5156-5164). The highest difference in bacteria clearance was observed on day 3. The bacteria count (CFU/mouse) from peritoneal fluid collected from infected mice in Vδ1 knockout mice was almost 2 logs higher than that of wild-type mice.

In the human Vδ mice at the present invention (Example 2), mouse Vδ1 (mouse TRDV4) was replaced by human Vδ1-8, leading to a mouse Vδ1 deficient mouse model, yet human Vδ expressed. Mostly following protocols provided in the article of Tagawa et al., 10*8 E. coli (DH5alpha) in 100 ul PBS was IP injected into wild-type control, heterozygous, and homozygous human Vδ mice. Three days later, 5 ml PBS was injected into the peritoneal cavity of these mice, and subsequently, peritoneal fluid containing bacteria was collected and 1 ul of the fluid was plated on an antibiotic blank LB plate. E coli. colonies were counted overnight. As shown in Table 13, there is no statistical significance among these three test groups, indicating human Vδ functionally compensated for the losses of mouse Vδ1. The human δ TCRs specific for the E coli. clearance can be cloned, sequenced, and transferred to a human cell for therapeutic purposes.

TABLE 13
Bacteria Colony Count for Human Vdelta and Wildtype Control Mice

| Group | Mouse ID NO | Geneotype | Bacteria Count |
|---|---|---|---|
| 1 | 613 | WT | 13 |
| | 617 | WT | 3 |
| | 621 | WT | 1 |
| 2 | 614 | Hetrozygous | 56 |
| | 616 | Hetrozygous | 12 |
| | 619 | Hetrozygous | 11 |
| 3 | 615 | Homozygous | 5 |
| | 618 | Homozygous | 2 |
| | 620 | Homozygous | 3 |

No statistical significance was detected between any of groups

### Example 8

Human TCR γδ Clonal Expansion and Focusing

In humans, TCR γδ can sense the changes on the cell surface of infected and malignant cells leading to the expansion and focusing of γδ repertoire well studied including Vδ1, Vδ2, and Vδ3 (Hunter., S. 2018 69:654-665). The expansion and particular focusing are, therefore, the hallmark of immune reaction and they can be used as a biomarker for many immune-related testing and development including cancer treatment, vaccine development, skin wound healing, lung and liver diseases, intestinal disease, and brain mal-functioning.

Humanized mice comprising TCR Vδ (homozygous) and Vγ (heterozygous) were generated through the breeding process detailed in Example 3. Pathogenes were introduced through IP injection and blood was collected through retro-orbital sinus after proper anesthetic procedures. Specifically, blood was collected before and 3 days after injection of 5×10*8 of E. coli (DH5alpha) in 50 ul PBS. PCR and repertoires of Vδ1-4, 6 were analyzed using procedures detailed in Example 6. Positive PCR bands were detected before injection in Vδ1, 2, 3, 4, and 6. However, after injection, only Vδ1 and 4 were positive indicating there was a downregulation of the expression of Vδ2, 3, and 6 below PCR detection level. Vδ1 and 4 before and after injection were subjected to further repertoire analysis and the results were presented in Table 14. The results positively show that there was a clonal focusing in Vδ1 and 4.

More specifically, for Vδ1, before the injection, clones are relatively even distributed as the top ten most prevalent clones have a percentage average between 2.0%-3.0%. In stark contrast, after E coli. injection, clones were highly focused as a single expanded clone (top clone) counted for 62.4%. The top clone with CDR3 sequence of ALGFYINIGTPYTDKL (SEQ ID No. 50867) ranked only 143 on the list of CDR3 in the blood sample collected before E coli. injection among 10,038 unique V δ 1 CDR3s and has a percentage of 0.053%. After E coli. injection, the mouse blood also had a reduced amount of unique clones with a total of only 3530, which is about ⅓ of that before injection.

Similarly, for Vδ4, before the injection, clones are relatively even distributed as the top ten most prevalent clones have a percentage average between 3.4%-5.1%. In stark contrast, after E coli. injection, clones were highly focused as three expanded clones (top three clones) counted for 41.0%, 36.4.0%, and 13.5%, respectively. These top three clones with CDR3 sequence of ANIREGGVYDKLI (SEQ ID No. 50877), AMRPSYYKLI (SEQ ID No. 50878), AMREGLPGGYARDKLI (SEQ ID No. 50879) ranked only 78, 75, and 240 on the list of CDR3 in the blood sample collected before E coli. injection among 3284 unique V δ 4 CDR3s and has a percentage of 0.043%, 0.050%, 0.011%, respectively. After E coli. injection, the mouse blood also had a reduced amount of unique clones with a total of only 880, which is about ¼ of that before injection.

A second pathogen was also tested in Humanized mice comprising V δ (homozygous) and V γ (heterozygous). Blood was collected before and 3 days after injection of 100 ul extract of Staphylococcus aureus (Wood 46 strain, sigma S2014), which is protein A deficient and spa negative. It shares 98% to 99% genome identity with S. aureus and shows a lower surface expression of cell wall-associated protein A. and was formalin-fixed crude cell suspension of essentially non-viable S. aureus (Wood 46 strain) in 0.05 M potassium phosphate buffer, pH 7.5, containing 0.2% sodium azide as described by the manufacture.

PCR and repertoires of Vδ1-4, 6 were analyzed using procedures detailed in Example 6. Positive PCR bands were detected before injection in Vδ1, 2, 3, 4, and 6. However, after injection except for Vδ3, the rest were positive indicating there was a downregulation of the expression of V δ 3 below the PCR detection level. Vδ1, 2, 4, and 6 before and after injection were subjected to further repertoire analysis and the results were presented in Table 15. The results positively show that there was a clonal focusing in all the V δ upon positive PCR products.

More specifically, for Vδ1, before staphylococcus injection, clones are relatively even distributed as the top ten most prevalent clones have a percentage average between 2.1%-3.9%. In stark contrast, after staphylococcus injection, clones were highly focused as three expanded clones (top three clones) counted for 19.9%, 19.90%, and 15.6%, respectively. These top three clones with CDR3 sequence of ALGELEGIRHKLI (SEQ ID No. 50905), ALGELLPGGYVDKL (SEQ ID No. 50965), and ALGELFLLGDTDKL (SEQ ID No. 23) ranked only 391, 347, and 646 on the list of CDR3 in the blood sample collected before staphylococcus injection among 10070 unique Vδ4 CDR3s and has a percentage of 0.016%, 0.018%, 0.011%, respectively. After staphylococcus injection, the mouse blood also had a reduced amount of unique clones with a total of only 5695, which is a little more than ½ of that before injection.

For Vδ2, before staphylococcus injection, some clones are already enriched as the top 4 clones count for 79.7% vs. spleen Vδ2 as shown in Example 6, which is still evenly distributed. The observation of highly enriched Vδ2 CDR3s was also described in human blood (Hunter., S. 2018. Journal of Hepatology 69:654-665). The top 4 CDR3s for Vδ2 in Table 13 are ACDTGGDYTDKLI (SEQ ID No. 29638), ACDTGGGYDTDKLI (SEQ ID No. 50896), ACDKYWGLYTDKLI (SEQ ID No. 50897), and ACDNTGAYTDKLI (SEQ ID No. 50898), which count for 27.9%, 24.8%, 15.2%, and 11.8%. In sharp contrast, after staphylococcus injection, clones were highly focused as a single expanded clone (top clone) counted for 78.9%. The top clone with CDR3 sequence of ACELLGDDDKLI (SEQ ID No. 50966) ranked only 85 on the list of CDR3 in the blood sample collected before staphylococcus injection among 3161 unique Vδ2 CDR3s and has a percentage of 0.06%. After staphylococcus injection, the mouse blood also had a reduced amount of unique clones with a total of only 2460, which is about ¾ of that before injection. Furthermore, the sequence of CDR3s from staphylococcus injection started predominantly with ACE instead of ACD in unstimulated blood, indicating the CDR3 conversion from ACD to ACE or other non-ACD is a clear immune marker that can be used in many immune activation processes.

For Vδ4, before staphylococcus injection, clones are relatively even distributed as the top ten most prevalent clones have a percentage average between 3.0%-3.7%. In stark contrast, after staphylococcus injection, clones were highly focused as only one clone (top clone) was expanded and counted for 89.0%, yet the second one was merely 0.6%. This top clone has a CDR3 sequence of AMIRERGSYTDKLI (SEQ ID No. 50940) ranked 62 on the list of CDR3 in the blood sample collected before staphylococcus injection among 3198 unique V δ 4 CDR3s and has a percentage of 0.03%. After staphylococcus injection, the mouse blood also had a reduced amount of unique clones with a total of only 466, which is a little more than ⅙ of that before injection, indicating a relatively larger scale reduction of unique TCR clones.

Lastly, for Vδ6, before staphylococcus injection, clones are relatively even distributed, yet slightly focused clones as the top ten most prevalent clones have a percentage average between 4.1%-8.0%. In sharp contrast, after staphylococcus injection, clones were highly focused as only two clones were expanded and counted for 38.4% and 36.3%, respectively, having a CDR3 sequence of AEGDTDKLI (SEQ ID No. 50950) and AATGSSWDTRQM (SEQ ID No. 50951). They are ranked 284 with a percentage of 0.02% and 219 with a percentage of 0.03% on the list of CDR3 in the blood sample collected before staphylococcus injection among 7552 unique V δ 6 CDR3s, respectively.

After staphylococcus injection, the mouse blood also had a reduced amount of unique clones with a total of only 3020, which is a little more than ⅖ of that before injection, indicating a relatively larger scale reduction of unique TCR clones.

### Example 9

Repertoire Analysis of TCRγ 9 and 10 from Humanized TCR γ/δ Mice

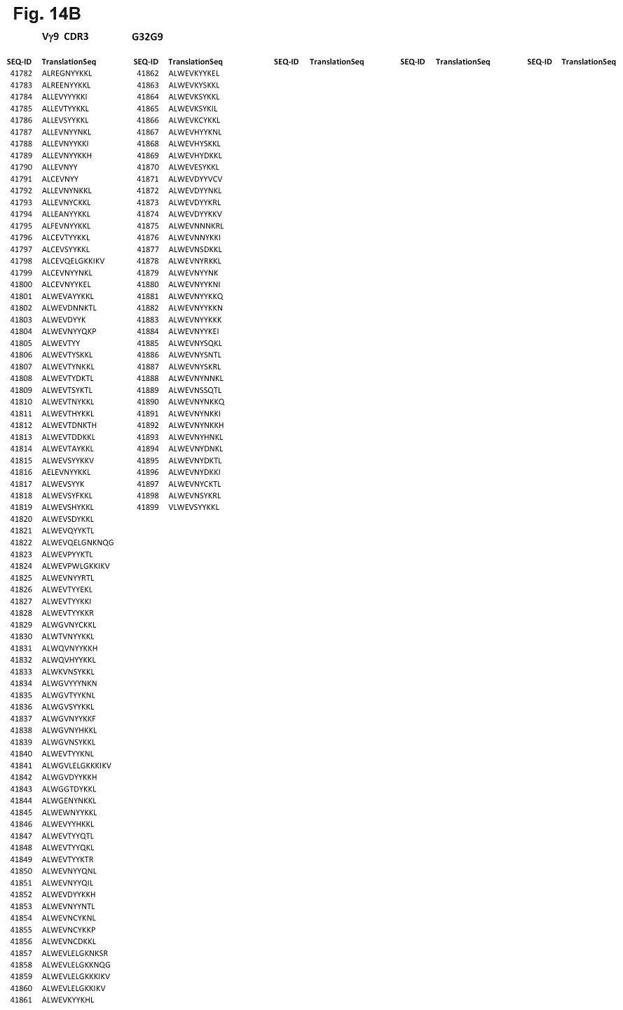

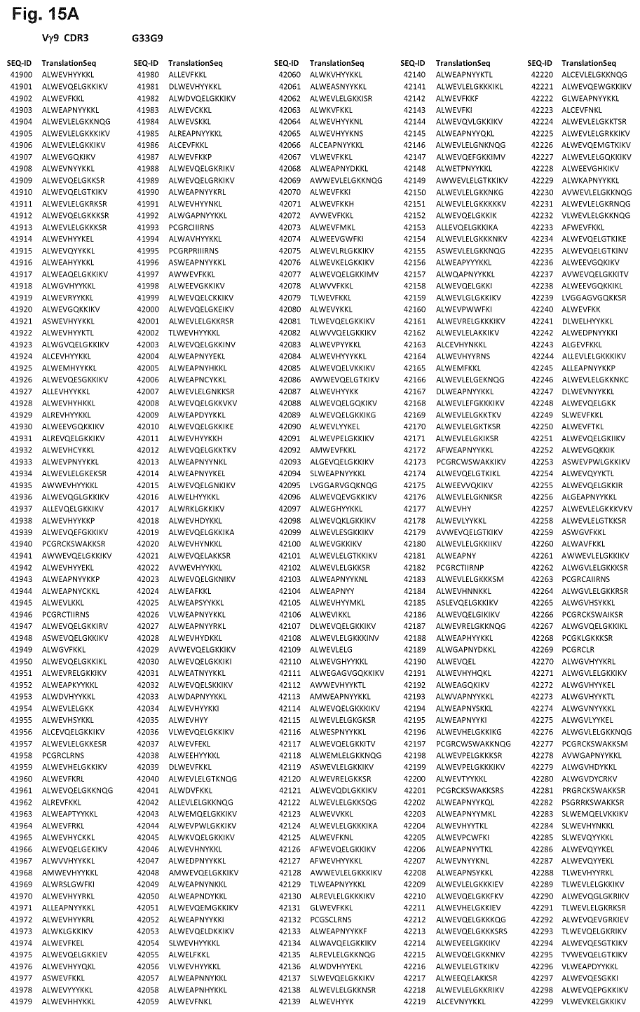

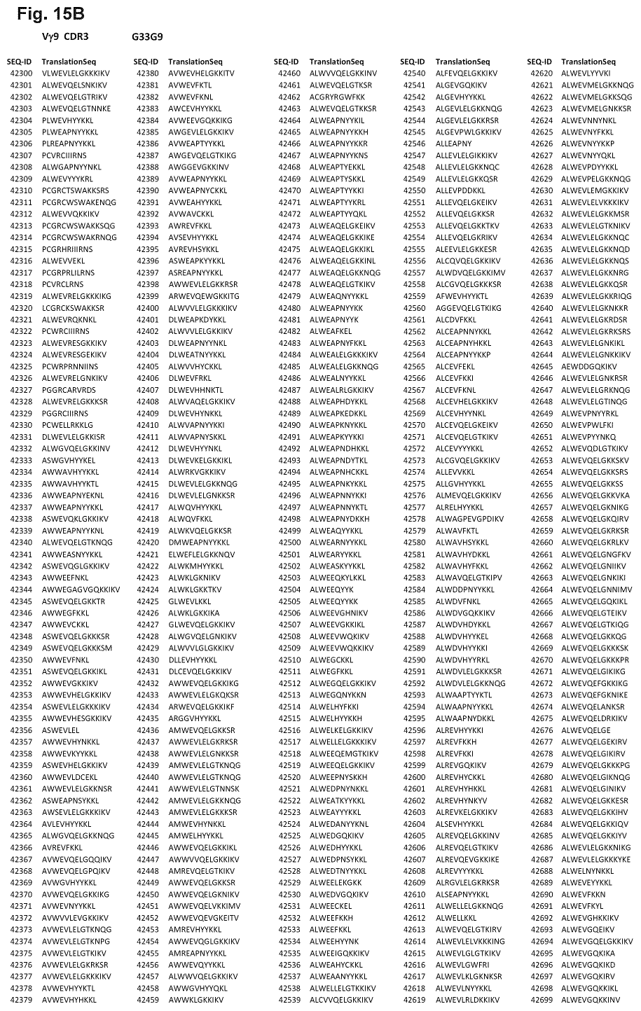

Humanized mice comprising TCR Vδ (homozygous) and Vγ (heterozygous) were generated through the breeding process detailed in Examples 3 and 8. TCRγ 9 repertoire analysis was done first by PCR using primer sets as shown in Tables 16 and 17. Strong positive PCR bands were observed in blood and thymus, but absent in lung, intestine, kidney, and spleen. Similar to the description in Example 5, the positive PCR bands were subjected to next-generation sequencing and results were shown in Table 18 and NGS Seq ID NO: 41382-41899 and 41900-42761 (FIGS. 14A-B, and 15A-C). In the thymus, TCR Vγ9 CDR3s were slightly focused with the top five CDR3 counted for 87.2% of total CDR3s, yet the repertoire has a relatively long tail with a total of 863 unique CDR3s. In sharp contrast, the mature CDR3 in blood had a very focused repertoire. The top CDR3 counted for 91% of total CDR3s and it ranked only 10 in the repertoire from thymus with a percentage of 0.3%. The focusing also brought down the diversification to only 519 unique clones in TCR Vγ9, a reduction to ⅗ of that of the thymus. Surprisely, in the thymus, the second-ranked clone with a sequence of ALWEVQELGKKIKV (SEQ ID No. 41433) (20.9%) has the same sequence in human blood of Vγ79/Vδ2 in many studies (van Diest., E. 2021. J Immunother Cancer 9:e003850). Particularly, in the paper published by Papadopoulou et al (PNAS 2020. 117:18638-18648), ALWEVQELGKKIKV (SEQ ID No. 41433) was the pre-dominant CDR3 (γ 9) in human blood from human Vγγ9/Vδ2 cells, which strongly indicated that the human TCR Vγγ9/Vδ2 mice can produce very similar clones and, in some cases, the same as that from humans.

TABLE 16
Primers for detecting RNA Expression of human Gamma V9-11 in humanized
mouse in C4 region
(PCR round 1)

| Human Vgamma | Primer sequence (5′-3′) | Seq ID NO | New SEQ ID No. |
|---|---|---|---|
| hGV9-11 | TACCTATGGGAGGAAACAGAATCTC | 36 | 50840 |
| | (common anti-sense) | | |
| hGV9 | TGCAGGTCACCTAGAGCAACCTC | 37 | 50841 |
| hGV10 | AAAGTGGAGCAGTTCCAGCTATCC | 38 | 50842 |
| hGV11 | TATCTTGGAAGGCATCCATCCAAGG | 39 | 50843 |

TABLE 17
Primers for detecting RNA Expression of human Gamma V9-11 in humanized
mouse in C4 region
(PCR round 2)

| Human Vgamma | Primer sequence (5′-3′) | Seq ID NO | New SEQ ID No. |
|---|---|---|---|
| hGV9-11 | AATCTTCAGTCACCGTCAGCCAAC | 40 | 50844 |
| | (common anti-sense) | | |
| hGV9 | CGCCTGGAATGTGTGGTGTCTGG | 41 | 50845 |
| hGV10 | CATACCTTGCAAGATATCGAGCAC | 42 | 50846 |
| hGV11 | CATACACTGGTACTGGCAGAAACC | 43 | 50847 |

TABLE 18
Top Ten TCR Gamma 9 CDR3s in Blood and Thymus

| SEQ ID No. | Thymus | | (%) | Blood | (%) | SEQ ID No. |
|---|---|---|---|---|---|---|
| 41424 | hGV9 | ALWEYHYYKKL | 34.0 | ALWEVNYYKKE | 91.0 | 41382 |
| 41433 | | ALWEVQELGKKIKV* | 20.9 | ALWEVTYYKKL | 0.6 | 41383 |
| 41440 | | ALWEVEKKL | 16.1 | ASWEVNYYKKL | 0.5 | 41384 |
| 41441 | | ALWEAPNYYKKL | 14.7 | ALLEVNYYKKL | 0.4 | 41385 |
| 41858 | | ALWEVLELGKKNQG | 1.5 | ALREVNYYKKL | 0.4 | 41386 |
| 41859 | | ALWEVLELGKKKIKV | 0.8 | ALCEVNYYKKL | 0.4 | 41387 |
| 41860 | | ALWEVLELGKKIKV | 0.6 | ALWEVDYYKKL | 0.3 | 41388 |
| 41907 | | ALWEVGQKIKV | 0.5 | ALWEVNYYKAL | 0.3 | 41389 |
| 41382 | | ALWEVNYYKKL | 0.3 | ALWGVNYYKKL | 0.3 | 41390 |
| 41909 | | ALWEVQELGKKSR | 0.3 | ALWEVSYYKKL | 0.3 | 41391 |

*The sequence of ALWEVQELGKKIKV has been reported in blood of many human studies

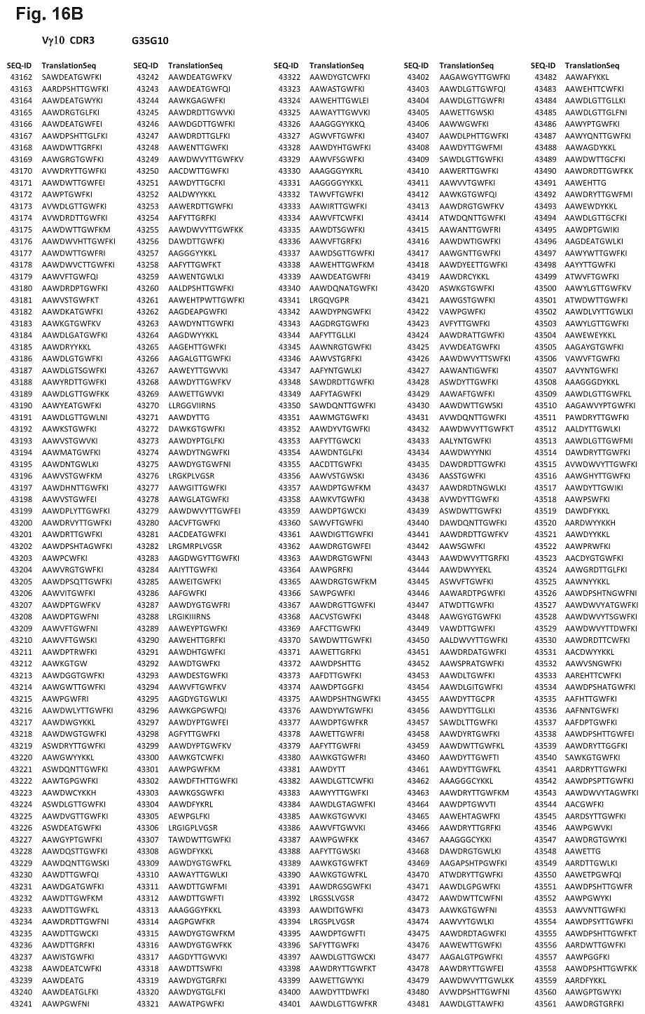

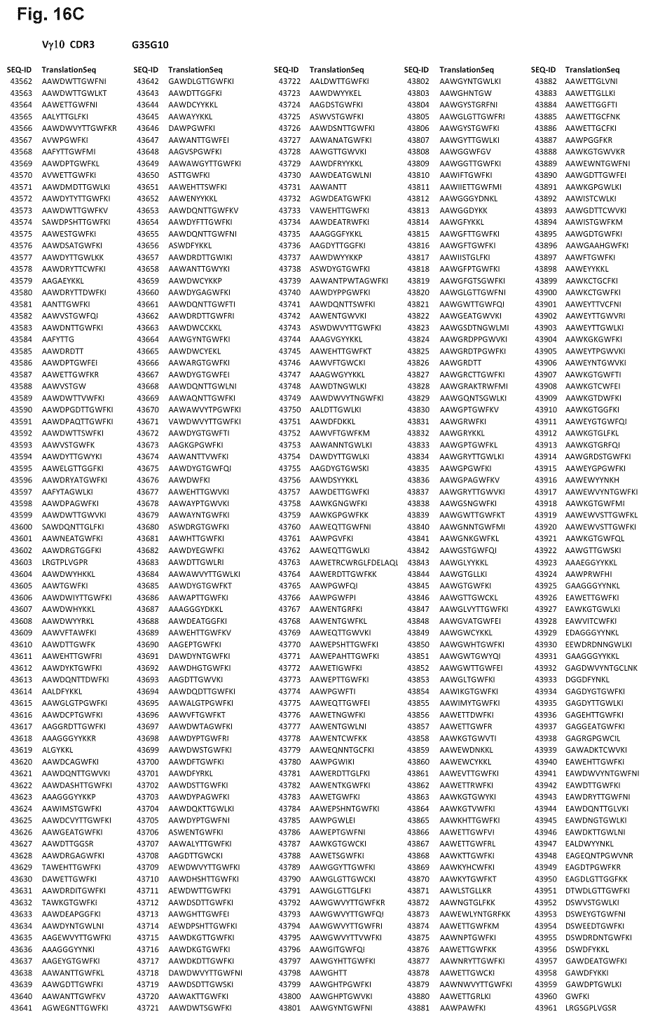

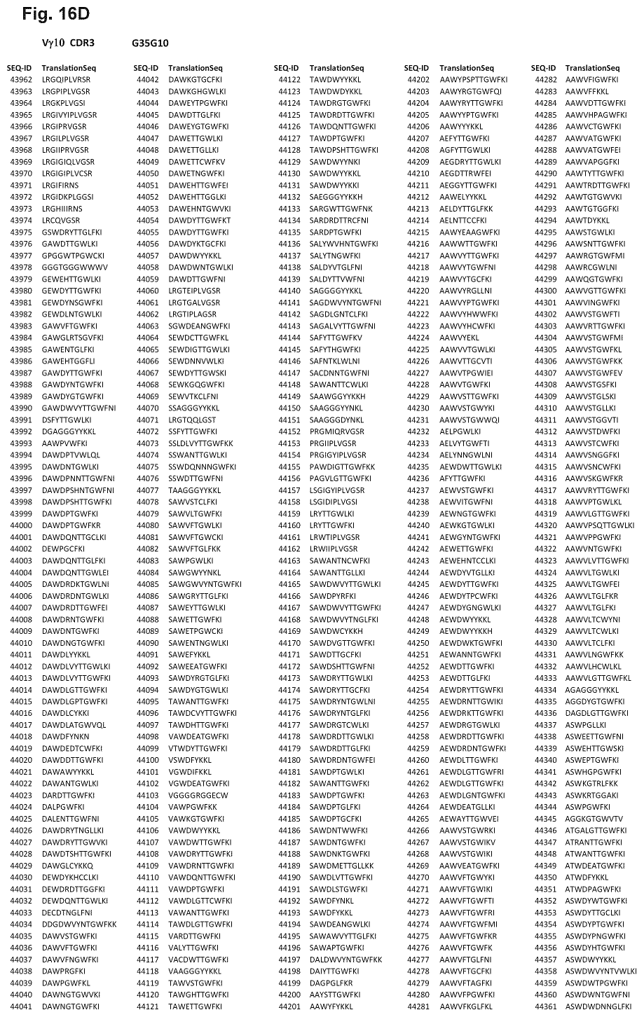

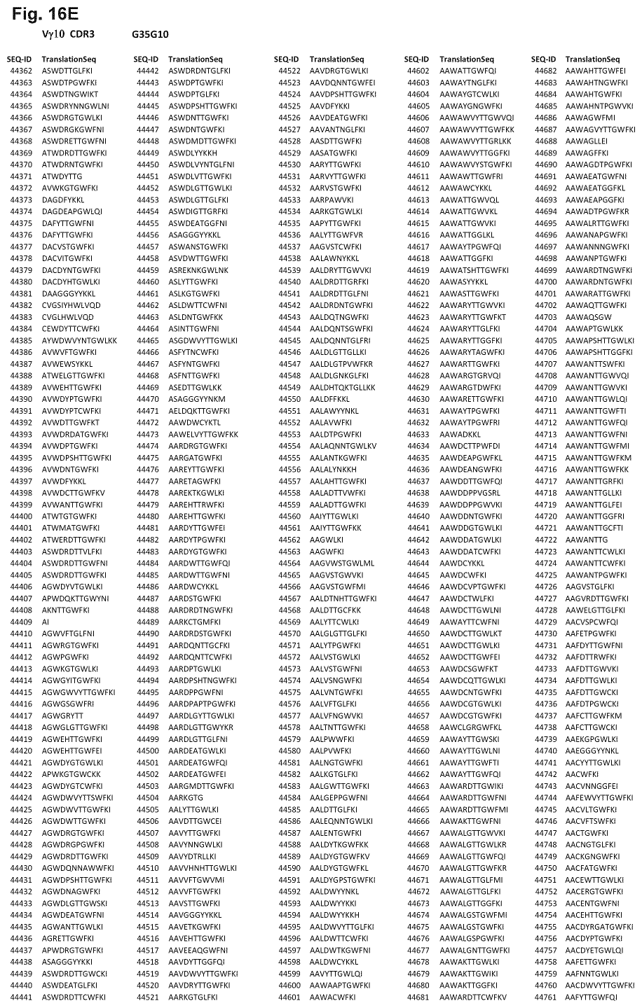

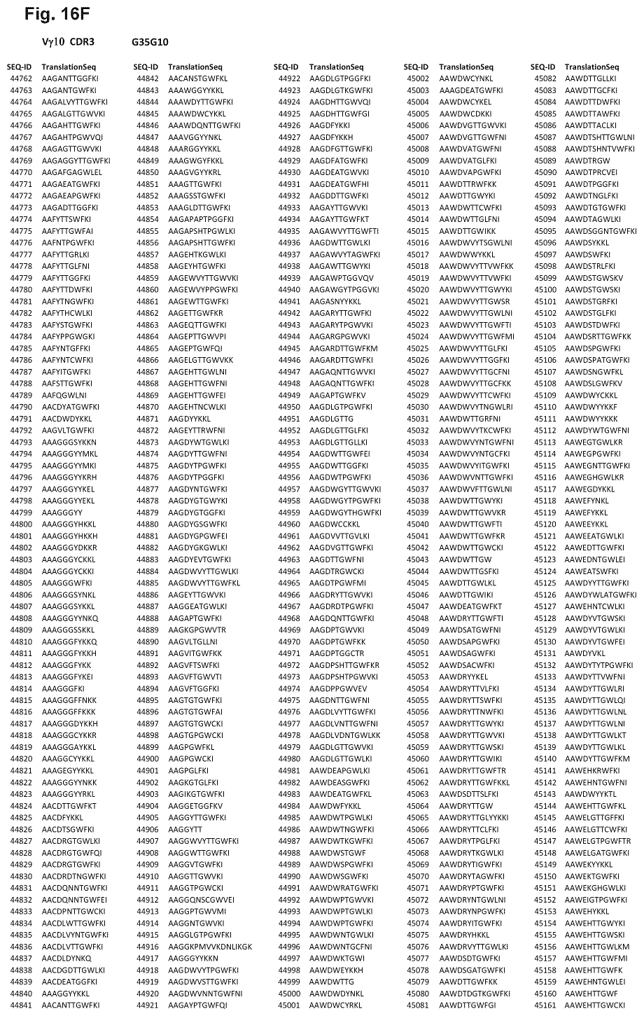

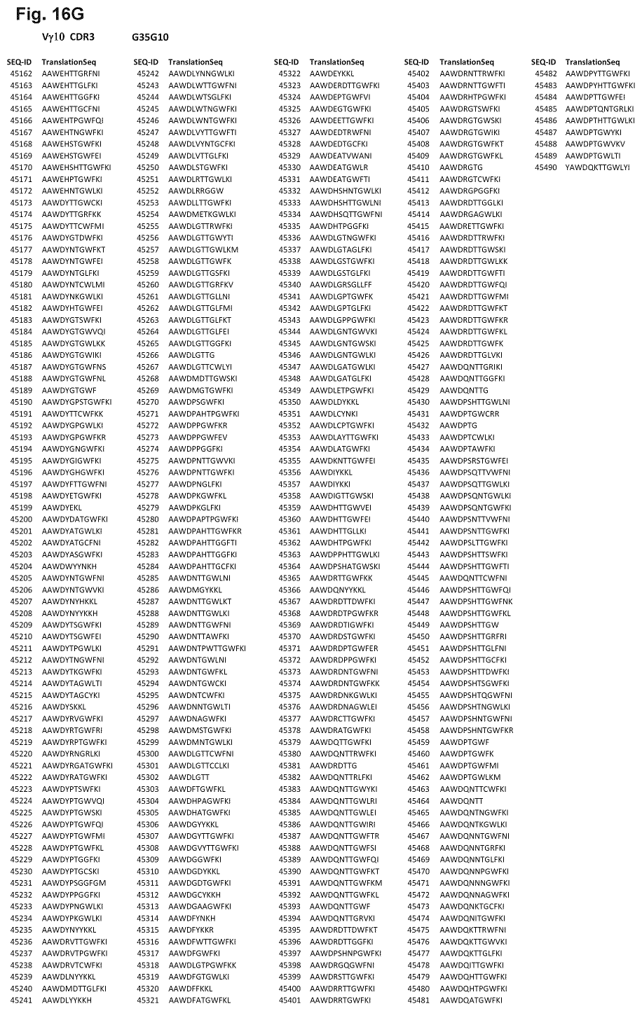

Limited access to human tissues largely hindered the study of human Vγ10. There were very few publications that described the sequence and function of human Vγ10. To explore the possibility of establishing a human Vγ10 in mice, tissues from humanized mice comprising of TCR Vδ (homozygous) and Vγ(heterozygous) were collected, PCR, NGS, data processed similar to the description of Vγ9, but utilizing different sets of PCR primers (Table 14 and 15) and NGS ID NO: 42762-45490, 4549149130, and 49131-50786 (FIGS. 16A-G, 17A-E and 18A-J). The data showed that Vγ10 was expressed in all the tissues tested including the lung, thymus, intestine, kidney, and spleen, and the top ten CDR3s of these tissues were listed in Table 19. There are different diversifications of CDR3 in different tissue. For example, in descending order, the thymus has 3641 unique CDR3s, lung has 2730, intestine has 1657, kidney has 1277, and spleen has 689. This order of abundance was comparable with the previous observation in human studies by others that the thymus has most individual clones as the thymus is where in the first place these clones are produced. The lung and intestine are two of the high-ranking tissues where human γδ T cells are expressed.

TABLE 14
Pathogenic focusing of TCR delta in Delta (homo)/gamma (hetero) Mice
(in Blood) (E. coli study)

| | Top ten CDR3 of E coli | | | Top ten CDR3 of E coli | | |
|---|---|---|---|---|---|---|
| SEQ ID No. | Infection at day 0 | | (%) | Infection at day 3 | (%) | SEQ ID No. |
| 50848 | hDV1 | ALGVTGGYPDKLI | 3.0 | AMGFYWGTPYTDKL | 62.4 | 50867 |
| 50849 | | ALAIPDKLI | 2.7 | ALGFYWGTPYTEKL | 3.1 | 50868 |
| 50850 | | ALGVYWGPLDKLI | 2.4 | ALGFYWGTPYTDQL | 3.1 | 50869 |
| 50851 | | ALGEGWGISDKLI | 2.3 | ALGFYWGTPYTDK | 2.8 | 50870 |
| 50852 | | ALGSRPGGYDKLI | 2.2 | ALGFYWGTPYTDK | 1.8 | 50871 |
| 50853 | | ALGELGVHTDKLI | 2.2 | ALGFYWGTPNTDKL | 1.7 | 50872 |
| 50854 | | ALGEGWGAYTDKLI | 2.1 | DLGFYWGTPYTDKL | 3.5 | 50873 |
| 1214 | | ALGGLGDTDKLI | 2.1 | ALGFYWGTPSTDKL | 1.3 | 50874 |
| 50855 | | ALGPSYTDKLI | 2.0 | ALGCYWGTPYTDKL | 1.1 | 50875 |
| 50856 | | ALGELPSSDKLI | 2.0 | LLFTGGPRTPINS | 1.1 | 50876 |
| 50857 | hDV4 | AMREGPHVGMGLI | 5.1 | AMREGGVYDKLI | 41.0 | 50877 |
| 50858 | | AMREGDTGLI | 4.6 | AMRPSYYKLI | 36.4 | 50878 |
| 50859 | | AMREGWGKLI | 4.3 | AMREGLPGGYARDKLI | 13.5 | 50879 |
| 50860 | | AMREGWGYTDKLI | 4.1 | AMREGFSNKLI | 1.0 | 50880 |
| 50861 | | AMRAYWARADKLI | 4.0 | AMREGGVYNKLI | 0.3 | 50881 |
| 50862 | | AMRARTGGCDKLI | 3.7 | AMREGGVYYKLI | 0.2 | 50882 |
| 50863 | | AMRESTGGYATDKLI | 3.6 | AMRPSYYELI | 0.2 | 50883 |
| 50864 | | AMRELGDTFDKLI | 3.5 | ATREGGVYDKLI | 0.2 | 50884 |
| 50865 | | AMREGWGILKLI | 3.5 | AMRGGGVYDKLI | 0.1 | 50885 |
| 50866 | | AMREGDTHTDKLI | 3.4 | AMREGGVYGKLI | 0.1 | 50886 |

TABLE 15
Pathogenic Expansion of TCR delta in Delta (homo)/gamma (hetero) Mice
(in Blood) (Staphylococcus study)

| | Top ten CDR3 of Staph | | | Top ten CDR3 of Staph. | | |
|---|---|---|---|---|---|---|
| SEQ ID No. | Infection at day 0 | | (%) | Infection at day 3 | (%) | SEQ ID No. |
| 50887 | hDV1 | ALGEDTADKLI | 3.9 | ALGELEGIRHKLI | 19.9 | 50905 |
| 50888 | | ALGVPGGYPDKLI | 3.1 | ALGELLPGGYVDKL | 19.7 | 50906 |
| 8851 | | ALGELGGYTDKLI | 2.6 | ALGELFLLGDTDKL | 15.6 | 23 |
| 50889 | | ALGELEGDTDKLI | 2.6 | ALGDLGIRHKLI | 5.32 | 50907 |
| 50890 | | ALGEPGDRSSWDTR | 2.5 | ALGDLLPGGYVDKL | 2.95 | 50908 |
| 50891 | | ALGEPWGPDKLI | 2.5 | ALGDLFLLGDTDKL | 2.87 | 110 |
| 50892 | | ALGDPLWGISYTDK | 2.5 | ALGELLHGGYVDKL | 0.93 | 50909 |
| 50893 | | ALGAWGGILKLI | 2.3 | ALGELFLLGDTDQL | 0.79 | 117 |
| 50894 | | ALGEYWGIQDDKLI | 2.2 | ALGELFLLGDTEKL | 0.77 | 138 |
| 50895 | | ALGELDWGINTDKL | 2.1 | ALGELFLLGDTDK | 0.68 | 250 |
| 29638 | hDV2 | ACDTGGYDTDKLI | 27.9 | ACELLGDDDKLI | 79.0 | 50910 |
| 50896 | | ACDTGGGYDTDKLI | 24.8 | ACELLGDDEKLI | 1.2 | 50911 |
| 50897 | | ACDKYWGLYTDKLI | 15.2 | ACELRGDDDKLI | 1.1 | 50912 |
| 50898 | | ACDNTGAYTDKLI | 11.8 | PVSYWGMTINSS | 0.7 | 50913 |
| 50899 | | ACDTGGGYDTEKLI | 0.5 | ACELLGDDDKLF | 0.7 | 50914 |
| 50900 | | ACDTGGGYDTDQLI | 0.5 | ACELLGDDDNLI | 0.7 | 50915 |
| 50901 | | ACDTGGGDDTDKLI | 0.4 | ARELLGDDDKLI | 0.6 | 50916 |
| 50902 | | ACDTGGDYTEKLI | 0.4 | AGELLGDDDKLI | 0.6 | 50917 |
| 50903 | | ACDKYWGLYTEKLI | 0.4 | ACELLGDDDQLI | 0.6 | 50918 |
| 50904 | | ACRDGGGDTDKLI | 0.4 | ACELLGDDYKLI | 0.6 | 50919 |
| 50920 | hDV4 | AMRDGLI | 3.7 | AMRERGSYTDKLI | 89.0 | 50940 |
| 50921 | | AMKGDTDKLI | 3.5 | AMRERGSYTDELI | 0.6 | 50941 |
| 50922 | | AMSPRWGIRPDKLI | 3.3 | AMRERGSYTDKLV | 0.4 | 50942 |
| 50923 | | AMRVTGGNTDKLI | 3.2 | AMREGGSYTDKLI | 0.4 | 50943 |
| 50924 | | AMREGPPSPINSS | 3.3 | AMRERGSYADKLI | 0.4 | 50944 |
| 50925 | | AMREGCGSHTDKLI | 3.0 | AMSHTGGWAINSS | 0.3 | 50945 |
| 50926 | | AMRLYWGEYTDKLI | 3.0 | ALRERGSYTDKLI | 0.3 | 50946 |
| 50927 | | AMREGLWGIRDKLI | 3.0 | AVRERGSYTDKLI | 0.3 | 50947 |
| 50928 | | AMREGLGDTSDKLI | 3.0 | AMRERGSYTDKLT | 0.3 | 50948 |
| 50929 | | AMREAGVTDKLI | 3.0 | AMRERGPYTDKLI | 0.3 | 50949 |
| 50930 | hDV6 | AASTGGQVP | 8.0 | AEGDTDKLI | 38.4 | 50950 |
| 50931 | | AANHWGPYTDKLI | 7.9 | AALSSWDTRQM | 36.3 | 50951 |
| 50932 | | AASSPGGGYDKLI | 7.8 | QHWGAPGTPDR | 3.09 | 50952 |
| 50933 | | AASREGYGAI | 7.2 | AEGDTEKLI | 1.5 | 50953 |
| 50934 | | AAVGYGNPDKLI | 6.9 | AEGDTDQLI | 1.2 | 50954 |
| 50935 | | AASMGGDKLI | 5.8 | QRPININSS | 0.7 | 50955 |
| 50936 | | AASGGGYTTDKLI | 5.3 | AALGSSWDTRRM | 0.6 | 50956 |
| 50937 | | AASRGDTGTDKLI | 4.8 | STGELLGHPTD | 0.5 | 50957 |
| 50938 | | AASVVGDTDKLI | 4.7 | AEGDTDKLN | 0.5 | 50958 |
| 50939 | | AASMAGWGIYTDKLI | 4.1 | AALSSWDTRQR | 0.4 | 50959 |

TABLE 19
Top Ten TCR Gamma 10 CDR3s in various tissues

| SEQ ID No. | hGV10 | | (%) | | | (%) | SEQ ID No. |
|---|---|---|---|---|---|---|---|
| 42762 | Lung | AAQDYTTGWFKI | 8.2 | Thymus | AAWDTTGWFKI | 7.8 | 42763 |
| 42763 | | AAWDTTGWFKI | 6.8 | | AAWDYTTGWFKI | 3.7 | 42762 |
| 42764 | | AAWDLGTTGWFKI | 5.7 | | AAWGYTTGWFKI | 3.7 | 42805 |
| 42765 | | AAWDEATGWFKI | 4.2 | | AAWGTGWFKI | 2.9 | 43845 |
| 50960 | | AAWKGTGWFKI | 4.1 | | AAWGTTGWFKI | 1.5 | 42797 |
| 42767 | | AAWPGWFI | 4.0 | | AAWAYTTGWFKI | 1.5 | 42795 |
| 42768 | | AAWGPTGWFKI | 4.0 | | AAWDDTTGWFKI | 1.4 | 42887 |
| 42769 | | AAWDRDTTGWFKI | 3.7 | | AAWDWTTGWFKI | 1.2 | 42773 |
| 50961 | | AAFYTTGWFKI | 3.2 | | AAWYTTGWFKI | 1.2 | 42899 |
| 42771 | | AAWDWVYTTGWFKI | 3.2 | | AAWEYTTGWFKI | 1.2 | 42863 |
| 42950 | Intestine | AAWDSHTGWFKI | 17.3 | Kidney | AAWDCSGWFKI | 17.9 | 42938 |
| 42762 | | AAWDYYGWFKI | 10.3 | | AAWADTTGWFKI | 16.1 | 43053 |
| 50962 | | AAWWGWFKI | 6.9 | | AAWCTGWFKI | 15.0 | 43135 |
| 52792 | | AAWDTSGWFKI | 6.2 | | AAWDTTGWFKI | 13.6 | 42763 |
| 43452 | | AAWSPRATGWFKI | 6.2 | | AAWDYTTGWFKI | 11.9 | 42762 |
| 45479 | | AAWDQHTTGWFKI | 6.1 | | AAWDYEETTGWFKI | 8.0 | 43418 |
| 50963 | | AAWDFATGWFKI | 6.0 | | AAWDSTGWFKI | 2.1 | 42792 |
| 43302 | | AAWFTHTTGWFKI | 5.0 | | LRGPIPLVGSR | 0.3 | 43963 |
| 45221 | | AAWDRYGATGWFKI | 4.6 | | AAWCDSGWLKI | 0.3 | 49323 |
| 43650 | | ASTTGWFKI | 3.6 | | AAWADTTGWLKI | 0.2 | 45970 |
| | | | | Spleen | AAWDSTGWFKI | 80.8 | 42792 |
| | | | | | AAWDSAGWFKI | 1.1 | 45051 |
| | | | | | AAWDTTGWFKI | 0.8 | 42763 |
| | | | | | AAWGCSGWFKI | 0.8 | 42938 |
| | | | | | AAWADTTGWFKI | 0.7 | 43053 |
| | | | | | AAWHYTGWFKI | 0.7 | 50964 |
| | | | | | AAWDCTGWFKI | 0.7 | 43135 |
| | | | | | AAWDSTGWLKI | 0.7 | 45831 |
| | | | | | AAWDYTTGWFKI | 0.6 | 42762 |
| | | | | | AAGDSTGWFKI | 0.6 | 43724 |

### Example 10

Human TCR Vγ9/Vδ2 and Vγ9/Vδ1 Surface Expression by Flow Cytometry Analysis

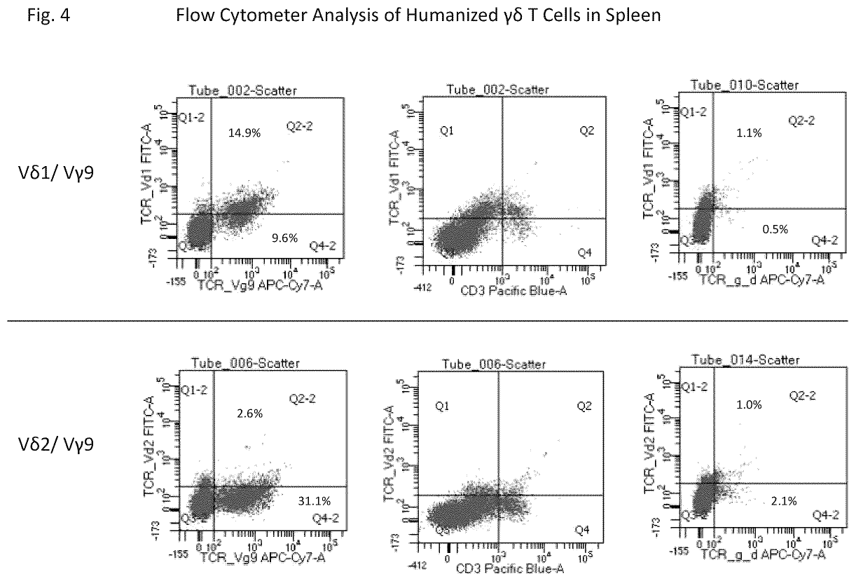

The existence of γδ TCR revealed by repertoire studies was further demonstrated by flow cytometry analysis, which utilizes fluorescently labeled antibodies that specifically bind to T cell receptors on the T cell surface in protein form. Blood was collected through the retro-orbital sinus of human double homozygous γδ TCR mice. Tissues were first treated with Red Blood Cell Lysis buffer (0.8 g NH4Cl and 0.11 g NaHCO₃ in 100 ml water-based solution) to remove red blood cells and white blood cells including T cells were collected by centrifugation and diluted to a proper concentration before subject to a flow cytometer (Becton Dickinson LSRFortessa). The antibodies and their suppliers are listed in Table 20. The flow cytometry analysis in Table 21 showed that the spleen was the highest site for γ/δ T cells (about 17%), followed by the thymus (5%) and lung (2.6%), which are well matched with the results from human studies. In humans, the spleen is also the highest site for γδ T cells with an abundance of 12.5%±8.1%, followed by the thymus, 1.4%±0.5% (Inghirami, G., 1990 Am J Pathol 136: 357-367). The results herein indicated that γδ T cells in human γδ TCR mouse was well developed and maintained the correct ratio among other CD3-positive cells. The data herein also revealed that in the spleen most of the γδ T cells were contributed from humanized Vδ1 and Vδ2 with very little from mouse Vδ. Also in spleen, majority of the γ/δ T cells are humanized Vδ1/humanized Vγ9 with an abundance of 61%, yet humanized Vδ2/humanized Vγ9 with an abundance of 7.6% (FIG. 4). The higher proposition of Vδ1 than Vδ2 in all categories was also consistent with the observations from others as tissue residing γδ T cells are generally 61 T cells, yet 62 mostly in blood (Mossow, G., 2021 Front. Immunol., https://doi.org/10.3389/fimmu.2021.741218). In sum, the human γ/δ TCR mice have normal development and maturation in multiple tissues, and subclasses of humanized γ and humanized δ T cells can keep similar ratios as represented in humans.

TABLE 20

| Antibody and Suppliers | | |
|---|---|---|
| Antibody Target | Dye | Supplier |
| Anti-human TCR V61 | FITC | Miltiny |
| Anti-human TCR V82 | FITC | Miltiny |
| Anti-mouse CD3 | Pacific Blue | Biolegend |
| Anti-mouse v/6 TCR | APC-Vio-770 | Miltiny |

TABLE 21
Human Gamma/Delta TCR Flow Study in Lung, Spleen, and
Thymus

| | % | % | % Vδ 1 in | % Vδ 2 in | % Vδ 1 in | % Vδ 2 in |
|---|---|---|---|---|---|---|
| | α/β | γ/δ | total γ/δ | total γ/δ | total γ9 | total γ9 |
| Lung | 97.4 | 2.6 | 66 | 7.8 | 24 | 2.3 |
| Spleen | 83 | 17 | 70 | 30 | 61 | 7.6 |
| Thymus | 95 | 5 | 33 | 2.6 | 38 | 16.5 |

### Example 11

Cancer Development in Human TCR γδ Mice (B16 Cancer)

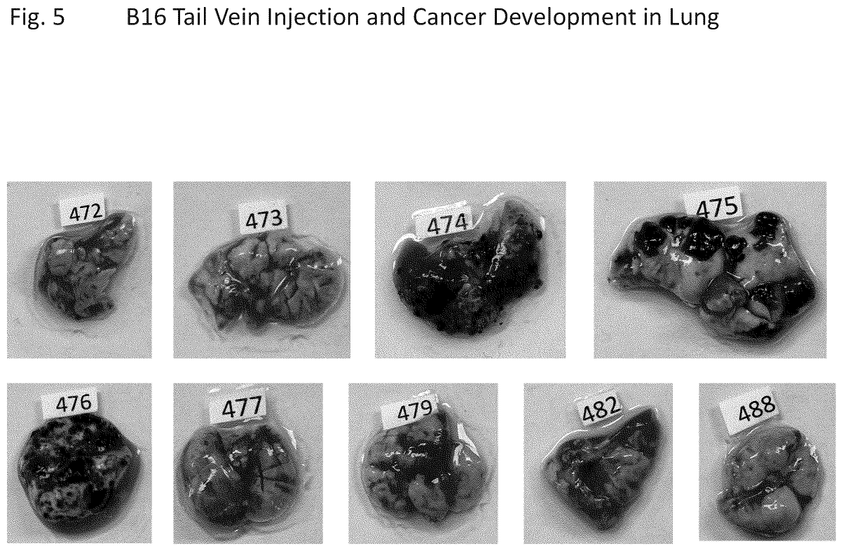

One of the big advantages of developing the human TCR γδ mouse model is this model has fully intact mouse immune systems, which provides researchers with a better model than immune-compromised mouse models (e.g., NSG and SCID) as compromised mice lacking B and T cells, which are the key players in immune therapy. The human TCR model, therefore, is suitable to accommodate syngeneic mouse cancer lines as many studies demonstrated their significance in cancer research. In the art, mouse B16 melanoma cell lines were isolated from B6 mice and proven to grow robustly in pure B6 and Balb/C mice strain, but not well in 129 mouse strain. The human TCR γ/δ mice were on a mixed background of B6 and 129 and mostly through a littermate mating scheme. Half a million B16 cells suspended in 250 ul of PBS were injected via the tail vein and the B16 primarily was proven to accumulate and metastasized in the lungs. Fourteen days later, the lungs were harvested and documented. As shown in Table 22 and FIG. 5, wild-type mice and mice with only one human TCR γ or TCR did not develop cancer at all. A large amount of B16 cancer was found in homozygous γ and homozygous δ mice, except for double homozygous mice. The results suggested that the copy number of TCR γ and/or δ played very important roles in melanoma cancer development.

TABLE 22
Cancer Development in Human TCR gamma/delta Mice (B16 cancer)

| Mouse | | Age | Genotyping | | Cancer |
|---|---|---|---|---|---|
| 1D | Sex | (Month) | Hu Delta | Hu Gamma | Occurrence |
| 472 | M | 2 | wt | wt | — |
| 473 | M | 2 | homo | homo | — |
| 474 | M | 2 | homo | wt | ++ |
| 475 | F | 2 | hetero | homo | +++ |
| 476 | F | 2 | homo | Hetero | — |
| 477 | F | 2 | hetero | wt | — |
| 479 | F | 2 | wt | hetero | — |
| 482 | M | 2 | wt | wt | — |
| 488 | F | 2 | wt | wt | — |

WT-wild type TCR gamma/delta direct from a mouse. Homo-homozygous
Hetero-heterozygous

### Example 12

Reduction and Elimination of Human Cancer Cells (Daudi) in Human γδ TCR Mice

It has already been demonstrated in the art that an important aspect of antigen specificity of human γδ TCR cells is their capacity to recognize and kill tumor targets. T cells expressing the Vγ9/Vδ2 heterodimer, the same TCR stimulated by bacterial phosphorylated metabolites, recognize bone marrow-derived tumor cells such as the non-Hodgkin B cell lymphoma line Daudi both in vitro (Fisch, P., et al. Science. 250:1269-1273.) and in vivo in a SCID animal model, an immune-compromised mouse, (Malkovska, V., et al. 1992 Cancer Res. 52:5610-5616.). Studies by Vyborova et al. further showed in vitro that only a fraction of expanded Vγ9/Vδ2 T cells was active against cancer cell lines and the clonal frequency was not associated with functional avidity of Vγ9/Vδ2 T cell receptors Clin Invest. 2020; 130:4637-4651), indicating a functional, yet complex, nature of human cancer killing by TCR based human γ/δ T cells. No human cancer studies have been done on the immune-competent mouse background with human γδ T cell receptors, besides intense effort in the last three decades.

In the present invention, 6 million in 200 ul PBS of human non-Hodgkin B cell lymphoma line Daudi (ATCC) was injected via tail vein into WT and human TCR γ (hetero)/δ (homo) mice. On day 4, multiple organs were collected including the lung, liver, spleen, intestine, and kidney, and treated with liberase (Roche) according to the manufacturer's suggestions. Cells from these tissues were labeled with PE-anti-human CD22 (Biolegend), which is a well-established marker in Daudi cells with high surface expression (Pop L., et al. 2014 Cancer Res. 74:263-271). Tissues with excessive blood were treated with a red blood cell lysis kit described in previous Examples. The CD22 labeled cells were subject to a flow cytometer (Becton Dickinson LSRFortessa). The results in Table 23 showed that there was a drastic reduction of Daudi cells (represented by CD22) in all the tissues examined from human TCR γ (hetero)/δ (homo) mouse vs. wildtype littermate control. There was a 3.4, 3, and 6.8 times reduction in lung, liver, and spleen, respectively. Particularly, There was total elimination of human Daudi cells in the intestine and kidney of human TCR (hetero)/δ (homo) mice, although very little amount of Daudi cells found wild type mice. (FIGS. 9-11)

TABLE 23
Number of CD22 Positive Human Cancer Cells
(Daudi) (per 500K Counting Events)

| Mouse Genotype | Lung | Liver | Spleen | Intestine | Kidney |
|---|---|---|---|---|---|
| WT | 55 | 27 | 190 | 33 | 0.3 |
| Delta(homo)/ | 16 | 9 | 28 | 0 | 0 |
| gamma (hetero) | | | | | |
| Ratio* | 3.4 | 3.0 | 6.8 | ∞ | ∞ |

*Ratio represents the ratio between wild type and human TCR gamma/delta mouse.
Homo—homozygous.
Hetero—heterozygous

### Example 13

Further Humanization of TCRγ Mice (ADδing Human Vγ1-8)

In this example, the humanized Vγ 9-11 mouse ES cell line was further humanized by introducing human Vγ1-8 on the same allele of humanized. Vγ9-11. A non-human animal (such as a mouse) was modified so that the non-human animal contained a nucleic acid sequence comprising human TCRγ, further comprising human TCR Vγ and Jγ. Specifically, an endogenous mouse sequence containing mouse TCR Vγ and Jγ was replaced with a sequence containing human TCR Vγ and Jγ and operably linked to a mouse TCR Cγ 1 region.

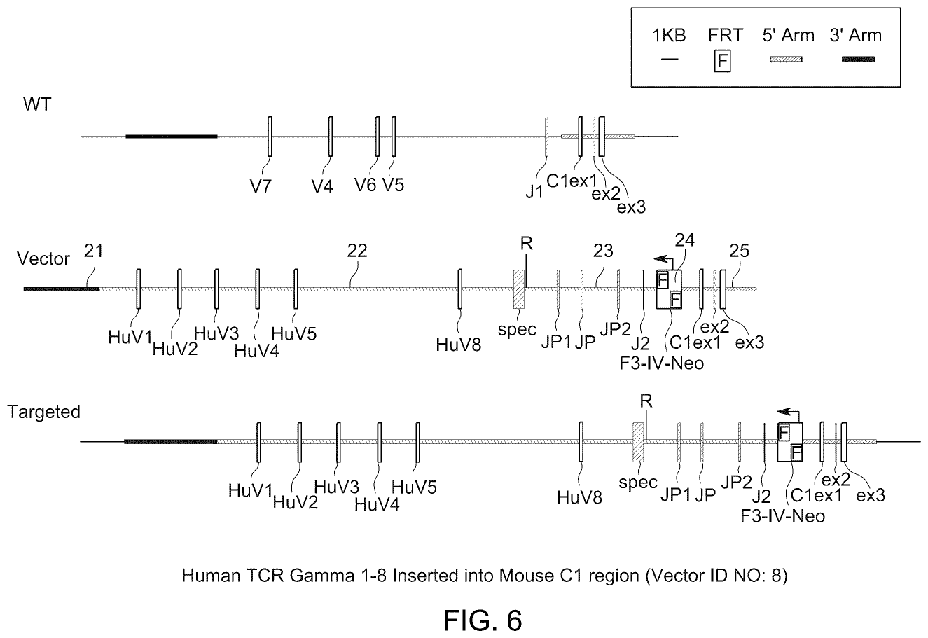

More specifically, the DNA construct comprised of 5′ mouse homology al n (10875 bp, DNA segment NO: 21), 48234 bp of human sequence including human TCR Vγ1-8, DNA segment NO: 22), an aDδitional human sequences with human JP1, JP, JP2, and J2 (15465 bp, DNA segment NO: 23), antibiotic selection cassette (DNA segment NO: 24, which is identical or substantially identical to DNA segment NO: 20), and 3′ mouse homology arm (8625 bp, DNA segment NO: 25). The human sequence ended with 5′ up-stream of the mouse endogenous Cγ 1 region, so human Vγs and Jγs was genetically linked to mouse Cγ1 region. The humanization strategy was summarized in FIG. 6. Junctional nucleic acid sequences inside the targeting vector among mouse, human, and prokaryotic/eukaryotic selection cassette are summarized in Table 24 and also provided in the Sequence Listing (Seq ID NO: 56-61).

TABLE 24
Junctional Nucleic acid sequences for Ygamma
Targeting Vector 2 (vector ID NO: 8)

| SEQ ID NO | Description |
|---|---|
| 56 | S' starting nucleic acid sequence of DNA segment |
| | NO: 21 of the 5'mouse homology arm inside |
| | mouse TCRgamma locus near C1 region |
| 57 | Junctional nucleic acid sequence between 3' end of |
| | mouse S'arm (DNA segment NO: 21) and the starting |
| | of the 5' end of human sequence (DNA |
| | segment NO: 22) |
| 58 | Junctional nucleic acid sequence between 3' end of |
| | human sequence (DNA segment NO: 22) and S' end |
| | of the human J region(DNA segment NO: 23) |
| 59 | Junctional nucleic acid sequence between 3' end of |
| | the human Jregion and 5' end of selection cassette |
| | (DNA segment NO: 24) |
| 60 | 3' ending nucleic acid sequence of DNA segment |
| | NO: 24 ofthe 5'mouse homology arm inside mouse |
| | TCRgamma C1 locus |
| 61 | 3' ending nucleic acid sequence of DNA segment |
| | NO: 25 |

TABLE 25
Primers for detecting RNA Expression of human Gamma V2-8 in humanized
mouse
(PCR round 1)

| Human Vgamma | Primer sequence (5′-3′) | Seq ID NO | New SEQ ID No. |
|---|---|---|---|
| hGV2-8 | CCCTTTCAGGCACTGTAAGCCAGC | 44 | 51020 |
| | (common anti-sense) | | |
| hGV2 | CTTGCTGAAGGAAGTAACGGCTAC | 45 | 51021 |
| hGV3 | CTTACTGTAACAAATACCTTCTAC | 46 | 51022 |
| hGV4 | TCTTGCTGAAGGAAGTACCGGCTAC | 47 | 51023 |
| hGV5 | TGTGACCTTACTGTAATAAATGCC | 48 | 51024 |
| hGV8 | TGATCTTCCTGTAGAAAATGCCGTC | 49 | 51025 |

TABLE 26
Primers for detecting RNA Expression of human Gamma V2-8 in humanized
mouse in C1 region
(PCR round 2)

| Human Vgamma | Primer sequence (5′-3′) | Seq ID NO | New SEQ ID No. |
|---|---|---|---|
| hGV2-8 | CCCCTTCCTGGGAGTCCAGGATAG | 50 | 51026 |
| | (comman anti-sense) | | |
| hGV2 | ACTATGACTCCTACAACTCCAAGG | 51 | 51027 |
| hGV3 | TACTATGACGTCTCCACCGCAAGGG | 52 | 51028 |
| hGV4 | ACTATGACTCCTACACCTCCAGCG | 53 | 51029 |
| hGV5 | ACTATGACGTCTCCAACTCAAAGG | 54 | 51030 |
| hGV8 | AAGTATCATACTTATGCAAGCACAGG | 55 | 51031 |

The bacteria homology recombination was helped by using bacterial Spec and Kan selection cassettes to finish the vector (Vector ID NO: 8). The linearized targeting vector (Vector ID NO: 8) was electroporated into ES cell line which contained humanized Vγ9-11 as described in Example 1. The targeted clones were selected as described in Example 1 and micro-injected into mouse blastocysts to generate chimera mice. The event of human Vγ1-8 and human Vγ9-11 on the same allele was confirmed by linkage analysis, in which all the progenies were positive for both human Vγ1-8 and human V. 9-11. There was no event that Human Vγ1-8 and human Vγ9-11 were in separate progenies.

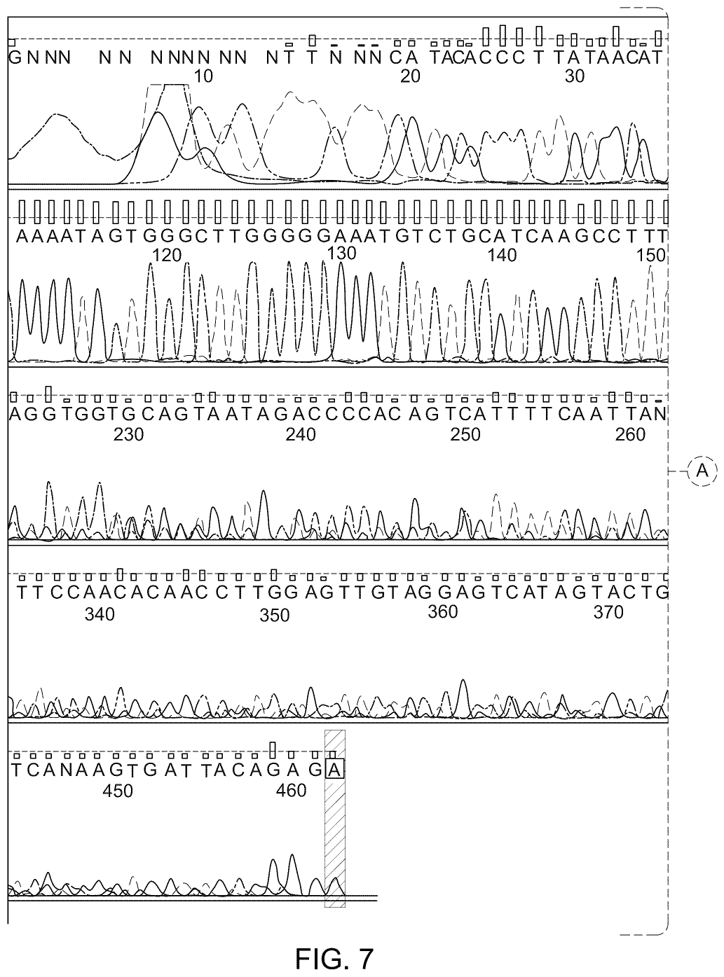

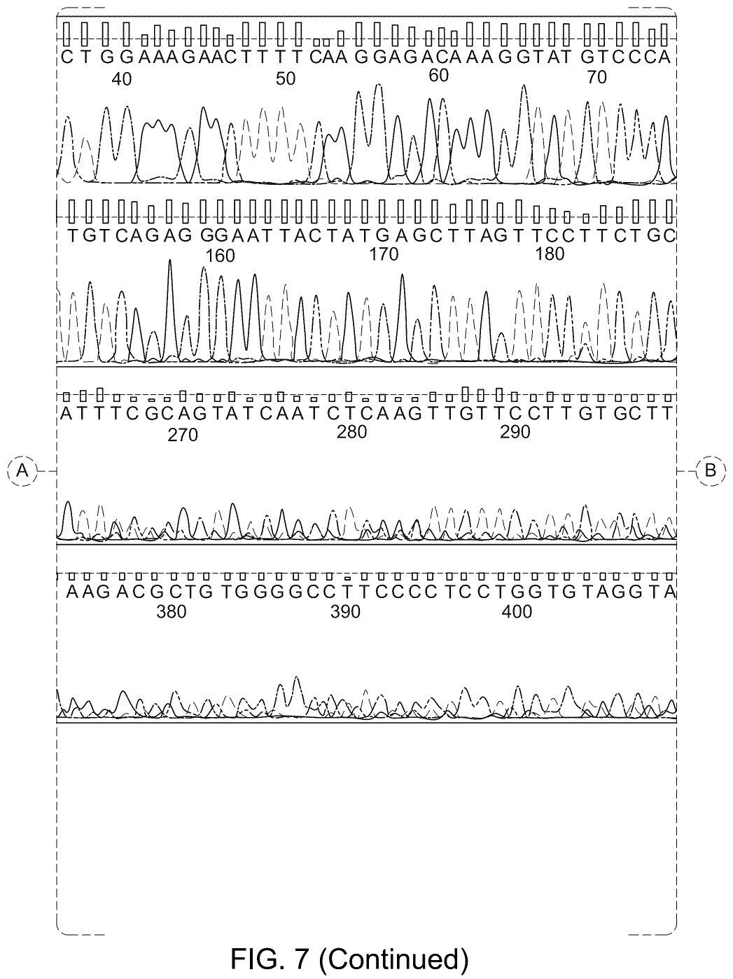

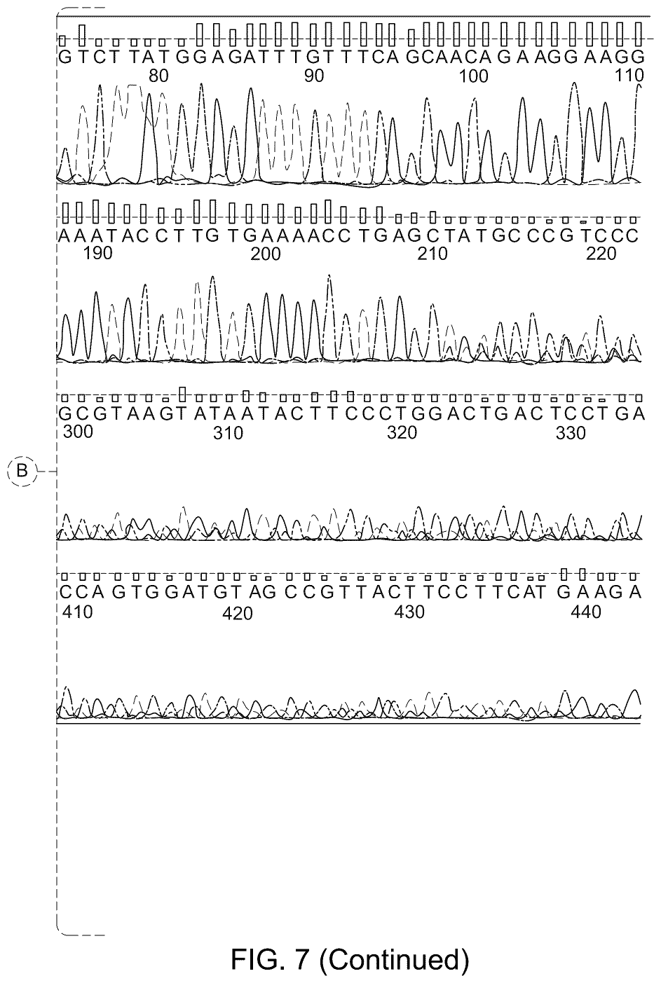

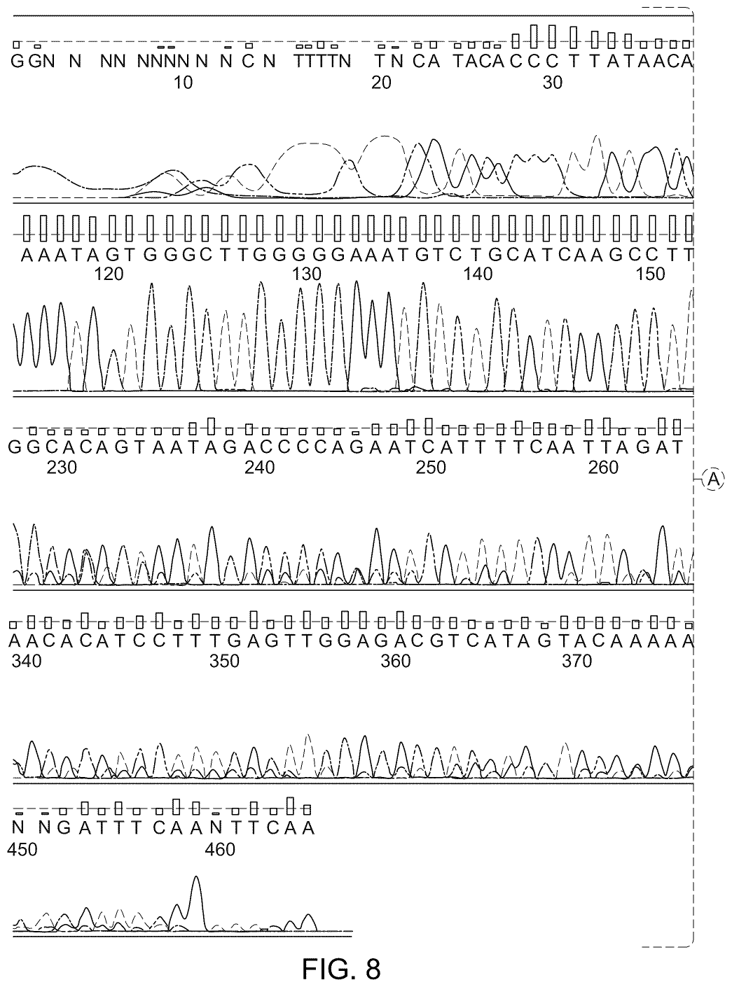

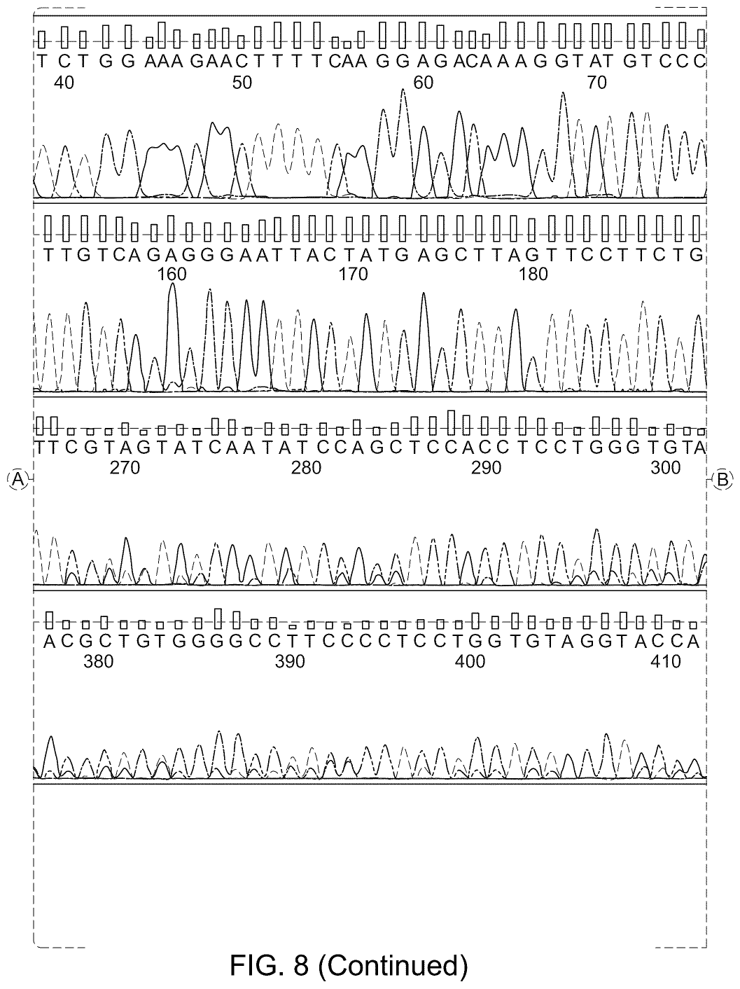

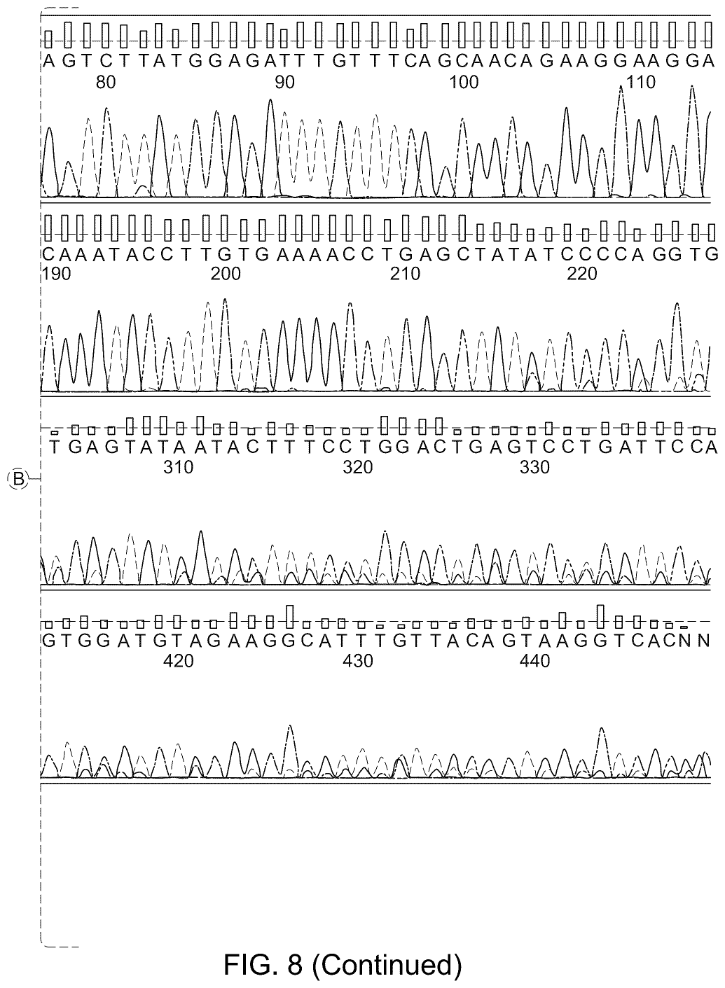

The germline transmission of human Vγ1-11 mice was achieved and mated with humanized Vδ mice to generate humanized γδ TCR mice. The tissues from these humanized mice were analyzed only for their Vγ2-10 TCR expression since human Vγ1, 6, 7, and 11 are pseudogenes. The PCR primers were listed in Tables 25 and 26. Two round of PCR was performed, and the expression patterns were listed in Table 27. Table 27 showed that human Vγ 2, 3, 4, 5, 8, 9, and 10 were expressed in various mouse tissues. Some of the PCR products were Sanger sequenced. FIGS. 7 and 8 also showed that the human gamma TCRs were highly diversified as the DNA sequences passed 230 nt became highly overlapped, indicating multiple TCRs were amplified using the same pair of PCR primers.

TABLE 27
Human Gamma Expression in Different Mouse Tissues

| Human | Tissues of Human TCRgamma Mice | | | | | | | | | | | | | | | | | | | | | |
|---|---|---|---|---|---|---|---|---|---|---|---|---|---|---|---|---|---|---|---|---|---|---|
| Vgamma | 1 | 2 | 3 | 4 | 5 | 6 | 7 | 8 | 9 | 10 | 11 | 12 | 13 | 14 | 15 | 16 | 17 | 18 | 19 | 20 | 21 | 22 |
| AGV2 | | | | | | + | | + | + | | | | | + | | | | | | | + | + |
| hGV3 | | | | | | | | | + | | | | | | | | + | | | | | + |
| hGV4 | | | + | | | + | | + | | + | + | | | | | | | | | | | + |
| hGV5 | + | | | | | | | | | | | | | | | | | | | | | + |
| hGV8 | | | | | | | | | + | | | | | | | | | | | | | |
| hGV9 | | | | | | | | | + | + | | | | | | | + | | | | | + |
| hGV10 | | | + | + | + | + | | + | | + | + | | | | + | + | + | | | + | + | + |

1-Peritoneal fluid, 2-brain (olfactory), 3-ear (middle part), 4-nose, S-tough, 6-lymph
nodes, 7-throat, 8-lung, 9-spleen, 10-bone marrow, 11-Eye (eve ball), 12, 13, 14
(three beginning, middle, end) parts of small intestine, 15-uterus track, 16-kidney,
17-thymus, 18-muscle, 19-heart, 20-liver, 21-skin, 22-blood. Positive sign
represents expression of human gamma CDR3.

SEQUENCES 1-74
SEQ ID NO: 1 (SEQ ID No. 50967)
TAGGCTATCACAGACAGGGAGAATTTACATCCACCCCAACTTTATACTTCTAATTAT
CC
SEQ ID NO: 2 (SEQ ID No. 50968)
CCTGAAATTCCAGCTTGCAGAGCACTTCCTGCCTCCCTGTGAAGCAGCCTGACCTTG
GGATGCTGTCACTGCTCCACACATCAACGCTGGCAGTCCTTGGGGCTCGTAAGTAGT
TTT
SEQ ID NO: 3 (SEQ ID No. 50969)
TCTCCAGAGGATTCAGCTGTACTTCTAACTGCTCAAATGGAAGGTTATCAACATCAG
CTCGTACGTTCGTGGGATTGTGTCCGTGTCGCGAAGTTCCTATACTTTCTAGAGAATA
GGAACTTC
SEQ ID NO: 4 (SEQ ID No. 50970)
GAAGTTCCTATACTTTCTAGAGAATAGGAACTTCGTTGGTACGAACCCGGGTTTGGG
CCCCTTCTCAGTGCCAGTGTCCCCTCAGTGAAAAGAGACCAGCTTGTGTACTGTCAT
GTAGA
SEQ ID NO: 5 (SEQ ID No. 50971)
TGCTTCTACAGTGTCCTATTAATCCCACTGTGCTAGCACTGGGTTTCCATAGTCCATG
T
SEQ ID NO: 6 (SEQ ID No. 50972)
CTGCTTGCCATGCTCAGGAGCTGCAATAAGTTCAGCTCTTCGTGTCGCTTTATGCCTT
C
SEQ ID NO: 7 (SEQ ID No. 50973)
GAGTACCTGTCAAATATAAACTATGCTCAGCAGGTACCAGCGCCTCACTTTTATCCT
TTACGCGCCATTTATCAACTCACACCTATGGGGACAGCTATTATCAGAAAAACAAAA
GGCAACAAGT ATTAGTAGGGATGTG
SEQ ID NO: 8 (SEQ ID No. 50974)
TCAGATATTCTAAGGATCACTGCTATCCAGAGAATCTTAGAGCTTGACCTAAAGCTG
GTACCGTACTCATGGCAACAGCATTACTGACCATTTGAATGTAGAAGACTCATGAAT
TCTCCCTTCCT AGAGAC
SEQ ID NO: 9 (SEQ ID No. 50975)
CTCAGAGTCATATCGCTGCATTCACTACTTTCTCTGTGAGTTTTCTATGGGATTCAGC
ATAGCTAATATATCTTCCAACCTGAGTGTCAGCACATTCAGACTTGATCCAGCCAGA
AGCTACATTTTTC AGAGTCAGAG
SEQ ID NO: 10 (SEQ ID No. 50976)
AGAGAGATGGTAGTCCTGTGGTTGCTTCATCTGTGGTCTGGATCAGAAGATTTAATT
CTAAATAAAAGCTCATATGTCAATCTAATTCTGTGTTGTTAAAGTAAAGATATCAAA
GGAATAGCTCTAC TCCCATGTTTCAG
SEQ ID NO: 11 (SEQ ID No. 50977)
CTTCTCATTGAGGTTTATTCTGATTGATTTCCAGCTTTGCCCAAGGGACCCTGAGTTA
TCTTACCAGTTGGCAACCCAAAAACATCCTATAAAATCTCCAGCAGGCCTTTGTTTC
CTTGCCATCAGC TCCTCTTCGGC
SEQ ID NO: 12 (SEQ ID No. 50978)
CAATTTTGTAAGAGTATTGATGGAAAATCGTTAGGCATCTACATAGCCTTGATTTGG
CTCCTTGGACCTCTGACCAATAAACACAGGAAAAGATGCTCTACATCAGGAGTCAA
CAAAATATGGCC CATGAGCCAAATTC
SEQ ID NO: 13 (SEQ ID No. 50979)
TCTGAACCAGTCAGTAACCTTCCCATGGTAGTAATCATGTGAGACAGGATTCAGCAG
GACTGGCTGAGATTATATCAAAGCTTTCATAGGGGTTCTATAACCACAGAATGTAGT
TAGTTCCCTGC CAAGTAAAACTTA
SEQ ID NO: 14 (SEQ ID No. 50980)
ATCCCTTTGGCTAAGCTAACTAGTATGCGTGCCTCCTTGCAAACTGAAAACAAGTGT
CCTACAGCTTGGACGTACGGTACCAACGAAGTTCCTATTCTCTAGAAAGTATAGGA
ACTTCATTCTACC GGGTAGGGGAGG
SEQ ID NO: 15 (SEQ ID No. 50981)
CGCGGGAAGTTCCTATTCTCTAGAAAGTATAGGAACTTCGCGACACGGACACAATCC
CACGAACGTACGCCTGCACATACCTTCGGCTGAGCTAACGTGTCTGCATGCCTCCTT
ACAGAGCACAA ATCTCCTACAGCT
SEQ ID NO: 16 (SEQ ID No. 50982)
AAACGAAACCAAGGTCTCAAGACTGACAGCCCAAAGTTACTGTCCCAGGGGCTCCC
T
SEQ ID NO: 56 (SEQ ID No. 50983)
CTGCTATTAACTCCTGATTCAGCCTTCTAG
SEQ ID NO: 57 (SEQ ID No. 50984)
AGGCTGGCACACTCTCACTGATAGATGGCTAAGCGATACAGAGAGCCCAGACAACC
ATTTA GAGGTGAGGA ATTAGTAGGGATGTG
SEQ ID NO: 58 (SEQ ID No. 50985)
TAGCCCCAGAATATCCCTCTGTCAAAACGTTCTGCTAGCTTTGTAT
TAACTTTAAATAATTGGCATTATTTAAAGTTAGCAGAAAGAGATAAATGCATAGTTA
GTGCCTGACTCA
SEQ ID NO: 59 (SEQ ID No. 50986)
TCTAGCTAAAGAAAGGGGCAAGAGACAATGAATGCTGTGGTACCGTACAGAAAATG
TCGTA CGGAAGTTCCTATACTATTTGAAGAATAGGAACTTCC AGAGTCAGAG
SEQ ID NO: 60 (SEQ ID No. 50987)
ACGCTGTGATGCGTCTTATGCTGTGTATCGTGTCATGGTACCCAAGGACATCTTGTCT
GAAAC AAACTGTAATCGAGCACTCGACCACGGCTGTCCTCTG
SEQ ID NO: 61 (SEQ ID No. 50988)
ACTGGGTTGCATGCTTATTCAGAGAACTGGCTT
(Vγ9)
SEQ ID NO: 62 (SEQ ID No. 50989)
CVYGAGHLEQPQISSTKTLSKTARLECVVSGITISATSVYWYRERPGEVIQFLVSI
SYDGTVRKESGIPSGKFEVDRIPETSTSTLTIHNVEKQDIATYYCALWEV
(Vγ10)
SEQ ID NO: 63 (SEQ ID No. 50990)
MFIGNSPLLLTVILGLSKVEQFQLSISTEVKKSIDIPCKISSTRFETDVIHWYRQKPNQ A
LEHLIYIVSTKSAARRSMGKTSNKVEARKNSQTLTSILTIKSVEKEDMAVYYCAAWD
(Vγ11)
SEQ ID NO: 64 (SEQ ID No. 50991)
LGQLEQPEISISRPANKSAHISWKASIQGFSSKIIHWYWQKPNKGLEYLLHVFLTISAQ D
CSGGKTKKLEVSKNAHTSTSTLKIKFLEKEDEVVYHCACWIRH
(Vδ1)
SEQ ID NO: 65 (SEQ ID No. 50992)
SSVAQKVTQAQSSVSMPVRKAVTLNCLYETSWWSYYIFWYKQLPSKEMIFLIR
QGSDEQNAKSGRYSVNFKKAAKSVALTISALQLEDSAKYFCALGE
(Vδ2)
SEQ ID NO: 66 (SEQ ID No. 50993)
VMSAIELVPEHQTVPVSIGVPATLRCSMKGEAIGNYYINWYRKTQGNTMTFIY
REKDIYGPGFKDNFQGDIDIAKNLAVLKILAPSERDEGSYYCACDT
(Vδ3)
SEQ ID NO: 67 (SEQ ID No. 50994)
RGTLCDKVTQSSPDQTVASGSEVVLLCTYDTVYSNPDLFWYRIRPDYSFQFVF
YGDNSRSEGADFTQGRFSVKHILTQKAFHLVISPVRTEDSATYYCAF
(Vδ4)
SEQ ID NO: 68 (SEQ ID No. 50995)
PGIAQKITQTQPGMFVQEKEAVTLDCTYDTSDPSYGLFWYKQPSSGEMIFLIY
QGSYDQQNATEGRYSLNFQKARKSANLVISASQLGDSAMYFCAMRE
(Vδ5)
SEQ ID NO: 69 (SEQ ID No. 50996)
VNSQQKNDAQQVKQNSPSLSVQEGRISILNCDYTNSMFDYFLWYKKYPAEGP
TFLISISSIKDKNEDGRFTVFLNKSAKHLSLHIVPSQPGDSAVYFCAAS
(Vδ6)
SEQ ID NO: 70 (SEQ ID No. 50997)
VSGQQKEKSDQQQVKQSPQSLIVQKGGISIINCAYENTAFDYFPWYQQFPGKG
PALLIAIRPDVSEKKEGRFTISFNKSAKQFSSHIMDSQPGDSATYFCAA
(Vδ7)
SEQ ID NO: 71 (SEQ ID No. 50998)
VSSEDKVVQSPLSLVVHEGDTVTLNCSYEVTNFRSLLWYKQEKKAPTFLFMLTSS
GIEKKSGRLSSILDKKELFSILNITATQTGDSAIYLCAVE
(Vδ8)
SEQ ID NO: 72 (SEQ ID No. 50999)
FSMAQTVTQSQPEMSVQEAETVTLSCTYDTSESDYYLFWYKQPPSRQMIL VIR
QEAYKQQNATENRFSVNFQKAAKSFSLKISDSQLGDAAMYFCAYRS
(constant region of a murine 8 TCR chain)
SEQ ID NO: 73 (SEQ ID No. 51000)
XSQPPAKPSVFIMKNGTNVACLVKDFYPKEVTISLRSSKKIVEFDPAIVISPSGKY
SAVKLGQYGDSNSVTCSVQHNSETVHSTDFEPYANSFNNEKLPEPENDTQISE
PCYGPRVTVHT EKVNMMSLTVLGLRLLFAKTIAINFLLTVKLFF
(constant region of a murine YTCR chain)
SEQ ID NO: 74 (SEQ ID No. 51001)
XKRTDSDFSPKPTIFLPSAAETNLHKAGTYLCLLEKFFPEVIRVYWKEKDGEKIL
ESQEGNTIKTNDRYMKFSWLTVTEDSMAKEHSCIVKHENNKRGVDQEILFPP
IGKAFTTINVNPRDSVLRHENVNNATDLEDCMKGRKDMLQLQVTTTYAFYT
YLILFFKSMVHLAFVVFCLFRRAAMSCDDQRS

What is claimed:

1. A genetically modified non-human animal, whose genome comprises: at least one unarranged human T cell receptor (TCR) V gene segment, at least one unarranged human TCR Dδ gene segment, and at least one unarranged human TCR Jδ gene segment, wherein the at least one unarranged human TCR Vδ gene segment, the at least one unarranged human TCR Dδ gene segment, and the at least one unarranged human TCR Jδ gene segment are operably linked to a functional non-human TCRδ constant gene sequence.

2. The animal of claim 1, wherein the at least one unarranged human TCR Vδ gene segment, the at least one unarranged human TCR Dδ gene segment, and the at least one unarranged human TCR Jδ gene segment are inserted into the endogenous TCRδ variable gene locus.

3. The animal of claim 1, wherein the at least one unarranged human TCR Vδ gene segment, the at least one unarranged human TCR Dδ gene segment, and the at least one unarranged human TCR Jδ gene segment replace the complete repertoire of unarranged endogenous TCR Vδ, Dδ and Jδ gene segments.

4. The animal of claim 1, wherein the at least one unarranged human TCR Vδ gene segment is selected from the unarranged repertoire of the human TCR Vδ gene segments VOL Vδ2, Vδ3, Vδ4, Vδ5, Vδ6, Vδ7, and Vδ8.

5. The animal of claim 1, wherein the amino acid sequence of the at least one unarranged human TCR Vδ segment has at least 90% sequence identity with a sequence selected from the group consisting of SEQ ID Nos: 65, 66, 67, 68, 69, 70, 71, 72.

6. The animal of claim 1, wherein the at least one unarranged human TCR Vδ gene segment, the at least one unarranged human TCR Dδ gene segment, and the at least one unarranged human TCR Jδ gene segment are capable of rearranging to form a rearranged human VDJ δ sequence.

7. The animal of claim 6, wherein the animal expresses a humanized TCRδ variable region comprising the rearranged human VDJ δ sequence on the surface of a γδ T cell population.

8. The animal of claim 7, wherein the sequence of the complementary determining region 3 (CDR3) of Vδ1 starts with amino acids ALGE, where E can be replaced by any of G, A, D, V or R; and/or ends with KLI or TRQM.

9. The animal of claim 7, wherein the CDR3 of Vδ2 starts with amino acids ACD, ACG, ACE, ARD, or ASD; and/or ends with KU or TRQM.

10. The animal of claim 7, wherein the CDR3 of Vδ3 starts with amino acids AL, AS, AC, AY, AT, or AV.

11. The animal of claim 7, wherein the first three amino acids of the CDR3 of Vδ4 are AMR, ASP, AMS, AMT, AMG, AMI, AIR, AKR, ATR, EMR, VMR, PMR, or ALP; and/or the last amino acids are KLI or TRQM.

12. The animal of claim 7, wherein the first three amino acids of the CDR3 of the Vδ6 are AAS or AAR; or the first four amino acids are AASP, where P can be replaced by T, G, M, V, L, or R.

13. The animal of claim 12, wherein the last three amino acids of the CDR3 of the Vδ6 are TRQ, KU, or KLN.

14. A genetically modified non-human animal, whose genome the genome comprises: at least one unarranged human TCR Vγ gene segment and at least one human TCR Jγ gene segment, wherein the at least one unarranged human TCR Vγ gene segment and the at least one human TCR Jγ gene segment are operably linked to a functional non-human TCRγ, constant gene.

15. The animal of claim 14, wherein the at least one unarranged human TCR Vγ gene segment, and the at least one unarranged human TCR Jγ gene segment are inserted into the endogenous TCRγ variable gene locus.

16. The animal of claim 14, wherein the at least one unarranged human TCR Vγ gene segment, and the at least one unarranged human TCR Jγ gene segment replace the complete repertoire of the unarranged endogenous TCR Vγ and Jγ gene segments.

17. The animal of claim 14, wherein the at least one unarranged human TCR Vγ gene segment is selected from the unarranged repertoire of the human TCR Vγ gene segments Vγ2, Vγ3, Vγ4, Vγ5, Vγ8, Vγ9, Vγ10, and Vγ11, preferably Vγ9, Vγ10, and Vγ11.

18. The animal of claim 17, wherein the amino acid sequence of the at least one unarranged human TCR Vγ segment has at least 90% sequence identity with SEQ ID NO: 62, SEQ ID NO: 63 or SEQ ID NO: 64.

19. The animal of claim 14, wherein the at least one unarranged human TCR Vγ gene segment, and the at least one unarranged human TCR Jγ gene segment are capable of rearranging to form a rearranged human VJ γ sequence.

20. The animal of claim 19, wherein the animal expresses a humanized TCRγ variable region comprising the rearranged human VJ γ sequence on the surface of a γδ T cell population.

21. The animal of claim 20, wherein:
the first four amino acids of the CDR3 of Vγ9 are selected from the group consisting of ALWE, ALWG, ASWE, AT CE, ALLE, ALRE, PCGR, AWWE, DLWE, ASWE, and AMWE; or
the last amino acids of the CDR3 of Vγ9 are selected from the group consisting of KTL, KEL, EKL, KNL, IKV, KSR, RNS, KNS, MKL, KRL, RKL, FKI, and KNQG; or
the first three amino acids of the CDR3 of Vγ10 are AAW; or
the first four amino acids of the CDR3 of the Vγ10 are AAWF, where the fourth position can be any of F, L, A, R, G, C, or V; or
the last three amino acids of Vγ10 are FKI.

22. A genetically modified non-human animal, whose genome comprises:
at least one unarranged human T cell receptor (TCR) Vδ gene segment, at least one unarranged human TCR Dδ gene segment, and at least one unarranged human TCR Jδ gene segment; and
at least one unarranged human TCR Vγ gene segment and at least one human TCR Jγ gene segment,
wherein the at least one unarranged human TCR Vδ gene segment, the at least one unarranged human TCR Dδ gene segment, and the at least one unarranged human TCR JE, gene segment are operably linked to a functional non-human TCRδ constant gene, and
wherein the at least one unarranged human TCR Vγ gene segment and the at least one human TCR Jγ gene segment are operably linked to a functional non-human TCRγ constant gene.

23. The animal of claim 22, wherein the at least one unarranged human TCR Vδ gene segment, the at least one unarranged human TCR DL gene segment, and the at least one unarranged human TCR Jδ gene segment are inserted into the endogenous TCR variable gene locus, and
wherein the at least one unarranged human TCR Vγ gene segment, and the at least one unarranged human TCR Jγ gene segment are inserted into the endogenous TCRγ variable gene locus.

24. The animal of claim 22, wherein the at least one unarranged human TCR Vδ gene segment, the at least one unarranged human TCR Dδ gene segment, and the at least one unarranged human TCR Jδ gene segment replace the complete repertoire of the unarranged endogenous TCR Vδ, Dδ and Jδ gene segments, and
wherein the at least one unarranged human TCR Vγ gene segment, and the at least one unarranged human TCR Jγ gene segment replace the complete repertoire of the unarranged endogenous TCR Vγ and Jγ gene segments.

25. The animal of claim 22, wherein the at least one unarranged human TCR Vδ gene segment is selected from the unarranged repertoire of the human TCR Vδ gene segments Vδ1, Vδ2, Vδ3, Vδ4, Vδ5, Vδ6, Vδ7, and Vδ8, and
wherein the at least one unarranged human TCR Vγ gene segment is selected from the unarranged repertoire of the human TCR Vγ gene segments Vγ2, Vγ3, Vγ4 Vγ5, Vγ8 Vγ9, Vγ10, and Vγ11, preferably Vγ9, Vγ10, and Vγ11.

26. The animal of claim 25, wherein the at least one unarranged human TCR Vδ gene segment, the at least one unarranged human TCR Dδ gene segment, and the at least one unarranged human TCR Jδ gene segment are capable of rearranging to form a rearranged human VDJ δ sequence, and
wherein the at least one unarranged human TCR Vγ gene segment, and the at least one unarranged human TCR Jγ gene segment are capable of rearranging to form a rearranged human VJγ sequence.

27. The animal of claim 26, wherein the animal expresses a humanized TCRγδ comprising the rearranged human VDJ δ sequence and the rearranged human VJ γ sequence on the surface of a γδ T cell population.

28. The animal of claim 22, wherein the amino acid sequence of the at least one Vγ segment has at least 90% sequence identity with a human Vγ sequence selected from the group consisting of SEQ ID Nos 62, 63 and 64 and wherein the amino acid sequence of the at least one Vδ segment has at least 90% sequence identity with a human Vδ sequence selected from the group consisting of SEQ ID Nos 64, 66, 67, 68, 69, 70, 71 and 72.

29. The animal of claim 22, wherein Dδ has at least 90% sequence identity with human D.

30. The animal of claim 22, wherein the Jγ and Jδ have at least 90% sequence identity with human Jγ and Jδ.

31. The animal of any one of claims 7, 20 and 27, wherein the humanized TCR is expressed with at least one mouse CD3 on the surface of a γδ T cell population.

32. The genetically modified animal of any of claims 1, 14 and 22, wherein the animal is a rodent.

33. The genetically modified animal of any of claims 1, 14 and 22, wherein the animal is a mouse.

34. The genetically modified animal of any of claims 1, 14 and 22, wherein the animal generates a population of central and effector memory γ/δ T cells to an internal or external antigen.

35. A method of producing a humanized TCR comprising: administering an antigen of interest to the genetically modified animal of any of claims 1, 14 and 22; and obtaining a humanized TCR that recognizes said antigen of interest.

36. A method of determining and/or analyzing the TCR repertoire of the humanized TCR produced in claim 35 by next-generation sequencing.

37. A method of establishing cancer comprising: administering a cancer cell to the genetically modified animal of any of claims 1, 14 and 22, and determining cancer growth.

38. A method of establishing an infection comprising: administering an antigen-derived pathogen to the genetically modified animal of any of claims 1, 14 and 22; and determining the repertoire of TCR expansion by sequencing.
